(19) United States
(12) Patent Application Publication
(10) Pub. No.: US 2003/0235882 A1
(43) Pub. Date: Dec. 25, 2003

(54) Novel nucleic acids and polypeptides and methods of use thereof

(76) Inventors: Richard A. Shimkets, Guilford, CT (US); Meera Patturajan, Branford, CT (US); Corine A.M. Vernet, Branford, CT (US); Stacie J. Casman, North Haven, CT (US); Uriel M. Malyankar, Branford, CT (US); Suresh G. Shenoy, Branford, CT (US); Kimberly A. Spytek, New Haven, CT (US); Esha A. Gangolli, Madison, CT (US); Charles E. Miller, Guilford, CT (US); Ferenc L. Boldog, North Haven, CT (US); Li Li, Branford, CT (US); Raymond J. Taupier JR., East Haven, CT (US); Ramesh Kekuda, Norwalk, CT (US); Glennda Smithson, Guilford, CT (US); Bryan D. Zerhusen, Branford, CT (US); Xiaohong Liu, Lexington, MA (US); Steven D. Colman, Guilford, CT (US); Velizar T. Tchernev, Branford, CT (US); Jingsheng Si, Cheshire, CT (US); Shlomit R. Edinger, New Haven, CT (US); David J. Stone, Guilford, CT (US); Paul Sciore, North Haven, CT (US); Isabelle Millet, Milford, CT (US); Mark E. Rothenberg, Clinton, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 10/028,248

(22) Filed: Dec. 19, 2001

Related U.S. Application Data
(60) Provisional application No. 60/256,619, filed on Dec. 19, 2000.
(60) Provisional application No. 60/262,959, filed on Jan. 19, 2001.
(60) Provisional application No. 60/272,408, filed on Feb. 28, 2001.
(60) Provisional application No. 60/285,189, filed on Apr. 20, 2001.
(60) Provisional application No. 60/308,039, filed on Jul. 26, 2001.
(60) Provisional application No. 60/311,266, filed on Aug. 9, 2001.

(51) Int. Cl. C12Q 1/68; G01N 33/53; G01N 33/567; C07H 21/04; C12N 9/00; C12P 21/02; C12N 5/06; C07K 14/47

(52) U.S. Cl. 435/69.1; 435/183; 435/320.1; 435/325; 530/350; 536/23.2; 435/6; 435/7.2; 530/388.1

(57) ABSTRACT

Disclosed are novel polypeptides and nucleic acids encoding same. Also disclosed are vectors, host cells, antibodies and recombinant methods for producing the polypeptides and polynucleotides, as well as methods for using same.

### RELATED APPLICATIONS

This application claims priority to U.S. Ser. No. 60/256,619 (Attorney Ref.: 21402-223), filed Dec. 19, 2000; U.S. Ser. No. 60/262,959 (Attorney Ref.: 21402-223A), filed Jan. 19, 2001; U.S. Ser. No. 60/272,408 (Attorney Ref.: 21402-223C1), filed Feb. 28, 2001; U.S. Ser. No. 60/285,189 (Attorney Ref.: 21402-222A), filed Apr. 20, 2001; U.S. Ser. No. 60/308,039 (Attorney Ref.: 21402-223D1), filed Jul. 26, 2001; and U.S. Ser. No. 60/311,266 (Attorney Ref.: 21402-223IFC-01), filed Aug. 9, 2001, each of which is incorporated by reference in its entirety.

### FIELD OF THE INVENTION

The present invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10, NOV11 and NOV 12 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, 23, 25, 27, 29, 31, 33, 35, and 37. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, 26, 28, 30, 32, 34, 36, and 38. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27, 29, 31, 33, 35, and 37.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, 27, 29, 31, 33, 35, and 37)or a complement of said oligonucleotide. Also included in the invention are substantially purified NOVX polypeptides (SEQ ID NOS: 2, 4, 6, 8, 10, 12, 14, 16, 18, 20, 22, 24, 26, 28, 30, 32, 34, 36, and 38). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., Cancer, Hodgkin disease, Von Hippel-Lindau (VHL) syndrome, hypercalceimia, Endometriosis, Crohn's Disease, Xerostomia, Inflammatory bowel disease, Diverticular disease, fertility, Infertility, CNS disorders, osteoporosis, atherosclerosis, hypertension, congenital heart defects, aortic stenosis, valve diseases, tuberous sclerosis, scleroderma, Hemophilia, obesity, Diabetes, Pancreatitis, transplantation recovery, Autoimmune disease, asthma, arthritis, Immunodeficiencies, Graft vesus host, Alzheimer's disease, Stroke, Parkinson's disease, Huntington's disease, Cerebral palsy, Epilepsy, Multiple sclerosis, Ataxia-telangiectasia, Behavioral disorders, Addiction, Anxiety, Pain, Muscular dystrophy, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | | Nucleic | |
|---|---|---|---|---|
| | | | Acid | Polypeptide |
| NOVX | | | SEQ ID | SEQ ID |
| No. | Internal Acc. No. | Homology | NO. | NO. |
| 1a | CG-AC084364.5/ | Stabilin | 1 | 2 |
| | AC084364.5 | | | |
| 1b | CG50736-10/11400078 | Stabilin | 3 | 4 |
| 1c | CG50736-09 | CD44-like Precursor/ | 210 | 211 |
| | | Fascilin domain | | |
| 2a | CG142106342/CG50646- | Polydom | 5 | 6 |
| | 04 | | | |
| 2b | CG50646-05 | Polydom | 7 | 8 |
| 3a | CG50273-01 | Transmembrane Protein | 9 | 10 |
| 3b | CG50273-02 | Transmembrane IIIb | 11 | 12 |
| | | Protein | | |
| 4 | CG50289-01 | Serine Protease | 13 | 14 |
| 5a | CG50353-01 | Wnt 7a Protein | 15 | 16 |
| 5b | 169475673 | Wnt 7a protein | 17 | 18 |
| | (insert assembly of | | | |
| | NOV5a) | | | |
| 6a | CG50221-01 | Apical Endosomal | 19 | 20 |
| | | Glycoprotein | | |
| 6b | 174308633 | Apical Endosomal | N/A | N/A |
| | (insert assembly of | Glycoprotein | | |
| | NOV6a) | | | |
| 7a | CG50367-01 | ADAM13 | 21 | 22 |
| 7b | CG50367-02 | ADAM13 | 23 | 24 |
| 7c | CG50367-03 | ADAM13 | 25 | 26 |
| 8 | CG50321-01 | Leucine Rich Containing F | 27 | 28 |
| | | Box | | |
| 9 | CG55902-01 | Steroid Binding | 29 | 30 |
| 10a | CG50307-01 | Steroid Dehydrogenase | 31 | 32 |
| 10b | CG50307-02 | Steroid Dehydrogenase | 33 | 34 |
| 11 | CG50311-01 | Myosin Heavy Chain | 35 | 36 |
| 12a | CG50323-01 | Pancreatitis-Associated | 37 | 38 |
| | | Protein | | |
| | | (PAP) | | |
| 12b | 169475472 | PAP | N/A | N/A |
| | (insert assembly of | | | |
| | NOV12a) | | | |
| 12c | 169475476 | PAP | N/A | N/A |
| | (Insert assembly of | | | |
| | NOV12a) | | | |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The present invention is based in part on nucleic acids encoding proteins that are novel members of the following protein families: Stabilin/Fascilin/CD-44 precursor FELL-like, Polydom, Transmembrane/IIIb, Serine Protease, Wnt-7a, Apical endosomal glycoprotein, ADAM13, Leucine-rich containing F-Box, Pancreatitis-Associated, Steroid Binding, Steroid dehydrogenase, and Myosin Heavy-chain-like proteins. More particularly, the invention relates to nucleic acids encoding novel polypeptides, as well as vectors, host cells, antibodies, and recombinant methods for producing these nucleic acids and polypeptides.

NOV1 is homologous to the Stabilin family of proteins. Thus, the NOV1 nucleic acids, polypeptides, antibodies and related compounds according to the invention will be useful in therapeutic and diagnostic applications implicated in, for example, cancer, particularly mechanisms of angiogenesis, inflammation, CNS disorders, metabolic disorders including obesity and diabetes and/or other pathologies/disorders.

Fascilin domain-containing proteins have been shown to be important for cell adhesion, which impacts a variety of diseases including cancer, inflammation, obesity and CNS disorders. Stabilin-1 is an endothelial-macrophage member of the fascilin domain containing protein family associated with angiogenesis.

NOV2 is homologous to the Polydom family of proteins. Thus NOV2 nucleic acids, polypeptides, antibodies and related compounds according to the invention will be useful in therapeutic and diagnostic applications implicated in, for example, inflammatory diseases, disorders of coagulation, cancer, obesity, diabetes, asthma, arthritis, osteoporosis, cardiovascular disease and/or other pathologies/disorders.

The mouse polydom protein appears to be important for the regulation of hematopoiesis and may play a role in cell adhesion or in the immune system. Domains within this protein and the human ortholog have been shown to be important in coagulation, growth, cell division, and other important cellular processes.

NOV3 is homologous to a transmembrane/IIIb protein. Thus, the NOV3 nucleic acids and polypeptides, antibodies and related compounds according to the invention will be useful in therapeutic and diagnostic applications implicated in, for example, cancer, trauma, regeneration (in vitro and in vivo), viral/bacterial/parasitic infections, neuroprotection, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, and/or other pathologies/disorders.

The human transmembrane protein described herein has homology to a mouse protein that causes growth inhibition of E. coli when expressed exogenously. Therefore, the disclosed transmembrane/IIIb protein of this invention will fulfill a similar function in humans.

NOV4 is homologous to a Serine protease family of proteins. Thus, NOV4 nucleic acids, polypeptides, antibodies and related compounds according to the invention will be useful in therapeutic and diagnostic applications implicated in, for example, cancer, trauma, regeneration (in vitro and in vivo), viral/bacterial/parasitic infections, infertility, and/or other pathologies/disorders.

Proteolytic enzymes that exploit serine in their catalytic activity are ubiquitous, being found in viruses, bacteria and eukaryotes. They include a wide range of peptidase activity, including exopeptidase, endopeptidase, oligopeptidase and omega-peptidase activity. Over 20 families of serine protease have been identified and although they have different evolutionary origins, there are similarities in the reaction mechanisms of several peptidases. Chymotrypsin, subtilisin and carboxypeptidase C clans have a catalytic triad of serine, aspartate and histidine in common: serine acts as a nucleophile, aspartate as an electrophile, and histidine as a base. The geometric orientations of the catalytic residues are similar between families, despite different protein folds. The trypsin family is almost totally confined to animals, although trypsin-like enzymes are found in actinomycetes of the genera Streptomyces and Saccharopolyspora, and in the fungus Fusarium oxysporum. The enzymes are inherently secreted, being synthesised with a signal peptide that targets them to the secretory pathway. Animal enzymes are either secreted directly, packaged into vesicles for regulated secretion, or are retained in leukocyte granules.

The NOV4 nucleic acid and polypeptide described in this application has a structure similar to TESP-1 and TESP-2; serine proteases isolated from mouse sperm acrosome. These enzymes are secreted as zymogens and released by the acrosome reaction induced by the calcium ionophore; A23187. These may play a role in fertilization and/or processing of other proteins during fertilization.

NOV5 is homologous to the Wnt-7a protein family. Thus NOV5 nucleic acids, polypeptides, antibodies and related compounds according to the invention will be useful in therapeutic and diagnostic applications implicated in, for example, atherosclerosis, aneurysm, hypertension, fibromuscular dysplasia, stroke, scleroderma, obesity, transplantation disorders, myocardial infarction, embolism, cardiovascular disorders, bypass surgery, endometriosis, infertility, polycystic ovary syndrome, Von Hippel-Lindau (VHL) syndrome, Alzheimer's disease, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, psoriasis, actinic keratosis, acne, hair growth/loss, allopecia, pigmentation disorders, endocrine disorders, pancreatitis, diabetes, and/or other pathologies/disorders.

Wnt proteins constitute a large family of molecules involved in cell proliferation, cell differentiation and embryonic patterning. They are known to interact with the Frizzled family of receptors to activate two main intracellular signaling pathways regulating intracellular calcium levels and gene transcription. Early studies on Wnts implicated them in cell proliferation and tumorigenesis, which have been borne out by recent work using transgenic and null mutant mice. Wnts are involved in processes involved in mammary gland development and cancer. Recent studies have demonstrated that these molecules are critical to organogenesis of several systems, such as the kidney and brain. Wnts regulate the early development, i.e. neural induction, and their role persists in later stages of development as well as in the mature organ. An example of this is seen in the brain, where the loss of certain Wnts leads to the absence of critical regions of the brain, e.g. the hippocampus, involved in learning and memory, or the cerebellum, involved in motor function. Wnts have also been implicated in the genesis of degenerative diseases such as Alzheimer's disease.

The NOV5 nucleic acid and polypeptide of the invention has a high degree of similarity to Wnt-7a. Wnt-7a is known to be involved in the development of the limbs, the female reproductive system and the brain. Mutations in Wnt-7a lead to limb patterning defects along with sterility in both males and females. Ectopic expression of this protein leads to inhibition of chondrogenesis. This novel gene may therefore have therapeutic importance in several kinds of developmental defects and cancer, among other pathologis/disorders described above.

NOV6 is homologous to the Apical endosomal glycoprotein family of proteins. Thus NOV6 nucleic acids, polypeptides, antibodies and related compounds according to the invention will be useful in therapeutic and diagnostic applications implicated in, for example, endometriosis, fertility, and/or other pathologies/disorders.

After endocytosis from the plasma membrane, internalized receptors and ligands are delivered to endosomes. The endosomal compartment performs a variety of functions, including the sorting of internalized receptors and ligands, and newly synthesized lysosomal membrane proteins and hydrolases. In polarized epithelial cells, the apical endosomal compartment has been implicated in both apical to basolateral and basolateral to apical transepithelial transport.

NOV7 is homologous to members of the A Disintegrin And Metalloprotease (ADAMs) family of proteins, and specifically domain 13 (ADAM13). Thus, the NOV7 nucleic acids, polypeptides, antibodies and related compounds according to the invention will be useful in therapeutic and diagnostic applications implicated in, for example, Xerostomia, Scleroderma, Hypercalceimia, Ulcers, Von Hippel-Lindau (VHL) syndrome, Cirrhosis,Transplantation, Cirrhosis, Inflammatory bowel disease, Diverticular disease, Hirschsprung's disease , Crohn's Disease, Appendicitis, Endometriosis,Fertility, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases,Tuberous sclerosis, Scleroderma, Obesity, Aneurysm, Fibromuscular dysplasia, Stroke, Bleeding disorders, Hemophilia, hypercoagulation, Idiopathic thrombocytopenic purpura, autoimmume disease,allergies, immunodeficiencies, Graft vesus host, Anemia, Ataxia-telangiectasia, Lymphedema, Allergies, Tonsilitis, and/or other pathologies/disorders.

The ADAM family includes proteins containing disintegrin-like and metalloprotease-like domains. They are also referred to as MDC (Metalloprotease, Disintegrin, Cysteine-rich) proteins. ADAMs are involved in diverse processes such as development, cell-cell interactions and protein ectodomain shedding. In Xenopus, ADAM13 (most closely related to ADAM12) may be involved in neural crest cell adhesion and migration as well as myoblast differentiation. ADAM12/Meltrin α is required for and provokes myogenesis (myoblast fusion).

NOV8 is homologous to the Leucine-rich containing F-Box family of proteins. Since the NOV8 protein of the invention is ubiquitously expressed in many tissues, the NOV8 nucleic acids and polypeptides, antibodies and related compounds according to the invention will be useful in therapeutic and diagnostic applications implicated in the treatment of patients suffering from diseases associated with these tissues, and/or other pathologies/disorders.

F-box proteins are an expanding family of eukaryotic proteins characterized by an approximately 40 amino acid motif, the F box (so named because cyclin F was one of the first proteins in which this motif was identified). Some F-box proteins have been shown to be critical for the controlled degradation of cellular regulatory proteins. In fact, F-box proteins are one of the four subunits of ubiquitin protein ligases called SCFs. The other three subunits are the Skp1 protein; one of the cullin proteins (Cul1 in metazoans and Cdc53 or Cul A in the yeast Saccbaromyces cerevisiae); and the recently identified Roc1 protein (also called Rbx1 or Hrt1). SCF ligases bring ubiquitin conjugating enzymes (either Ubc3 or Ubc4) to substrates that are specifically recruited by the different F-box proteins. The need for high substrate specificity and the large number of known F-box proteins in yeast and worms suggest the existence of a large family of mammalian F-box proteins. There are 26 human F-box proteins. Some of these proteins contain WD-40 domains or leucine-rich repeats; others contain either different protein-protein interaction modules or no recognizable motifs. F-box proteins that contain WD-40 domains Fbws, those containing leucine-rich repeats, Fbls, and the remaining ones Fbxs. The marked differences in F-box gene expression in human tissues suggest their distinct role in ubiquitin-dependent protein degradation.

NOV9 is homologous to a Steroid binding family of proteins. Thus, the NOV9 nucleic acids, polypeptides, antibodies and related compounds according to the invention will be useful in therapeutic and diagnostic applications implicated in, for example, cancer, cataracts, obesity, diabetes, hyperlipidemia, infertility, inflammation, CNS disorders, and/or other pathologies/disorders.

Steroid binding proteins involve reproductive behavior, cell cycle progression and various important physiologic pathologies. Steroid hormones control many normal biological processes but can also cause several disease processes including hormone-dependent cancers of male and female reproductive tissues.

NOV10 is homologous to members of the steroid dehydrogenase family of proteins. Thus, the NOV10 nucleic acids, polypeptides, antibodies and related compounds according to the invention will be useful in therapeutic and diagnostic applications implicated in, for example, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, adrenoleukodystrophy, congenital adrenal hyperplasia, diabetes, Von Hippel-Lindau (VHL) syndrome, cirrhosis, pancreatitis, endometriosis, fertility, hemophilia, hypercoagulation, idiopathic thrombocytopenic purpura, autoimmume disease, allergies, immunodeficiencies, transplantation, graft versus host disease, osteoporosis, hypercalceimia, arthritis, ankylosing spondylitis, scoliosis, muscular dystrophy, Lesch-Nyhan syndrome, myasthenia gravis, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, psoriasis, actinic keratosis, acne, hair growth/loss, allopecia, pigmentation disorders, endocrine disorders, and/or other pathologies/disorders.

Steroid dehydrogenase enzymes influence mammalian reproduction, hypertension, neoplasia, and Digestion. The three-dimensional structures of steroid dehydrogenase enzymes reveal the position of the catalytic triad, a possible mechanism of keto-hydroxyl interconversion, a molecular mechanism of inhibition, and the basis for selectivity. Glycyrrhizic acid, the active ingredient in licorice, and its metabolite carbenoxolone are potent inhibitors of human 11 beta-hydroxysteroid dehydrogenase and bacterial 3 alpha, 20 beta-hydroxysteroid dehydrogenase (3 alpha, 20 beta-HSD). The three-dimensional structure of the 3 alpha, 20 beta-HSD carbenoxolone complex unequivocally verifies the postulated active site of the enzyme, shows that inhibition is a result of direct competition with the substrate for binding, and provides a plausible model for the mechanism of inhibition of 11 beta-hydroxysteroid dehydrogenase by carbenoxolone. The structure of the ternary complex of human 17 beta-hydroxysteroid dehydrogenase type 1 (17 beta-HSD) with the cofactor NADP+ and the antiestrogen equilin reveals the details of binding of an inhibitor in the active site of the enzyme and the possible roles of various amino acids in the catalytic cleft. The short-chain dehydrogenase reductase (SDR) family includes these steroid dehydrogenase enzymes and more than 60 other proteins from human, mammalian, insect, and bacterial sources. Most members of the family contain the tyrosine and lysine of the catalytic triad in a YxxxK sequence. X-ray crystal structures of 13 members of the family have been completed. When the alpha-carbon backbone of the cofactor binding domains of the structures are superimposed, the conserved residues are at the core of the structure and in the cofactor binding domain, but not in the substrate binding pocket.

Mutations of steroid dehydrogenases have been found to cause various developmental, reproductive or metabolic disorders. For example, Defects in the conversion of androstenedione to testosterone in the fetal testes by the enzyme 17 beta-hydroxysteroid dehydrogenase (17 beta-HSD) give rise to genetic males with female external genitalia. Missense and splice junction mutations severely compromised the activity of the 17 beta-HSD type 3 isozyme and cause male pseudohermaphroditism. Mutations in the NSDHL gene, encoding a 3beta-hydroxysteroid dehydrogenase, cause CHILD syndrome. Deficient or impaired 11 beta-hydroxy steroid dehydrogenase in the apparent mineralocorticoid excess syndrome or after licorice ingestion retards the conversion of cortisol to inactive cortisone in the kidney, leading to mineralocorticoid hypertension; this leads to suppression of the renin system and subsequently of aldosterone. In addition, steroid dehydrogenases have been implicated to regulate steroid induced renal reabsorption of sodium. Not only may they control the access of glucocorticoids to MR, but control the access of glucocorticoids to glucocorticoid receptors (GR) as well as access of mineralocorticoids to their own receptors. Finally, steroid dehydrogenases have also been found in neurons and astrocytes, suggesting that these enzymes may be involved in the regulation of brain function. Given their important biological functions, steroid dehydrogenases present excellent small molecule drug targets for therapeutic intervention.

NOV11 is homologous to a Myosin heavy-chain family of proteins. Thus, the NOV11 nucleic acids, polypeptides, antibodies and related compounds according to the invention will be ED useful in therapeutic and diagnostic applications implicated in, for example, restenosis, neurological, glomerular diseases, and/or other pathologies/disorders.

Myosins are molecular motors that upon interaction with actin filaments convert energy from ATP hydrolysis into mechanical force. Evidence has emerged for the existence of a large, widely expressed and evolutionarily ancient superfamily of myosin genes. In addition to the well-catheterized conventional, filament-forming, two-headed myosin-II of muscle and nonmuscle cells, at least ten additional classes of myosins have been identified. In vertebrates, at least seven of the eleven classes are expressed, and many myosins can be expressed in a single cell type. Distance matrix and maximum parsimony methods have been used to study the evolutionary relationships between members of the myosin superfamily of molecular motors. Amino acid sequences of the conserved core of the motor region were used in the analysis. Myosins can be divided into at least three main classes, with two types of unconventional myosin being no more related to each other than they are to conventional myosin. Myosins have traditionally been classified as conventional or unconventional, with many of the unconventional myosin proteins thought to be distributed in a narrow range of organisms. It has been found that members of all three of these main classes are likely to be present in most (or all) eukaryotes. Three proteins do not cluster within the three main groups and may each represent additional classes. The structure of the trees suggests that these ungrouped proteins and some of the subclasses of the main classes are also likely to be widely distributed, implying that most eukaryotic cells contain many different myosin proteins. The groupings derived from phylogenetic analysis of myosin head sequences agree strongly with those based on tail structure, developmental expression, and (where available) enzymology, suggesting that specific head sequences have been tightly coupled to specific tail sequences throughout evolution. Analysis of the relationships within each class has interesting implications. For example, smooth muscle myosin and striated muscle myosin seem to have independently evolved from nonmuscle myosin. Furthermore, brush border myosin I, a type of protein initially thought to be specific to specialized metazoan tissues, probably has relatives that are much more broadly distributed. Myosin II, the conventional two-headed myosin that forms bipolar filaments, is directly involved in regulating cytokinesis, cell motility and cell morphology in nonmuscle cells. To understand the mechanisms by which nonmuscle myosin-II regulates these processes, investigators are looking at the regulation of this molecule in vertebrate nonmuscle cells. The identification of multiple isoforms of nonmuscle myosin-II, whose activities and regulation differ from that of smooth muscle myosin-II, suggests that, in addition to regulatory light chain phosphorylation, other regulatory mechanisms control vertebrate nonmuscle myosin-II activity. It has been shown that nonmuscle myosin II, along with other myosins and cytoskeletal proteins, assembles on Golgi membranes. Nonmuscle myosin II associates transiently with membranes of the trans-Golgi network during the budding of a subpopulation of transport vesicles. The exact role of myosin II in vesicular trafficking is not yet understood, but its participation heralds a novel role for actin-based motors in vesicle budding.

In the aortic wall of mammalian species, the maturation phase of smooth muscle cell (SMC) lineage is characterized by two temporally correlated but opposite regulatory processes of gene expression: upregulation of SM type SM2 myosin isoform and down-regulation of brain (myosin heavy chain B)- and platelet (myosin heavy chain A(pla))-type nonmuscle myosins. There is propensity of the immature type SMC population to be activated in experimental models and human vascular diseases that are characterized by proliferation and migration of medial SMCs into the subendothelial space. Neointimal proliferation leading to restenosis frequently develops after coronary angioplasty. This process is associated with a change in vascular smooth-muscle cells from a contractile (quiescent) phenotype to a synthetic or proliferating (activated) one. The expression of the B isoform of nonmuscle myosin heavy chain is increased in some coronary atherosclerotic plaques and that this increase in expression identifies a group of lesions at high risk for restenosis after atherectomy.

The human homologue of the mouse dilute gene combines elements from both nonmuscle myosin type I and nonmuscle myosin type II. Mutations in the mouse dilute gene result not only in the lightening of coat color, but also in the onset of severe neurological defects shortly after birth, indicating that this gene is important in maintaining the normal neuronal function.

NOV12 is homologous to a Pancreatitis-associated family of proteins. Thus, the NOV12 nucleic acids, polypeptides, antibodies and related compounds according to the invention will be useful in therapeutic and diagnostic applications implicated in, for example, acute pancreatitis, chronic pancreatitis, and/or other pathologies/disorders.

Human Pancreatitis-associated protein (PAP) is a secretory protein that is strongly expressed in the pancreas with pancreatitis, but not in a healthy pancreas. Thus, synthesis 2.6 increases during inflammation of the pancreas, and a direct relationship between severity of pancreatitis and serum levels of PAP exists. As a result, PAP may be used as a biological marker of acute or chronic pancreatitis.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

One NOVX protein of the invention, referred to herein as NOV 1, includes stabilin-like proteins. The disclosed proteins have been named NOV1a, NOV1b, and NOV1c.

Stabilin is a member of the fascilin domain containing protein family, which has been shown to be important for cell adhesion. Although such cell adhesion molecules are typically localized at the neuromuscular junction in Drosophila, where they function in the growth and plasticity of the synapse, the protein predicted here is likely to be localized extracellularly in the plasma membrane. Thus, it is likely that the stabilin-like protein of the invention is accessible to a diagnostic probe and for the various therapeutic applications described herein.

The NOV1a protein maps to chromosome 3, whereas the NOV1b protein of the invention maps to chromosome 12. This information was assigned using OMIM, the electronic northern bioinformatic tool implemented by CuraGen Corporation, public ESTs, public literature references and/or genomic clone homologies.

NOV1a

In one embodiment, a NOV1 variant is NOV1a (alternatively referred to herein as CG-AC084364.5), which encodes a novel stabilin-like protein and includes the 8444 nucleotide sequence (SEQ ID NO: 1) shown in Table 1A. An open reading frame for the mature protein was identified beginning with an ATG initiation codon at nucleotides 1-3 and ending with a TGA stop codon at nucleotides 8026-8028. Putative untranslated regions downstream from the termination codon are underlined in Table 1A, and the start and stop codons are in bold letters.

TABLE 1A
NOV1a Nucleotide Sequence

(SEQ ID NO:1)

ATGGGCCTGCGCAGTCTGGGGCTCCTGGCTGTGCTGCCACTTCCTGAGTCAAGCACTGGACAGTGTGCAGTGGCC
AATGCTGGAGGGAGCTGAGCTCTGCAGGAACCCGGCACTGGAAAAAGCCATGTGGGGCTAAGAAACAGAGAAAAG
CTGTTTTTCGGGNNNNNNATGAATGAAATGGAGAGGCAAAGAACTGGAAATAGCAAGACGAGGTATCATGCTACT
GCAATAGTCCAGGCAAAACATGATAAAGGCCTCAACAAGAATGGCACCAGTGGAGATGAAGAGCAGAAGATCAAG
GTGGGAGACAGAGAACAGAGAACAAAGGATTTGATGGCTTATTAGATGTTTGGAAATACTTTAAACTTTATTCAT
CCTTGCTTTGCTGTGTGCAAACTGTGTGCATGGGTGTGCAACAGTGGACTAGATGGCGATGGAACCTGTGAGTGC
TACTCTGCGTACACTGGCCCCAAGTGTGACAAGCTCACAGAAAACTTTCACACCTCTCATCTGACACTGTGGCCT
GTGCACGACTCCAAGCACTGGGGAAGCCTTCGACATCAGAATATGAATGGCACCTGTTCTTCCGGGGGCGGCAAG
GGGGATCCCGATGTTTATCAAAATGGGTTGATTTTCCACGGAGGGGGTACTTCTGGAGGTCTATCGTCATCACGA
AACAGACGAAGTAGTGTCAAGCGTCCTGAGAAGTGGAAGGGGGACGATCGAGATGGAGGTGGCAAGGAAGGCCAG
CAGCGGCGGGCGGGGCAGACACAGAGTCGAGTCTTCAGAGGTCACATCAAAACGCCCCTGCCCCACAGGCAAGGT
GAAGCGCGGATCACGGAGACAACGGGGAATTGTGTTTCTGCTGGCATGACTGGAACCAATGCCAATCACACAAAA
GTTCACCCTACGGTTCAGTCCTTGACAGAATATGATTCCTTTCAGACTCATTCCACCAGCAGACTGAAGGAATTT
GAGAAACAGCAGGTGAAGGAAGATTTTTCTGACCCTCCCCTAATGCAGGCTATAAAACCCTCACATGAGAAGTAC
CCTCCTTATGCCCAGAGATAAGGAACATCTTTGTCTCCAAGACACAGGGACACGGAGATGATGAAACAGGCCTTG
CTAAGTTTCCTCCACTCTATTACCCTTAGCTTGTACCTTTATCCAACCACATTCTTCCATGACTCTCCAGTCTTC
ATCAAACCTGGCATAAACACTCAGACTTAACCACTTCTTTGGGTCTTCATTTCCTTATGTTAAGGCTCCAGTGTC
ATANNNNNNNATGGGAATTGAGGTTTGGAAAACTGGTGCCAAAATGCTGATACCCTGGCTGCTGCCCCTGCTCCA
TCCCTGAATGTGCAGCCTTGCTCTGCCCAGAAAATTCCAGATGTTCGCCTTTCCACTGAAGATGAAAACAACTGG
AATGCAAATGCCTTCCCAATTACCGAGGCGATGGCAATACTGCGACCCCATCAATCCATGTTTACGAAAAAATCT
GCCACCCTCATGCTCATTGTACGTACCTGGGACCAAATCGGCACAGTTGTACATGCCAAGAAGGCTACCGTGGGG
ATGGCCAAGTGTGCTTGCCTGTGGACCCCTGCCAAATTAACTTTGGAAACTGCCCTACAAAGTCTACAGTGTGCA
AATATGATGGGCCTGGACAGATGCATTTGCCAGAAAGGTTACGTGGGTGATGGCTTAACGTGTTATGGAAACATT
ATGGAGCGACTCAGAGAATTAAATACTGAACCCAGAGGAAAATGGCAAGGAAGGCTGACCTCTTTCATCTCACTC
CTAGAAAGTATACAAATTGTAAGTGTACAACTCAGTGAATTTTCCCAACGTGAACCTACTTGTGTAAACACCAAG
TCCATTGCCAGCAACCTAGAAGGCCCCCTGGTCCCCCTTTCCAATCATTACCCTCTACAGGTAAATGAGCTTTTG
GTGGATAATAAAGCTGCTCAATACTTTGAAATGCTCCACATAATTGCTGGTCAGATGAACATCGAATATATGAAT
AACACAGACATGTTCTACACCTTGACTGGAAAGTCGGGGGAAATCTTCAACAGCGATAAGGACAATCAAATAAAG
CTTAAACTCCATGGAGGCAAAAAGAAGGTAAAAATTATACAAGGGGACATCATTGCTTCCAATGGGCTTCTGCAC
ATCCTTGACAGAGCCATGGACAAGTTAGAACCCACATTTGAGAGCAACAATGAGGAAACCAATTTGGGACATGCC
TTAGATGAGGATGGAGTTGGTGGACCATACACCATTTTTGTTCCAAATAATGAAGCATTGAATAACATGAAGGAC
GGCACTCTCGATTACCTCCTTTCTCCAGAQCTTGAAGTGGCCACTCTCATCTCCACCCCTCACATAAGGAGCATG
GCCAACCAGCTCATACAGTTCAACACCACCGACAATGGACAGATTCTGGCAAATGATGTGGCAATGGAAGAAATT
GAGATCACTGCCAAAAATGGCCGAATTTACACACTGACAGGAGTTCTCATTCCTCCCTCCATTGTCCCGATTCTG
CCCCATCGATGTGATGAAACAAGAGAGAGATGAAAACTGGGCACTTGTGTGAGCTGTTCTCTGGTGTACTGGAGC
AGATGTCCTGCTAACTCTGAGCCCACAGCACTCTTCACACACAGATGTGTCTACAGTGGCAGGTTTGGGAGCCTG
AAGAGCGGCTGTGCCCGGTACTGCAATGCCACTGTGAAGTGTGCAGATAGCCTCGGCGGCAACGGGACATGCATT
TGTGAGGAGGGCTTCCAAGGCTCCCAGTGTCAGTTCTGCTCTGATCCCAATAAATACGGACCTCGGTGTAACAAA
AAATGCCTGTGCGTTCACGGAACATGCAATAACAGGATAGACAGCGATGGGGCCTGCCTCACTGGCACATGCAGA
GACGCTCTGCCGGGAGACTCTGTGATAAAGCAGACCTCAGCCTGTGGGCCCTACGTGCAGTTCTGTCAAATCCAC
GCCACCTGTGAATACAGCAATGGGACAGCCAGTTGTATTTGCAAAGCAGGATATGAAGGAGATGGAACTCTGTGT
TCTGAGATGGACCCTTGCACAGGACTAACTCCAGGAGGCTGTAGCCGCAATGCAGAATGCATCAAAACTGGCACG
GGCACCCACACCTGCGTGTGTCAGCAGGGTTGGACAGGGAATGGGAGAGACTGCTCGGAGATCAACAACTGCCTG
CTGCCCAGTGCAGGCGGCTGCCACGACAACGCATCCTGTTTGTATGTGGGTCCCGGGCAGAATGAGTGTGAGTGC
AAGAAAGGATTTCGAGGAAATGGGATTGACTGTGAACCAATAACTTCATGCTTGGAACAAACCGGGAAATGTCAT
CCATTGGCAAGCTGTCAATCTACTTCGTCTGGTGTCTGGAGCTGTGTTTGTCAAGAGGGCTATGAAGGAGATGGC
TTTCTGTGCTATGGAAACGCAGCAGTGGAATTGTCATTTCTCTCCGAAGCAGCTATATTTAACCGATGGATAAAT
AATGCTTCTCTACAACCCACACTGTCAGCCACCTCAAACCTCACTGTCCTCGTGCCTTCCCAACAAGCTACTGAG
GACATGGACCAGGATGAGAAAAAGCTTCTGGTTGTCACAGAGCAATATTCCAGCCCTAATAAGTACCATATGCTA
CTAGGCACATACAGAGTGGCAGATCTGCAGACCCTGTCTTCTTCTGACATGTTGGCAACATCTTTGCAGGGCAAC
TTCCTTCACTTGGCAAAGGTGGATGGGAATATCACAATTGAAGGGGGCCTCCATTGTCGATGGGGACAACGAGCC
ACAAATGGAGTGATACACATCATCAACAAGGTGCTGGTCCCACAAAGACGTCTAACTGGCTCCTTACCAAACCTG
CTCATGCGGCTGGAACAGATGCCTGACTATTCCATCTTCCGGGGCTACATCATTCAATATAATCTGGCGAATGCA
ATTGAGGCTGCCGATGCCTACACAGTGTTTGCTCCAAACAACAATGCCATCGAGAATTACATCAGGGAGAAGAAA
GTCTTGTCTCTAGAGGAGGACGTCCTCCGGTATCATGTGGTCCTGGAGGAGAAACTCCTGAAAAGAATGACCTGC
AATGGCATGCATCGTGAGACCATGCTGGGTTTCTCCTATTTCCTTAGCTTCTTTCTCCATAATGACCAGCTCTAT
GTAAATGAGGCTCCAATAACTACACCAAATGTAGCCACTGATAAGGGAGTGATCCATGGTTTGGGAAAAGTTCTG
GAAATTCAGAAGAACAGATGTGATAATAATGACACTACTATTATACGAGGAAGATGTAGGACATGCTCCTCAGAG
CTGACCTGCCCATTCGGAACTAAATCTCTAGGTAATGAGAAGAGGAGATGCATCTATACCTCCTATTTCATGGGA
AGACGAACCCTGTTTATTGGGTGCCAGCCAAAATGTGTGAGAACCGTCATTACGAGAGAATGCTGTGCCGGCTTC
TTTGGCCCCCAATGCCAGCCCTGTCCAGGGAATGCCCAGAATGTCTGCTTTGGTAATGGCATCTGTTTGGATGGA
GTGAATGGCACAGGTGTGTGTGAGTGTGGGGAGGGCTTCAGCGGCACAGCCTGCGAGACCTGCACCGAGGGCAAG
TACGGCATCCACTGTGACCAGCATGTTCTTGTGTCCATGGGAGATGAACCAAGGACCCTTGGGAGATGGCTCTCC
TGTGACTGTGATGTTGGCTGGCGAGGAGTGCATTGTGACAATGCAACCACAGAAGACAACTGCAATGGGACATGC
CATACCAGCGCCAACTGCCTCACCAACTCAGATGGTACAGCTTCATGCAAGTGTGCAGAAGGATTCCAAGGAAAC
GGQACCATCTGCACAGCAATCAATGCCTGTGAGATCAGCAATGGAGGTTGCTCTGCCAAGGCTGACTGTAAGAGA
ACCACCCCAGGAAGGCGAGTGTGCACGTGCAAAGCAGGCTACACGGGTGATGGAATTGTGTGCCTGGAAATCAAC
CCGTGTTTGGAGAACCATGGTGGCTGTGACAAGAATGCGGAGTGCACACAGACAGGACCCAACCAGGCTGCCTGT
AACTGTTTGCCAGCATACACTGGAGATGGAAAGGTCTGCACACTCATCAATGTCTGCTTAACTAAAAATGGCGGC
TGTGGTGAATTTGCCATCTGCAACCACACTGGGCAAGTAGAAAGGACTTGTACTTGCAAGCCAAACTACATTGGA
GATGGATTTACCTGCCGCGGCAGCATTTATCAGGAGCTTCCCAAGAACCCGAAAACTTCCCAGTATTTCTTCCAG
TTGCAGGAGCATTTCGTGAAAGATCTGGTCGGCCCAGGCCCCTTCACTGTTTTTGCACCTTTATCTGCAGCCTTT
GATGAGGAAGCTCGGGTTAAAGACTGGGACAAATACGGTTTAATGCCCCAGGTTCTTCGGTACCATGTGGTCGCC
TGCCACCAGCTGCTTCTGGAAACCTGAAATTGATCTCAAAATGCTACTTCCCTCCAAGGAGAGCCAATAGTCATC
TCCGTCTCTCAGAGCAACGGTGTATATAATAATAAGGCTAAGATCATATCCAGTGATATCATCAGTACTAATGGG
ATTGTTCATATCATAGACAAATTGCTATCTCCCAAAAATTTGCTTATCACTCCCAAAGACAACTCTGGAAGAATT
CTGCAAAATCTTACGACTTTGGCAACAAAACAATGGCTACATCAAATTTAGCAACTTAATACAGGACTCAGGTTTG
CTGAGTGTCATCACCGATCCCATCCACACCCCAGTCACTCTCTTCTGGCCCACCGACCAAGCCCTCCATGCCCTA
CCTGCTGACAACAGGACTTCCTGTTCAACCAAGACAACAAGGACAAGCTGAAGGAGTATTTGAAGTTTCATGTG
ATACGAGATGCCAAAGGTTTTAGCTGTGGATCTTCCCACATCCACTGCCTGGAAGACCCTGCAAGGTTCAGAGCTG
AGTGTGAATGTGGAGCTGGCAGGGACATCGGTGACCTCTTTCTGAATGGCCAAACCTGCAGATTTGTGCAGCGG
GAGCTCTTGTTTGACCTGGGTGTGGCCTACGGCATTGACTGTCTGCTGATTGATCCCACCCTGGGGGGCCGCTGT
GACACCTTTACTACTTTCGATGCCTCGGGGGAGTGTGGGAGCTGTGTCAATACTCCCAGCTGCCCAAGGTGGAGT
AAACCAAAGGGTGTGAAGCAGAAGTGTCTCTACAACCTGCCCTTCAAGAGGAACCTGGAAGGCTQCCGGGAGCGG
TGCAGCCTGGTGATACAGATCCCCAGGTGCTQCAAQGGCTACTTCGGGCGAGACTGTCAGGCCTGCCCTGGAGGA
CCAGATGCCCCGTGTAATAACCGGGGTGTCTGCCTTGATCAGTACTCGGCCACCGGAGAGTGTAAATGCAACACC
GGCTTCAATGGGACGGCGTGTGAGATGTGCTGGCCGGGGAGATTTGGGCCTGATTGTCTGCCCTGTGGCTGCTCA
GACCACGGACAGTGCGATGATGGCATCACGGQCTCCGGGCAGTGCCTCTGTGAAACGGGGTGGACAGGCCCCTCG
TGTGACACTCAGGCAQTTTTGCCTGCAGTGTGTACGCCTCCTTGTTCTGCTCATGCCACCTGTAAGGAGAACAAC
ACGTGTGAGTGTAACCTGGATTATGAAGGTGACGGATCACATGCACAGTTGTGGAATTTCTGCAACAGQACAAC
GGGGGCTGTGCAAAGGTGGCCAGATGCTCCCAGAAGGGCACGAAGGTCTCCTGCAGCTGCCAGAAGGGATACAAA
GGGGACGGGCACAGCTGCACAGAGATAGACCCCTGTGCAGACGGCCTTAACGGAGGGTGTCACGAGCACGCCACC
TGTAAGATGACAGGCCCGGGCAAGCACAAGTGTGAGTGTAAAAGTCACTATGTCGGAGATGGGCTGAACTGTGAG
CCGGAGCAGCTGCCCATTGACCGCTGCTTACAGGACAATGGGCAGTGCCATGCAGACGCCAAATGTGTCGACCTC
CACTTCCAGGATACCACTGTTGGGGTGTTCCATCTACGCTCCCCACTGGGCCAGTATAAGCTGACCTTTGACAAA
GCCAGAGAGGCCTGTGCCAACGAAGCTGCGACCATGGCAACCTACAACCAGCTCTCCTATGCCCAGAAGGCCAAG
TACCACCTGTGCTCAQCAGGCTGGCTGGAGACCGGGCGGGTTGCCTACCCCACAGCCTTCGCCTCCCAGAACTGT
GGCTCTGGTGTGGTTGGGATAGTGGACTATGQACCTAGACCCAACA1AAGAGTGAAATGTGGGATGTCTTCTGCTAT
CGGATGAAAGGAAGTGCTGGCCTATTCCAACAGCTCAGCTCGAGGCCGTGCATTTCTAGAACACCTGACTGACCTG
TCCATCCGCGGCACCCTCTTTGTGCCACAGAACAGTGGGCTGGGGGAGAATGAGACCTTGTCTGGGCGGGACATC
GAGCACCACCTCGCCAATGTCAGCATGTTTTTCTACAATGACCTTGTCAATGGCACCACCCTGCAAACGAGGCTG
GGAAGCAAGCTGCTCATCACTGCCAGCCAGGACCCACTCCAACCGGTACAAAAGTAGGTTTGTTGATCGAAGAGCC
ATTCTGCAGTGGGACATCTTTGCCTCCAATGGGATCATTCATGTCATTTCCAGGCCTTTAAAAGCACCCCCTGCC
CCCGTGACCTTGACCCACACTGGCTTGGGAGCAGGGATCTTCTTTTGCATCATCCTGGTGACTGGGGCTCTTGCC
TTGGCTGCTTACTCCTACTTTCGGATAAACCGGAGAACAATCGGCTACCAGCATTTTGAGTCGGAAGAGGACATT
AATGTTGCAGCTCTTGGCAAGCAGCAGCCTGAGAAATATCTCGAACCCCTTGTATGAGAGCACAACCTCAGCTCCC
CCAGAACCTTCCTACGACCCCTTCACGGACTCTGAAGAAACQGCAGCTTGAGGGCAATGACCCCTTGAGACACTG
TGAGGGCCTGGACGGGAGATGCCAGCCATCACTCACTGCCACCTGGGCCATCAAACTGTGAATTCTCAGCACCAGT
TGCCTTTTAGGAACGTAAAGTCCTTTAAGCACTCAGAAGCCATACCTCATCTCTCTGGCTGATCTGGGGGTTGTT
TCTGTGGGTGAGAGATGTGTTGCTGTGCCCACCCAGTACAGCTTCCTCCTCTGACCCTTTGGCTCTTCTTCCTTT
GTACTCTTCAGCTGGCACCTGCTCCATTCTGCCCTACATGATGGGTAACTGTGATCTTTCTTCCCTGTTAGATTG
TAAGCCTCCGTCTTTGTATCCCAGCCCCTAGCCCAGTGCCTGACACAGGAACTGTGCACAATAAAGGTTTATGGA
ACAGAAACAAAGTCAAAAAAAAAAAAAAAAAAAAAAAAAAAAAA

The sequence of NOV1a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel stabilin-like gene were obtained by SeqCallingTM Technology and are reported here as NOV1a. These methods used to amplify NOV1a cDNA are described in Example 2.

The NOV1a polypeptide (SEQ ID NO: 2) encoded by SEQ ID NO: 1 is 2675 amino acid residues in length and is presented using the one-letter amino acid code in Table 1B. The SignalP, Psort and/or Hydropathy results predict that NOV1a has a signal peptide and is likely to be localized extracellularly in the plasma membrane with a certainty of 0.6760. In alternative embodiments, a NOV1a polypeptide is located to the endoplasmic reticulum (membrane) with a certainty of 0.1000, the endoplasmic reticulum (lumen) with a certainty of 0.1000, or outside the cell with a certainty of 0.1000. The SignalP predicts a likely cleavage site for a NOV1a peptide between amino acid positions 20 and 21, i.e. at the dash in the sequence STG-QC.

TABLE 1B
Encoded NOVia Protein Sequence

(SEQ ID NO:2)

MGLRSLGLLAVLPLPESSTGQCAVAICCWRELSSAGTRHWRNHVGLRNREKLFFGXXMNEMERQETGNSKTRYHAT
AIVQAHDKGLNKAAGTSGDEEQKIKVGDRDRENKGFDGLLDWNTLNFIHPCFAVCNCVHGVCNSGLDGDGTCEC
YSAYTGPKCDKLTENFHTSHLTWPVHDSKHWGSLRHQNMNGTCSSGGGKDGPDVYQNGLIFHGGGTSGGLSSSR
NRRSSVKRPEKWKGDDRDGGGKEGQQRRPAADTESSLQRGHIKTPLPHRQGEAAITETTGNCVSAGMTGAAAATK
VHPTVQSLTEYDSFQTHSTSRLKEFEKQQVKERFSDPPLMQAIKPSHEKYPPYAQRKGTSLSPKTQGHGDDEQAL
LSFLHSITLSLYLYPTTFFHDSPVFIKPGIKTLRLAAFFGSSFPYECSSVIXXMGIEAAAACQNADTLAAAPAP
SLNVQPCSAQKIPDVRLPLKMKTNWNANAFPITEAMANTATPSIHVYEKSATLMLIVRTWDQIGTVVHAKKATVG
MAKCACLWTPAKLTLETALQSLQCANAAGLDRCICQKGYVGDGLTCYGNIMERLRELNTEPRGKWQGRLTSFISL
LESIQIVSVQLSEFSQREPTCVNTKSIASNLEGPLVPLSHNYPLQVNELLVDNKAAQYFVKLIHIIAGQMNIEYMN
NTDMFYTLTGKSGEIFNSDKDNQIYAAKLHGGKKKVKIIQGDIIASNGLLHILDRAMDKLEPTFESNNEETNLGAA
LDEDGVGGPYTIFVPNNEALNNMKDGTLDYLLSPELEVATLISTPHIRSMANQLIOFNTTDNGQILANDVAMEEI
EITAKNGRIYTLTGVLIPPSIVPILPHRCDETKREMKLGTCVSCSLVYWSRCPANSEPTALFTHRCVYSGRFGSL
KSGCARYCNATVKCADSLGGNGTCICEEGFQGSQCQFCSDPNKYGPRCNKKCLCVHGTCNNRIDSDGACLTGTCR
DGSAGRLCDKQTSACGPYVQFCHIAATCEYSNGTASCICKAGYEGDGTLCSEMDPCTGLTPGGCSPAANAECIKTGT
GTHTCVCQQGWTGNGRDCSEINNCLLPSAGGCHDNASCLYVGPGQNECECKKGFRGNGIDCEPITSCLEQTGKCH
PLASCQSTSSGVWSCVCQEGYEGDGFLCYGNAAVELSFLSEAAIFNRWINNASLQPTLSATSNLTVLPSQQATE
DMDQDEKSFWLSOSNIPALIKYHMLLGTYRVADLQTLSSSDMLATSLQGNFLHLAKVDGNITIEGASIVDGDNAA
TNGVIHIINKVLVPQRRLTGSLPNLLMRLEQMPDYSIFRGYIIQYNLANAIEAADAYTVFAPNNNAIENYIREKK
VLSLEEDVLRYHVVLEEKLLKNDLHNGMHRETMLGFSYFLSFFLHNDQLYVNEAPINTYNVATDKGVIHGLGKVL
EIQKNRCDNNDTTIIRGRCRTCSSELTCPFGTKSLGNEKRRCIYTSYFMGRRTLFIGCQPKCVRTVITRECCAGF
FGPQCQPCPGNAQNVCFGNGICLDGVNGTGVCECGEGFSGTACETCTEGKYGIHCDQACSCVHGRCNQGPLGDGS
CDCDVGWRGVHCDNATTEDNCNGTCHTSANCLTNSDGTASCKCAAGFQGNGTICTAINACEISNGGCSAKADCKR
TTPGRRVCTCKAGYTGDGIVCLEINPCLENHGGCDKNAECTQTGPNQAACNCLPAYTGDKVCTLINVCLTKNGG
CGEFAICNHTGQVERTCTCKPNYIGDGFTCRGSTYQELPKNPKTSQYFFQLQEHFVLVGPGPFTVFAPLSAAF
DEEARVKDWDKYGLMPQVLRYIZACHQLLLENLAAISNATSLQGEPIVISVSQSTAAIAAAATSSDIISTNG
IVHIIDKLLSPKNLLITPKDDNSGRILQNLTTLATNNGYIKFSNLIQDSGLLSVITDPIHTPVTLFWPTDQALHAL
PAEQQDFLNQDNKDKLKEYLKFHVIRDAKVLAVDLPTSTAWKTLQGSSELSVKCGAGRDIGDLFLNGQTCRIVQR
ELLFDLGVAYGIDCLLIDPTLGGRCDTFTTFDASGECGSCVNTPSCPRWSKPKGVKQKCLYNLPFKRNLEGCRER
CSLVIQIPRCCKGYFGRDCQACPGGPDAPCAAGVCLDQYSATGECKCNTGAAGTACEMCWPGRFGPDCLPCGCS
DHGQCDDGITGSGQCLCETGWTGPSCDTQAVLPAVCTPPCSAHATCKENNTCECNLDYEGDGITCTVVDFCKQDN
GGCAKVARCSQKGTKVSCSCQKGYKGDGHSCTEIDPCADGLNGGCHEHATCKMTGPGKHKCECKSHYVGDGLNCE
PEQLPIDRCLQDNGQCHADAKCVDLHFQDTTVGVFHLRSFLGQYKLTFDKAREACANEAATMATYNQLSYAQKAK
YHLCSAGWLETGRVAYPTAFASQNCGSGVVGIVDYGPRPNKSEMWDVFCYRMKEVLAYSNSSARGRAFLEHLTDL
SIRGTLFVPQNSGLGENETLSGRDIEHHLANVSMFFYNDLVNGTTLQTRLGSKLLITASQDPLQPVQSRFVDGRA
ILQWDIFASNGIIHVISRPLKAPPAPVTLTHTGLGAGIFFCIILVTGAVALAAYSYFRINPRTIGYQHFESEEDI
NVAALGKQQPENISNPLYESTTSAPPEPSYDPFTDSEERQLEGNDPLRTL

NOV1b

In an alternative embodiment, a NOV1 variant is NOV1b (alternatively referred to herein as CG50736-10), which includes the 8495 nucleotide sequence (SEQ ID NO: 3) shown in Table 1C. An open reading frame for the mature protein was identified beginning at nucleotides 201-203 and ending at nucleotides 7461-7463. The start and stop codons of the open reading frame are highlighted in bold type. Putative untranslated regions, found upstream from the initiation codon and downstream from the termination codon, are underlined.

TABLE 1C
NOV1b Nucleotide Sequence

(SEQ ID NO:3)

AAATCATCCCACATGCTAAGAATCTAAGATGTATAAAATAAAGTGGTAGAAAGATGAAAATGAAATTTTATCAAG
GTTAGAGTCAGGTTGGAGTGGCCATTGTTTACCACTGAGAAATCTAAATTTTATTTGGTTGGTAATTGAGA
GTCATTGAGATATTTTGGGGAAGGTCACCCTGATGCCTTTGCTAATCAAATGAAATGAATGAAATGGAGAGGC
AAGAAACTGGAAATAGCAAGACGAGGTATCATGCTACTGCAATAGTCCAGGCAAAACATGATAAAGGCCTCAA
CAAGAATGGCACCAGTGGAGATGAAGAGCAGAAGATCAAGGTCGGACCACAGAGACAGAGAAAAACAAAGGATTT
GATGGCTTATTAGATGTTTGGAATACTTTAAACTTTATTCATCCTTGCTTTGCTGTGTGCAACTGTGTGCATG
GGGTGTGCAACAGTGGACTAGATGGCGATGGAACCTGTGAGTGCTACTCTGCGTACACTGGCCCCAAGTGTGA
CAAGCTCACAGAAAAACTTTCACACCTCTCATCTGACACTGTGGCCTGTGCACGACTCCAAGCACTGGGGAAGC
CTTCGACATCAGAATATGAATGGCACCTGTTCTTCCGGGGGCGGCAAGGGGGATCCCGATGTTTATCAAAATG
GGTTGATTTTCCACGGAGGGGGTACTTCTGGAGGTCTATCGTCATCACGAAACAGACGAACTACTGTCAAAACCG
TCCTGAGAAGTGGAAGGGGGACGATCGAGATGGAGGTGGCAAGGAAGGCCAGCAGCGGCGGCGGGCAGACACA
GAGTCGAGTCTTCAAGAGGTCACATCAACGCCCCTGCCCCACAGGCAAAGGTGAAGCGCGGATCACGGAGA
CAACGGGGAATTGTGTTTCTGCTGGCATGACTGGAACCAATGCCAATCACACAAAAGTTCACCCTACGGTTCA
GTCCTTGACAGAATATGATTCCTTTCAGACTCATTCCACCAGCAGACTGAAGGAAATTTGAGAACAGCAAGGTG
AAGGAAAGATTTTCTGACCCTCCCCTAATGCAGGCTATAAAAACCCTCACATGAGAAGTACCCTCCTTATGCCC
AGAGAAAAGGAACATCTTTGTCTCCAAAGACACAGGGACACGGAGATGATGAACAGGCCTTGCTAAGTTTCCT
CCACTCTATTACCCTTAGCTTGTACCTTTATCCAAACCACATTCTTCCATGACTCTCCAGTCTTCATCAAACCT
GGCATAAAAACACTCAGACTTAACCACTTCTTTGGGTCTTCATTTCCTTATGAAGGCTCCAGTGTCATAANAA
NNATGGGAATTGAGGTTTGGAAAAACTGGTGCCAAAATGCTGATACCCTGGCTGCTGCCCCTGCTCCATCCCT
GAATGTGCAGCCTTGCTCTGCCCAGAAAATTCCAGATGTTCGCCTTCCACTGAAGATGAAAACAAACTGGAAT
GCAAAATGCCTTCCCAATTACCGAGGCGATGGCAAATACTGCGACCCCATCAATCCATGTTTACGAAAAATCTG
CCAACCCTGATGCTCATTGTACGTACCTGGGACCAAATCGGCACAGTTGTACATGCCAAGAAGGCTACCGTGGG
GATGGCCAAGTGTGCTTGCCTGTGGACCCCCTGCCAAATTAACTTTGGAAACTGCCCTACAAAGTCTACAGTGT
GCAAATATGATGGGCCTGGACAGATGCATTTGCCAGAAAGGGTTACGTGGGTGATGGCTTAACGTGTTATGGAA
ACATTATGGAGCGACTCAGAGAATTAAATACTGAACCCAGAGGAAAAATGGCAAGGAAGGCTGACCTCTTTCAT
CTCACTCCTAGAAAGTATACAAATTGTAAGTGTACAACTCAGTGAATTTTTCCCAACGTGAACCTACTTGTGTA
AACACCAAGTCCATTGCCAGCAACCTAGAAGGCCCCCCTGGTCCCCCTTTCCAATCATTACCCTCTACAGGTAA
ATGAGCTTTTTGGTGGATAATAAGCTGCAATCAATACTTTGTGAACTCCACATAATTGCTGGTCAGATGAACAT
CGAATATATGAATAACACAGACATGTTCTACACCTTGACTGGAAAGTCGGGGGGAAATCTTCAACAGCGATAAG
GACAATCAAATAAAGCTTAAACTCCATGGAGGCAAAAAGAAGGTAAAAATTATACAAGGGGACATCATTGCTT
CCAATGGGCTTCTGCACATCCTTGACAGAGCCATGGACAAGTTAGAACCCACATTTGAGAGCAACAATGAGGA
AACCAATTTGGGACATGCCTTAATGAGGATGGAGTTGGTGGACCATACACCATTTTTGTTCCAAATAATGAAA
GCATTGAATAACATGAAGGACGGCACTCTCGATTACCTCCTTTCTCCAGAGCTTGAAGTGGCCACTCTCATCT
CCACCCCTCACATCAGGAGCATGGCCAACCAGCTCATACAGTTCAACACCACCGACAATGGACAGATTCTGGC
AATGATGTGGCAATGGAAGAAATTGAGATCACTGCCAAAAATGCCGAAGAATTACACACTGACAGGAGTTCTC
GTGTGAGCTGTTCTCTGGTGTACTGGAGCAGATGTCCTGCTAACTCTGAGCCCACAGCACTCTTCACACACAG
GTGTGAGCTGTTCTCTGGTGTACTGGAGCAGATGTCCTGCTAACTCTGAGCCCACAGCACTCTTCACACACAG
ATGTGTCTACAGTGGCAGGTTTGGGAGCCTGAAGAGCGGCTGTGCCCGGTACTGCAATGCCACTGTGAAGTGT
GCAGATAGCCTCGGCGGCAACGGGACATGCATTTGTGAGGAGGGCTTCCAAGGCTCCCAGTGTCAGTTCTGCT
CTGATCCCAATAAATACGGACCTCGGTGTAACAAAAAATGCCTGTGCGTTCACGGAACATGCAATAACAGGAT
AGACAGCGATGGGGCCTGCCTCACTGGCACATGCAGAGACGGCTCTGCCGGGAGACTCTGTGATAAGCAGACC
TCAGCCTGTGGGCCCTACGTGCAGTTCTGTCACATCCACGCCACCTGTGAATACAGCAATGGGACAGCCAGTT
GTATTTGCAAGCAGGATATGAAGGAGATGGAACTCTGTGTTCTGAGATGGACCCTTGCACAGGACTAAACTCC
AGGAGGCTGTAGCCGCAATGCAGAATGCATCAAAACTGGCACGGGCACCCACACCTGCGTGTGTCAGCAGGGT
TGGACAGGGAATGGCAGAGACTGCTCGGAGATCAACAACTGCCTGCTGCCCAGTGCAGGCGGCTGCCACGACA
ACGCATCCTGTTTGTATGTGGGTCCCGGGCAGAATGAGTGTGAGTGCAAGAAGGATTTCGAGGAAAATGGGAT
TGACTGTGAACCAATAACTTCATGCTTGGAACAAACCGGGAAATGTCATCCATTGGCAAGCTGTCAATCTACT
TCGTCTGGTGTCTGGAGCTGTGTTTGTCAAGAGGGCTATGAAGGAGATGGCTTTCTGTGCTATGGAAACGCAG
CAGTGGAATTGTCATTTCTCTCCGAAGCAGCTATATTTAACCGATGGATAAATAATGCTTCTCTACAACCCAC
ACTGTCAGCCACCTCAAACCTCACTGTCCTCGTGCCTTCCCAACAAGCTACTGAGGACATGGACCAGGATGAG
AAAAGCTTCTGGTTGTCACAGAGCAATATTCCAGCCCTAATAAAGTACCATATGCTACTAGGCACATACAGAG
TGGCAGATCTGCAGACCCTGTCTTCTTCTGACATGTTGGCAACATCTTTGCAGGGCAACTTCCTTCACTTGGC
AAAGGTGGATGCGAATATCACAAATTGAAGGGGCCTCCATTGTCGATGGGGACAACGCAGCCACAATGGAGTG
ATACACATCATCAACAAGGTGCTGGTCCCACAAAGACGTCTAACTGGCTCCTTACCAAACCTCCTCATGCGGC
TGGAACAGATGCCTGACTATTCCATCTTCCGGGGCTACATCATTCAATATAATCTGGCGAATGCAATTGAGGC
TGCCGATGCCTACACAGTGTTTGCTCCACAACAATGCCATCGAGAATTACATCAGGGAGAAGAAAAAGTCTTG
TCTCTAGAGGAGGACGTCCTCCGGTATCATGTGGTCCTGGAAGGAAGCTCCTGAAGAATGACCTGCAACAATG
GCATGCATCGTGAGACCATGCTGGGTTTCTCCTATTTCCTTAGCTTCTTTCTCCATAATGACCAGCTCTATGT
AAATGAGGCTCCAATAAACTACACCAATGTAGCCACTGATAAGGGAGTGATCCATGGTTTGGGAAAAGTTCTG
GAATTCAGAAGAACAGATGTGATAATAATGACACTACTATTATACGAGGAAAGATGTAGGACATGCTCCTAAG
AGCTGACCTGCCCATTCGGAACTAAATCTCTAGGTAATGAGAAGAGGAGATGCATCTATACCTCCTATTTCAT
GGGAAGACGAACCCTGTTTATTGGGTGCCAGCCAAAATGTGTGAGAACCGTCATTACGAGAGAATGCTGTGCC
GGCTTCTTTGGCCCCCAATGCCAGCCCTGCCCAGGGAATGCCCAGAATGTCTGCTTTGGTAATGGCATCTGTT
TGGATGGAGTGAATGGCACAGGTGTGTGTGAGTGTGGGGAGGGCTTCAGCGGCACAGCCTGCGAGACCTGAAC
CGAGGGCAAGTACGGCATCCACTGTGACCAAGCATGTTCTTGTGTCCATGGGAGATGC1ACCAAGGACCCTTG
GGAGATGGCTCCTGTGACTGTGATGTTGGCTGGCGAGGAGTGCATTGTGACAATGCAACCACAGAAGACAACT
GCAATGGGACATGCCATACCAGCGCCAACTGCCTCACCAACTCAGATGGTACAGCTTCATGCAAGTGTGCAGC
AGGATTCCAAGGAACGGGACCATCTGCACAGCAATCAATGCCTGTGAGATCAGCAAATGGAGGTTGCTCTGCC
AAGGCTGACTGTAAGAGAACCACCCCAGGAAGGCGAGTGTGCACGTGCAAAGCAGGCTACACGGGTGATGGCA
TTGTGTGCCTGGAATCAACCCGTGTTTGGAGACCATGGTGGCTGTGACAAGAAATGCGGAGTGCACACAGAC
AGGACCCACCAGGCTGCCTGTAAACTGTTTGCCAGCATACACTGGAGATGGAAGGTCTGCACACTCATCAAT
GTCTGCTTAACTAAAATGGCGGCTGTAGTGAATTTGCCATCTGCAACCACACTGGGCAAGTAGAAAGGACTT
GTACTTGCAAGCCAACTACATTGGAGATGGATTTACCTGCCGCGGCAGCATTTATCAGGAGCTTCCCAAGAA
CCCGAAAACTTCCCAGTATTTCTTCCAGTTGCAGGAGCATTTCGTGAAGATCTGGTCGGCCCAGGCCCCTTC
ACTGTTTTTGCACCTTTATCTGCAGCCTTTGATGAGGAAGCTCGGGTTAAGACTGGGACAATACGGTTTAAAA
TGCCCCAGGTTCTTCGGTACCATGTGGTCGCCTGCCACCAGCTGCTTCTGGAAAACCTGAAATTGATCTCAA
TGCTACTTCCCTCCAAGGAGAGCCAATAGTCATCTCCGTCTCTCAGAGCACGGTGTATATAACAATAAGGCT
AAGATCATATCCAGTGATATCATCAGTACTAATGGGATTGTTCATATCATAGACAAATTGCTATCTCCCAAAA
ATTTGCTTATCACTCCCAAAGACAACTCTGGAAGAATTCTGCAAAATCTTACGACTTTGGCAACAAACAATGG
CTACATCAAATTTAGCAACTTAATACAGGACTCAGGTTTGCTGAGTGTCATCACCGATCCCATCCACACCCCA
GTCACTCTCTTCTGGCCCACCGACCAAGCCTCCATGCCCTCCATGCCCTACCTGCTGAACAAACAGGACTTCC
TGTTCAACCAAGACAACAAGGACAAGCTGAAGGAGTATTTGAAGTTTCATGTGATACGAGATGCCAAGGTTTT
AGCTGTGGATCTTCCCACATCCACTGCCTGGAAGACCCTGCAAGGTTCAGAGCTGAGTGTGAAATGTGGAGCT
GGCAGGGACATCCGTGACCTCTTTCTGAATGGCCAAAQCTACAGAATTGTGCAGCGGGAGCTCTTGTTTGACC
TGGGTGTGGCCTACGGCATTGACTGTCTGCTGATTGATCCCACCCTGGGGGGCCGCTGTGACACCTTTACTAC
TTTCGATGCCTCGGGGGAGTGTGGGAGCTGTGTCAATACTCCCAGCTGCCCAGGTGGAGTAAACCAAAGGGGT
GTGAAGCAGAAGTGTCTCTACAACCTGCCTTCAAGAGGAACCTGGAAGGCTGCCCGGGAGCGGTGCAGCCTGG
TTATACAGATCCCCAGGTGCTGCAAGGGCTACTTCGGGCGAGACTGTCAGGCCTGCCCTGGAGGACCAGATGC
CCCGTGTAATAACCGGGGGTGTCTGCCTTGATCAGTACTCGGCCACCGGAGAGTGTAATGCAACACCGGCTTC
AATGGGACGGCGTGTGAGATGTGCTGGCCGGGGAGATTTGGGCCTGATTGTCTGCCCTGTGGCTGCTCAGACC
ACGGACAGTGCGATGATGGCATCACGGGCTCCGGGCAGTGCCTCTGTGAACGGGGTGGACAGGCCCCCTCGTG
TGACACTCAGGCAQTTTTGCCTGCAGTGTGTACGCCTCCTTGTTCTGCTCATGCCACCTGTAAGGAGAACAAC
ACGTGTGAGTGTAACCTGGATTATGAAGGTGACGGAATCACATGCACAGTTGTGGATTTCTGCAAACAGGACA
ACGGGGGCTGTGCAAAGGTGGCCAGATGCTCCCAGAAGGGCACGAAGGTCTCCTGCAGCTGCCAGAAGGGATA
CAAAGGGGACGGGCACAGCTGCACGAGATAGACCCCTGTGCAGACGGCCTTAAACGGAGGGTGTCACGAGCAC
GCCACCTGTAAGATGACAGGCCCGGGCAAGCACAAGTGTGAGTGTAAAAGTCACTATGTCGGAGATGGGCTGA
ACTGTGAGCCGGAGCAGCTGCCCATTGACCGCTGCTTACAGGACAATGGGCAGTGCCATGCAGACCCAAAATG
TGTCGACCTCCACTTCCAGGATACCACTGTTGGGGTGTTCAATCTACGCTCCCAACTGGGCCAGTATAAGCTG
ACCTTTGACAAAGCCAGAGAGGCCTGTGCCAACGAAGCTGCGACAATGGCAACCTACAACCAGCTCTCCTATG
CCCAGAAGGCCAAGTACCACCTGTGCTCAGCAGGCTGGCTGGAGACCGGGCGGGTTGCCTACCCCACAGCCTT
CGCCTCCCAGAACTGTGGCTCTGGTGTGGTTGGGATAGTGGACTATGGACCTAGACCCAACAAGAGTGAAATG
TGGGATGTCTTCTGCTATCGGATGAAGGAAGTGCTGGCCTATTCCAACAGCTCAGCTCGAGGCCGGTGCATTT
CTAGAACACCTGACTGACCTGTCCATCCGCGGCACCCTCTTTGTGCCACAGAACAGTGGGCTGGGGGAGAATG
AGACCTTGTCTGGGCGGGACATCGAGCACCACCTCGCCAATGTCAGCATGTTTTTTCTAAATGACCTTGTCAA
TGGCACCACCCTGCAAACGAGGCTGGGAAGCAAAGCTGCTCATCACTGCCAGCCAGGACCCACTCCAACCGACG
GAGACCAGGTTTGTTGATGGAAGAGCCATTCTGCAGTGGGACATCTTTGCCTCCAATGGGATCATTCATGTCA
TTTCCAGGCCTTTAAAAGCACCCCCTGCCCCCGTGACCTTGACCCACACTGGCTTGGGAGGAAGGGATCTTC
TGCCATCATCCTGGTGACTGGGGCTGTTGCCTTGGCTGCTTACTCCTACTTTCGGATAAACCGGAGAACAA
GGCTTCCAGCATTTTGAGTCGGAAGAGGACATTAATGTTGCAGCTCTTGGCAAGCAGCAGCCTGAGAATATCT
CGAACCCCTTGTATGAGAGCACAACCTCAGCTCCCCCAGAACCTTCCTACGACCCCTTCACGGACTCTGAAG
ACGGCAGCTTGAGGGCAATGACCCCTTGAGGACACTGTGAGGGCCTGGACGGGAGATGCAAGCCATCACTCAC
TGCCACCTGGGCCATCAACTGTGAATTCTCAGCACCAGTTGCCTTTTAGGAACGTAAAGTCCTTTAAGCACTC
AGAAGCCATACCTCATCTCTCTGGCTGATCTGGGGGTTGTTTCTGTGGGTGAGAGATGTGTTGCTGTGCCCCAC
CCAGTACAGCTTCCTCCTCTGACCCTTTGGCTCTTCTTCCTTTGTACTCTTCAGCTGGCACCTGCTCCATTCT
GCCCTACATGATGGGTAACTGTGATCTTTCTTCCCTGTTAGATTGTAAGCCTCCGTGTCTTGTATCCCAGCCCC
TAGCCCAGTGCCTGACACAGGAACTGTGCACAATAAAGGTTTATGGAACAGAAACAAAGTCAAAAAAAAAAAAA
AAAAAAAAAAAAAAAAAAAAAAAAAC

The sequence of NOV1b was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel stabilin-like gene were obtained by SeqCallingTM Technology and are reported here as NOV1b. These methods used to amplify NOV1b cDNA are described in Example 2.

The NOV1b polypeptide (SEQ ID NO: 4) encoded by SEQ ID NO: 3 is 2420 amino acid residues in length and is presented using the one-letter amino acid code in Table 1D. The SignalP, Psort and/or Hydropathy results predict that NOV1b has no known signal peptide and is likely to be localized in the cytoplasm with a certainty of 0.4500. In alternative embodiments, a NOV1b polypeptide is located to the microbody (peroxisome) with a certainty of 0.3000, the mitochondrial matrix space with a certainty of 0.1000, or the lysosome (lumen) with a certainty of 0.1000.

TABLE 1D
Encoded NOV1b Protein Sequence

(SEQ ID NO:4)

MNEMERQETGNSKTRYHATAIVQAKHDKGLNKNGTSGDEEQKIKVGDRDRENKGFDGLLDVWNTLNFIHPCFAV
CNCVHGVCNSGLDGDGTCECYSAYTGPKCDKLTENFHTSHLTLWPVHDSKHWGSLRHQNAAGTCSSGGGKGDPD
VYQNGLIFHGGGTSGGLSSSRNRRSSVKRPEKWKGDDRDGGGKEGQQRRRADTESSLQRGHIKTPLPHRQGEAA
ITETTGNCVSAGMTGTNANAHTKVHPTVQSLTEYDSFQTHSTSRLKEFEKQQVKERFSDPPLMQAIKPSHEKYPP
YAQRKGTSLSPKTQGHGDDEQAALSFLHSITLSLYLYPTTFFHDSPVFIKPGIKTLRLAAFFGSSFPYEGSSVI
XXMGIEVWKNWCQNADTLAAAPAPSLNVQPCSAQKIPDVRLPLKMKTNWNANAFPITEAMANTATPSIHVYEKS
ATLMLIVRTWDQIGTVVHAKKATVGMAKCACLWTPAKLTETALQSLQCANMMGLDRCICQKGYVGDGLTCYGN
IMERLRELNTEPRGKWQGRLTSFISLLESIQIVSVQLSEFSQREPTCVNTKSIASNLEGPLVPLSNHYPLQVNE
LLVDNKAAQYFVKLHIIAGQMNIEYMNNTDMFYTLTGKSGEIFNSDKDNQIKLKLHGGKKKVKIIQGDIIASNG
LLHILDRAMDKLEPTFESNNEETNLGHALDEDGVGGPYTIFVPNNEALNNMKDGTLDYLLSPELEVATLISTPH
IRSMANQLIQFNTTDNGQILDVAAEEIEITAAAGRIYTLTGAALIPPSIVPILPHRCDETKREMKAAGTCVSCS
LVYWSRCPANSEPTALFTHRCVYSGRFGSLKSGCARYCNATVKCADSLGGNGTCICEEGFQGSQCQFCSDPNKY
GPRCNKKCLCVHGTCNNRIDSDGACLTGTCRDGSAGRLCDKQTSACGPYVQFCHIHATCEYSNGTASCICKAGY
EGDGTLCSEMDPCTGLTPGGCSRNAECIKTGTGTHTCVCQQGWTGNGRDCSEINNCLLPSAGGCHDNASCLYVG
PGQNECECKKGFRGNGIDCEPITSCLEQTGKCHPLASCQSTSSGVWSCVCQEGYEGDGFLCYGNAAVELSFLSE
AAIFNRWINNASLQPTLSATSNLTVLVPSQQATEDMDQDEKSFWLSQSNIPALIKYHMLLGTYRVADLQTLSSS
DMLATSLQGNFLHLAKVDGNITIEGASIVDGDNAATNGVIHIINKVLVPQRRLTGSLPNLMRLEGMPDYSIFR
GYTIQYNLANAIEAADAYTVFAPNNNAIENYTREKVLSLEEDVLRYVLEEAALAALAAGAARETMLGPSY
FLSFFLHDQLYNEAPINYTNATDKGVIHGLGAALEIQAACDAADTTIIRGRCRTCSSELTCPFGTKSLGN
EKRRCIYTSYFMGRRTLFIGCQPKCAAAATTRECCAGFFGPQCQPCPGNAQAACFGNGICLDGAAGTGVCECG
GFSGTACETCTEGKYGThCDQACSCAAGRCNQGPLGDGSCDCDVGWRGAACDNATTEDNCNGTCHTSAACLTNS
DGTASCKCAAGFQGNGTICTAINACEISNGGCSAKADCKRTTPGRRVCTCKJAAGYTGDGIVCLEINPCLEAAGGC
DKNAECTQTGPNQAACNCLPAYTGDGKVCTLINVCLTKNGGCSEFAICNHTGQVERTCTCKPNYIGDGFTCRGS
IYQELPKNPKTSQYFFQLQEHFVKDLVGPGPFTVFAPLSAAFDEEARVKDWDKYGLMPQVLRYHVVACHQLLLE
NLKLISNATSLQGEPIVISVSQSTVYINNKAKIISSDIISTNGIVHIIDKLLSPKNLLITPKDNSGRILQNLTT
LATNNGYIKFSNLIQDSGLLSVITDPIHTPVTLFWPTDQALHALHALPAEQQDFLFNQDNKDKLKEYLKFHVIR
DAKVLAVDLPTSTAWKTLQGSELSVKCGAGPAAIGDLFLNCQTYRIVQRELLFDLGVAYGIDCLLIDPTLGRCD
TFTTFDASGECGSCVNTPSCPRWSKPKGVKQKCLYNLPFKRNLEGCRERCSLVIQIPRCCKGFGRDCQACPGG
PDAPCNNRGVCLDQYSATGECKCNTGFNGTACEMCWPGRFGPDCLPCGCSDHGQCDDGITGSGQCLCETGWTG
SCDTQAVLPAVCTPPCSAHATCKENNTCECNLDYEGDGITCTVVDFCKQDNGGCAKVARCSQKGTKVSCSCQKG
YKGDGHSCTEIDPCADGLNGGCHEHATCKMTGPGKHKCECKSHYVGDGLNCEPEQLPIDRCLQDNGQCHADAKC
VDLHFQDTTVGVFHLRSPLGQYKLTFDKAREACANEAATMATYNQLSYAQKAKYHLCSAGWLETGRVAYPTAFA
SQNCGSGVVGIVDYGPRPNKSEMWDVFCYRMKGSAGLFQQLSSRPCISRTPD

NOV1c

A NOV1 variant includes NOV1c (alternatively referred to as CG 50736-09), which includes the 3260 nucleotide sequence (SEQ ID NO: 210) shown in Table 1E.

TABLE 1E
NOV1C NuCleotide Sequence

(SEQ ID NO:210)

GGCACGAGCAGGAGCTTCCCAAGAACCCGAAAAACTTCCCAGTATTTCTTCCAGTTGCAGGAGCATTTCGTGAA
AGATCTGGTCGGCCCAGGCCCCCTTCACTGTTTTTGCACCTTTATCTGCAGCCTTTGATGAGGAAGCTCGGGTT
AAAGACTGGGACAAATACGGTTTAATGCCCCAGGTTCTTCGGTACCATGTGGTCGCCTGCCACCAGCTGCTTC
TGGAAACCTGAATTGATCTCAATGCTACTTCCCTCCAAGGAGAGCCAAAAAATAGTCATCTCCGTCTCTCAGAG
CACGGGTGTATATAAATAATAAGGCTAAGATCATATCCAGTGATATCATCAGTACTAATGGGATTGTTCATATC
ATAGACAAATTGCTATCTCCCAAAATTTGCTTATCACTCCCAAAGACAACTCTGGAAGAATTCTGCAAAATC
TTACGACTTTGGCAACAAACAATGGCTACATCAAATTTAGAACTTAATACAGGACTCAGGTTTGCTGAGTGT
CATCACCGATCCCATCCACACCCCAGTCACTCTCTTCTGGCCCACCGACCAAGCCCTCCATGCCCTACCTGCT
GAACAACAGGACTTCCTGTTCAACCAAGACAACAAGGACAAGCTGAAGGAGTATTTGAAGTTTCATGTGATAC
GAGATGCCAAGGTTTTAGCTGTGGATCTTCCCACATCCACTGCCTGGAAGACCCTGCAAGGTTCAGAGCTGAG
TGTGAATGTGGAGCTGGCAGGGACATCGGTGACCTCTTTCTGAATGGCCAACCTGCAGAATTGTGCAGCAGG
GAGCTCTTGTTTGACCTGGGTGTGGCCTACGGCATTGACTGTCTGCTGATTGATCCCACCCTGGGGGGCCGCT
GTGACACCTTTACTACTTTCGATGCCTCGGGGGAGTGTGGGAGCTGTGTCAATACTCCCAGCTGCCCAAGGTG
GAGTAAACCAAAGGGTGTGAAGCAGAAGTGTCTCTACAACCTGCCCTTCAAGAGGAACCTGGAAGGCTGCCGG
GAGCGGTGCAGCCTGGTGATACAGATCCCAAGGTGCTGCAAGGGCTACTTCGGGCGAGACTGTCAGGCCTGCC
CTGGAGGACCAGATGCCCCGTGTAATAACCGGGGTGTCTGCCTTGATCAGTACTCGGCCACCGGAGAGTGTAA
ATGCAACACCGGCTTCAAATGGGACGGCGTGTGAGATGTGCTGGCCGGGGAGATTCGGGCCTGATTGTCTGCCC
TGTGGCTGCTCAGACCACGGACAGTGCGATGATGGCATCACGGGCTCCGGGCAGTGCCTCTGTGAAAACGGGGT
GGACAGGCCCCCTCGTGTGACACTCAGGCAGTTTTGCCTGCAGTGTGTACGCCTCCTTGTTCTGCTCATGCCAC
CTGTAAGGAGAACAACACGTGTGAGTGTAACCTGGATTATGAAGGTGACGGGAATCACATGCACAGTTGTGGAT
TTCTGCAAACAGGACAACGGGGGGCTGTGCAAAGGTGGCCAGATGCTCCCAGAAGGGCACGAAGGTCTCCTGCA
GCTGCCAGAAGGGATACAAAAGGGGACGGGCACAGCTGCACAGAGATAGACCCCTGTGCAGACGGCCTTAACGG
AGGGTGTCACGAGCACGCCACCTGTAAGATGACAGGCCCGGGGCAAGCACAAGTGTGAGTGTAAAAGTCACTAT
GTCGGAGATGGGCTGAACTGTGAGCCGGAGAAGCTGCCCCATTGACCGCTGCTTACAGGACAATGGGCAGTGCC
ATGCAGACGCCAAAATGTGTCGACCTCCACTTCCAGGATACCACTGTTGGGGTGTTCCATCTACGCTCCCCACT
GGGCCAGTATAGCTGACCTTTGACAAGCCAGGAGAGGCCTGTGCAACAACGAAGCTGCGACCATGGCAACCTAC
AACCAGCTCTCCTATGCCCAGAAAAGGCCAAGTACCACCTGTGCTCAGCAGGCTGGCTGGAGACCGGGCGGTTG
CCTACCCCACAGCCTTCGCCTCCCAGAACTGTGGCTCTGGTGTGGTTGGGATAGTGGACTATGGACCTAGACC
CAACAAGAGTGAAATGTGGGATGTCTTCTGCTATCGGATGAAAGATGTGAACTGCACCTGCAAGGTGGGCTAT
GTGGGAGATGGCTTCTCATGCAGTGGGAAACCTGCTGCAGGTCCTGATGTCCTTCCCCTCACTAACAACTTCC
TGACGGAAGTGCTGGCCTATTCCAACAGCTCAGCTCGAGGCCGTGCATTTCTAGAACACCTGACTGACCTG
CATCCGCGGCACCCTCTTTGTGCCACAGAACAGTGGGCTGGGGGAGAATGAGACCTTGTCTGGGCGGGACATC
GAGCACCACCTCGCCAATGTCAGCATGTTTTTCTACAATGACCTTGTCAATGGCACCACCCTGCAACGAGGG
TGGGAAGCAGCTGCTCATCCTGCCAGCCAGGACCCACTCCAACCGACGGAGACCAGGTTTGTTGATGGAAG
AGCCATTCTGCAGTGGGACATCTTTGCCTCCAATGGGATCATTCATGTCATTTCCAGGCCTTTAAAAGCACC
CCTGCCCCCGTGACCTTGACCCACACTGGCTTGGGAGCAGGGATCTTCTTTGCCATCATCCTGGTGACT
CTGTTGCCTTGGCTGCTTACTCCTACTTTCGGATCCGGAGAACAATCGGCTTCCAGCATTTTGAGTCGGA
AGAGGACATTAATGTTGCAGCTCTTGGCAAGCAGCAGCCTGAGAATATCTCGAACCCCTTGTATGAGAGCACA
ACCTCAGCTCCCCCAGAACCTTCCTACGACCCCTTCACGGACTCTGAAGAAACGGCAGCTTGAGGGCAATGACC
CCTTGAGGACACTGTGAGGGCCTGGACGGGAGATGCAAGCCATCACTCACTGCCACCTGGGCCATCAACTG
AATTCTCAGCACCAGTTGCCTTTTAGGAAJAACGTGTCCTTTAAGCACTCAGAAGCCATACCTCATCTCTG
GCTGATCTGGGGGTTGTTTCTGTGGGTGAGAGATGTGTTGCTGTGCCCACCCAGTACAGCTTCCCTCTGAC
CCTTTGGCTCTTCTTCCTTTGTACTCTTCAGCTGGCACCTGCTCCATTCTGCCCTACATGATGGGTAAGTG
ATCTTTCTTCCCTGTTAGATTGTAAGCCTCCNTCTTTGTATCCCAGCCCCTAGCCCAGTGCCTGACAGGAA
CTGTGCACAAATAGGTTTATGGAACAGAAAAAAAAAAAAAAAAA

The NOV1c polypeptide (SEQ ID NO: 211) encoded by SEQ ID NO: 210 is 897 amino acid residues in length and is presented using the one letter amino acid code in Table 1F.

TABLE 1F
Encoded NOV1C Protein Sequence

(SEQ ID NO:211)

MPQVLRYHVVACHQLLLENLKLISNATSLQGEPIVISVSQSTVYINNKAKIISSDIISTNGIVHIIDKLLSPKN
LLITPKDNSGRILQNLTTLATNNGYIKFSAAIQDSGLLSVITDPIHTPVTLFWPTDQALAALPAAQQDFLFNQD
NKDKLKEYLKFHVIRDAKVLAVDLPTSTAWKTLQGSELSVKCGAGRDIGDLFLNGQTCRIVQRELLFDLGVAYG
IDCLLIDPTLGGRCDTFTTFDASGECGSCVNTPSCPRWSKPKGVKQKCLAALPFAANLEGCRERCSLVIQIPRC
CKGYFGRDCQACPGGPDAPCNNRGVCLDQYSATGECKCNTGFNGTACEMCWPGRFGPDCLPCGCSDHGQCDDGI
TGSGQCLCETGWTGPSCDTQAVLPAVCTPPCSAHATCKENNTCECNLDYEGDGITCTVVDFCKQDNGGCAKVAR
CSQKGTKVSCSCQKGYKGDGHSCTEIDPCADGLNGGCHEHATCKMTGPGKHKCECKSHYVGDGLNCEPEQLPID
RCLQDNGQCHADAKCVDLHFQDTTVGVFHLRSPLGQYKLTFDKAREACANEAATMATYNQLSYAQKAKYHLCSA
GWLETGRVAYPTAFASQNCGSGVVGIVDYGPRPNKSEMWDVFCYAACTCKAAJGAAAAGDGFSCSGNLLQVLMS
FPSLTNFLTEVLAYSNSSARGRAFLEHLTDLSIRGTLFVPQAASGLGENETLSGPLIEHHAAVSMFFYAALAAG
TTLQTRVGSKLLITASQDPLQPTETRFVDGRAILQWDIFASNGIIHVISRPLKAPPAPVTLTHTGLGAGIFFAI
ILVTGAVALAAYSYFRINRRTIGFQHFESEEDINVAALGKQQPENISNPLYESTTSAPPEPSYDPFTDSEERQL
EGNDPLRTL

Searches of the sequence databases revealed that NOV1c has 99% homolgy to a CD44-like precursor FELL-like protein. Included in the invention are variants of the parent clone NOV1c as shown below in Table 1G. These novel variants were derived by laboratory cloning of cDNA fragments coding for a domain of the full length form of NOV1c (CG50736-09), between residues 85 and 636 (Fascilin domain). The cDNA coding for the variant sequences was cloned by the polymerase chain reaction (PCR). Primers were designed based on in silico predictions of the full length or some portion (one or more exons) of the cDNA/protein sequence of the invention, or by translated homology of the predicted exons to closely related human sequences or to sequences from other species. These primers and methods used to amplify the variant cDNA are described in Example 2.

TABLE 1G
Variants of NOV1c

| Nov1c | | | |
|---|---|---|---|
| Variant | Alternate | Change in SEQ ID | Change in SEQ |
| No. | Reference | NO: 210 | ID NO: 211 |
| 1 | 169487446 | T → C at bp 887; and | Q → L at aa 325 |
| | | A → T at bp 1144; | |
| 2 | 169487460 | C → T at bp 1034; and | No change |
| | | T → C at bp 1244; | |
| 3 | 169487473 | C → T at bp 1223; | N → Y at aa 416; and |
| | | A → T at bp 1416; and | C → R at aa 487 |
| | | T → C at bp 1629 | |
| 4 | 169487491 | G → A at bp 1534; and | S → N at aa 455 |
| | | A → G at bp 1547; | |
| 5 | 169487497 | A → G at bp 976; and | K → R at aa 269; and |
| | | G → A at bp 2010; | G → S at aa 614 |
| 6 | 169487533 | A → G at bp 832; | Y → C at aa 221 |
| | | C → T at bp 1223; and | |
| | | T → C at bp 2003 | |
| 7 | 169487538 | A → G at bp 513; and | I → V at aa 115; and |
| | | T → C at bp 1888; | M → T at aa 573 |
| 8 | 169487577 | G → T at bp 712 | No change |

SNP variants of NOV1 are disclosed in Example 3.

NOV1 Clones

Unless specifically addressed as NOV1a, NOV1b, NOV1c, or variants of NOV1c, any to NOV1 is assumed to encompass all variants.

The amino acid sequnce of NOV1 has high homology to other proteins as shown in Table 1H

TABLE 1H
BLASTX Results from Patp Database for NOV1

| | | | Smallest |
|---|---|---|---|
| | | High | Sum |
| Sequences Producing High-Scoring Segment Pairs: | | Score | Prob P (N) |
| patp: AAY93910 | A human hyaluronan-binding | 2493 | 1.2e−290 |
| | protein, designated WF-HABP | | |
| patp: AAY93913 | A human hyaluronan-binding | 848 | 1.9e−157 |
| | protein, designated BM-HABP | | |
| patp: AAB42164 | Human ORFX 0RF1928 | 1017 | 1.9e−138 |
| | polypeptide sequence | | |
| patp: AAY93911 | A human hyaluronan-binding | 536 | 6.1e−75 |
| | protein, designated WF-HABP | | |
| patp: AAR05222 | Antigen GX5401FL encoded by | 353 | 4.3e−54 |
| | Eimeria tenella genomic DNA | | |

In a search of sequence databases, it was found, for example, that the NOV1a nucleic acid sequence has 1593 of 2797 bases (56%) identical to a gb:GENBANK-ID:HSA275213|acc:AJ275213.1 mRNA from Homo sapiens (Homo sapiens mRNA for stabilin-1 (Stab1 gene)). Further, the full amino acid sequence of the disclosed NOV1a protein of the invention has 543 of 1391 amino acid residues (39%) identical to, and 760 of 1391 amino acid residues (54%) similar to, the 2570 amino acid residue ptnr:SPTREMBL-ACC:Q9NY15 protein from Homo sapiens (Human) (STABILIN-1).

In a similar search of sequence databses, it was found, for example, that the NOV1b nucleic acid sequence has 2654 of 2678 bases (99%) identical to a gb:GENBANK-ID:HSM801377|acc:AL133021.1 mRNA from Homo sapiens (Homo sapiens mRNA; cDNA DKFZp434E0321 (from clone DKFZp434E0321)). Further, the full amino acid sequence of the disclosed NOV1b protein of the invention has 638 of 642 amino acid residues (99%) identical to, and 638 of 642 amino acid residues (99%) similar to, the 897 amino acid residue ptnr:SPTREMBL-ACC:Q9NRY3 protein from Homo sapiens (Human) (CD44-LIKE PRECURSOR FELL).

Additional BLASTP results are shown in Table 1I.

TABLE 11
NOV1 BLASTP Results

| Gene Index/ | | Length of | | | |
|---|---|---|---|---|---|
| Identifier | Protein/Organism | aa | Identity (%) | Positives (%) | Expect Value |
| Q9UF98 | HYPOTHETICAL 115.7 | 1069 | 1038/1064 | 1042/1064 | 0.0 |
| | KDA PROTEIN - Homo | | (97%) | (97%) | |
| | sapiens (Human) | | | | |
| Q9H7H7 | FLJ00112 PROTEIN - | 1192 | 926/929 | 928/929 | 0.0 |
| | Homo sapiens (Human) | | (99%) | (99%) | |
| Q9NRY3 | CD44-LIKE PRECURSOR | 897 | 640/641 | 641/641 | 0.0 |
| | FELL - Homo sapiens | | (99%) | (100%) | |
| | (Human) | | | | |
| Q9NY15 | STABILIN-1 - Homo | 2570 | 543/1391 | 760/1391 | 0.0 |
| | sapiens (Human) | | (39%) | (54%) | |
| Q93072 | MYELOBLAST | 2212 | 614/1740 | 897/1740 | 0.0 |
| | KIAA0246 PROTEIN — | | (35%) | (51%) | |
| | Homo sapiens (Human) | | | | |

A multiple sequence alignment is given in Table 1J, with the NOV1a and NOV1b proteins of the invention being shown in lines 1 and 2 , in a ClustalW analysis comparing NOV1 with related protein sequences of Table 1I.

[image]

[image]

[image]

[image]

[image]

[image]

[image]

[image]

Domain results for NOV1 were collected from the Pfam database, and then identified by the InterPro domain accession number. The results are listed in Table 1K with the statistics and domain description. These results indicatee that the NOV1 polypeptides have properties similar to those of other proteins known to contain these domains.

TABLE 1K

Domain Analysis of NOV1

| | | |
|---|---|---|
| | Score | E |
| PSSMs Producing Significant Alignments | (bits) | Value |

| | | |
|---|---|---|
| FasCiclin; domain 3 of 4, from 1756 to 1886 | 53.1 | 6.3e−12 |

| | | |
|---|---|---|
| Fasciclin | agtvmeklktdprfstlvaaleaadLvetlnnsgdfTVFAPTNdAFq | (SEQ ID NO:44) |
| | +++ + ++ +\| +++ +++++ +++++\|\|\|\|\| +\|\|+ | |
| NOV1a | RGSIYQElPKNPKTSQYFFQlQEH-FVKDlVGPGPFTVFAPlSAAFD | (SEQ ID NO:2) |
| | klpagdlktldeLlnkedakqLakILtYH.Vvagklstadllslstpvlt | |
| | +++ +++ + ++\| \|\| \|+ +++ ++ ++ + | |
| NOV1a | E-EAR---VKDWDKY----GLMPQVLRYHvVACHQLLLENLKLLSN--AT | |
| | slqGskitvsgkndtellkdvnvlkVnnatvivesDiettNGviHViDrV | |
| | +++\|+++ ++++ + + ++ + ++++\|+ ++\|\|++\|++\|++ | |
| NOV1a | SLQGEPIVISVSQST--------VYINNKAKIISSDIISTNGIVHIIDKL | |
| | LlP | |
| | \| \| | |
| NOV1a | LSP | |

| | | |
|---|---|---|
| Fasciclin: domain 4 of 4, from 1900 to 2043 | 41.9 | 1.5e−08 |

| | | |
|---|---|---|
| Fasciclin | agtvmekiktdprtStlvaaleaadLvetlnnsg..dfTVFAPTNdA | (SEQ ID NO:45) |
| | +++++++ +++ +++ ++\|+++ +++ ++++\|+\| \|\|+ \| | |
| NOV1a | ILQNLTTLATNNGYIKFSNLIQDSGLLSVITDPIhtPVTlFWPTDQA | (SEQ ID NO: 2) |
| | FqkLpagdlktldeLlnkedakqLakILtYHVvagklstadllslstpvl | |
| | + +\|+++ ++ \|++++++++\|++ \|++\|\|+ + ++ + +++++ | |
| NOV1a | LHAlPAE---QQDFLFNQDNKDKLKEYLKFHVIRDAKVLAVDLPTSTA-W | |
| | tslqGskitvsgkndtellkdvnvlkVnnat.vivesDiettNGviHViD | |
| | ++++\|+++++++ ++ ++ + ++ + +++ + + \|+ + +\| | |
| NOV1a | KTLQGSELSVKCGAGR----DIGDLFLNGQTCRIVQRELLFDLGVAYGID | |
| | rVLlP | |
| | +\| | |
| NOV1a | CLLID | |

| | | |
|---|---|---|
| Xlink: domain 1 of 1, from 2358 to 2450 | 100.8 | 4.1e−43 |

| | | |
|---|---|---|
| Xlink | GeVFhyrapsgRYkltFeEAqaaCirqgAriATtgQlyAAwkgafdq | (SEQ ID NO:46) |
| | +\|\|+++++ + \|+++\|+ \|+++\|+++ \|+ \|\|+ \|\| \| ++ ++ | |
| NOV1a | -GVFHLRSPLGQYKLTFDKAREACANEAATMATYNQLSYAQKAKYHL | (SEQ ID NO:2) |
| | CdAGWLADgsVRYPIvkPRenCgGdkdgfpGVRtyYlfpNQTGfpddpss | |
| | \|+\|\|\|\| ++\| \|\| ++++\|+ + \|+ ++ \| + ++++ | |
| NOV1a | CSAGWLETGRVAYPTAFASQNCGSGV---VGIVDY-------GPRPNKSE | |
| | rYDvYCF | |
| | +\|++\|+ | |
| NOV1a | MWDVFCY | |

The NOV1 proteins disclosed in this invention is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, Public EST sources, Literature sources, and/or RACE sources.

The protein similarity information, expression pattern, cellular localization, and map location for the NOV1 proteins and nucleic acids disclosed herein suggest that this Stabilin-like protein may have important structural and/or physiological functions characteristic of the Stabilin and/or epidermal growth factor (EGF) families. Therefore, the nucleic acids and proteins of the invention are useful in potential diagnostic and therapeutic applications. For example, the compositions of the present invention will have efficacy for treatment of patients suffering from: heart diseases (particularly mechanisms of angiogenesis), cancers such as, for example, erythroid-megakaryocytic leukaemia, breast cancer, fibrosarcoma, neoplasia, such as T-cell acute lymphoblastic leukemia/lymphoma and mammary carcinomas, chronic contact dermatitis, familial and congenital cholestatic diseases, Hereditary vascular dementia, neurological diseases, CNS disorders, autoimmune disease, inflammation, immunodeficiencies, systemic lupus erythematosus, metabolic disorders (obesity and/or diabetes), asthma, emphysema, scleroderma, allergies, and other diseases, disorders and conditions of the like.

The novel nucleic acid encoding the Stabilin/Fascilin-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 45 to 125. In another embodiment, a contemplated NOV1 epitope is from about amino acids 200 to 375. In other specific embodiments, contemplated NOV1 epitopes are from about amino acids 400 to 2700.

NOV2

Another NOVX protein of the invention, referred to herein as NOV2, includes two novel polydom-like proteins. The disclosed proteins have been named NOV2a and NOV2b. Polydom-like proteins are important for the regulation of hematopoiesis and may play a role in cell adhesion or in the immune system. Domains within this protein have been shown to be important in coagulation, growth, cell division, and other important cellular processes.

Although some members of the polydom-like protein family may be localized in the lysosome, the protein predicted here is similar to the mouse polydom protein which is localized extracellularly. Therefore, it is likely that this polydom-like protein is available at the same localization, and hence accessible to a diagnostic probe, and for the various therapeutic applications described herein.

The NOV2a and NOV2b proteins disclosed in this invention map to chromosome 9. This information was assigned using OMIM, the electronic northern bioinformatic tool implemented by CuraGen Corporation, public ESTs, public literature references and/or genomic clone homologies.

NOV2a

In one embodiment, a NOV2 variant is NOV2a (alternatively referred to herein as CG142106342), which encodes a novel polydom-like protein and includes the 11158 nucleotide sequence (SEQ ID NO: 5) shown in Table 2A. An open reading frame for the mature protein was identified beginning with an ATG codon at nucleotides 77-79 and ending with a TAA codon at nucleotides 10787-10789. Putative untranslated regions downstream from the termination codon and upstream from the initiation codon are underlined in Table 2A, and the start and stop codons are in bold letters.

TABLE 2A
NOV2a Nucleotide Sequence

(SEQ ID NO:5)

CAATTGGTCTAGGGTCTCCCCCATTGGAATATCCATCAGTGATGAGAATACAACGTTTGTTGAGTTTTCTCTAGC
ATGAGAAGAATTTGCGCGGCTTGCTGGGGTCTGGCGCTCGTTTCGGGCTGGGCGACCTTTCAGCAGATGTCCCCGT
CGCGCAATTTCAGCTTCCGCCTCTTCCCCGAGACCGCGCCCGGGGCCCCCGGGAGTATCCCCGCGCCGCCCGCTCC
TGGCGACGAAGCGGCGGGGAGCAGAGTGGAGCGGCTGGGCCAGGCGTTCCGCGTGCGGCTGCTGCGGGAGCTCAGC
GAGCGCCTGGAGCTTGTCTTCCTGGTGGATGATTCGTCCAGCGTGGGCGAAGTCAACTTCCGCAQCGAGCTCATGT
TCGTCCGCAAGCTGCTGTCCGACTTCCCCGTGGTGCCCACGGCCACGCGCGTGGCCATCGTGACCTTCTCGTCCAA
GACTACGTGGTGCCGCCGTCGATTACATCTCCACCCGCCGCGCGCGCAAGCACAAAAGTGCGCGCTGCTCCTCCAA
GAGATCCCTGCCATCTCCTACCGAGGTGGCGGCACCTACACCAAGGGCGCCTTCCAGCAAGCCGCGAAAATTCTTC
TTCATGCTAGAGAACTCAACAAAAGTTGTATTTCTCATCACTGATGGATATTCCAAAATGGGGGAGACCCTAGACC
AATTGCAGCGTCACTGCGAGATTCAGGAGTGGAGATCTTCACTTTTGGCATATGGCAAGGGAACATTCGAGAGCTG
AATGACATGGCTTCCACCCCAAGGAGGAGCACTGTTACCTGCTACACAGTTTTGAAGAATTTGAGGCTTTTAGTCG
CCCTCTGTCATATGTTATTTGTAGATCTACCTTCTGGGAGTTTTATTCAAGATGATATGGTCCACTGCTAATATCT
TTGTGATGAAGGCAAGGACTGCTGTGACCGAATGGGAAGCTCAAATGTGGGGAAACACACAGGCCATTTTGAGTGC
ATCTGTGAAGGGGTATAACGGGAAAGGTAACTGCAGTATGACTGCACAGTTTGCCCATCGGTGGACATACAACCTG
AAGGCTCACCAGGAGGAATCAGCAGTTGCATTCCATGTCCTGATGAATCACACCTCTCCACCTGGAAGCACATC
CCCTGAAAGACTGTGTCTGCAGAGAGTACAGGGCATCTGGCCAGACCTGTGAAAAGTTGTCCACTGCCCTGCCCTG
AAGCCTCCCGAAAATGGTTACTTTATCCAAAACACTTGCAACAACCACTTCAATGCAGCCTGTGGGGTCCGATGTC
ACCCTGGATTTGATCTTGTGGGAAGCAGCATCATCTTATGTCTACCCAATGGTTTGTGGTCCGGTTCAGACAGCTA
CTGCAGAGTAAGAACATGTCCTCATCTCCGCCAGCCGAACATGGCCACATCAGCTGTTCTACAAGGGAATGTTA
TATAAGACAACATGTTTGGTTGCCTGTGATGAAGGGTACAGGCTAGAAGGCAGTGATAAGCTTACTTGTCAAGGAA
ACAGCCAGTGGGATGGGCCAGAACCCCGGTGTGTGGAGCGCCACTGTTCCACCTTTCAGATGCCCAAGATGTCAT
CATATCCCCCCACAACTGTGGCAAGCAGCCAGCCAATTTGCGACGGATCTGCTATGTAAGTTGCCGCAAGGGTTC
ATTTTATCTGGAGTCAAGAATGCTGAGATGTACCACTTCTGGAAAATGGAAATGTCGGAGTTCAGGCAGCTGTGT
GTAAAGACGTGGAGGCTCCTCAATCAACTGTCCTAAGGACATAGAGGCTAAGACTCTGGAACAGCAAGATTCTGC
CAATGTTACCTGGCAGATTCCAACAGCTAAAGACAACTCTGGTGAAAGGTGTCAGTCCACGTTCATCCAGCTTTC
ACCCCACCTTACCTTTTCCCAATTGGAGATGTTGCTATCGTATACACGGAACTGACCTATCCGGCAACCAGGCCA
GCTGCATTTTCCATATCAAGGTTATTGATGCAGAACCACCTGTAATAGACTGGTGCAGATCTCCACCTCCCGTCAA
GGTCTCGGAGAAGGTACATGCCGCAAGCTGGGATGAGCCTCAGTTCTCAGACAACTCAGGTGCTGAATTGGTCA
ACCAGAiAAGTCATACACAAGGAGACCTTTTCCCTCAAGGGGAGACTATAGTACAGTATACAGCCACTGACCCC
GCAATAACAGGACATGTGATATCCATATTGTCATGGTTCTCCCTGTGAAJAATTCCATTCACACCTGTAATGG
GGATTTTATATGCACTCCAGATAATACTGGAGTCAACTGTACATTAACTTGCTTGGAGGGCTATGATTTCACAGAA
GGGTCTACTGACAAGTATTATTGTGCTTATGAAGATGGCGTCTGGAAACCAACATATACCACTGAATGGCCAGACT
GTGCCAGTAAAGCGTTTTGCACCACGGGTTAAGTCCTTTGAGATGTTCTACAAAAAGCAGCTCGTTGTGATGACAC
AGATCTGATGAAGAAAGTTTTCTGAAGCATTTGAGACGACCCTGGGAAAATGGTCCCATCATTTTGTAGTGATGCA
GAGGACATTGACTGCAGACTGGAGGAGAACCTGACCAAAAAATATTGCCTAGAATATAATTATGACTATGAAAATG
GCTTTGCAATTGGTCCAGGTGGCTGGGGTGCAGCTAATAGGCTGGATTACTCTTACGATGACTTCCTGGACTGT
GCAAGAAACAGCCACAAGCATCGGCAATGCCAAGTCCTCACGGATTAAAAGAAGTGCCCCATTATCTGACTATAAA
ATTAAGTTAATTTTTTAACATCACAGCTAGTGTGCCATTACCCGATGAAAGAATGATACCCTTGAATGGGAAAATC
AGCAACGACTCCTTCAGACATTGGAAACTATCACAAATAAACTGAAAAGGACTCTCAACAAAGACCCCATGTATTC
CTTTCAGCTTGCATCAGAAATACTTATAGCCGACAGCAATTCATTAGAAACAAAAGGCTTCCCCCCCTTCTGCAGA
CCAGGCTCAGTGCTGAGAGGGCGTATGTGTGTCAATTGCCCTTTGGGAACCTATTATAATCTGGAACATTTCACCT
GTGAAAGCTGCCGGATCGGATCCTATCAAGATGAAGAAGGGCAACTTGAGTGCAAGCTTTGCCCCTCTGGGATGTA
CACGGAATATATCCATTCAAGAAACATCTCTGATTGTAGCTTCAGTGTAAAACAAGGCACCTACTCATACAGTGGA
CTTGAGACTTGTGAATCGTGTCCACTGGGCACTTATCAGCCAAAATTTGGTTCCCGGAGCTGCCTCTCGTGTCCAG
AAAACACCTCAACTGTGAAAAGAGGAGCCGTGAACATTTCTGCATGTGGAGTTCCTTGTCCAGAAGGAAAATTCTC
GCGTTCTGGGTTAATGCCCTGTCACCCATGTCCTCGTGACTATTACCAACCTAATGCAGGGAAGGCCTTCTGCCTG
GCCTGTCCCCTTTTATGGAACTACCCATTCGCTGGTTCCAGATCCATCACAGAATGTTCAAGTTTTAGTTCAACTT
TCTCAGCGGCAGAGGAAGTGTGGTGCCCCCTGCCTCTCTTGGACATATTAAAAJAAGAGGCATGAATCAGCAGTCA
GGCAAGTCATGAATGCTTCTTTAACCCTTGCCACAATAGTGGAACCTGCCAGCAACTTGGGCGTGGTTATGTTTGT
CTCTGTCCACTTGGATATACAGGTTTAAAGTGTGAAACAGACATCGATGAGTGCAGCCCACTGCCTTGCCTCAACA
ATGGAGTTTGTAGACCTAGTTGGGGAATTCATTTGTGAGTGCCCCATCAGGTTACACAGGTTAAGCACTGTGAATT
GAACATCAATGAATGTCAGTCTAATCCATGTAGAAATCAGGCCACCTGTGTGGATGAATTAAATTCATACAGTTGT
AAATGTCAGCCAGGATTTTCAGGCAAAGGTGTGAAAACAGGTATGTATCAACTCAGTGTTATTAATAACCTTAATA
ATGCAGTCTGTGAAGACCAGGTTGGGGGATTCTTGTGCAAATGCCCACCTGGATTTTTGGGTACCCGATGTGGAA
GAACGTCGATGAGTGTCTCAGTCAGCCATGCAAATGGAGCTACCTGTAAGACGGTGCCAATAGCTTCAGGTGC
CTGTGTGCAGCTGGCTTCACAGGATCACACTGTGAATTGAACATCAATGAATGTCAGTCTAATCCATGTAGAAATC
AGGCCACCTGTGTGGATGAATTAAATTCATACAGTTGTAAATGTCAGCCAGGATTTTCAGGCAAAAGGTGTGAAAC
AGAACAGTCTACAGGCTTTAACCTGGATTTTGAAGTTTCTGGCATCTATGGATATGTCATGCTAGATGGCATGCTC
CCATCTCTCCATGCTCTAACCTGTACCTTCTGGATGTCCTCTGACGACATGAACTATGGAAACACGGAAATCTCCT
ATGCAGTTGATAACGGCAGCGACAATACCTTGCTCCTGACTGATTATAACGGGGTGGGTTCTTTATGAAATGGCAG
GGAAGATAACAACTGTCCCTCGGTGAATGATGGCAGATGGCATCATATTGCAATCACTTGGACAAAAAGTACTGGT
GGAGCCTGGAGGGTCTATATATGGGGAAJAATTATCTGACGGTGGTACTGGCCTCTCCATTGGCAAGCCATACCTG
GTGGCGGTGCATTAGTTCTTGGGCAAGAGCAAGACAAAAGGAGAGGGGTTCAACCCGGCTGAGTCTTTTTGTGGGG
CTCCATAAGCCAGCTCAACCTCTGGGACTATGTCCTGTCTCCACAGCAGGTGAAGTCACTGGCTACCTCCTGCCCA
GAGGAACTCAGTAAAGGAAACGTGTTAGCATTGGCCTGATTTCTTGTCAGGAATTGTGGGAAAGTGAAGATCGATT
CTAAGAGCATATTTTGTTCTGATTGCCCACGCTTGGGAGGGTCAGTGCCTAATCTGAGAACTGCATCTGAAGATTT
AAAACCAGGTTCAAAGTCAAATCTGTTCTGTGAACCAGGCTTCCAGCTGGTCGGGAACCCTGTGCAGTACTGTCTG
AATCAAGGACAGTGGACACAACCACTCCCCCACTGTGAACGCATTCGCTGTGGGGTGCCACCTCCTTTGGAGAATG
GCTTCCATTCAGCCGATGACTTCTATGCTGGCAGCAAAGTAACCTACCAGTGCAACAATGGCTACTATCTATTGGG
TGACTCAAGGATGTTCTGTACAGATAATGGGAGCTGGAACGGCGTTTCACCATCCTGCTTAGATGTCGATGAGTGT
GCAGTTGGATCAGATTGTAGTGAGCATGCTTCTTGCCTGAACGTAGATGGATCCTACATATGTTCATGTGTCCAAC
CGTACACAGGATGGGAAAAAACTGTGCAGAACCTATAAAATGTAAAGGCTCCAGGAAATCCGGAAAATGGCCACTC
CTCAGGTGAGATTTATACAGTAGGTGCCGAAGTCACATTTTCGTGTCAGGAAGGATACCAGTTGATGGGAGTAACC
AAAATCACATGTTTGGAGTCTGGAGAATGGAAATCATCTAATACCATATTGTAAGCTGTTTCATGTGGTAAACCGG
CTATTCCAGAAAATGGTTGCATTGAGGAGTTAGCATTTACTTTTGGCAGCAAAGTGACATATAGGTGTAATAAAGG
ATATACTCTGGCCGGTGATAAAGAATCATCCTGTCTTGCTAACAGTTCTTGGAGTCATTCCCCTCCTGTGTGTGAA
CCAGTGAAGTGTTCTAGTCCGGAAATATAATAATGGAAATATATTTTGAGTGGGCTTACCTACCTTTCTACTGGGA
CATCATATTCATGCGATACAGGATACAGCTTACAGGGCCCTTCCATTATTGAATGCACGGCTTCTGGCATCTGGGA
CAGAGCGCCACCTGCCTGTCACCTCGTCTTCTGTGGAGAACCACCTGCCATCAAGATGCTGTCATTACGGGGAAAT
AACTTCACTTTCAGGAACACCGTCACTTACACTTGCAAAGAAGGCTATACTCTTGCTGGTCTTGACACCATTGAAT
GCCTGGCCGACGGCAAGTGGAGTAGAAGTGACCAGCAGTGCCTGGCTGTCTCCTGTGATGAGCCACCAATTGTGGA
CCACGCCTCTCCAGAGACTGCCCATCGGCTCTTTGGAGACATTGCATTCTACTACTGCTCTGATGGTTACAGCCTA
GCAGACAATTCCCAGCTTCTCTGCAATGCCCAGGGCAAGTGGGTACCCCCAGAAGGTCAAGACATGCCCCGTTGTA
TAGCTCATTTCTGTGAAAAACCTCCATCGGTTTCCTATAGCATCTTGGAATCTGTGAGCAAAGCAAAATTTGCAGC
TGGCTCAGTTGTGAGCTTTAAATGCATGGAAAGGCTTTGTACTGAACACCTCAGCAAGATTGAATGTATGAGAGGT
GGGCAGTGGAACCCTTCCCCCATCTCAATCCAGTGCATCCCTGTGCGGTGTGGAGAGCCACCAAGCATCATGAATG
GCTATGCAAGTGGATCAAACTACAGTTTTGGAGCCATGGTGGCTTACAGCTGCAAAAGGGGGTTCTACATCAAAGG
GGAAAAGAAGAGCACCTGCGAAGCCACAGGGCAGTGGAGTAGTCCTATACCGACGTGCCACCCGGTATCTTGTGGT
GAACCACCTAAGGTTGAGAATGGCTTTTCTGGAGCATAAACTGGCAGGATCTTTGAGAGTGAAGTGAGGTATCAGT
GTAACCCGGGCTATAAGTCAGTCGGAAGTCCTGTATTTGTCTGCCAAGCCAATCGCCACTGGCACAGTGAATCCCC
TCTGATGTGTGTTCCTCGACTGTGGAAAAACCTCCCCCCGATCCAGAATGGCTTCATGAAAGGAGAAAACTTTGAA
GTAGGGTCCAAGGTTCAGTTTTTCTGTAATGAGGGTTATGAGCTTGTTGGTGACAGTTCTTGGACATGTCAGAAAT
CTGGCAAATGGAATAAGAAGTCAAATCCAAAGTGCATGCCTGCCAAGTGCCCAGAGCCGCCCCTCTTGGAAAACCA
GCTAGTATTAAAGGAGTTGACCACCGAGGTAGGAGTTGTGACATTTTCCTGTAAAGAAGGGCATGTCCTGCAAGGC
CCCTCTGTCCTGAATAGCTTGCCATCCCAGCAATGGAATGACTCTTTCCCTGTTTGTAAGATTGTTCTTTGTACCC
CACCTCCCCTAATTTCCTTTGGTGTCCCCATTCCTTCTTCTGCTCTTCATTTTGGAAGTACTGTCAAGTATTCTTG
TGTAGGTGGGTTTTTCCTAAGAGGAAATTCTACCACCCTCTGCCAACCTGATGGCACCTGGAGCTCTCCACTGCCA
GAATGTGTTCCAGTAGAATGTCCCCAACCTGAGGAAATCCCCAATGGAATCATTGATGTGCAAGGCCTTGCCTATC
TCAGCACAGCTCTCTATACCTGCAAGCCAGGCTTTGAATTGGTGGGAAATACTACCACCCTTTGTGGAGAAAATGG
TCACTGGCTTGGAAGGAAAACCAACATGTAAAGCCATGAGTGCCTGAAACCCAAGGAGATTTTGAATGGCAAATTC
TCTTACACGGACCTACACTATGGACAGACCGTTACCTACTCTTGCAACCGAGGCTTTCGGCTCGAAGGTCCCAGTG
CCTTGACCTGTTTAGAGACAGGTGATTGGGATGTAGATGCCCCATCTTGCAATGCCATCCACTGTGATTCCCCACA
ACCCATTGAAAATGGTTTTGTAGAAGGTGCAGATTACAGCTATGGTGCCATAATCATCTACAGTTGCTTCCCTGGG
TTTCAGGTGGCTGGTCATGCCATGCAGACCTGTGAAGAGTCAGGATGGTCAAGTTCCATCCCAACATGTATGCCAA
TAGACTGTGGCCTCCCTCCTCATATAGATTTTTGGAGACTGTACTAACTCAAGATGACCAGGGGATATTTTGAGCA
AGAAGACGACATGATGGAAGTTCCATATGTGACTCCTCACCCTCCTTATCATTTGGGAGCAGTGGCTAAAACCTGG
GAAAAATACAAGGAGTCTCCTGCTACACATTCATCAACTTTCTGTATGGTACAATGGTTTCATACACCTGTAATC
CAGGATATGAACTTCTGGGGAACCCTGTGCTGATCTGCCAGGAAGATGGAACTTGGAATGGCAGTGCACCATCCTG
CATTTCAATTGAATGTGACTTGCCTACTGCTCCTGAAAATGGCTTTTTGCGTTTTACAGAGACTAGCATGGGAAGT
GCTGTGCAGTATAGCTGTAAACCTGGACACATTCTAGCAGGCTCTGACTTAAGGCTTTGTCTAGAGAATAGAAAGT
GGAGTGGTGCCTCCCCACGCTGTGAAGCCATTTCATGCAAAAAGCCAAATCCAGTCATGAATGGATCCATCAAAGG
AAGCAACTACACATACCTGAGCACGTTGTACTATGAGTGTGACCCCGGATATGTGCTGAATGGCACTGAGAGGAGA
ACATGCCAGGATGACAAAAACTGGGATGAGGATGAGCCCATTTGAATTCCTGTGGACTGCAGTTCACCCCCAGTCT
CAGCCAATGGCCAGGTGAGGAGGAGACGAGTACACATTCCAAAAGAGATTGAATACACTTGCAATGAAGGGTTCTT
GCTTGAGGGAGCCAGGAGTCGGGTTTGTCTTGCCAAAATGGAGTTGGAGTGAGCCACTCCCGACTGTGTGCCTGTC
AGATGTGCCACCCCGCCACAACTGGCCAATGGGGTGACGGAAGGCCTGGACTATGGCTTCATGAAGGAAGTAACAT
TCCACTGTCACGAGGGCTACATCTTGCACGGTGCTCCAAAACTCACCTGTCAGTCAGATGGCAACTGGGATGCAGA
GATTCCTCTCTGTAAACCAGTCACTGTGGACCTCCTGAAGATCTTGCCCATGGTTTCCCTAATGGTTTTTTCCTTT
ATTCATGGGGGCCATATACAGTATCAGTGCTTTCCTGGTTATAAGCTCCATGGATTCATCAAGGGAAGGTGCCTCT
CCAATGGCTCCTGGAGTGGCAGCTCACCTTCCTGCCTGCCTTGCAGATGTTCCACACCAGTAATTGAATATGGAAC
TGTCAATGGGGACAGATTTTGACTGTGGAAGGCAGCCCGGATTCAGTGCTTCAAGGCTTCAAGCTCCTAGGACTT
TCTGAAATCACCTGTGAAGCCGATGGCCAGTGGAGCTCTGGGTTCCCCCACTGTGAACACACTTCTTGTGGTTCTC
TTCCAATGATACCAAATGCGTTCATCAGTGAGACCAGCTCTTGGAAGGAAAATGTGATAACTTACAGCTGCAGGTC
TGGATATGTCATACAAGGCAGTTCAGATCTGATTTGTACAGAGAAGGGGTATGGAGCCAGCCTTATCCAGTCTGT
GAGCCCTTGTCCTGTGGGTCCCCACCGTCTGTCGCCAATGCAGTGGCAACTGGAGAGGCAAACACCTATGAAGTG
AAGTGAACTCAGATGTCTGGAAGGTTATACGATGGATACAGATACAGATACATTCACCTGTCAGAAAAGATGGTCG
CTGGTTCCCTGAGAGAATCTCCTGCAGTCCTAAAAAATGTCCTCTCCCGGAAAACATAACACATATACTTGTACAT
GGGGACGATTTCAGTGTGAATAGGCAAGTTTCTGTGTCATGTGCAGAAGGGTATACCTTTGAGGGAGTTAACATAT
CAGTATGTCAGCTTGATGGAACCTGGGAGCCACCATTCTCCGATGAATCTTGCAGTCCAGTTTCTTGTGGGAAACC
TGAAAGTCCAGAACATGGATTTGTGGTTGGCAGTTAAATACACCTTTGAAGCACAATTATTTATCAGTGTGAGCCT
GGCTATGACTAGAGGGGGAACAGGGAACGTGTCTGCCAGGAGAACAGACAGTGGAGTGGAGGGGTGGCAATATGCA
AAGAGACCAGGTGTGAAACTCCACTTGAATTTCTCAATGGGAAAGCTGACATTGAAAACAGGACGACTGGACCCAA
CGTGGTATATTCCTGCCAAGAGGCTACAGTCTTGAAGGGCCATCTGAAGGAACACTGCACAGAAAATGGAACCTGG
AGCCACCCAGTCCCTCTCTGCAACCAAATCCATGCCCTGTTCCTTTTGTGATTCCCGAGAATGCTCTGCTGTCTG
AAAAGGAGTTTTATGTTGATCAGAATGTGTCCATCAAATGTAGGGAAGGTTTTCTGCTGCAGGGCCACGGCATCAT
TACCTGCAACCCCGACGAGACGTGGACACAGACAAGCGCCAAATGTGAAAAAATCTCATGTGGTCCACCAGCTCAC
GTAGAAAAATGCAATTGCTCGAGGCGTACATTATCAATATGGAGACATGATCACTACTCATGTTACAGTGGATACA
TGTTGGAGGGTTTCCTGAGGAGTGTTTGTTTAGAAAATGGAACATGGACATCACCTCCTATTTGCAGAGCTGTCTG
TCGATTTCCATGTCAGAATGGGGGCATCTGCCAACGCCCAAATGCTTGTTCCTGTCCAGAAAGCTGGATGGGGCGC
CTCTGTGAAGAACCAATCTGCATTCTTCCCTGTCTGAACGGAGGTCGCTGTGTGGCCCCTTACCAGTGTGACTGCC
CGCCTGGCTGGACGGGGTCTCGCTGTCATACAGCTGTTTTGCCAGTCTCCCTGCTTAATGGTGGAAAATGTGTAAG
ACCAAACCGATGTCACTGTCTTTCTTCTTGGACGGGACATAACTGTTCCAGGAAAAGGAGGACTGGGTTTTAACCA
CTGCACGACCATCTGGCTCTCCCAAAAGCAGGATCATCTCTCCTCGGTAGTGCCTGGGCATCCTGGAACTTATGCA
AAGAAAGTCCAACATGGTGCTGGGTCTTGTTTAGTAAACTTGTTACTTGGGGTTACTTTTTTTATTTTGTGATATA
TTTTGTTATTCCTTGTGACATACTTTCTTACATGTTTCCATTTTTATATGCCTGTATTTTCTATATAAAAAAATTA
TATTAAATAGATGCTGCTCTACCCTCACACAAAATGTACATATTCTGCTGTCTATTGGGAAAGTTCCTGGTACACATT
TTTATTCAGTTACTTAAAATGATTTTTCCATTAAAGTATATTTTGCTACTAAATAAAAAAAAAA

The sequence of NOV2a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof

The DNA sequence and protein sequence for a novel polydom-like gene were obtained by SeqCallingTM Technology and are reported here as NOV2a. These methods used to amplify NOV2a cDNA are described in Example 2.

The NOV2a polypeptide (SEQ ID NO: 6) encoded by SEQ ID NO: 5 is 3570 amino acid residues in length and is presented using the one-letter amino acid code in Table 2B. The SignalP, Psort and/or Hydropathy results predict that NOV2a has a signal peptide and is likely to be localized extracellularly with a certainty of 0.3846. In alternative embodiments, a NOV2a polypeptide is located to the lysosome (lumen) with a certainty of 0.1900, the endoplasmic reticulum (membrane) with a certainty of 0.1000, or the endoplasmic reticulum (lumen) with a certainty of 0.1000. The SignalP predicts a likely cleavage site for a NOV2a peptide between amino acid positions 16 and 17, i.e. at the dash in the sequence VSG-WA.

TABLE 2B
Encoded NOV2a Protein Sequence

(SEQ ID NO:6)

MRRTCAACWGLALVSGWATFQQMSPSRNFSFRLFPETAPGAAGSIPAPPAPGDEAAGSRVERLGQAFRVRLLR
ELSERLELVFLVDDSSSVGEVNFRSELMFVRKLLSDFPVVPTATRVAIVTFSSKNYVVPRVDYISTRRRARQHK
CALLLQEIPAISYRGGGTYTKGAFQQAAQILLHENSTKXJAAJFLITDGYSNGGDPRPIAASLRDSGVEIFTFG
IWQGNIRELNDMASTPKEEHCYLLHSFEEFEALVALCHMLFVDLPSGSFIQDDMVHCSYLCDEGKDCCDRMGS
CKCGKHTGHFECICEKGYNGKGLQYDCTVCPSGTYKPEGSPGGISSCIPCPDENHTSPPGSTSPEDCVCREGY
RASGQTCEVVHCPALKPPENGYFIQNTCNNHFNAACGVRCHPGAALVGSSIILCLPNGLWSGSESYCRVRTCP
HLRQPKHGHISCSTREMLYKTTCLVACDEGYRLEGSDKLTCQGNSQAAGPEPRCVERHCSTFQMPKDVIISPH
NCGKQPAKFGTICYVSCRQGFILSGVKEMLRCTTSGKVGVQAAVCKDVEAPQINCPKDIEAKTLEQQDSAN
VTWQIPTAKDNSGEKVSVHVHPAFTPPYLFPIGDVAIVYTATDLSGNQASCIFHIKVIDAAPPVIDWCRSPPP
VQVSEKVHAASWDEPQFSDNSGAELVITRSHTQGDLFPQGETIVQYTATDPSGAARTCDIHIVIKGSPCEIPF
TPVNGDFICTPDNTGVNCTLTCLEGYDFTEGSTDKYYCAYEDGAAKPTYTTEWFDCASKRFANHGFKSFEMFY
KAARCDDTDLMKKFSEAFETTLGKMVPSFCSDAEDIDCRLEENLTKKYCLENYDYENGFAIGPGGWGAANRL
DYSYDDFLDTVQETATSIGNAKSSRIKRSAPLSDYKIKLIFNITASVPLPDERNDTLEWENQQRLLQTLETIT
NKLKRTLNKDPMYSFQLASEILIADSNSLETKKASPFCRPGSVLRGRMCVNCPLGTYYNLEHFTCESCRIGSY
QDEEGQLECKLCPSGMYTEYIHSRNISDCKAQCKQGTYSYSGLETCESCPLGTYQPKFGSRSCLSCPENTSTV
KRGAVNISACGVPCPEGKFSRSGLMPCHPCPRDYYQPNAGKAFCLACPFYGTTPFAGSRSITECSSFSSTFSA
AEESVVPPASLGHIKKRHEISSQASHECFFNPCAASGTCQQLGRGYVCLCPLGYTGLKCETDIDECSPLPCLN
NGVCKDLVGEFICECPSGYTGKHCELNINECQSNPCRNQATCVDELNSYSCKCQPGFSGKRCETGMYQLSVIN
NLNNAVCEDQVGGFLCKCPPGFLGTRCGKVDECLSQPCYAAGATCAAGAASFRCLCAAGFTGSHCELNINECQ
SNPCRNQATCAAELNSYSCKCQPGFSGAACETEQSTGAALDFEVSGIYGLDGMLPSLHALTCTFWMKSSD
DMNYGTPISYAVDNGSDNTLLLTDYNGWVLYVNGREKITNCPSVNDGRWHHIAITWTSTGGAWRVYINGELSD
GGTGLSIGKAIPGGGALVLGQEQDKKGEGFNPAESFVGSISQLAALWVLSPQQVKSAATSCPEELSKGNVLA
WPDFLSGIVGKVKIDSKSIFCSDCPRLGGSVPHLRTASEDLKPGSKVNLFCEPGFQLVGNPVQYCLNQGQWTQ
PLPHCERIRCGVPPPLENGFHSADDFYAGSTVTYQCNNGYYLLGDSRMFCTDNGSWNGVSPSCLDVDECAVGS
DCSEHASCLNVDGSYICSCVPPYTGDGKNCAEPIKCKAPGNPENGHSSGEIYTVGAEVTFSCQEGYQLMGVTK
ITCLESGEWHLIPYCKAVSCGKPAIPENGCIEELAFTFGSAATYRCNKGYTAAGDKESSCLAANSSWSHSPPV
CEPVKCSSPENTNNGKYILSGLTYLSTASYSCDTGYSLQGPSIIECTASGIWDRAPPACHLVFCGEPPAIKDA
VITGNNFTFRNTVTYTCKEGYTLAGLDTIECLADGKWSRSDOOCLAVSCDEPPIVDHASPETAHRLFGDIAFY
YCSDGYSLADNSQLLCNAQGKWVPPEGQDMPRCIAHFCEKPPSVSYSILESVSKAKFAAGSVVSFKCMEGFVL
NTSAKIECMRGGQWNPSPMSIQCIPVRCGEPPSIMNGYASGSNYSFGAMVAYSCNKGFYIKGEKKSTCEATGQ
WSSPIPTCHPVSCGEPPKVENGFLEHTTGRIFESEVRYQCNPGYKSVGSPVFVCQANRHWHSESPLMCVPLDC
GKPPPIQNGFMKGEAAFEVGSKVQFFCNEGYELVGDSSWTCQKSGKAAKSNPKCMPAACPEPPLLENQLVLKE
LTTEVGVVTFSCKEGHVLQGPSVLKCLPSQQWNDSFPVCKIVLCTPPPLISFGVPIPSSALHFGSTVKYSCVG
GFFLRGNSTTLCQPDGTWSSPLPECVPVECPQPEEIPNGIIDVQGLAYLSTALYTCKPGFELVGNTTTLCGEN
GHWLGGKPTCKAIECLKPKEILNGKFSYTDLHYGQTVTYSCNRGFRLEGPSALTCLETGDWDVDAPSCNAIHC
DSPQPIENGFVEGAAAYSYGAIIIYSCFPGFQVAGAAQTCEESGWSSSIPTCMPIDCGLPPHIDFGDCTKLKD
DQGYFEQEDDMMEVPYVTPHPPYHLGAVAKTWENTKESPATHSSNFLYGTMVSYTCNPGYELLGNPVLICQED
GTWNGSAPSCISIECDLPTAPENGFLRFTETSMGSAVQYSCKPGHIAAGSDLRLCLEAAAASGASPRCEAISC
KKPNPVMNGSIKGSNYTYLSTLYYECDPGYVLNGTERRTCQDDYAAEDEPICIPAACSSPPVSANGQVRGDE
YTFQKEIEYTCNEGFLLEGAASRVCLAAGSWSGATPDCVPAACATPPQLAAGVTEGLDYGFMKEVTFHCHEGY
ILHGAPKLTCQSDGNWDAEIPLCKPVNCGPPEDLAHGFPNGFSFIHGGHIQYQCFPGYKLHGNSSRRCLSNGS
WSGSSPSCLPCRCSTPVIEYGTVNGTDFDCGYIQCFKGFKLLGLSEITCEADGQWSSGFPHCEHTSCGSL
PMIPNAFISETSSWKENVITYSCRSGYVIQGSSDLICTEKGVWSQPPVCEPLSCGSPPSVANAVATGEAHTY
ESEVKLRCLEGYTMDTDTDTFTCQKDGRWFPERISCSPKKCPLPENITHILVHGDDFSVNRQVSVSCAEGYTF
EGVNISVCQLDGTWEPPFSDESCSPVSCGKPESPEHGFVVGSKYTFESTIIYQCEPGYELEGNRERVCQENRQ
WSGGVAICKETRCETPLEFLNGKADIENRTTGPNVVYSCNRGYSLEGPSEAHCTENGTWSHPVPLCKPNPCPV
PFVIPENALLSEKEFYVDQNVSIKCREGFLLQGHGIITCNPDETWTQTSAKCEKISCGPPAHVENAIARGVHY
QYGMITYSCYSGYMLEGFLRSVCLENGTWTSPPICRAVCRFPCQNGGICQRPNACSCPEGWMGRLCEEPICI
LPCLGGRCVAPYQCDCPPGWTGSRCHTAVCQSPCLNGGKCAAPAACHCLSSWTGAACSRAARTGF

NOV2b

In an alternative embodiment, a NOV2 variant is NOV2b (alternatively referred to herein as CG50646-05), which includes the 11152 nucleotide sequence (SEQ ID NO: 7) shown in Table 2C. An open reading frame for the mature protein was identified beginning with an ATG codon at nucleotides 77-79 and ending with a termination codon at nucleotides 10781-10783. The start and stop codons of the open reading frame are highlighted in bold type. Putative untranslated regions are underlined and found upstream from the initiation codon and downstream from the termination codon.

TABLE 2C
NOV2b Nucleotide Sequence

(SEQ ID NO:7)

CAATTGGTCTAGGGTCTCCCCCCATTGGAATATCCATCAGTGATGAGAAATACAACGTTTGTTGAGTTTTC
TCTAGCATGAGAAGAAATTTGCGCGGCTTGCTGGGGTCTGGCGCTCGTTTCGGGCTGGGCGACCTTTCAGC
AGATGTCCCCGTCGCGAAATTTCAGCTTCCGCCTCTTCCCCGAGACCGCGCCCGGGGCCCCCGGGAGTAT
CCCCGCGCCGCCCGCTCCTGGCGACGAAGCGGCGGGGAGCAGAGTGGAGCGGCTGGGCAAGGCGTTCCGC
GTGCGGCTGCTGCGGGAGCTCAGCGAGCGCCTGGAGCTTGTCTTCCTGGTGGATGATTCGTCCAGCGTGG
GCGAAGTCAACTTCCGCAGCGAGCTCATGTTCGTCCGCAAGCTGCTGTCCGACTTCCCCGTGGTGCCCAC
GGCCACGCGCGTGGCCATCGTGACCTTCTCGTCCAAGAACTACGTGGTGCCGCGCGTCGATTACATCTCC
ACCCGCCGCGCGCGCCAGCACAAGTGCGCGCTGCTCCTCCAAGAGATCCCTGCCATCTCCTACCGAGGTG
GCGGCACCTACACCAAGGGCGCCTTCCAGCAAGCCGCGCAAATTCTTCTTCATGCTAGAGAAAACTCAAC
AAAAGTTGTATTTCTCATCACTGATGGATATTCCAATGGGGGAGACCCTAGACCAATTGCAGCGTCACTG
CGAGATTCAGGAGTGGAGATCTTCACTTTTGGCATATGGCAAGGGAACATTCGAGAGCTGAATGACATGG
CTTCCACCCCAAGGAGGAGCACTGTTACCTGCTACACAGTTTTTGAAGAATTTGAGGCTTTAGCTCGCCG
GGCATTGCATGAAGATCTACCTTCTGGGAGTTTTATTCAAGATGATATGGTCCACTGCTCATATCTTTGT
GATGAGGGCAAGGACTGCTGTGACCGAATGGGAAGCTGAAAATGTGGGACACACACAGGCCATTTTGAGT
GCATCTGTGAAAGGGGTATTACGGGAAGGTCTGCAGTATGAAAATGCACAGCTTGCCCATCGGGGACATA
CAACCTGAAGCCTCACCAGGAGGAATCAGCAGTTGCATTCCATGTCCCGATGAAAATCACAACCTCTCCA
CCTCGAAGCACATCCCCTGAAGACTGTGTCTGCAGAGAGGGATACAGGGCATCTGGCCAGACCTGTGAAC
TTGTCCACTGCCCTGCCCTGAAGCCTCCCGAAAATGGTTACTTTATCCAAAACACTTGCAACAACCACTT
CAATGCAGCCTGTGGGGTCCGATGTCACCCTGGATTTGATCTTGTGGGAAGCAGCATCATCTTATGTCTA
CCCAATGGTTTGTGGTCCGGTTCAGAGAGCTACTGCAGAGTAAGAACATGTCCTCATCTCCGCCAGCCGA
AACATGGCCACATCAGCTGTTCTACAAGGGAAATGTTATATAAGACAACATGTTTGGTTGCCTGTGATGA
AGGGTACAGACTAGAAGGCAGTGATAAGCTTACTTGTCAAGGAAACAGCCAGTGGGATGGGCCAGAACCC
CGGTGTGTGGAGCGCCACTGTTCCACCTTTCAGATGCCCAAGATGTCATCATATCCCCCCCACAACTGTG
GCAAGCAGCCAGCCAAATTTGGGACGATCTGCTATGTAAGTTGCCGCCAAGGGTTCATTTTATCTGGAGT
CAAAGAAATGCTGAGATGTACCACTTCTGGAAAATGGAATGTCGGAGTTCAGGCAGCTGTGTGTAAAGAC
GTGGAGGCTCCTCAAATCAACTGTCCTAAGGACATAGAGGCTAAGACTCTGGAACAGCAAGATTCTGCCA
ATGTTACCTGGCAGATTCCAACAGCTAAGACAACTCTGGTGAAAAAGGTGTAAGTCCGCQTTCATCCAGC
TTTCACCCCACCTTACCTTTTCCCAAATTGGAATGTTCGTATCGTATACACGGCAACTGACCTATCCGGC
AACCAGGCCAGCTGCATTTTCCATATCAAGGTTATTGATGCAGAACCACCTGTCATAGACTGGTGAAGAT
CTCCACCTCCCGTCCAGGTCTCGGAGAAGGTACATGCCGCAAGCTGGGATGAGCCTCAGTTCTCAGACAA
CTCAGGGGCTGAATTGGTCATTACCAGAAGTCATACACAAGGAGACCTTTTCCCTCAAGGGGAGACTATA
GTACAGTATACAGCCACTGACCCCTCAGGTAATAACAAGGATATGTGATATCCATATTGTCATGAAGGTT
CTCCCTGGGTGAATTCCATTCACACCTGTAATGGGGATTTTATATGCACTCCAGATAATACTGGAGTCAA
CTGTACATTAACTTGCTTGGAGGGCTACGATTTCACAGAAGGGTCTACTACAAAGTATTATTGTGCTTAT
GAAGATGGCGTCTGGAAACCAACATATACCACTGAATGGCCAGACTGTTGCCAAAAACGTTTTGCAAACC
ACGGGTTCAAGTCCTTTGAGATGTTCTACAAAGCAGCTCGTTGTGATGACTCAGATCTGATGAAGAAGTT
TTCTGAAGCATTTGAGACGACCCTGGGAAAAATGGTCCCATCATTTTGTAGTGATGCAGAGGACATTGAC
TGCAGACTGGAGGAGAACCTGACCAAAAATATTGCCTAGAATATAATTATGACTATGAAAAATGGCTTTG
CAATTGGTCCAGGTGGCTGGGGTGCAGCTAATAGGCTGGATTACTCTTACGATGACTTCCTGGACACTGT
GCAAGAAACAGCCACAAGCATCGGCAATGCCAAGTCCTCACGGATTAAAAGAAGTGCCCCATTATCTGAC
TATAATTAAGTTAATTTTTAACATCACAGCTAGTGTGCCATTACCCGATGAAAAAGAAATGATACCCTTG
AATGGGAAAATCAGCAACGACTCCTTCAGACATTGGAAACTATCACAAATAAACTGAAAAGGACTCTCAA
CAAAGACCCCCATGTATTCCTTTCAGCTTGCATCAGAAATACTTATAGCCGACAGCATTCAATTAGAACA
AAAAAGGCTTCCCCCTTCTGCAGACCAGGCTCAGTGCTGAGAGGGCGTATGTGTGTCAATTGCCCTTTGG
GAACCTATTATAATCTGGAACATTTCACCTGTGAAAGCTGCCGGATCGGATCCTATCAAGATGAAGAAGG
GCAACTTGAGTGCAAGCTTTGCCCCTCTGGGATGTACACGAATATATCCATTCAAAACAACATCTCTGAT
TGTAAAGCTCAGTGTAAACAAGGCACCTACTCATACAGTGGACTTGAGACTTGTGAATCGTGTCCACTGG
GCACTTATCAGCCAAAATTTGGTTCCCGGAGCTGCCTCTCGTGTCCAGAAAACACCTCAACTGTGAAAAG
AGGAGCCGTGAACATTTCTGCATGTGGAGTTCCTTGTCCAGAAGGAAAATTCTCGCGTTCTGGGTTAATG
CCCTGTCACCCATGTCCTCGTGACTATTACCAACCTAATGCAGGGAAGGCCTTCTGCCTGGCCTGTCCCT
TTTATGGAACTACCCCATTCGCTGGTTCCAGATCCATCACAGAATGTTCAAGTTTTAGTTCAACTTTCTC
AGCGGCAGAGGAAAGTGTGGTGCCCCCTGCCTCTCTTGGACATATTAAAAAGAGGCATGAAATCAGCAGT
CAGGCAGTCATGAATGCTTCTTTAACCCTTGCCACAATAGTGGAACCTGCCAGCTAACTTGGGCGTGGTT
ATGTTTGTCTCTGTCCACTTGGATATACAGGTTTAAAGTGTGAAACAGACATCGATGAGTGCAGCCCACT
GCCTTGCCTCAACATGGAGTTTGTAAAGACCTAGTTGGGGGAATTCATTTGTGAGTGCCAATCAGGTTAC
ACAGGTAAGCACTGTGAATTGAACATCAATGAATGTCAGTCTAATCCATGTAGAAATCAGGCCACCTGTG
TGGATGAATTAAATTCATACAGTTGTAATGTCAGCCAGGATTTTCAAGGCAAAAGGTGTGAAACAGGTAT
GTATCAACTCAGTGTTATTAATAACCTTAATAATGCAGTCTGTGAAGACCAGGTTGGGGGATTCTTGTGC
AAATGCCCACCTGGATTTTTGGGGTACCCGATGTGGAAGAACGTCGATGAGTGTCTCAGTCAGCCATGCA
AAAATGGAGCTACCTGTAATGACGGTGCCAATAGCTTCAGGTGCCTGTGTGCAGCTGGCTTCACAGGATC
ACACTGTGAATTGAACATCAATGAATGTCAGTCTAATCCATGTAGAAATCAGGCCACCTGTGTGGATGAA
TTAAATTCATACAGTTGTAAATGTCAGCCAGGATTTTCAGGCAAAAGGTGTGAAACAGAACAGTCTACAG
GCTTTAACCTGGATTTTGAAGTTTCTGGCATCTATGGATATGTCATGCTAGATGGCATGCTCCCATCTCT
CCATGCTCTAACCTGTACCTTCTGGATGAAATCCTCTGACGACATGAACTATGGAACACCAATCTCCTAT
GCAGTTGATAACGGCAGCGACAATACCTTGCTCCTGACTGATTATAACGGGTGGGTTCTTTATGTGAATG
GCAGGGAAAAGATAACAAACTGTCCCTCGGTGAATGATGGCAGATGGCATCATATTGCAATAACTTGGAC
AAGTACTGGTGGGAGCCTGGAGGGTCTATATAATGGGGAATTATCTGACGGTGGTACTGGCCTCTCCATT
GGCAAAGCCATACCTGGTGGCGGTGCATTAGTTCTTGGGCAAGAGCAAGACAAAAAAGGAGAGGGGTTCA
ACCCGGCTGAGTCTTTTGTGGGCTCCATAAGCCAGCTCAACCTCTGGGACTATGTCCTGTCTCAACAGAA
GGTGAAGTCACTGGCTACCTCCTGCCCAGAGGAACTCAGTAAAGGAAACGTGTTAGCATGGCCTGATTTC
TTGTCAGGAATTGTGGGGGAAGTGAAGATCGATTCTAAGAGCATATTTTGTTCTGATTGCCCACGCTTGG
GAGGGTCAGTGCCTCATCTGAGAACTGCATCTGAAGATTTAAAACCAGGTTCCAAAGTCAATCTGTTCTG
TGAACCAGGCTTCCAGCTGGTCGGGAACCCTGTGCAGTACTGTCTGAATCAAGGACAGTGGACACAACCA
CTCCCCCACTGTGAACGCATTCGCTGTGGGGTGCCACCTCCTTTGGAGAATGGCTTCCATTCAGCCGATG
ACTTCTATGCTGGCAGCACAGTAACCTACCAGTGCAACAATGGCTACTATCTATTGGGTGACTCAAGGAT
GTTCTGTACAGATAATGGGAGCTGGAACGGCGTTTAACCATCCTGCTTAGATGTCGATGAGTGTGCAGTT
GGATCAGATTGTAGTGAGCATGCTTCTTGCCTGAACGTAGATGGATCCTACATATGTTCATGTGTCCCAC
CGTACACAGGAGATGGGAAAAACTGTGCAGAACCTATAAAATGTAAGGCTCCAGGAAATCCGGAAAATGG
CCACTCCTCAGGTGAGATTTATACAGTAGGTGCCGAAGTAACATTTTCGTGTCAGGAAGGATACCAGTTG
ATGGGAGTAACCAAAATCACATGTTTGGAGTCTGGAGAATGGAATCATCTAATACCATATTGTAAAGCTG
TTTCATGTGGTAAACCGGCTATTCCAGAAAATGGTTGCATTGAGGAGTTAGCATTTACTTTTGGCAGCAA
AGTGACATATAGGTGTAATAAAAGGATATACTCTGGCCGGTGATAAGAATCATCCTGTCTTGCTAACAGT
TCTTGGAGTCATTCCCCTCCTGTGTGTGAACCAGTGAAGTGTTCTAGTCCGGAAAAATATAATAATGGAA
AATATATTTTGAGTGGGCTTACCTACCTTTCTACTGCATCATATTCATGCGATACAGGATACAGCTTACA
GGGCCCTTCCATTATTGAATGCACGGCTTCTGGCATCTGGGACAGAGCGCCACCTGCCTGTCACCTCGTC
TTCTGTGGAGAACCACCTGCCATCAAAGATGCTGTCATTACGGGGAATAACTTCACTTTCAGGAACACCG
TCACTTACACTTGCAAGAAGGCTATACTCTTGCTGGTCTTGACACCATTGAATGCCTGGCCGACGGCAAA
GTGGAGTAGAAGTGACCAQCAGTGCCTGGCTGTCTCCTGTGATGAGCCACCCATTGTGGACCACGCCTCT
CCAGAGACTGCCCATCGGCTCTTTGGAGACATTGCATTCTACTACTGCTCTGATGGTTACAGCCTAGCAG
ACAATTCCCAGCTTCTCTGCAATGCCCAGGGCAAGTGGGTACCCCCAGAAGGTCAAGACATGCCCCGTTG
TATAGCTCATTTCTGTGAAAAACCTCCATCGGTTTCCTATAGCATCTTGGAATCTGTGAGCAAAGCAAAA
TTTGCAGCTGGCTCAGTTGTGAGCTTTAAATGCATGGAAGGCTTTGTACTGAACACCTCAGCAAAGATTG
AATGTATGAGAGGTGGGCAGTGGAACCCTTCCCCCATGTCCATCCAGTGCATCCCTGTGCGGTGTGGAGA
GCCACCAAAGCATCATGAATGGCTATGCAAGTGGATCAACTACAGTTTTGGAGCCATGGTGGCTTACAGC
TGCAACAAGGGGTTCTACATCAAAGGGGAAAAGAAGAGCACCTGCGAAGCCACAGGGCAGTGGAGTAGTC
CTATACCGACGTGCCACCCGGTATCTTGTGGTGAACCACCTAAGGTTGAGAATGGCTTTCTGGAGCATAC
AACTGGCAGGATCTTTGAGAGTGAAGTGAGGTATCAGTGTAACCCGGGCTATAAGTCAGTCGGAAGTCCT
GTATTTGTCTGCCAAGCCAATCGCCACTGGCACAGTGAATCCCCTCTGATGTGTGTTCCTCTCGACTGTG
GAAAACCTCCCCCCCGATCCAGAATGGCTTCATGAGGAAAGAACTTTGAAGTAGGGTCCAAGGTTCAGTT
TTTCTGTAATGAGGGTTATGAGCTTGTTGGTGACAGTTCTTGGACATGTAAGAAATCTGGCAAATGGAAT
AAGAAGTCAAATCCAAAGTGCATGCCTGCCAAGTGCCCAGAGCCGCCCCTCTTGGAAAACCAGCTAGTAT
TAAAGGAGTTGACCACCGAGGTAGGAGTTGTGACATTTTCCTGTAAGGAAGGGAATGTCCTGCAAGGCCC
CTCTGTCCTGAATGCTTGCCATCCCAGCAATGGAATGACTCTTTCCCTGTTTGTAAGAATTGTTCTTTGT
ACCCCACCTCCCCTAATTTCCTTTGGTGTCCCCATTCCTTCTTCTGCTCTTAATTTTGGAAGTACTGTCA
AGTATTCTTGTGTAGGTGGGTTTTCCTAAGAGGAAATTCTACCACCCTCTGCCAAACCTGATGGCACCTG
GAGCTCTCCACTGCCAGAATGTGTTCCAGTAGAATGTCCCCAACCTGAGGAAATCCCCAATGGAATCATT
GATGTGCAAGGCCTTGCCTATCTCAGCACAGCTCTCTATACCTGCAAGCAAGGCTTTGAATTGGTGGGAA
ATACTACCACCCTTTGTGGAAAAGATGGTCACTGGCTTGGAGGAAAACCAACATGTAAAGCCATTGAGTG
CCTGAAACCCAAGGAGATTTGAAATGGCAAATTCTCTTACACGGACCTACACTATGGACAGACCGTTACC
TACTCTTGCAACCGAGGCTTTCGGCTCGAAGGTCCCAGTGCCTTGACCTGTTTACAGACAGGTGATTGGG
ATGTAGATGCCCCATCTTGCAATGCCATCCACTGTGATTCCCCACAACCCATTGAAAATGGTTTTGTAGA
AGGTGCAGATTACAGCTATGGTGCCATAATCATCTACAGTTGCTTCCCTGAATTTCAGGTGGCTGGTCAT
GCCATGCAGACCTGTGAAGAGTCAGGATGGTCAAGTTCCATCCCAAAATGTATGCCAATAGACTGTGGCC
TCCCTCCTCATATAGATTTTGGAGACTGTACTACTCAAGATGACCAGGGATATTTTGAGCAAGAAGACAG
CGACATGATGGAAGTTCCATATGTGACTCCTCACCCTCCTTATCATTTGGGAGCAGTGGCTAAAACCTGG
GAAAATACAAAGGAGTCTCCTGCTACACATTCATCAAACTTTCTGTATGGTACCATGGTTTCATACACCT
GTAATCCAGGATATGAACTTCTGGGGAACCCTGTGCTGATCTGCCAGGAAGATGGAACTTGGAATGGCAG
TGCACCATCCTGCATTTCAATTGAATGTGACTTGCCTACTGCTCCTGAAAATGGCTTTTTGCGTTTTACA
GAGACTAGCATGGGAAGTGCTGTGCAGTATAGCTGTAAACCTGGACACATTCTAGCAGGCTCTGACTTAA
GGCTTTGTCTAGAGAATAGAAGTGGAGTGGTGCCTCCCCACGCTGTGAAGCCATTTCATGCAAAAAAGCC
AAATCCAGTCATGAATGGATCCATCAAAGGAAGCAACTACACATACCTGAGCACGTTGTACTATGAGTGT
GACCCCGGATATGTGCTGAATGGCACTGAGAGGAGAACATGCAAGGATGACAAAACTGGGGATGAGGATG
AGCCCATTTGCATTCCTGTGGACTGCAGTTCACCCCCATCTCAGCCAAATGGCCAGGTGAGAGGAGACGA
GTACACATTCCAAAGAGATTGAATACACTTGCAATGAAGGGTTTCTTGGCTTGAGGGAGCCAGGAGTCGG
GTTTGTCTTGCCAATGGAAGTTGGAGTGGAGCCACTCCCGACTGTGTGCCTGTCAGATGTGCCACCCCGC
CACAACTGGCCAATGGGGTGACGGAAGGCCTGGACTATGGCTTCATGAAGGAAGTAACATTCCACTGTCA
CGAGGGCTACATCTTGCACGGTGCTCCAAAACTCACCTGTCAGTCAGATGGAACTGGGATGCAGAGATT
CCTCTCTGTAAACCAGTCAACTGTGGACCTCCTGAAGATCTTGCCCATGGTTTCCCTAATGGTTTTTCCT
TTATTCATGGGGGCCATATACAGTATCAGTGCTTTCCTGGTTATAAGCTCCATGGTTCATCAAGAAGAAG
GTGCCTCTCCAATGGCTCCTGGAGTGGCAGCTAACCTTCCTGCCTGCCTTGCAGATGTTCCACACCAGTA
ATTGAATATGGAACTGTCAATGGGACAGATTTTGACTGTGGAAGGCAGCCCGGATTCAGTGCTTCAAG
GCTTCAAGCTCCTAGGACTTTCTGAAATCACCTGTGAAGCCGATGGCCAGTGGAGCTCTGGGTTCCCCCA
CTGTGAACACACTTCTTGTGGTTCTCTTCCAATGATACCAAATGCGTTCATCAGTGAGACCAGCTCTTGG
AAGGAAAATGTGATAACTTACAGCTGCAGGTCTGGATATGTCATACAAGGCAGTTCAGATCTGATTTGTA
CAGAGAAAGGGGTATGGAGCCAGCCTTATCCAGTCTGTGAGCCCTTGTCCTGTGGGTCCCCACCGTCTGT
CGCCAATGCAGTGGCAACTGGAGAGGCACACACCTATGAAAGTGAAGTGAAACTCAGATGTCTGGAAGGT
TATACGATGGATACAGATACAGATACATTCACCTGTCAGAAGATGGTCGCTGGTTCCCTGAAGAGAATCT
CCTGCAGTCCTAAAAAATGTCCTCTCCCGGAAAAACATAACACATATACTTGTACATGGGACGATTTCAG
TGTGAATAGGCAAGTTTCTGTGTCATGTGCAGAAGGGTATACCTTTGAGGGAGTTAACATATCAGTATGT
CAGCTTGATGGAACCTGGGAGCCACCATTCTCCGATGAATCTTGCAGTCCAGTTTCTTGTGGGAAACCTG
AAAGTCCAGAACATGGATTTGTGGTTGGCAGTAAATACACCTTTGAAAGCACAATTATTTATCAGTGTGA
GCCTGGCTATGAACTAGAGGGGAACAGGGAACGTGTCTGCCAGGAGAACAGACAGTGGAGTGGAGGGGTG
GCAATATGCAAAGAGACCAGGTGTGAAAACTCCACTTGAATTTCTCAATGGGAAAGCTGACATTGAAACA
GGACGACTGGACCCAACGTGGTATATTCCTGCAACAGAGGCTACAGTCTTGAAGGGCCATCTGAGGCACA
CTGCACAGAAATGGAACCTGGAGCCACCCAGTCCCTCTCTGCAAAAACCAATCCATGCCCTGTTCCTTTT
GTGATTCCCGAGAATGCTCTGCTGTCTGAAAAGGAGTTTTATGTTGATCAGAATGTGTCCATCAAATGTA
GGGAAGGTTTTCTGCTGCAGGGCCACGGCATCATTACCTGCAACCCCGACGAGACGTGGACACAGACAAG
CGCCAAATGTGAAAAAATCTCATGTGGTCCACCAGCTCACGTAGAAAATGCAATTGCTCGAGGCGTACAT
TATCAATATGGAGACATGATCACCTACTCATGTTACAGTGGATACATGTTGGAGGGTTTCCTGAGGAGTG
TTTGTTTAGAAATGGAACATGGACATCACCTCCTATTTGCAGAGCTGTCTGTCGATTTCCATGTCAGAAA
TGGGGGGGCATCTGCCAACGCCCATGCTTGTTCCTGTCCAGAGCGCTGGATGGGGCGCCTCTGTGAAGAA
CCAATCTGCATTCTTCCCTGTCTGAACGGAGGTCGCTQTGTGGCCCCTTACCAGTGTGACTGCCCGCCTG
GCTGGACGGGGTCTCGCTGTCATACAGCTGTTTGCCAGTCTCCCTGCTTAATGGTGGAAAAATGTGTAAG
ACCAACCGATGTCACTGTCTTTCTTCTTGGACGGGAAAATAACTGTTCCAGGAAAAGGAGGACTGGGTTT
TAACCACTGCACGACCATCTGGCTCTCCCAAAAGCAGGATCATCTCTCCTCGGTAGTGCCTGGGCATCCT
GGAACTTATGCAAAGAAAGTCCAACATGGTGCTGGGTCTTGTTTAGTAAACTTGTTACTTGGGGTTACTT
TTTTTATTTTGTGATATATTTTGTTATTCCTTGTGACATACTTTCTTACATGTTTCAATTTTTAAATATG
CCTGTATTTTCTATATAAAAATTATATTAAATAGATGCTGCTCTACCCTCACAAAATGTACATATTCTGC
TGTCTATTGGGAAGTTCCTGGTACACATTTTTATTCAGTTACTTAAAATGATTTTTCCATTAAAAGTATA
TTTTGCTACTAAATAAAAAAAA

The sequence of NOV2b was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel polydom-like gene were obtained by SeqCallingTM Technology and are reported here as NOV2b. These methods used to amplify NOV2b cDNA are described in the Example 2.

The NOV2b polypeptide (SEQ ID NO: 8) encoded by SEQ ID NO: 7 is 3568 amino acid residues in length and is presented using the one-letter amino acid code in Table 2D. The SignalP, Psort and/or Hydropathy results predict that NOV2b has a signal peptide and is likely to be localized extracellularly with a certainty of 0.3846. In alternative embodiments, a NOV2b polypeptide is located to the lysosome (lumen) with a certainty of 0.1900, the endoplasmic reticulum (membrane) with a certainty of 0.1000, or the endoplasmic reticulum (lumen) with a certainty of 0.1000. The SignalP predicts a likely cleavage site for a NOV2b peptide between amino acid positions 16 and 17, i.e. at the dash in the sequence VSG-WA.

TABLE 2D
Encoded NOV2b Protein Sequence

(SEQ ID NO:8)

MRRICAACWGLALVSGWATFQQMSPSRNFSFRLFPETAPGAPGSIPAPPAPGDEAAGSRVERLGQAFRVRLLRELS
ERLELVFLVDDSSSVGEVNFRSELMFVRKLLSDFPVVPTATRVAIVTFSSKNYVVPRVDYISTRRARQHKCALLLQ
EIPAISYRGGGTYTKGAFQQAAQILLHARENSTKVVFLITDGYSNGGDPRPIAASLRDSGVEIFTFGIWQGNIREL
NDMASTPKEEHCYLLHSFEEFEALARRALHEDLPSGSFIQDDMVHCSYLCDEGKDCCDRMGSCKCGTHTGHFECIC
EKGYYGKGLQYECTACPSGTYKPEASPGGISSCIPCPDENHTSPPGSTSPEDCVCREGYRASGQTCELVHCPALKP
PENGYFIQNTCNNHFNAACGVRCHPGFDLVGSSIILCLPNGLWSGSESYCRVRTCPHLRQPKHGHISCSTREMLYK
TTCLVACDEGYRLEGSDKLTCQGNSQWDGPEPRCVERHCSTFQMPKDVIISPHNCGKQPAKFGTICYVSCRQGFIL
SGVKEMLRCTTSGKWNVGVQAAVCKDVEAPQINCPKDIEAKTLEQQDSANVTWQIPTAKDNSGEKVSVRVHPAFTP
PYLFPIGDVAIVYTATDLSGNQASCIFHIKVIDAEPPVIDWCRSPPPVQVSEKVHAASWDEPQFSDNSGAELVITR
SHTQGDLFPQGETIVQYTATDPSGNNRICDIHIVMKGSPCEIPFTPVNGDFICTPDNTGVNCTLTCLEGYDFTEGS
TDKYYCAYEDGVWKPTYTTEWPDCAKKRFANHGFKSFEMFYKAARCDDSDLMKKFSEAFETTLGKMVPSFCSDAED
IDCRLEENLTKKYCLEYNYDYENGFAIGPGGWGAANRLDYSYDDFLDTVQETATSIGNAKSSRIKRSAPLSDYKIK
LIFNITASVPLPDERNDTLEWENQQRLLQTLETITNKLKRTLNKDPMYSFQLASEILIADSNSLETKKASPFCRPG
SVLRGRMCVNCPLGTYYNLEHFTCESCRIGSYQDEEGQLECKLCPSGMYTEYIHSRNISDCKAQCKQGTYSYSGLE
TCESCPLGTYQPKFGSRSCLSCPENTSTVKRGAVNISACGVPCPEGKFSRSGLMPCHPCPRDYYQPNAGKAFCLAC
PFYGTTPFAGSRSITECSSFSSTFSAAEESVVPPASLGHIKKRHEISSQASHECFFNPCHNSGTCQQLGRGYVCLC
PLGYTGLKCETDIDECSPLPCLNNGVCKDLVGEFICECPSGYTGKHCELNINECQSNPCRNQATCVDELNSYSCKC
QPGFSGKRCETGMYQLSVINNLNNAVCEDQVGGFLCKCPPGFLGTRCGKNVDECLSQPCKNGATCKDGANSFRCLC
AAGFTGSHCELNINECQSNPCRNQATCVDELNSYSCKCQPGFSGKRCETEQSTGFNLDFEVSGIYGYVMLDGMLPS
LHALTCTFWMKSSDDMNYGTPISYAVDNGSDNTLLLTDYNGWVLYVNGREKITNCPSVNDGRWHHIAITWTSTGGA
WRVYINGELSDGGTGLSIGKAIPGGGALVLGQEQDKKGEGFNPAESFVGSISQLNLWDYVLSPQQVKSLATSCPEE
LSKGNVLAWPDFLSGIVGKVKIDSKSIFCSDCPRLGGSVPHLRTASEDLKPGSKVNLFCEPGFQLVGNPVQYCLNQ
GQWTQPLPHCERIRCGVPPPLENGFHSADDFYAGSTVTYQCNNGYYLLGDSRMFCTDNGSWNGVSPSCLDVDECAV
GSDCSEHASCLNVDGSYICSCVPPYTGDGKNCAEPIKCKAPGNPENGHSSGEIYTVGAEVTFSCQEGYQLMGVTKI
TCLESGEWNHLIPYCKAVSCGKPAIPENGCIEELAFTFGSKVTYRCNKGYTLAGDKESSCLANSSWSHSPPVCEPV
KCSSPENINNGKYILSGLTYLSTASYSCDTGYSLQGPSIIECTASGIWDRAPPACHLVFCGEPPAIKDAVITGNNF
TFRNTVTYTCKEGYTLAGLDTIECLADGKWSRSDQQCLAVSCDEPPIVDHASPETAHRLFGDIAFYYCSDGYSLAD
NSQLLCNAQGKWVPPEGQDMPRCIAHFCEKPPSVSYSILESVSKAKFAAGSVVSFKCMEGFVLNTSAKIECMRGGQ
WNPSPMSIQCIPVRCGEPPSIMNGYASGSNYSFGAMVAYSCNKGFYIKGEKKSTCEATGQWSSPIPTCHPVSCGEP
PKVENGFLEHTTGRIFESEVRYQCNPGYKSVGSPVFVCQANRHWHSESPLMCVPLDCGKPPPIQNGFMKGENFEVG
SKVQFFCNEGYELVGDSSWTCQKSGKWNKKSNPKCMPAKCPEPPLLENQLVLKELTTEVGVVTFSCKEGHVLQGPS
VLKCLPSQQWNDSFPVCKIVLCTPPPLISFGVPIPSSALHFGSTVKYSCVGGFFLRGNSTTLCQPDGTWSSPLPEC
VPVECPQPEEIPNGIIDVQGLAYLSTALYTCKPGFELVGNTTTLCGENGHWLGGKPTCKAIECLKPKEILNGKFSY
TDLHYGQTVTYSCNRGFRLEGPSALTCLETGDWDVDAPSCNAIHCDSPQPIENGFVEGADYSYGAIIIYSCFPGFQ
VAGHAMQTCEESGWSSSIPTCMPIDCGLPPHIDFGDCTKLKDDQGYFEQEDDMMEVPYVTPHPPYHLGAVAKTWEN
TKESPATHSSNFLYGTMVSYTCNPGYELLGNPVLICQEDGTWNGSAPSCISIECDLPTAPENGFLRFTETSMGSAV
QYSCKPGHILAGSDLRLCLENRKWSGASPRCEAISCKKPNPVMNGSIKGSNYTYLSTLYYECDPGYVLNGTERRTC
QDDKNWDEDEPICIPVDCSSPPVSANGQVRGDEYTFQKEIEYTCNEGFLLEGARSRVCLANGSWSGATPDCVPVRC
ATPPQLANGVTEGLDYGFMKEVTFHCHEGYILHGAPKLTCQSDGNWDAEIPLCKPVNCGPPEDLAHGFPNGFSFIH
GGHIQYQCFPGYKLHGNSSRRCLSNGSWSGSSPSCLPCRCSTPVIEYGTVNGTDFDCGKAARIQCFKGFKLLGLSE
ITCEADGQWSSGFPHCEHTSCGSLPMIPNAFISETSSWKENVITYSCRSGYVIQGSSDLICTEKGVWSQPYPVCEP
LSCGSPPSVANAVATGEAHTYESEVKLRCLEGYTMDTDTDTFTCQKDGRWFPERISCSPKKCPLPENITHILVHGD
DFSVNRQVSVSCAEGYTFEGVNISVCQLDGTWEPPFSDESCSPVSCGKPESPEHGFVVGSKYTFESTIIYQCEPGY
ELEGNRERVCQENRQWSGGVAICKETRCETPLEFLNGKADIENRTTGPNVVYSCNRGYSLEGPSEAHCTENGTWSH
PVPLCKPNPCPVPFVIPENALLSEKEFYVDQNVSIKCREGFLLQGHGIITCNPDETWTQTSAKCEKISCGPPAHVE
NAIARGVHYQYGDMITYSCYSGYMLEGFLRSVCLENGTWTSPPICRAVCRFPCQNGGICQRPNACSCPEGWMGRLC
EEPICILPCLNGGRCVAPYQCDCPPGWTGSRCHTAVCQSPCLNGGKCVRPNRCHCLSSWTGHNCSRKRRTGF

SNP variants of NOV2 are disclosed in Example 3.

NOV2 Clones

Unless specifically addressed as NOV2a or NOV2b, any reference to NOV2 is assumed to encompass all variants.

The amino acid sequence of NOV2 has high homolgy to other proteins as shown in Table 2E.

TABLE 2E
BLASTX Results from Patp Database for NOV2

| | | | Smallest |
|---|---|---|---|
| | | High | Sum |
| Sequences Producing High-Scoring Segment Pairs: | | Score | Prob P (N) |
| patp: AAM93954 | Human polypeptide, | 8375 | 0.0 |
| patp: AAB94754 | Human protein sequence | 7012 | 0.0 |
| patp: AAU16963 | Human novel secreted protein | 6452 | 0.0 |
| patp: AAU18126 | Novel human uterine motility-association po . . . | 6452 | 0.0 |
| patp: AAG66398 | Receptor 222 - Unidentified | 5577 | 0.0 |

In a search of sequence databases, it was found, for example, that the NOV2a nucleic acid sequence has 2414 of 2422 bases (99%) identical to a gb:GENBANK-ID:HST000009|acc:AL079279.1 mRNA from Homo sapiens (Homo sapiens mRNA full length insert cDNA clone EUROIMAGE 248114). Further, the full amino acid sequence of the disclosed NOV2a protein of the invention has 2895 of 3567 amino acid residues (81%) identical to, and 3181 of 3567 amino acid residues (89%) similar to, the 3567 amino acid residue ptnr:TREMBLNEW-ACC:AAG32160 protein from Mus musculus (Mouse) (POLYDOM PROTEIN PRECURSOR).

In a similar search of sequence databases, it was found, for example, that the NOV2b nucleic acid sequence has 7556 of 9127 bases (82%) identical to a gb:GENBANK-ID:AF206329|acc:AF206329.1 mRNA from Mus musculus (Mus musculus polydom protein mRNA, complete cds). Further, the full amino acid sequence of the disclosed NOV2b protein of the invention has 2902 of 3565 amino acid residues (81%) identical to, and 3189 of 3565 amino acid residues (89%) similar to, the 3567 amino acid residue ptnr:SPTREMBL-ACC:Q9ES77 protein from Mus musculus (Mouse) (POLYDOM PROTEIN PRECURSOR).

Additional BLASTP results are shown in Table 2F.

TABLE 2F
NOV2 BLASTP Results

| Gene Index/ | | Length of | Identity | Positives | Expect |
|---|---|---|---|---|---|
| Identifier | Protein/Organism | aa | (%) | (%) | Value |
| Q9ES77 | POLYDOM PROTEIN | 3567 | 289/3567 | 3181/3567 | 0.0 |
| | PRECURSOR - | | (81%) | (89%) | |
| | Mus musculus (Mouse) | | | | |
| BAB55420 | CDNA FLJ14964 FIS, | 1316 | 1255/1316 | 1267/1316 | 0.0 |
| | CLONE PLACE4000581, | | (95%) | (96%) | |
| | MODERATELY SIMILAR | | | | |
| | TO FIBROPELLIN I | | | | |
| | PRECURSOR - | | | | |
| | Homo sapiens (Human) | | | | |
| AAH08135 | POLYDOMAIN PROTEIN - | 669 | 534/668 | 594/668 | 0.0 |
| | Mus musculus (Mouse) | | (79%) | (88%0 | |
| Q9CUT3 | 4833413O10RIK PROTEIN - | 601 | 483/601 | 538/601 | 2.4e−298 |
| | Mus musculus (Mouse) | | (80%) | (89%) | |
| Q9H284 | SEROLOGICALLY | 481 | 458/482 | 462/482 | 1.8e−261 |
| | DEFINED BREAST | | (95%) | (95%) | |
| | CANCER ANTIGEN NY- | | | | |
| | BR-38 - Homo sapiens | | | | |
| | (Human) | | | | |

A multiple sequence alignment is given in Table 2G, with the NOV2 protein of the invention being shown in lines 1 and 2, in a ClustalW analysis comparing NOV2 with related protien sequences of Table 2F.

[image]

[image]

[image]

[image]

[image]

[image]

[image]

[image]

[image]

[image]

[image]

[image]

[image]

[image]

[image]

[image]

Domain results for NOV2 were collected from the Pfam database, and then identified by the Interpro domain accession number. The results are listed in Table 2H with the statistics and domain description. These results indicatee that the NOV2 polypeptides have properties similar to those of other proteins known to contain these domains.

TABLE 2H

Domain Analysis of NOV2

| | Score | E |
|---|---|---|
| PSSMs Producing Significant Alignments | (bits) | Value |
| Von Willebrand Factor Type A (vwa): domain 1 of 1, from | 86.8 | 4.5e−22 |
| from 80 to 256 | | |

| | | |
|---|---|---|
| vwa | DivFLlDGSgSigsqnFervKdFvervverLdvgprdkkeedavrVg | (SEQ ID NO:52) |
| | +++\|\|+\| \|+\|+'0 + +\| + \|++++ +++ + + + ++\|+ | |
| NOV2a | ELVFLVDDSSSVGEVNFRSELMFVRKLLSDFPVVP-TA-----TRVA | (SEQ ID NO:6) |
| | lvQYSdnvrtEikfklndyqnk........devlqalqkiryedyygggg | |
| | ++++\|++ + ++ ++ + ++ ++++ + +++ + ++ + +++ | |
| NOV2a | IVTFSSKNYV---VPRVDYISTrrarqhkcALLLQEIPAIS----YRGGG | |
| | tnTgaALqyvvrnlfteasGsRiepvaeegapkvlVvlTDGrsqddpspT | |
| | + \| +\| + ++ + + +\| ++ ++++ ++\|\|\| ++++ | |
| NOV2a | TYTKGAFQQAAQILLH----AR------ENSTKVVFLITDGYSNGG---- | |
| | idirdvlnelkkeagvevfaiGvGnadnnnleeLreIAskpd.dhvfkvs | |
| | + + +++++++ +++++++\|+ + + ++\|+ +\|+ + +++ + ++ | |
| NOV2a | -DPRPIAASLRD-SGVEIFTFGIWQG-N--IRELNDMASTPKeEHCYLLH | |
| | dfeaLdtlqelL | |
| | ++++ + ++++ | |
| NOV2a | SFEEFEALVALC | |

| | | |
|---|---|---|
| pentaxin: domain 1 of 1, from 1469 to 1607 | 75.5 | 7.5e−21 |

| | | |
|---|---|---|
| Pentaxin | SYaTkkPlkDNElLifkekdgqYslyvggaPqLevtfkvkeefvaPv | (SEQ ID NO:53) |
| | \|\|++ + \|\| +\|+ ++ +++++ +++ + + ++ | |
| NOV2a | SYAVDN-GSDNTLLL--TDYNGWVLYVNGR--EKITNCPSVNDGRWH | (SEQ ID NO:6) |
| | HiCtSWeSssGiaEfWVDGkhCpwvrkglkkGytvgaepsIiLGQEQDSy | |
| | \|+ +\| \| \| ++\| +++ \| + ++ +\|\|\|\|\|\| | |
| NOV2a | HIAITWTSTGGAWRVYINGE-LSDGGTGLSIGKAIPGGGALVLGQEQDKK | |
| | GGgFdksQSlVGEigdlnMWDyVLtPeeIktvykgagplerhiypNILdw | |
| | \| +\|+ \|+\|\| ++ +++\|\|+\|\|+\|++++++ + + +\|+\| \| | |
| NOV2a | GEGFNPAESFVGSISQLNLWDYVLSPQQVKSLATS-CPEE-LSKGNVLAW | |

| | | |
|---|---|---|
| sushi: domain 13 of 34, from 2145 to 2198 | 73.7 | 3.8e−18 |

| | | |
|---|---|---|
| sushi | Cp.pPdieNGrvsssgtyeypvGdtvtytCneGYrlvGsssitCted | (SEQ ID NO:54) |
| | \|+++\| ++\|\|+ + + ++++ \| + ++\|++\|++++\|++ ++\| ++ | |
| NOV2a | CGePPSIMNGYASGS-NYSF--GAMVAYSCNKGFYIKGEKKSTCEAT | (SEQ ID NO:6) |
| | ggGgWsppllGelPkC | |
| | \|+\|++++ \|+\| | |
| NOV2a | --GQWSSPI----PTC | |

The NOV2 disclosed in this invention is expressed in at least the following tissues: adipose, adrenal gland, bone marrow, brain—amygdala, brain—cerebellum, brain—hippocampus, brain—substantia nigra, brain—thalamus, brain—whole, fetal brain, fetal kidney, liver, lung, heart, kidney, ascending colon, lymphoma—Raji, mammary gland/breast, pancreas, nasoepithelium, pituitary gland, placenta, prostate, cervix, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, Literature sources, and/or RACE sources.

The protein similarity information, expression pattern, and map location for the Polydom-like protein and nucleic acid disclosed herein suggest that this Polydom may have important structural and/or physiological functions characteristic of the epidermal growth factor (EGF) family. Therefore, the nucleic acids and proteins of the invention are useful in potential diagnostic and therapeutic applications and as a research tool. For example, the compositions of the presnet invention will have efficacy for treatment of patients suffering from: cancers, congenital heart disease, inflammatory disorders, erythroid-megakaryocytic leukaemia, Vacuoliting megalencephalic leukoencephalopathy, chronic contact dermatitis, fibrosarcoma, wound healing, neoplasia, such as T-cell acute lymphoblastic leukemia/lymphoma, reproductive disorders, fetal arrhythmias, immune system disorders, disorders of coagulation, obesity, diabetes, asthma, arthritis, osteoporosis, and other diseases, disorders and conditions of the like.

The novel nucleic acid encoding the polydom-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 0 to 125. In another embodiment, a contemplated NOV2 epitope is from about amino acids 130 to 250. In other specific embodiments, contemplated NOV2 epitopes are from about amino acids 250 to 3600.

NOV3

Another NOVX protein of the invention, referred to herein as NOV3, includes two novel transmembrane/IIIb-like protein. The disclosed proteins have been named NOV3a and NOV3b. The NOV3a and NOV3b proteins of the invention cause growth inhibition of E. coli when expressed exogenously.

The NOV3a and NOV3b protein predicted here are localized extracellularly. Therefore, it is likely that they are accessible to a diagnostic probe, and for the various therapeutic applications described herein.

At least the NOV3b transmembrane-like protein disclosed in this invention maps to chromosome 20. This information was assigned using OMIM, the electronic northern bioinformatic tool implemented by CuraGen Corporation, public ESTs, public literature references and/or genomic clone homologies.

NOV3a

In one embodiment, a NOV3 variant is NOV3 a (alternatively referred to herein as CG50273-01), which encodes a novel transmembrane-like protein and includes the 870 nucleotide sequence (SEQ ID NO: 9) shown in Table 3A. An open reading frame for the mature protein was identified beginning with an ATG codon at nucleotides 1-3 and ending with a TAA codon at nucleotides 628-630. Putative untranslated regions downstream from the termination codon and upstream from the initiation codon are underlined in Table 3A, and the start and stop codons are in bold letters.

TABLE 3A
NOV3a Nucleotide Sequence

(SEQ ID NO:9)

ATGGGCTCCTGCTCCGGCCGCTGCGCGCTCGTCGTCCTCTGCGCTTTTCAGCTGGTGGTCGCCGCCCTGGAGAGGC
AGGTGTTTGACTTCCTGGGCTACCAGTGGGCGCCCATCCTGGCCAACTTTGTCCACATCATCATCGTCATCCTGGG
ACTCTTCGGCACCATCCAGTACCGGCTGCGCTATGTCATGGTGTACACGCTGTGGGCAGCCGTCTGGGTCACCTGG
AACGTCTTCATCATCTGCTTCTACCTGGAAGTCGGTGGCCTCTTAAAGGACAGCGAGCTACTGACCTTCAGCCTCT
CCCGGCATCGCTCCTGGTGGCGTGAGCGCTGGCCAGGCTGTCTGCATGAGGAGGTGCCAGCAGTGGGCCTCGGGGC
CCCCCATGGCCAGGCCCTGGTGTCAGGTGCTGGCTGTGCCCTGGAGCCCAGCTATGTGGAGGCCCTACACAGTTGC
CTGCAGATCCTGATCGCGCTTCTGGGCTTTGTCTGTGGCTGCCAGGTGGTCAGCGTGTTTACGGAGGAAGAGGACA
GCTTTGATTTCATTGGTGGATTTGATCCATTTCCTCTCTACCATGTCAATGAAAAGCCATCCAGTCTCTTGTCCAA
GCAGGTGTACTTGCCTGCGTAAGTGAGGAAACAGCTGATCCTGCTCCTGTGGCCTCCAGCCTTCAGCGACCGACCA
GTGACAATGACAGGAGCTCCCAGGCCTTGGGACGCGCCCCCACCCAGCACCCCCCAGGCGGCCGGCAGCACCTGCC
CTGGGTTTTAAGTACTGGACACCAGCCAGGGCGGCAGGGCAGTGCCACGGCTGGCTGCAGCGTCAAGAGAGTTTGT
AATTTCCTTTCTCTTAAAAAAAAAAAAAAAAAAA

The sequence of NOV3a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel transmembrane-like gene were obtained by SeqCallingTM Technology and are reported here as NOV3a. These methods used to amplify NOV3a cDNA are described in Example 2.

The NOV3a polypeptide (SEQ ID NO: 10) encoded by SEQ ID NO: 9 is 209 amino acid residues in length and is presented using the one-letter amino acid code in Table 3B. The SignalP, Psort and/or Hydropathy results predict that NOV3a has a signal peptide and is likely to be localized extracellularly at the plasma membrane with a certainty of 0.4600. In alternative embodiments, a NOV3a polypeptide is located to the microbody (peroxisome) with a certainty of 0.1026, the endoplasmic reticulum (membrane) with a certainty of 0.1000, or the endoplasmic reticulum (lumen) with a certainty of 0.1000. The SignalP predicts a likely cleavage site for a NOV3a peptide between amino acid positions 29 and 30, i.e. at the dash in the sequence GAG-VL.

TABLE 3B
Encoded NOV3a Protein Sequence

(SEQ ID NO:10)

MGSCSGRCALVVLCAFQLVVAALERQVFDFLGYQWAPILANFVHIIIVILGLFGTIQYRLRYVMVYTLWAAVWVTW
NVFIICFYLEVGGLLKDSELLTFSLSRHRSWWRERWPGCLHEEVPAVGLGAPHGQALVSGAGCALEPSYVEALHSC
LQILIALLGFVCGCQVVSVFTEEEDSFDFIGGFDPFPLYHVNEKPSSLLSKQVYLPA

NOV3b

In an alternative embodiment, a NOV3 variant is NOV3b (alternatively referred to herein as CG50273-02), which includes the 632 nucleotide sequence (SEQ ID NO: 11) shown in Table 3C. An open reading frame for the mature protein was identified beginning with an GTC codon at nucleotides 2-4 and ending with a TAA codon at nucleotides 593-595. The start and stop codons of the open reading frame are highlighted in bold type. Putative untranslated regions are underlined and found upstream from the initiation codon and downstream from the termination codon.

TABLE 3C
NOV3b Nucleotide Sequence

(SEQ ID NO:11)

CGTCCTCTGCGCTTTTCAGCTGGTCGCCGCCCTGGAGAGGCAGGTGTTTGACTTCCTGGGCTACCAGTGGGCGCC
CATCCTGGCCAACTTTGTCCACATCATCATCGTCATCCTGGGACTCTTCGGCACCATCCAGTACCGGCTGCGCTA
TGTCATGGTGTACACGCTGTGGGCAGCCGTCTGGGTCACCTGGAACGTCTTCATCATCTGCTTCTACCTGGAAGT
CGGTGGCCTCTTAAAGGACAGCGAGCTACTGACCTTCAGCCTCTCCCGGCATCGCTCCTGGTGGCGTGAGCGCTG
GCCAGGCTGTCTGCATGAGGAGGTGCCAGCAGTGGGCCTCGGGGCCCCCCATGGCCAGGCCCTGGTGTCAGGTGC
TGGCTGTGCCCTGGAGCCCAGCTATGTGGAGGCCCTACACAGTTGCCTGCAGATCCTGATCGCGCTTCTGGGCTT
TGTCTGTGGCTGCCAGGTGGTCAGCGTGTTTACGGAGGAAGAGGACAGCTTTGATTTCATTGGTGGATTTGATCC
ATTTCCTCTCTACCATGTCAATGAAAAGCCATCCAGTCTCTTGTCCAAGCAGGTGTACTTGCCTGCGTAAGTGAG
GAAACAGCTGATCCTGCTCCTGTGGCCTCCAC

The sequence of NOV3b was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The cDNA coding for the NOV3b sequence was cloned by the polymerase chain reaction (PCR). Primers were designed based on in silico predictions of the full length or some portion (one or more exons) of the cDNA/protein sequence of the invention, or by translated homology of the predicted exons to closely related human sequences or to sequences from other species. The DNA sequence and protein sequence for a novel transmembrane-like gene were obtyained by exon linking and are reported here as NOV3b. These primers and methods used to amplify NOV3b cDNA are described in Example 2.

The NOV3b polypeptide (SEQ ID NO: 12) encoded by SEQ ID NO: 11 is 197 amino acid residues in length and is presented using the one-letter amino acid code in Table 3D. The SignalP, Psort and/or Hydropathy results predict that NOV3b has a signal peptide and is likely to be localized in the membrane of the endoplasmic reticulum with a certainty of 0.6850. In alternative embodiments, a NOV3b polypeptide is located to the plasma membrane with a certainty of 0.6400, the Golgi body with a certainty of 0.4600, or the endoplasmic reticulum (lumen) with a certainty of 0.1000. The SignalP predicts a likely cleavage site for a NOV3b peptide between amino acid positions 13 and 14, i.e. at the dash in the sequence LER-QV.

TABLE 3D
Encoded NOV3b Protein Sequence

(SEQ ID NO:12)

VLCAFQLVAALERQVFDFLGYQWAPILANFVHIIIVILGLFGTIQYRLRYVMVYTLWAAVWVTWNVFIICFYLEVG
GLLKDSELLTFSLSRHRSWWRERWPGCLHEEVPAVGLGAPHGQALVSGAGCALEPSYVEALHSCLQILIALLGFVC
GCQVVSVFTEEEDSFDFIGGFDPFPLYHVNEKPSSLLSKQVYLPA

SNP variants of NOV3 are disclosed in Example 3.

NOV3 Clones

Unless specifically addressed as NOV3a or NOV3b, any reference to NOV3 is assumed to encompass all variants.

The amino acid sequence of NOV3 has high homolgy to other proteins as shown in Table 3E.

TABLE 3E
BLASTX Results from Patp Database for NOV3

| | | | Smallest |
|---|---|---|---|
| | | High | Sum |
| Sequences Producing High-Scoring Segment Pairs: | | Score | Prob P |
| patp: AAB62810 | Human nervous system associated protein NSPRT3 | 1092 | 2.3e−110 |
| patp: AAY94954 | Human secreted protein clone iw66_1 | 619 | 3.0e−60 |
| patp: AAG78000 | Human actin 14 | 466 | 4.9e−44 |
| patp: AAB94211 | Human protein sequence | 425 | 1.1e−39 |
| patp: AAB25811 | Human secreted protein | 317 | 3.0e−28 |

In a search of sequence databases, it was found, for example, that the NOV3a nucleic acid sequence has 572 of 704 bases (81%) identical to a gb:GENBANK-ID:AB030182|acc:AB030182.1 mRNA from Mus musculus (Mus musculus mRNA, complete cds, clone:1-107). Further, the full amino acid sequence of the protein of the disclosed NOV3a protein of the invention has 173 of 209 amino acid residues (82%) identical to, and 182 of 209 amino acid residues (87%) similar to, the 208 amino acid residue ptnr:SPTREMBL-ACC:Q9JMG4 protein from Mus musculus (Mouse) (MRNA, COMPLETE CDS, CLONE:1-107).

In a similar search of sequence databases, it was found, for example, that the NOV3b nucleic acid sequence has 514 of 618 bases (83%) identical to a gb:GENBANK-ID:ABO30182|acc:AB030182.1 mRNA from Mus musculus (Mus musculus mRNA, complete cds, clone: 1-107). Further, the full amino acid sequence of the disclosed NOV3b protein of the invention has 165 of 196 amino acid residues (84%) identical to, and 173 of 196 amino acid residues (88%) similar to, the 208 amino acid residue ptnr:SPTREMBL-ACC:Q9JMG4 protein from Mus musculus (Mouse) (MRNA, COMPLETE CDS, CLONE:1-107).

Additional BLASTP results are shown in Table 3F.

TABLE 3F
NOV3 BLASTP Results

| Gene Index/ | | Length of | Identity | Positives | Expect |
|---|---|---|---|---|---|
| Identifier | Protein/Organism | aa | (%) | (%) | Value |
| Q9BQU8 | BA261N11.2.1 (NOVEL | 207 | 207/209 | 207/209 | 1.4e−110 |
| | PROTEIN, ISOFORM 1) - | | (99%) | (99%) | |
| | Homo sapiens | | | | |
| Q9JMG4 | MRNA, COMPLETE CDS, | 208 | 173/209 | 182/209 | 1.0e−91 |
| | CLONE: 1-107 | | (82%) | (87%) | |
| | (C030019F02RIK | | | | |
| | PROTEIN) - Mus musculus | | | | |
| | (Mouse) | | | | |
| Q9D8W0 | C030019F02RIK PROTEIN - | 208 | 172/209 | 181/209 | 3.5e−91 |
| | Mus musculus (Mouse) | | (82%) | (86%) | |
| Q9D1V9 | C030019F02RIK PROTEIN - | 208 | 172/209 | 181/209 | 3.5e−91 |
| | Mus musculus (Mouse) | | (82%) | (86%) | |
| Q9D0Q6 | 2610200G18RIK PROTEIN - | 207 | 120/206 | 144/206 | 8.0e−60 |
| | Mus musculus (Mouse) | | (58%) | (69%) | |

A multiple sequence alignment is given in Table 3G, with the NOV3 protein of the invention being shown in lines 1 and 2, in a ClustalW analysis comparing NOV3 with related protien sequences of Table 3F.

[image]

[image]

In a search of the Pfam database, there were no known domain results for NOV3.

The NOV3 disclosed in this invention is expressed in at least the following tissues: bone marrow, brain—substantia nigra, brain—temporal lobe, brain—whole, heart, kidney, pancreas, astrocytoma, CNS, multiple sclerosis lesions, and uterus. This information was derived by determining the tissue sources of the sequences that were included in the invention including but not limited to SeqCalling sources, Public EST sources, Literature sources, and/or RACE sources.

The protein similarity information, expression pattern, and map location for the transmembrane-like protein and nucleic acid disclosed herein suggest that this protein may have important structural and/or physiological functions characteristic of the transmembrane family. Therefore, the nucleic acids and proteins of the invention are useful in potential diagnostic and therapeutic applications and as a research tool. For example, the compositions of the present invention will have efficacy for treatment of patients suffering from: cancer, trauma, regeneration (in vitro and in vivo), viral/bacterial/parasitic infections, neuroprotection, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain and other diseases, disorders and conditions of the like.

The novel nucleic acid encoding the transmembrane-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 protein has multiple hydrophilic regions, each of which can be used as an immunogen. In one embodiment, a contemplated NOV3 epitope is from about amino acids 85 to 130. In another embodiment, a contemplated NOV3 epitope is from about amino acids 165 to 210.

NOV4

Still another NOVX protein of the invention, referred to herein as NOV4 (alternatively referred to as CG50289-01), is a serine protease-like protein.

Proteolytic enzymes that exploit serine in their catalytic activity are ubiquitous, being found in viruses, bacteria and eukaryotes. They include a wide range of peptidase activity, including exopeptidase, endopeptidase, oligopeptidase and omega-peptidase activity. Over 20 families of serine protease have been identified and although they have different evolutionary origins, there are similarities in the reaction mechanisms of several peptidases. Chymotrypsin, subtilisin and carboxypeptidase C clans have a catalytic triad of serine, aspartate and histidine in common: serine acts as a nucleophile, aspartate as an electrophile, and histidine as a base. The geometric orientations of the catalytic residues are similar between families, despite different protein folds. The enzymes are inherently secreted, being synthesised with a signal peptide that targets them to the secretory pathway. Animal enzymes are either secreted directly, packaged into vesicles for regulated secretion, or are retained in leukocyte granules.

Although SignalP, Psort and/or hydropathy suggest that the Serine Protease-like protein may be localized at the plasma membrane, the protein predicted here is similar to the Serine Protease family, some members of which are secreted. Therefore it is likely that this novel Serine Protease-like protein is available at the same sub-cellular localization and hence accessible to a diagnostic probe and for various therapeutic applications.

The NOV4 nucleic acid and polypeptide described in this application has a structure similar to Testicular Serine Protease-1 (TESP-1) and TESP-2, serine proteases isolated from the mouse sperm acrosome. These proteins may play a role in fertilization and/or processing of other proteins during fertilization.

The NOV4 protein disclosed in this invention maps to chromosome 2. This information was assigned using OMIM, the electronic northern bioinformatic tool implemented by CuraGen Corporation, public ESTs, public literature references and/or genomic clone homologies.

The NOV4 nucleic acid (SEQ ID NO: 13) of 909 nucleotides encodes a novel serine protease-like protein and is shown in Table 4A. An open reading frame for the mature protein was identified beginning with a ATG initiation codon at nucleotides 14-16 and ending with a TGA codon at nucleotides 899-901. Putative untranslated regions upstream from the start codon and downstream from the termination codon are underlined in Table 4A. The start and stop codons are in bold letters.

TABLE 4A
NOV4 Nucleotide Sequence

(SEQ ID NO:13)

GGCCACCGGCCTGATGAGGGAAGCAGGGGCAGAGCGCTCAGGCCAGCCGGCGGGGGCACTGCGCACTGGCCGCCTC
CCTCCTCTGGCCAATCCTCCTGCCGCTGCGCGTCTAGTCCACCTCGTCCCTCTCTGCAGGTCCACTAACCCATCTG
ATTACCGGATCCTGCTTGGGTATGACCAGCAAAGCCATCCCACAGAGCACAGCAAGCAGATGACAGTGAATAAGAT
CATGGTGCACGCTGACTATAACGAGTTGCACCGCATGGGGAGTGACATCACCCTGCTGCAGCTGCACCGTCATGTG
GAATTCAGCTCCCACATCCTCCCCGCCTGCCTTCCGGAACCAACCACGTGGCTGGCCCCTGACAGCTCCTGCTGGA
TATCTGGTTGGGGAATGGTCACCGAGGATGTCTTCCTGCCTGAGCCCTTCCAACTTCAGGAGGCAGAGGTCGGTGT
CATGGACAACACTGTCTGCGGATCCTTTTTCCAGCCCCAGTACCCCGGCCAGCCAAGCAGCAGTGACTACACCATC
CACGAGGACATGCTGTGCGCTGGGGACCTCATAACAGGAAAGGCCATTTGCCGACGAGACTCCAGGGGTCCCCTCG
TCTGCCCATTAAATGGCACCTGGTTCCTGATGGGGCTGTCTAGTTGGAGCCTCGACTGCTGCTCACCCGTCGGTCC
CAGGGTCTTCACCAGGCTCCCCTACTTCACCAACTGGATCAGCCAGAAGAAGAGGGAGAGCACCCCTCCAGATCCC
GCCTTGGCTCCTCCTCAGGAAACACCCCCAGCCCTGGACAGCATGACCTCTCAGGGCATCGTCCACAAGCCCGGGC
TCTGCGCAGCCCTTCTGGCTGCTCACATGTTCCTCCTGCTGCTGATTCTCCTGGGGAGCCTGTGAAGGGCCAG

The sequence of NOV4 was derived by laboratory cloning of cDNA fragments covering the full length and/or part of the DNA sequence of the invention, and/or by in silico prediction of the full length and/or part of the DNA sequence of the invention from public human sequence databases.

The DNA sequence and protein sequence for a novel polydom-like gene were obtained by SeqCallingTM Technology and are reported here as NOV4. These methods used to amplify NOV4 cDNA are described in Example 2.

The NOV4 polypeptide (SEQ ID NO: 14) encoded by SEQ ID NO: 13 is 295 amino acid residues in length and is presented using the one-letter amino acid code in Table 4B. The SignalP, Psort and/or Hydropathy results predict that NOV4 has no known signal peptide and is likely to be localized in the endoplasmic reticulum membrane with a certainty of 0.8500. In alternative embodiments, a NOV4 polypeptide is located to the plasma membrane with a certainty of 0.4400, the microbody (peroxisome) with a certainty of 0.3313, or the mitochondrial inner membrane with a certainty of 0.1000.

TABLE 4B
Encoded NOV4 Protein Sequence

(SEQ ID NO:14)

MREAGAERSGQPAGALRTGRLPPLANPPAAARLVHLVPLCRSTNPSDYRILLGYDQQSHPTEHSKQMTVNKIMVH
ADYNELHRMGSDITLLQLHRHVEFSSHILPACLPEPTTWLAPDSSCWISGWGMVTEDVFLPEPFQLQEAEVGVMD
NTVCGSFFQPQYPGQPSSSDYTIHEDMLCAGDLITGKAICRRDSRGPLVCPLNGTWFLMGLSSWSLDCCSPVGPR
VFTRLPYFTNWISQKKRESTPPDPALAPPQETPPALDSMTSQGIVHKPGLCAALLAAHMFLLLLILLGSL

SNP variants of NOV4 are disclosed in Example 3.

The amino acid sequence of NOV4 has high homology to other proteins as shown in Table 4C.

TABLE 4C
BLASTX Results from Patp Database for NOV4

| | | | Smallest |
|---|---|---|---|
| | | High | Sum |
| Sequences Producing High-Scoring Segment Pairs: | | Score | Prob P (N) |
| patp: AAW64239 | Gerbil homologue of mouse mMCP-7 zymogen - Meriones | 344 | 4.2e−31 |
| patp: AAW64240 | Human mast cell tryptase II/beta | 342 | 6.8e−31 |
| patp: AAW64241 | Human mast cell tryptase III | 342 | 6.8e−31 |
| patp: AAW63175 | Human mast cell tryptase II/beta polypeptide | 342 | 68e−31 |
| patp: AAW63176 | Human mast cell tryptase III polypeptide | 342 | 6.8e−31 |

In a search of sequence databases, it was found, for example, that the NOV4 nucleic acid sequence has 583 of 885 bases (65%) identical to a gb:GENBANK-ID:AB0089110|acc:AB008910.1 mRNA from Mus musculus (Mus musculus mRNA for TESP1, complete cds). Further, the full amino acid sequence of the disclosed NOV4 protein of the invention has 120 of 253 amino acid residues (47%) identical to, and 172 of 253 amino acid residues (67%) similar to, the 367 amino acid residue ptnr:SPTREMBL-ACC:O70169 protein from Mus musculus (Mouse) (TESTICULAR SERINE PROTEASE 1 (TESP1)).

Additional BLASTP results are shown in Table 4D.

TABLE 4D
NOV4 BLASTP Results

| Gene Index/ | | Length of | | | Expect |
|---|---|---|---|---|---|
| Identifier | Protein/Organism | aa | Identity (%) | Positives (%) | Value |
| O70169 | TESTICULAR SERINE | 367 | 120/253 | 172/253 | 2.1e−59 |
| | PROTEASE 1 (TESP1) - | | (47%) | (67%) | |
| | Mus musculus (Mouse) | | | | |
| O70170 | TESTICULAR SERINE | 366 | 120/252 | 157/252 | 4.0e−58 |
| | PROTEASE 2 (TESP2) - | | (47%) | (62%) | |
| | Mus musculus (Mouse) | | | | |
| Q9D9S6 | TESTICULAR SERINE | 143 | 69/140 | 90/140 | 4.5e−34 |
| | PROTEASE 2 - | | (49%) | (64%) | |
| | Mus musculus (Mouse) | | | | |
| Q9XSM2 | Tryptase 2 precursor (EC | 273 | 72/195 | 112/195 | 7.6e−32 |
| | 3.4.21.59) - | | (36%) | (57%) | |
| | Ovis aries (Sheep) | | | | |
| Q9XSM1 | TRYPTASE (EC 3.4.21.59) - | 273 | 73/195 | 112/195 | 9.7e−32 |
| | Ovis aries (Sheep) | | (37%) | (57%) | |

A multiple sequence alignment is given in Table 4E in a ClustalW analysis comparing NOV4 with related protein sequences disclosed in Table 4D.

[image]

[image]

Domain results for NOV4 were collected from the Pfam database, and then identified by the Interpro domain accession number. The results are listed in Table 4F with the statistics and domain description. These results indicatee that the NOV4 polypeptide has properties similar to those of other proteins known to contain these domains.

TABLE 4F

Domain Analysis of NOV4

| | | |
|---|---|---|
| | Score | E |
| PSSMs Producing Significant Alignments | (bits) | Value |

| | | |
|---|---|---|
| trypsin: domain 1 of 1, from 42 to 237 | 119.2 | 5.3e−37 |

| | | |
|---|---|---|
| Trypsin | sapassvrVSlsvrlGehnlsltegteqkfdvkktiivHpnynpdt. | (SEQ ID NO:65) |
| | ++ ++++++ +\| ++ +++ + ++++ + +\| +++ + | |
| N0V4 | STNPSDYRI----LLGYDQQSHPTEHSKQMTVNK-IMVHADYNELHr | (SEQ ID NO:14) |
| | ldngaYdnDiALlkLkspgvtlgdtvrpicLpsassdlpvGttctvsGwG | |
| | ++ \|+ \|++\| + +++++++ +++\|+++ +++ ++++++\|+\| | |
| NOV4 | MG-----SDITLLQLHRH-VEFSSHILPACLPEPTTWLAPDSSCWISGWG | |
| | rrptknlg...lsdtLqevvvpvvsretCrsaye..yggt......dDkv | |
| | + ++ + + +\|++++++++++ +\| + +++++++ +++++ + | |
| NOV4 | M--VTEDVflpEPFQLQEAEVGVMDNTVCGSFFQpqYPGQpsssdyT--- | |
| | efvtdnmiCagal.ggkdaCqGDSGGPLvcsdgnrdgrwelvGivSwGsy | |
| | + ++++\|++ + +++++\|+ \|\| \|\|\|+++ + ++++++\| \|++ | |
| NOV4 | --IHEDMLCAGDLiTGKAICRRDSRGPLVCPLN---GTWFLMGLSSWS-L | |
| | gCargnkPGvytrVssyldWI | |
| | \| ++ \| ++++ ++ +\|\| | |
| NOV4 | DCCSPVGPRVFTRLPYFTNWI | |

The Serine Protease disclosed in this invention is expressed in at least the following tissues: testis. This information was derived by determining the tissue sources of the sequences that were included in the invention.

The protein similarity information, expression pattern, and map location for the serine protease-like protein and nucleic acid disclosed herein suggest that this protein may have important structural and/or physiological functions characteristic of the serine protease family. Therefore, the NOV4 nucleic acids and proteins of the invention are useful in potential diagnostic and therapeutic applications and as a research tool. For example, the compositions of the present invention will have efficacy for treatment of patients suffering from: cancer, trauma, regeneration (in vitro and in vivo), viral/bacterial/parasitic infections, infertility and other diseases, disorders and conditions of the like.

The novel nucleic acid encoding the serine protease-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV4 protein has multiple hydrophilic regions, each of which can be used as an immunogen. In one embodiment, a contemplated NOV4 epitope is from about amino acids 10 to 30. In another embodiment, a contemplated NOV4 epitope is from about amino acids 35 to 40. In other specific embodiments, contemplated NOV4 epitopes are from about amino acids 45 to 90, 105 to 112, 115 to 120, 127 to 145, 152 to 180, 180 to 195, and 225 to 265.

NOV5

A further NOVX protein of the invention, referred to herein as NOV5, includes two novel Wnt-7a-like proteins. The disclosed proteins have been named NOV5a and NOV5b.

Wnt proteins constitute a large family of molecules involved in cell proliferation, cell differentiation and embryonic patterning. They are known to interact with the Frizzled family of receptors to activate two main intracellular signaling pathways regulating intracellular calcium levels and gene transcription. Wnts play a role in cell proliferation and tumorigenesis, and are also involved in processes involved in mammary gland development and cancer. Furthermore, Wnts are critical to organogenesis of several systems, such as the kidney and brain. Wnts regulate the early development, i.e. neural induction, and their role persists in later stages of development as well as in the mature organ.

The NOV5 proteins predicted here are localized extracellularly. Therefore, it is likely that these Wnt-7a-like proteins are accessible to a diagnostic probe, and for the various therapeutic applications described herein.

At least the NOV5a protein disclosed in this invention maps to chromosome 3. This information was assigned using the electronic northern bioinformatic tool implemented by CuraGen Corporation, public ESTs, public literature references and/or genomic clone homologies.

NOV5a

In one embodiment, a NOV5 variant is NOV5a (alternatively referred to herein as CG50353-01), which encodes a novel Wnt-7a-like protein and includes the 1628 nucleotide sequence (SEQ ID NO: 15) shown in Table 5A. An open reading frame for the mature protein was identified beginning with a ATG initiation codon at nucleotides 1-3 and ending with a TGA codon at nucleotides 1048-1050. Putative untranslated regions upstream from the start codon and downstream from the termination codon are underlined in Table 5A. The start and stop codons are in bold letters.

TABLE 5A
NOV5a Nucleotide Sequence

(SEQ ID NO:15)

ATGAACCGGAAAGCGCGGCGCTGCCTGGGCCACCTCTTTCTCAGCCTGGGCATGGTCTGTCTCCTAGCATGTGGC
TTCTCCTCAGTGGTAGCTCTGGGCGCAACGGTCATCTGTAACAAGATCCCAGGCCTGGCTCCCAGACAGCGGGCG
ATCTGCCAGAGCCGGCCCGACGCCATCATCGTCATAGGAGAAGGCTCACAAATGGGCCTGGACGAGTGTCAGTTT
CAGTTCCGCAATGGCCGCTGGAACTGCTCTGCACTGGGAGAGCGCACCGTCTTCGGGAAGGAGCTCAAAGTGGGG
AGCCGGGACGGTGCGTTCACCTACGCCATCATTGCCGCCGGCGTGGCCCACGCCATCACAGCTGCCTGTACCCAT
GGCAACCTGAGCGACTGTGGCTGCGACAAAGAGAAGCAAGGCCAGTACCACCGGGACGAGGGCTGGAAGTGGGGT
GGCTGCTCTGCCGACATCCGCTACGGCATCGGCTTCGCCAAGGTCTTCGTGGACGCTCGGGAGATCATGAAGAAC
GCGCGGCGCCTCATGAACCTGCATAACAATGAGGCCGGCAGGAAGGTTCTAGAGGACCGGATGCAGCTGGAGTGC
AAGTGCCACGGCGTGTCTGGCTCCTGCACCACCAAAACCTGCTGGACCACGCTGCCCAAGTTCCGAGAGGTGGGC
CACCTGCTGAAGGAGAAGTACAACGCGGCCGTGCAGGTGGAGGTGGTGCGGGCCAGCCGTCTGCGGCAGCCCACC
TTCCTGCGCATCAAACAGCTGCGCAGCTATCGCAAGCCCATGAAGACGGACCTGGTGTACATCGAGAAGTCGCCC
AACTACTGCGAGGAGGACCCGGTGACCGGCAGTGTGGGCACGCAGGGCCGCGCCTGCAACAAGACGGCTCCCCAG
GCCAGCGGCTGTGACCTCATGTGCTGTGGGCGTGGCTACAACACCCACCAGTACGCCCGCGTGTGGCAGTGCAAC
TGTAAGTTCCACTGGTGCTGCTATGTCAAGTGCAACACGTGCAGCGAGCGCACGGAGATGTACACGTGCAAGTGA
GCCCCGTGTGCACACCACCCTCCCGCTGCAAGTCAGATTGCTGGGAGGACTGGACCGTTTCCAAGCTGCGGGCTC
CCTGGCAGGATGCTGAGCTTGTCTTTTCTGCTGAGGAGGGTACTTTTCCTGGGTTTCCTGCAGGCATCCGTGGGG
GAAAAAAAATCTCTCAGAGCCCTCAACTATTCTGTTCCACACCCAATGCTGCTCCACCCTCCCCCAGACACAGCC
CAGGTCCCTCCGCGGCTGGAGCGAAGCCTTCTGCAGCAGGAACTCTGGACCCCTGGGCCTCATCACAGCAATATT
TAACAATTTATTCTGATAAAAATAATATTAATTTATTTAATTAAAAAGAATTCTTCCACCTCGTCGGGATCCGTT
TTCTGCAATCAAAGTGGACTGCTTGCTTTCCTAGCAGGATGATTTTGTTGCTAGGACAAGGAGCCGTGTAGAAGT
GTACATAACTATTCTTTATGCAGATATTTCTACTAGCTGATTTTGCAGGTACCCACCTTGCAGCACTAGATGTTT
AAGTACAAGAGGAGACATCTTTTATGCATATATAGATATACACACACGAAAAA

The sequence of NOV5a was derived by laboratory cloning of cDNA fragments covering the full length and/or part of the DNA sequence of the invention, and/or by in silico prediction of the full length and/or part of the DNA sequence of the invention from public human sequence databases.

The DNA sequence and protein sequence for a novel Wnt-7a-like gene were obtained by SeqCallingTM Technology and are reported here as NOV5a. These methods used to amplify NOV5a cDNA are described in Example 2.

The NOV5a polypeptide (SEQ ID NO: 16) encoded by SEQ ID NO: 15 is 349 amino acid residues in length and is presented using the one-letter amino acid code in Table 5B. The SignalP, Psort and/or Hydropathy results predict that NOV5a has a signal peptide and is likely to be localized extracellularly with a certainty of 0.8200. In alternative embodiments, a NOV5a polypeptide is located to the lysosome (lumen) with a certainty of 0.1900, the endoplasmic reticulum (membrane) with a certainty of 0.1000, or the endoplasmic reticulum (lumen) with a certainty of 0.1000.

TABLE 5B
Encoded NOV5a Protein Sequence

(SEQ ID NO:16)

MNRKARRCLGHLFLSLGMVCLLACGFSSVVALGATVICNKIPGLAPRQTAICQSRPDAIIVIGEGSQMGLDECQ
FQFRNGRWNCSALGERTVFGKELKVGSRDGAFTYAIIAAGVAHAITAACTHGNLSDCGCDKEKQGQYHRDEGWK
WGGCSADIRYGIGFAKVFVDAREIMKNARRLMNLHNNEAGRKVLEDRMQLECKCHGVSGSCTTKTCWTTLPKFR
EVGHLLKEKYNAAVQVEVVRASRLRQPTFLRIKQLRSYRKPMKTDLVYIEKSPNYCEEDPVTGSVGTQGRACNK
TAPQSGCDLMCCRGYNTHQYARVXQCNCKFHWCCYVKCNTCSERTEMYTCK

NOV5b

In alternative embodiments, a NOV5 variant is NOV5b (alternatively referred to herein as 169475673), which includes a 966 nucleotide sequence (SEQ ID NO: 17) shown in Table 5C below.

TABLE 5C
NOV5b Nucleotide Sequence

(SEQ ID NO:17)

AGATCTCTGGGCGCAACGGTCATCTGTAACAAGATCCCAGGCCTGGCTCCCAGACAGCGAACGATCTGCCAGAG
CCGGCCCGACGCCATCATCGTCATAGGAGAAAGGCTCACAATCGGCCTGGACGAGTGTCAGTTTCAGTTCCGCA
ATGGCCGCTGGAACTGCTCTGCACTGGGAGAGCGCACCGTCTTCGGGAAGGAGCTCAAAGTGGGGAGCCGGGAG
GCTGCGTTCACCTACGCCATCATTGCCGCCGGCGTGGCCCACGCCATCACAGCTGCCTGTACCCAGGGCAACCT
GAGCGACTGTGGCTGCGACAAAGAGAAGCAAGGCCAGTACCACCGGGACGAGAACTGGAAGTGGGGTGGCTGCT
CTGCCGACATCCGCTACGGCATCGGCTTCGCCAAGGTCTTTGTGGATGCCCGGGAGATCAAGCAGAATGCCCGG
ACTCTCATGAACTTGCACAACAACGAGGCAGGCCGAAAGATCCTGGAGGAGAACATGAAGCTGGAATGTAAGTG
CCACGGCGTGTCAGGCTCGTGCACCACCAAGACGTGCTGGACAACACTGCCAAAGTTTCGGGAGCTGGGCTACG
TGCTCAAGGACAAGTACAACGAGGCCGTTCACGTGGAGCCTGTGCGTGCCAGCCGCAACAAGCGGCCCACCTTC
CTGAAGATCAAGAAGCCACTGTCGTACCGCAAGCCCATGGACACGGACCTGGTGTACATCGAGAAGTCGCCCAA
CTACTGCGAGGAGGACCCGGTGACCGGCAGTGTGGGCACCCAGGGCCGCGCCTGCAACAAGACGGCTCCCCAGG
CCAGCGGCTGTGACCTCATGTGCTGTGGGCGTGGCTACAACACCCACCAGTACGCCCGCGTGTGGCAGTGCAAC
TGTAAGTTCCACTGGTGCTGCTATGTCAAGTGCAACACGTGCAGCGAGCGCACGGAGATGTACACGTGCAAGCT
CGAG

NOV5b is an insert assembly whose sequence was derived by laboratory cloning of cDNA fragments coding for a domain of the full length form of NOV5a (CG50353-01), between residues 32 to 349. The cDNA coding for the NOV5b sequence was cloned by the polymerase chain reaction (PCR). The PCR template is the previoisly identified plasma (NOV5a) when available or human cDNA. These primers and methods used to amplify NOV5b cDNA are described in Example 2.

The NOV5b polypeptide (SEQ ID NO: 18) encoded by SEQ ID NO: 17 is 322 amino acid residues in length and is presented using the one-letter amino acid code in Table 5D.

TABLE 5D
Encoded NOV5b Protein Sequence

(SEQ ID NO:18)

RSLGATVICNKIPGLAPRQRAICQSRPDAIIVIGEGSQMGLDECQFQFRNGRWNCSALGERTCFGKELKCGSREA
AFTYAIIAAGCAHAITAACTQGNLSDCGCDEKEKQGQYHRDEGWKWGGCSADIRYGIGFAKVFDAREIKQNARTL
MNLHNNEAGRKILEENMKLECKCHGVSGSCTTKTCWTTLPQFRELGYVLKDKYNEAVHVEPVRASRNKRPTFLKI
KKPLSYRKPMDTDLVYIEKSPNYCEEDPVTGSVGTQGRACNKTAPQASGCDIMCCGEGYNTHOYARVWQCNCKFH
WCCYVKCNTCSERTEMYTCKLE

SNP variants of NOV5 are disclosed in Example 3.

NOV5 Clones

Unless specifically addressed as NOV5a or NOV5b, any reference to NOV5 is assumed to encompass all variants.

The amino acid sequence of NOV5 has high homology to other proteins as shown in Table 5E.

TABLE 5E
BLASTX Results from Patp Database for NOV5

| | | | Smallest |
|---|---|---|---|
| | | High | Sum |
| Sequences Producing High-Scoring Segment Pairs: | | Score | Prob P (N) |
| patp: AAB19789 | Human Wnt-7a protein involved in kidney tubulogenesis | 1784 | 1.1e−183 |
| patp: AAY70737 | Human Wnt-7a protein | 1784 | 1.1e−183 |
| patp: AAY57598 | Human Wnt-7a protein | 1784 | 1.1e−183 |
| patp: AAY93965 | Amino acid sequence of a human WNT-7A polypeptide | 1758 | 6.1e−181 |
| patp: AAR75881 | Human Wnt-x | 887 | 1.2e−88 |

In a search of sequence databases, it was found, for example, that the NOV5a nucleic acid sequence has 1336 of 1412 bases (94%) identical to a gb:GENBANK-ID:HSU53476|acc:U53476.1 mRNA from Homo sapiens (Human proto-oncogene Wnt7a mRNA, complete cds). Further, the full amino acid sequence of the disclosed NOV5a protein of the invention has 321 of 349 amino acid residues (91%) identical to, and 335 of 349 amino acid residues (95%) similar to, the 349 amino acid residue ptnr:SWISSPROT-ACC:O00755 protein from Homo sapiens (Human) (WNT-7A PROTEIN PRECURSOR).

Additional BLASTP results are shown in Table 5F.

TABLE 5F
NOV5 BLASTP Results

| Gene Index/ | | Length of | | | Expect |
|---|---|---|---|---|---|
| Identifier | Protein/Organism | aa | Identity (%) | Positives (%) | Value |
| O00755 | WNT-7A protein precursor - | 349 | 321/349 | 335/349 | 1.4e−183 |
| | Homo sapiens (Human) | | (91%) | (95%) | |
| AAH08811 | UNKNOWN (PROTEIN | 349 | 317/349 | 333/349 | 7.8e−181 |
| | FOR MGC: 10346) - | | (90%) | (95%) | |
| | Homo sapiens | | | | |
| | (Human) | | | | |
| Q9DBY3 | WINGLESS-RELATED | 349 | 315/349 | 332/349 | 8.9e−180 |
| | MMTV INTEGRATION | | (90%) | (95%) | |
| | SITE 7A - | | | | |
| | Mus musculus (Mouse) | | | | |
| P24383 | WNT-7A protein | 349 | 313/349 | 330/349 | 3.5e−178 |
| | precursor - | | (89%) | (94%) | |
| | Mus musculus (Mouse) | | | | |
| Q9DEB8 | WNT-7A - Gallus gallus | 349 | 302/349 | 329/349 | 4.7e−174 |
| | (Chicken) | | (86%) | (94%) | |

A multiple sequence alignment is given in Table 5G in a ClustalW analysis comparing NOV5 with related protein sequences disclosed in Table 5F.

[image]

[image]

Domain results for NOV5 were collected from BLAST sample domains found in the Smart and Pfam collections, and then identified by the Interpro domain accession number. The results are listed in Table 5H with the statistics and domain description. These results indicate that the NOV5 polypeptides have properties similar to those of other proteins known to contain these domains and similar to the properties of these domains.

TABLE 5H
Domain Analysis of NOV5

| | Score | E |
|---|---|---|
| PSSMs Producing Significant Alignments | (bits) | Value |
| writ: domain 1 of 1, from 37 to 349 | 716.5 | 3e−260 |

| | | |
|---|---|---|
| Wnt | lCrslPGLsprQrqlcrrnpdvmasvseGaqlaiqEcQhQFRgrRWN | (SEQ ID NO:71) |
| | +\|+++\|\|\|+++\|+++\|++++++++++++\|++++ +\|\|\|+\|\|\|++\|\|\| | |
| NOV5a | ICNKIPGLAPRQRAICQSRPDAIIVIGEGSQMGLDECQFQFRNGRWN | (SEQ ID NO:16) |
| | CStldslnersvfgkvlkkgtREtAFvyAIsSAGVahaVTRaCseGeles | |
| | \|\|+++ +++++++++++++\|++\|\|++\|\| +\|\|\|++++\|++\| \|++++ | |
| NOVSa | CSALG---ERTVFGKELKVGSRDGAFTYAIIAAGVAHAITAACTHGNLSD | |
| | CGCDdkRkadeerlrikLepkgpggpqgswkwGGcsDNvefGirfSReFV | |
| | \|\|\|\|+ +++++++ +++ + +++++ +++++ \|\| | |
| NOV5a | CGCDK-------------EKQGQYHRDEGWKWGGCSADIRYGIGFAKVFV | |
| | DarEreklmtksrdrdaRsLMNLHNNEAGRkaVkshmrreCKCHGvSGSC | |
| | ++ + ++ +\|+\|\|\|\|\|\|\|\|\|\|\|+++++++ ++\|\|\|\|\|+\|\|\|\| | |
| NOV5a | DAREIM------KN--ARRLMNLHNNEAGRKVLEDRMQLECKCHGVSGSC | |
| | slKTCWlsLPdFReVGdlLKeKYdgAievevnkrgkgqrslssrkqasal | |
| | ++\|\|\|\|++\|\|+\|\|+\|\|++\|\|+\|\|+ \|++\|+++++++ +++++++ | |
| NOVSa | TTKTCWTTLPKFREVGHLLKEKYNAAVQVEVVPASR------LRQPTFLR | |
| | eaanerfkkptrnQYTDLvylEkSPDYCerdretQslGTqcRvcnktskG | |
| | +++ +++++\|+++ \|\|\|\|+\|+\|\|+\|\|++++ +\|++\|\|+\|\|+\|++++++ | |
| NOV5a | IKQLRSYRKPMKT---DLVYIEKSPNYCEEDPVTGSVGTQGRACNKTAPQ | |
| | lqWRDgCelLCCGRGYnteqKvertekCnCkFHNGWCCyVkCeeCtevve | |
| | + ++\|+++\|\|\|\|\|\|++++ + ++++\|+\|+\|\|++\|\|\|+\|+\|++\|+++++ | |
| NOV5a | A---SGCDLMCCGRGYNTHQ-YARVWQCNCKFH--WCCYVKCNTCSERTE | |
| | vhtCK | |
| | +++\|\| | |
| NOV5a | MYTCK | |

The Wnt-7a-like protein disclosed in this invention is expressed in at least the following tissues: testis, pancreas, brain, coronary artery, dermis, prostate, uterus and ovary. This information was derived by determining the tissue sources of the sequences that were included in the invention, including but not limited to, SeqCalling sources, PublicEST sources, RACE sources, and publicly available reference material from OMIM and Pubmed.

The protein similarity information, expression pattern, and map location for the Wnt-7a-like protein and nucleic acid disclosed herein suggest that this protein may have important structural and/or physiological functions characteristic of the Wnt family. Therefore, the NOV5 nucleic acids and proteins of the invention are useful in potential diagnostic and therapeutic applications and as a research tool. For example, the compositions of the present invention will have efficacy for treatment of patients suffering from: atherosclerosis, aneurysm, hypertension, fibromuscular dysplasia, stroke, scleroderma, obesity, transplantation disorders, myocardial infarction, embolism, cardiovascular disorders, bypass surgery, endometriosis, infertility, polycystic ovary syndrome, Von Hippel-Lindau (VHL) syndrome, Alzheimer's disease, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, psoriasis, actinic keratosis, acne, hair growth/loss, allopecia, pigmentation disorders, endocrine disorders, pancreatitis, diabetes and other diseases, disorders and conditions of the like.

The novel nucleic acid encoding the Wnt-7a-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV5 protein has multiple hydrophilic regions, each of which can be used as an immunogen. In one embodiment, a contemplated NOV5 epitope is from about amino acids 40 to 50. In another embodiment, a contemplated NOV5 epitope is from about amino acids 52 to 57. In other specific embodiments, contemplated NOV5 epitopes are from about amino acids 57 to 60, 65 to 100, 125 to 150, 165 to 210, 210 to 230, 230 to 240, 240 to 295, 300 to 325, and 325 to 340.

NOV6

Another NOVX protein of the invention, referred to herein as NOV6, includes two novel apical endosomal glycoprotein (AEG)-like proteins. The disclosed proteins have been named NOV6a and NOV6b.

After endocytosis from the plasma membrane, internalized receptors and ligands are delivered to endosomes. The endosomal compartment performs a variety of functions, including the sorting of internalized receptors and ligands, and newly synthesized lysosomal membrane proteins and hydrolases. In polarized epithelial cells, the apical endosomal compartment plays a role in both apical to basolateral and basolateral to apical transepithelial transport.

The NOV6 proteins disclosed here are predicted to localize at the plasma membrane. Therefore, it is likely that these proteins are accessible to a diagnostic probe, and for the various therapeutic applications described herein.

At least the NOV6a protein of the invention maps to chromosome 9. This information was assigned using OMIM, the electronic northern bioinformatic tool implemented by CuraGen Corporation, public ESTs, public literature references and/or genomic clone homologies.

NOV6a

In one embodiment, a NOV6 variant is NOV6a (alternatively referred to herein as CG50221-01), which encodes a novel apical endosomal glycoprotein (AEG)-like protein and includes the 3731 nucleotide sequence (SEQ ID NO: 19) shown in Table 6A. An open reading frame for the mature protein was identified beginning with an ATG codon at nucleotides 39-41 and ending with a TAG codon at nucleotides 3699-3701. Putative untranslated regions downstream from the termination codon and upstream from the initiation codon are underlined in Table 6A, and the start and stop codons are in bold letters. Table 6A. NOV6a Nucleotide Sequence (SEQ ID NO: 19)

TABLE 6A
NOV6a Nucleotide Sequence

(SEQ ID NO:19)

GCACCCTGTGTGGCCGCACTGCTCCCTCTGGCCCAACCATGCCTCTGTCCAGCCACCTGCTGCCCGCCTTGGTCCT
GTTCCTGGCAGCAGGGTCCTCAGGCTGGGCCTGGGTCCCCAACCACTGCAGGAGCCCTGGCCAGGCCGTGTGCAAC
TTCGTGTGTGACTGCAGGGACTGCTCAGATGAGGCCCAGTGTGGTTACCACGGGGCCTCGCCCACCCTGGGCGCCC
CCTTCGCCTGTGACTTCGAGCAGGACCCCTGCGGCTGGCGGGACATTAGTACCTCAGGCTACAGCTGGCTCCGAGA
CAGGGCAGGGGCCGCACTGGAGGGTCCTGGGCCTCACTCAGACCACACACTGGGCACCGACTTGGGCTGGTACATG
GCCGTTGGAACCCACCGAGGGAAAGACGCATCCACCGCAGCCCTGCGCTCGCCAACCCTGCGAGAGGCAGCCTCCT
CTTGCAAGCTGAGGCTCTGGTACCACGCGGCCTCTGGAGATGTGGCTGAACTGCGGGTGGAGCTGACCCATGGCGC
AGAGACCCTGACCCTGTGGCAGAGCACAGGGCCCTGGGGCCCTGGCTGGCAGGAGTTGGCAGTGACCACAGGCCGC
ATCCGGGGTGACTTCCGAGTGACCTTCTCTGCCACCCGAAATGCCACCCACAGGGGCGCTGTAACTCTAGATGACC
TAGAGTTCTGGGACTGTGGTCTGCCCACCCCCCAGGCCAACTGTCCCCCGGGACACCACCACTGCCAGAACAAGGT
CTGCGTGGAGCCCCAGCAGCTGTGCGACGGGGAAGACAACTGCGGGGACCTGTCTGATGAGAACCCACTCACCTGT
GGCCGCCACATAGCCACCGACTTTGAGACAGGCCTGGGCCCATGGAACCGCTCGGAAGGCTGGTCCCGGAACCACC
GCGCTGGTGGTCCTGAGCGCCCCTCCTGGCAACGCCGTGACCACAGCCGGAACAGTGCACAGGGCTCCTTCCTGGT
CTCCGTGGCCGAGCCTGGAACCCCTGCTATACTCTCCAGCCCCGAATTCCAAGCCTCAGGCACCTCCAACTGCTCG
GTGAGATGGCTGGTCTTCTATCAGTACCTGAGTGGGTCTGAGGCTGGCTGCCTCCAGCTGTTCCTGCAGACTCTGG
GGCCCGGCGCCCCCCGGGCCCCCGTCCTGCTGCGGAGGCGCCGAGGGGAGCTGGGGACCGCCTGGGTCCGAGACCG
TGTTGACATCCAGAGCGCCTACCCCTTCCAGATCCTCCTGGCCGGGCAGACAGGCCCGGGGGGCGTCGTGGGTCTG
GACGACCTCATCCTGTCTGACCACTGCAGACCAGTCTCGGAGGTGTCCACCCTGCAGCCGCTGCCTCCTGGGCCCC
GGGCCCCAGCCCCCCAGCCCCTGCCGCCCAGCTCGCGGCTCAAGGATTCCTGCAAGCAGGGGCATCTTGCCTGCGG
GGACCTGTGTGTGCCCCCGGAACAACTGTGTGACTTCGAGGAGCAGTGCGCAGGGGGCGAGGACGAGCAGGCCTGT
GGCACCACAGACTTTGAGTCCCCCGAGGCTGGGGGCTGGGAGGACGCCAGCGTGGGGCGGCTGCAGTGGCGGCGTG
TCTCAGCCCAGGAGAGCCAGGGGTCCAGTGCAGCTGCTGCTGGGCACTTCCTGTCTCTGCAGCGGGCCTGGGGGCA
GCTAGGCGCTGAGGCCCGGGTCCTCACACCCCTCCTTGGCCCTTCTGGCCCCAGCTGTGAACTCCACCTGGCTTAT
TATTTACAGAGCCAGCCCCGAGGCTTCCTGGCACTAGTTGTGGTGGACAACGGCTCCCGGGAGCTGGCATGGCAGG
CCCTGAGCAGCAGTGCAGGCATCTGGAAGGTGGACAAGGTCCTTCTAGGGGCCCGCCGCCGGCCCTTCCGGCTGGA
GTTTGTCGGTTTGGTGGACTTGGATGGCCCTGACCAGCAGGGAGCTGGGGTGGACAACGTGACCCTGAGGGACTGT
AGCCCCACAGTGACCACCGAGAGAGACAGAGAGGTCTCCTGTAACTTTGAGCGGGACACATGCAGCTGGTACCCAG
GCCACCTCTCAGACACACACTGGCGCTGGGTGGAGAGCCGCGGCCCTGACCACGACCACACCACAGGCCAAGGCCA
CTTTGTGCTCCTGGACCCCACAGACCCCCTGGCCTGGGGCCACAGTGCCCACCTGCTCTCCAGGCCCCAGGTGCCA
GCAGCACCCACGGAQTGTCTCAGCTTCTGGTACCACCTCCATGGGCCCCAGATTGGGACTCTGCGCCTAGCCATGA
GACGGGAAGGGGAGGAGACACACCTGTGGTCGCGGTCAGGCACCCAGGGCAACCGCTGGCACGAGGCCTGGGCCAC
CCTTTCCCACCAGCCTGGCTCCCATGCCCAGTACCAGCTGCTGTTCGAGGGCCTCCGGGACGGATACCACGGCACC
ATGGCGCTGGACGATGTGGCCGTGCGGCCGGGCCCCTGCTGGGCCCCTAATTACTGCTCCTTTGAGGACTCAGACT
GCGGCTTCTCCCCTGGAGGCCAAGGTCTCTGGAGGCGGCAGGCCAATGCCTCGGGCCATGCTGCCTGGGGCCCCCC
AACAGACCATACCACTGAGACAGCCCAAGGGCACTACATGGTGGTGGACACAAGCCCAGACGCACTACCCCGGGGC
CAGACGGCCTCCCTGACCTCCAAGGAGCACAGGCCCCTGGCCCAGCCTGCTTGTCTGACCTTCTGGTACCACGGGA
GCCTCCGCAGCCCAGGCACCCTGCGGGTCTACCTGGAGGAGCGCGGGAGGCACCAGGTGCTCAGCCTCAGTGCCCA
CGGCGGGCTTGCCTGGCGCCTGGGCAGCATGGACGTGCAGGCCGAGCGAGCCTGGAGGGTGGTGTTTGAGGCAGTG
GCCGCAGGCGTGGCACACTCCTACGTGGCTCTGGATGATCTGCTCCTCCAGGACGGGCCCTGCCCTCAGCCAGGTT
CCTGTCATTTTGAGTCTGGCCTGTGTGGCTGGAGCCACCTGGCCTGGCCCGGCCTGGGCGGATACAGCTGGGACTG
GGGCGGGGGAGCCACCCCCTCTCGTTACCCCCAGCCCCCTGTGGACCACACCCTGGGCACAGAGGCAGGCCACTTT
GCCTTCTTTGAAACTGGCGTGCTGGGCCCCGGGGGCCGGGCCGCCTGGCTGCGCAGCGAGCCTCTGCCGGCCACCC
CAGCCTCCTGCCTCCGCTTCTGGTACCACATGGGTTTTCCTGAGCACTTCTACAAGGGGGAGCTGAAGGTACTGCT
GCACAGTGCTCAGGGCCAGCTGGCTGTGTGGGGCGCAGGCGGGCATCGGCGGCACCAGTGGCTGGAGGCCCAGGTG
GAGGTAGCCAGTGCCAAGGAGTTCCAGATCGTGTTTGAAGCCACTCTGGGCGGCCAGCCAGCCCTGGGGCCCATTG
CCCTGGATGACGTGGAGTATCTGGCTGGGCAGCATTGCCAGCAGCCTGCCCCCAGCCCGGGGAACACAGCCGCACC
CGGGTCTGTGCCAGCTGTGGTTGGCAGTGCCCTCCTATTGCTCATGCTCCTGGTGCTGCTGGGACTTGGGGGACGG
CGCTGGCTGCAGAAGAAGGGGAGCTGCCCCTTCCAGAGCAACACAGAGGCCACAGCCCCTGGCTTTGACAACATCC
TTTTCAATGCGGATGGTGTCACCCTCCCGGCATCTGTCACCAGTGATCCGTAGACCACCCCAGACAAGGCCC
CGCTTCCTCAC

The sequence of NOV6a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The NOV6a polypeptide (SEQ ID NO: 20) encoded by SEQ ID NO: 19 is 1220 amino acid residues in length and is presented using the one-letter amino acid code in Table 6B. The SignalP, Psort and/or Hydropathy results predict that NOV6a has a signal peptide and is likely to be localized extracellularly at the plasma membrane with a certainty of 0.4600. In alternative embodiments, a NOV6a polypeptide is located to the microbody (peroxisome) with a certainty of 0.2742, the endoplasmic reticulum (membrane) with a certainty of 0.1000, or the endoplasmic reticulum (lumen) with a certainty of 0.1000. The SignalP predicts a likely cleavage site for a NOV6a peptide between amino acid positions 23 and 24, i.e. at the dash in the sequence GWA-WV.

TABLE 6B
Encoded NOV6a Protein Sequence

(SEQ ID NO:20)

MPLSSHLLPALVLFLAAGSSGWANVPNHCRSPGGAVCNFVCDCRDCSDEAQCGYHGASPTLGAPFACDFEQDPC
GWRDISTSGYSWLRDRAGAALEGPGPHSDHTLGTDLGWYMAVGTHRGKEASTAALRSPTLREAASSCKLRLWYH
AASGDVAELRVELTHGAETLTLWQSTGPWGPGWQELAVTTGRIRGDFRVTFSATRNATHRGAVALDDLEFWDCG
LPTPQANCPPGHHHCQNKVCVEPQQLCDGEDNCGDLSDENPLTCGRHIATDFETGLGPWNRSEGWSRNHRAGGP
ERPSWPRRDHSRNSAQGSFLVSVAEPGTPAILSSPEFQASGTSNCSVRWLVFYQYLSGSEAGCLQLFLQTLGPG
APRAPVLLRRRRGELGTAWVRDRVDIQSAYPFQILLAGQTGPGGVVGLDDLILSDHCRPVSEVSTLQPLPPGPR
APAPQPLPPSSRLQDSCKQGHLACGDLCVPPEQLCDFEEQCAGGEDEQACGTTDFESPEAGGWEDASVGRLQWR
RVSAQESQGSSAAAAGHFLSLQRAWGQLGAEARVLTPLLGPSGPSCELHALYYLQSQPRGFLALVVVDNGSREL
AWQALSSSAGIWKVDKVLLGARRRPFRLEFVGLVDLDGPDQQGAGVDNTLRDCSPAAJTTERDREVSCNFERDT
CSWYPGHLSDTHWRWVESRGPDHDHTTGQGHFVLLDPTDPLAWGHSAHLLSRPQVPAAPTECLSFWYHAAGPQI
GTLRLAMRREGEETHLWSRSGTQGNRWHEAWATLSHQPGSHAQYQLLFEGLRDGYHGTMALKKVAVRPGPCWAP
NYCSFEDSDCGFSPGGGGLWRRQANASGHAAWGPPTDHTTETAQGHYIVAATSPDAAPRGGTASLTSKEHRPAA
QPACLTFWYHGSLRSPGTLRVYLEERGRHQVLSLSAHGGLAWRLGSMDVQAERAWRVVFEAVAAGVAHSYVALD
DLLLQDGPCPQPGSCDFESGLCGWSHAAWPGLGGYSAAWGGGATPSRYPQPPAAHTLGTAAGHFAFFETGVLGP
GGRAAWLRSEPLPATPASCLRFWYAAGFPEHFYKGELAALLESAQGQLAVWGAGGHRRHQWLEAQVEVASAKEF
QIVFEATLGGQPALGPIALDDVEYLAGGHCGGPAPSPGAATAAPGSVPAWGSALLLLMLLVLLGLGGRRWLQKK
GSCPFQSNTEATAPGFDNTLFNADQVTLPASVTSDP

NOV6b

In an alternative embodiment, a NOV6 variant is NOV6b (alternatively referred to herein as 174308633), which includes 1857 nucleotides. NOV6b is an insert assembly that was found to encode an open reading frame between residues 31 and 648 of the target sequence of NOV6a. NOV6b differs from NOV6a at 4 nucleotide and 4 amino acid positions. It also contains a 3 amino acid deletion, and a 9 nucleotide deletion in comparison with NOV6a. Table 6C notes the changes in nucleotide and amino acid sequences from the parent clone, NOV6a.

TABLE 6C

| | | | Change in |
|---|---|---|---|
| Nov | Alternate | Change in DNA Seq. | Protein Seq. from |
| No. | Reference | from NOV6a | NOV6a |
| 6b | 174308633 | T → C at bp 385; | V → A at aa 116; |
| | | C → T at bp 914; | A → T at aa 326; |
| | | C → T at bp 1007; and | T → L at aa 649; and |
| | | G → A at bp 1014 | V → E at aa 650 |

The sequence of NOV6b was derived by laboratory cloning of cDNA fragments coding for a domain of the full length form of NOV6a, between residues 31 and 648. The cDNA coding for the NOV6b sequence was cloned by the polymerase chain reaction (PCR). The PCR template is the previoisly identified plasma (NOV6a) when available or human cDNA. These primers and methods used to amplify NOV6b cDNA are described in Example 2.

SNP variants of NOV6 are disclosed in Example 3.

NOV6 Clones

Unless specifically addressed as NOV6a or NOV6b, any reference to NOV6 is assumed to encompass all variants.

The amino acid sequence of NOV6 has high homolgy to other proteins as shown in Table 6D.

TABLE 6D
BLASTX Results from Patp Database for NOV6

| | | | Smallest |
|---|---|---|---|
| | | High | Sum |
| Sequences Producing High-Scoring Segment Pairs: | | Score | Prob P (N) |
| patp: AAB42780 | Human ORFX ORF2544 polypeptide | 1274 | 2.5e−230 |
| patp: AAB01432 | Human TANGO 239 (form 2) | 377 | 2.4e−33 |
| patp: AAB00036 | Human TANGO 239 partial sequence | 281 | 4.9e−21 |
| patp: AAB01426 | Human TANGO 239 | 271 | 2.5e−19 |
| patp: AAE00585 | Human nuclear cell adhesion molecule homologue | 225 | 2.3e−14 |

In a search of sequence databases, it was found, for example, that the NOV6a nucleic acid sequence of this invention has 913 of 945 bases (96%) identical to a gb:GENBANK-ID:HSM801957|acc:AL137659.1 mRNA from Homo sapiens (Homo sapiens mRNA; cDNA DKFZp434I1716 (from clone DKFZp434I1716)). Further, the full amino acid sequence of the disclosed protein of the invention has 885 of 1220 amino acid residues (72%) identical to, and 990 of 1220 amino acid residues (81%) similar to, the 1216 amino acid residue ptnr:SWISSPROT-ACC:Q63191 protein from Rattus norvegicus (Rat) (APICAL ENDOSOMAL GLYCOPROTEIN PRECURSOR).

Additional BLASTP results are shown in Table 6E.

TABLE 6E
NOV6 BLASTP Results

| Gene Index/ | | Length of | | | Expect |
|---|---|---|---|---|---|
| Identifier | Protein/Organism | aa | Identity (%) | Positives (%) | Value |
| Q63191 | Apical endosomal | 1216 | 885/1220 | 990/1220 | 0.0 |
| | glycoprotein precursor - | | (72%) | (81%) | |
| | Rattus norvegicus (Rat) | | | | |
| Q91641 | Thyroid hormone-induced | 668 | 131/452 | 212/452 | 1.4e−31 |
| | protein B precursor - | | (28%) | (46%) | |
| | Xenopus laevis (African | | | | |
| | clawed frog) | | | | |
| O88799 | Zonadhesin precursor - | 5376 | 146/502 | 228/502 | 9.8e−28 |
| | Mus musculus (Mouse) | | (29%) | (45%) | |
| Q99ND0 | ZAN - Mus musculus | 5374 | 146/502 | 227/502 | 1.3e−27 |
| | (Mouse) | | (29%) | (45%) | |
| Q9BZ84 | ZONADHESIN VARIANT | 2601 | 149/491 | 215/491 | 5.4e−23 |
| | 5 - Homo sapiens (Human) | | (30%) | (43%) | |

A multiple sequence alignment is given in Table 6F, with the NOV6 protein of the invention being shown in lines 1 and 2, in a ClustalW analysis comparing NOV6 with related protien sequences of Table 6E.

[image]

[image]

[image]

[image]

[image]

[image]

[image]

[image]

[image]

[image]

[image]

[image]

Domain results for NOV6 were collected from the Pfam database, and then identified by the Interpro domain accession number. The results are listed in Table 6G with the statistics and domain description. These results indicate that the NOV6 polypeptides have properties similar to those of other proteins known to contain these domains.

TABLE 6G
Domain Analysis of NOV6

| | Score | E |
|---|---|---|
| PSSMs Producing Significant Alignments | (bits) | Value |
| MAM: domain 4 of 6, from 660 to 813 | 192.5 | 6.7e−54 |

| | | |
|---|---|---|
| MAM | CdFEdgshFfCgWsqdsgddgddlqwtrvnsatggstgprgdhttGn | (SEQ ID NO:77) |
| | \|+\|\| + \| \| +++++ +\| ++ ++ ++ +++++\|+ | |
| NOVGa | CNFERDT---CSWYPGHLSD---THWRVESR-----GPDHDHTTGQ | (SEQ ID NO:20) |
| | GhymyvdtssgllqeGqkArLlSpplppnrspeccLtFwYhmyGsgvgtp | |
| | \|++ ++++++ + + \|++\|+\|+\|++ ++++ \|\|+\|+\|+++\|++ ++ | |
| NOV6a | GHFVLLDPTDPL-AWGHSAHLLSRPQVPAAPT-ECLSFWYHLHGPQIGT- | |
| | gLnvyvrenge.tllWsrsGhqggqWllaevtlpt..fstkptqvvFegt | |
| | \|+++++++++++ +\|+++\|++++++\|++++ ++++++++ +++++\|+++ | |
| NOV6a | -LRLAMRREGEeTHLWSRSGTQGNRWHEAWATLSHqpGSHAQYQLLFEGL | |
| | rgggsrGgIAlDDIslsthiegpcnq | |
| | + +++\|++\|+\|\|+ ++ +++\|++ | |
| NOV6a | R-DGYHGTMALDDVAVR---PGPCWA | |

| | | |
|---|---|---|
| MAM: domain 6 of 6, from 977 to 1142 | 199.2 | 6.4e−56 |

| | | |
|---|---|---|
| MAM | CdFEdgshPfCgWsqdsgddgddlqWtrvnsatgg.stgprgdhttG | (SEQ ID NO:78) |
| | \|+\|\|++ +\|+\|++ +++++++ \| ++ ++++++++ ++++++ \| | |
| | CDFESG---LCGWSHLAWPGLGGYSWDWGGGATPSrYPQPPVDHTLG | (SEQ ID NO:20) |
| | n..GhymyvdtssgllqeGqkArLlspplppnrspeccLtFwyhmyGsgv | |
| | ++ \|++ ++++ + + ++\|++\|+\|+\|++++++ + \|\|+\|+\|++ ++++ | |
| NOV6a | TeaGHFAFFETGVLG-PGGRAAWLRSEPLPATPAS--CLRFWYHMGFPEH | |
| | gtpg.Lnvyvrenge.tllWsrsGhqggqwllaevtlptfstkpfqvvFe | |
| | ++++\| +++ + +++ +\| ++\|+ ++\|+++++++ + ++++++\|+ | |
| NOV6a | FYKGeLKVLLHSAQGqLAVWGAGGHRPHQWLEAQVEVA--SAKEFQIVFE | |
| | gtrg.ggsrGgIAlDDlslsthiegpcnq | |
| | ++ ++++ +\| \|\|+\|\|+++++ +++\| + | |
| NOV6a | ATLGgQPALGPIALDDvEyLA-GGHcQQ | |

The NOV6 disclosed in this invention may be expressed in a variety of tissues.

The protein similarity information, expression pattern, and map location for the apical endosomal glycoprotein-like protein and nucleic acid disclosed herein suggest that this protein may have important structural and/or physiological functions characteristic of the apical endosomal glycoprotein family. Therefore, the nucleic acids and proteins of the invention are useful in potential diagnostic and therapeutic applications and as a research tool. For example, the compositions of the present invention will have efficacy for treatment of patients suffering from: endometriosis, fertility and other diseases, disorders and conditions of the like.

The novel nucleic acid encoding the apical endosomal glycoprotein-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV6 protein has multiple hydrophilic regions, each of which can be used as an immunogen. In one embodiment, a contemplated NOV6 epitope is from about amino acids 20 to 150. In another embodiment, a contemplated NOV6 epitope is from about amino acids 150 to 200. In alternative embodiments, contemplated NOV6 epitopes include from about amino acids 205 to 310, 320 to 355, 375 to 410, 410 to 440, 440 to 550, 570 to 740, 740 to 800, 800 to 950, 950 to 990, 995 to 1025, 1045 to 10 1070, 1100to 1120, 1125 to 1160, and 1175 to 1210.

NOV7

Another NOVX protein of the invention, referred to herein as NOV7, includes three novel ADAM13-like proteins. The disclosed proteins have been named NOV7a, NOV7b, and NOV7c. The ADAM family proteins contain a metalloprotease domain, a disintegrin domain, and a cystein-rich domain. The proteins are human homolgs of mouse meltrin-alpha, which are involved in mytube formation

The NOV7 proteins disclosed herein are predicted to localize extracellularly. Therefore, it is likely that these proteins are accessible to a diagnostic probe, and for the various therapeutic applications described herein.

At least the NOV7a protein disclosed in this invention maps to chromosome 20. This information was assigned using OMIM, the electronic northern bioinformatic tool implemented by CuraGen Corporation, public ESTs, public literature references and/or genomic clone homologies.

NOV7a

In one embodiment, a NOV7 variant is NOV7a (alternatively referred to herein as CG50367-01), which encodes a novel ADAM13-like protein and includes the 2762 nucleotide sequence (SEQ ID NO: 21) shown in Table 7A. An open reading frame for the mature protein was identified beginning with an ATG codon at nucleotides 3-5 and ending with a TGA codon at nucleotides 2745-2747. Putative untranslated regions downstream from the termination codon and upstream from the initiation codon are underlined in Table 7A, and the start and stop codons are in bold letters.

TABLE 7A
NOV7a NuCleotide Sequence

(SEQ ID NO:21)

CTATGGGCTGGAGGCCCCGGAGAGCTCGGGOGACCCCGTTGCTGCTGCTGCTACTACTGCTGCTGCTCTGGCCAG
TGCCAGGCGCCGGGGTGCTTCAAGGACATATCCCTGGGCAGCCAGTCACCCCGCACTGGGTCCTGGATGGACAAC
CCTGGCGCACCGTCAGCCTGGAGGAGCCGGTCTCGAAGCCAGACATQGGGCTGCTGGCCCTGGAGGCTGAAGGCC
AGGAGCTCCTGCTTGAGCTGGAGAAGAACCACAGGCTGCTGGCCCCAGGATACATAGAAACCCACTACGGCCCAG
ATGGGCAGCCAGTGGTGCTGGCCCCCAACCACACGGATCATTGCCACTACCAAGGGCGAGTAAGGGGCTTCCCCG
ACTCCTGGGTAGTCCTCTGCACCTGCTCTGGGATGAGTGGCCTGATCACCCTCAGCAGGAATGCCAGCTATTATC
TGCGTCCCTGGCCACCCCGGGGCTCCAAGGACTTCTCAACCCACGAGATCTTTCGGATGGAGCAGCTGCTCACCT
GGAAAGGAACCTGTGGCCACAGGGATCCTGGGAACAAAGCGGGCATGACCAGCCTTCCTGGTGGTCCCCAGAGCA
GGGGCAGGCGAGAAGCGCGCAGGACCCGGAAGTACCTGGAACTGTACATTGTGGCAGACCACACCCTGTTCTTGA
CTCGGCACCGAAACTTGAACCACACCAAACAGCGTCTCCTGGAAGTCGCCAACTACGTGGACCAGCTTCTCAGGA
CTCTGGACATTCAGGTGGCGCTGACCGGCCTGGAGGTGTGGACCGAGCGGGACCGCAGCCGCGTCACGCAGGACG
CCAACGCCACGCTCTGGGCCTTCCTGCAGTGGCGCCGGGGGCTGTGGGCGCAGCGGCCCCACGACTCCGCGCAGC
TGCTCACGGGCCGCGCCTTCCAGGGCGCCACAGTGGGCCTGGCGCCCGTCGAGGGCATGTGCCGCGCCGAGAGCT
CGGGAGGCGTGAGCACGGACCACTCGGAGCTCCCCATCGGCGCCGCAGCCACCATGGCCCATGAGATCGGCCACA
GCCTCGGCCTCAGCCACGACCCCGACGGCTGCTGCGTGGAGGCTGCGGCCGAGTCCGGAGGCTGCGTCATGGCTG
CGGCCACCGGGCACCCGTTTCCGCGCGTGTTCAGCGCCTGCAGCCGCCGCCAGCTGCGCGCCTTCTTCCGCAAGG
GGGGCGGCGCTTGCCTCTCCAATGCCCCGGACCCCGGACTCCCGGTGCCGCCGGCGCTCTGCGGGAACGGCTTCG
TGGAAGCGGGCGAGGAGTGTGACTGCGGCCCTGGCCAGGAGTGCCGCGACCTCTGCTGCTTTGCTCACAACTGCT
CGCTGCGCCCGGGGGCCCAGTGCGCCCACGGGGACTGCTGCGTGCGCTGCCTGCTGAAGCCGGCTGGAGCGCTGT
GCCGCCAGGCCATGGGTGACTGTGACCTCCCTGAGTTTTGCACGGGCACCTCCTCCCACTGTCCCCCAGACCTTT
ACCTACTGGACGGCTCACCCTGTGCCAGGGGCAGTGGCTACTGCTGGGATGGCGCATGTCCCACGCTGGAGCAGC
AGTGCCAGCAGCTCTGGGGGCCTGGCTCCCACCCAGCTCCCGAGGCCTGTTTCCAGGTGGTGAACTCTGCGGGAG
ATGCTCATGGAAACTQCGGCCAGGACAGCGAGGGCCACTTCCTGCCCTGTGCAGGGAGGGATGCCCTGTGTGGGA
AGCTGCAGTGCCAGGGTGGAAAGCCCAGCCTGCTCGCACCGCACATGGTGCCAGTGGACTCTACCGTTCACCTAG
ATGGCCAGGAAGTGACTTGTCGGGGAGCCTTGGCACTCCCCAQTGCCCAGCTGGACCTGCTTGGCCTGGGCCTGG
TAGAGCCAGGCACCCAGTGTGGACCTAGAATGGTGTGCCAGAGCAGGCGCTGCAGGAAGAATGCCTTCCAGGAGC
TTCAGCGCTGCCTGACTGCCTGCCACAGCCACGGGGTTTGCAATAGCAACCATAACTGCCACTGTGCTCCAGGCT
GGGCTCCACCCTTCTGTGACAAGCCAGGCTTTGGTGGCAGCATGGACAGTGGCCCTGTGCAGGCTGAAAACCATG
ACACCTTCCTGCTGGCCATGCTCCTCAGCGTCCTGCTGCCTCTGCTCCCAGGGGCCGGCCTGGCCTGGTGTTGCT
ACCGACTCCCAGGAGCCCATCTGCAGCGATGCAGCTGGGGCTGCAGAAGGGACCCTGCGTGCAGTGGCCCCAAAG
ATGGCCCACACAGGGACCACCCCCTGGGCGGCGTTCACCCCATGGAGTTGGGCCCCACAGCCACTGGACAGCCCT
GGCCCCTGGCCCCAGGGGCTCCTGCTGACCATATTCACAACATTTACCCTCCACCATTTCTCCCAGACCCTGAGA
ACTCTCATGAGCCCAGCAGCCACCCTGAGAAGCCTCTGCCAGCAGTCTCGCCTGACCCCCAAGGTGGTTCCCTTG
CAGCCTGGGGCCCCAGTCCTTTAGGGGACAACATATCCTCCTCATTCTCAGCAGATCAAGTCCAGATGCCAAGAT
CCTGCCTCTGTGGCGAACCCTGGGGAGGCCACGTGGGAAGGAAAGAGGGCTCTAAGAGGGGAGGCCCCAGACTGG
GGGAGAGGCCTGTCTGGAGCCCAGGATCACCTGGCTGTGCTGCAGAACTGGAGAAGAGAAGCTCAGCAGAAAGGA
GCTGGCATGGGGCCAACAGCAGAAAAGCAGGAGGCACGCAGAAGTGACTGGGAAGCAGGAGG

The sequence of NOV7a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel transmembrane-like gene were obtained by SeqCallingTM Technology and are reported here as NOV7a. These methods used to amplify NOV7a cDNA are described in Example 2.

The NOV7a polypeptide (SEQ ID NO: 22) encoded by SEQ ID NO: 21 is 914 amino acid residues in length and is presented using the one-letter amino acid code in Table 7B. The SignalP, Psort and/or Hydropathy results predict that NOV7a has a signal peptide and is likely to be localized extracellularly at the plasma membrane with a certainty of 0.4600. In alternative embodiments, a NOV7a polypeptide is located to the microbody (peroxisome) with a certainty of 0.1026, the endoplasmic reticulum (membrane) with a certainty of 0.1000, or the endoplasmic reticulum (lumen) with a certainty of 0.1000. The SignalP predicts a likely cleavage site for a NOV7a peptide between amino acid positions 29 and 30, i.e. at the dash in the sequence GAG-VL.

TABLE 7B
EnCoded NOV7a Protein Sequence

(SEQ ID NO:22)

MGWRPRRARGTPLLLLLLLLLLWPVPGAGVLQGHIPGQPVTPHWVLDGQPWRTVSLEEPVSKPDMGLVALEAEG
QELLLELEKNHRLLAPGYIETHYGPDGQPVVLAPNHTDHCHYQGRVRGFPDSWVVLCTCSGMSGLLTLSRNASY
YLRPWPPRGSKDFSTHEIFRMEQLLTWKGTCGHRDPGNKAGMTSLPGGPQSRGRREARRTRKYLELYIVADHTL
FLTRHRNLNHTKQRLLEVANYVDQLLRTLDIQVALTGLEVWTERDRSRVTQDANATLWAFLQWRRGLWAQRPHD
SAQLLTGRAFQGATVGLAPVEGMCRAESSGGVSTDHSELPIGAAATMAHEIGHSLGLSHDPDGCCVEAAAESGG
CVMAAATGHPFPRVFSACSRRQLRAFFRKGGGACLSNAPDPGLPVPPALCGNGFVEAGEECDCGPGGECRDLCC
FAHNCSLRPGAQCAHGDCCVRCLLKPAGALCRQAMGDCDLPEFCTGTSSHCPPDVYLLDGSPCARGSGYCWDGA
CPTLEQQCGGLWGPGSHPAPEACFQVVNSAGDAHGNCGGDSEGHFLPCAGRDALCGKLQCGGGKPSLLAPHMVP
VDSTVHLDGQEVTCRGALALPSAQLDLLGLGLVEPGTQCGPRMVCGSRRCRKNAFQELQRCLTACHSHGVCNSN
HNCHCAPGWAPPFCDKPGFGGSMDSGPVQAENHDTFLLAMLLSVLLPLLPGAGLAWCCYRLPGAHLQRCSWGCR
RDPACSGPKDGPHRDHPLGGVHPMELGPTATGGPWPLAPGAPADHIHNIYPPPFLPDPENSHEPSSHPEKPLPA
VSPDPQGGSLAAWGPSPLGDNISSSFSADQVQMPRSCLCGEPWGGHVGRKEGSKRGGPRLGERPVWSPGSPGCA
AELEKRSSAERSWHGANSRKAGGTQK

NOV7b

In an alternative embodiment, a NOV7 variant is NOV7b (alternatively referred to herein as CG50367-02), which includes the 2705 nucleotide sequence (SEQ ID NO: 23) shown in Table 7C. An open reading frame for the mature protein was identified beginning with an ATG codon at nucleotides 3-5 and ending with a TGA codon at nucleotides 2688-2690. The start and stop codons of the open reading frame are highlighted in bold type. Putative untranslated regions are underlined and found upstream from the initiation codon and downstream from the termination codon.

TABLE 7C
NOV7b NuCleotide Sequence

(SEQ ID NO:23)

CTATGGGCTGGAGGCCCCGGAGAGCTCGGGGGACCCCGTTGCTGCTGCTGCTACTACTGCTGCTGCTCTGGCCAG
TGCCAGOCGCCGGGGTGCTTCAAGGACATATCCCTGGGCAGCCAGTCACCCCGCACTGGGTCCTGGATGGACAAC
CCTGGCGCACCGTCAGCCTGGAGGAGCCGGTCTCGAAGCCAGACATGGGGCTGGTGGCCCTGGAGGCTGAAGGCC
AGGAGCTCCTGCTTGAGCTGGAGAAGAACCACAGGCTGCTGGCCCCAGGATACATAGAAACCCACTACGGCCCAG
ATGGGCAGCCAGTGGTGCTGGCCCCCAACCACACGGATCATTGCCACTACCAAGGGCGAGTAAGGGGCTTCCCCG
ACTCCTGGGTAGTCCTCTGCACCTGCTCTGGGATGAGTGGCCTGATCACCCTCAGCAGGAATGCCAGCTATTATC
TGCGTCCCTGGCCACCCCGGGGCTCCAAGGACTTCTCAACCCACGAGATCTTTCGGATGGAGCAGCTGCTCACCT
GGAAAGGAACCTGTGGCCACAGGGATCCTGGGAACAAAGCGGGCATGACCAGCCTTCCTGGTGGTCCCCAGAGCA
GGGGCAGGCGAGAAGCGCGCAGGACCCGGAAGTACCTGGAACTGTACATTGTGGCAGACCACACCCTGTTCTTGA
CTCGGCACCGAAACTTGAACCACACCAAACAGCGTCTCCTGGAAGTCGCCAACTACGTGGACCAGCTTCTCAGGA
CTCTGGACATTCAGGTGGCGCTGACCGGCCTGGAGGTGT3GACCGAGCGGGACCGCAGCCGCGTCACGCAGGACG
CCAACGCCACGCTCTGGGCCTTCCTGCAGTGGCGCCGGGGGCTGTGGCCGCAGCGGCCCCACGACTCCGCGCAGC
TGCTCACGGGCCGCGCCTTCCAGGGCGCCACAGTGGGCCTGGCGCCCGTCGAGGGCATGTGCCGCGCCGAGAGCT
CGGGAGGCGTGAGCACGGACCACTCGGAGCTCCCCATCGGCGCCGCAGCCACCATGGCCCATGAGATCGGCCACA
GCCTCGGCCTCAGCCACGACCCCGACGGCTGCTGCGTGGAGGCTGCGGCCGAGTCCGGAGGCTGCGTCATGGCTG
CGGCCACCGGGCACCCGTTTCCGCGCGTGTTCAGCGCCTGCAGCCGCCGCCAGCTGCGCGCCTTCTTCCGCAAGG
GGGGCGGCGCTTGCCTCTCCAATGCCCCGGACCCCGGACTCCCGGTGCCGCCGGCGCTCTGCGGGAACGGCTTCG
TGGAAGCGGGCGAGGAGTGTGACTGCGGCCCTGGCCAGGAGTGCCGCGACCTCTGCTGCTTTGCTCACAACTGCT
CGCTGCGCCCGGGGGCCCAGTGCGCCCACGGGGACTGCTGCGTGCGCTGCCTGCTAAAGCCGGCTGGAGCGCTGT
GCCGCCAGGCCATGACTGACTGTGACCTCCCTGAGTTTTGCACGGGCACCTCCTCCCACTGTCCCCCAGACGTTT
ACCTACTGGACGGCTCACCCTGTGCCAAGGGCAGTGGCTACTGCTGGGATGGCGCATGTCCCACGCTGGAGCAGC
AGTGCCAGCAGCTCTGGGGGCCTGGCTCCCACCCAGCTCCCGAGGCCTGTTTCCAGGTGGTGAACTCTGCGGGAG
ATGCTCATGGAAACTGCGGCCAGGACAGCGAGGGCCACTTCCTGCCCTGTGCAGGGAGGGATGCCCTGTGTGGGA
AGCTGCAGTGCCAGGGTGGAAAGCCCAGCCTGCTCGCACCGCACATGGTGCCAGTGGACTCTACCGTTCACCTAG
ATGGCCAGGAAGTGACTTGTCGGGGAGCCTTGGCACTCCCCAGTGCCCAGCTGGACCTGCTTGGCCTGGGCCTGG
TAGAGCCAGGCACCCAGTGTGGACCTAGAATGGTGTGCCAGAGCAGGCGCTGCAGGAAGAATGCCTTCCAGGAGC
TTCAGCGCTGCCTGACTGCCTGCCACAGCCACGGGGTTTGCAATAGCAACCATAACTGCCACTGTGCTCCAGGCT
GGGCTCCACCCTTCTGTGACAAGCCAGGCTTTGGTGGCAGCATGGACAGTGGCCCTGTGCAGGCTGAAAACCATG
ACACCTTCCTGCTGGCCATGCTCCTCAGCGTCCTGCTGCCTCTGCTCCCAGGCGCCGGCCTGGCCTGGTGTTGCT
ACCGACTCCCAGGAGCCCATCTGCAGCGATGCAGCTCGGGCTGCAGAAGGGACCCTGCGTGCAGTGGCCCCAAAG
ATGGCCCACACAGAGACCACCCCCTGGGCGGCGTTCACCCCATGGAGTTGGGCCCCACAGCCACTGGACAGCCCT
GGCCCCTGGACCCTGAGAACTCTCATGAGCCCAGCAGCCACCCTGAGAAGCCTCTGCCAGCAGTCTCGCCTGACC
CCCAAGGTGGTTCCCTTGCAGCCTGGGGCCCCAGTCCTTTAGGGGACAACATATCCTCCTCATTCTCAGCAGATC
AAGTCCAGATGCCAAGATCCTGCCTCTGTGGCGAACCCTGGGGAGGCCACGTGGGAAGGAAAGAGGGCTCTAAGA
GGGGAGGCCCCAGACTGGGGGAGAGGCCTGTCTGGAGCCCAGGATCACCTGGCTGTGCTGCAGAACTGGAGAAGA
GAAGCTCAGCAGAAAGGAGCTGGCATGGGGCCAACAGCAGAAAAGCAGGAGGCACGCAGAAGTGACTGGGAAGCA
GGAGG

The sequence of NOV7b was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel stabilin-like gene were obtained by SeqCallingTM Technology and are reported here as NOV7b. These methods used to amplify NOV7b cDNA are described in Example 2.

The NOV7b polypeptide (SEQ ID NO: 24) encoded by SEQ ID NO: 23 is 895 amino acid residues in length and is presented using the one-letter amino acid code in Table 7D. The SignalP, Psort and/or Hydropathy results predict that NOV7b has a signal peptide and is likely to be localized extracellularly at the plasma membrane with a certainty of 0.4600. In alternative embodiments, a NOV7b polypeptide is located to the microbody (peroxisome) with a certainty of 0.1011, the endoplasmic reticulum (membrane) with a certainty of 0.1000, or the endoplasmic reticulum (lumen) with a certainty of 0.1000. The SignalP predicts a likely cleavage site for a NOV7b peptide between amino acid positions 29 and 30, i.e. at the dash in the sequence GAG-VL.

TABLE 7D
Encoded NOV7b Protein Sequence

(SEQ ID NO:24)

MGWRPRRARGTPLLLLLLLLLLWPVPGAGVLQGHIPGQPVTPHWVLDGQPWRTVSLEEPVSKPDMGLVALEAEG
QELLLELEKNHRLLAPGYIETHYGPDGQPVVLAPNHTDHCHYQGRVRGFPDSWVVLCTCSGMSGLITLSRNASY
YLRPWPPRGSKDFSTHEIFRMEQLLTWKGTCGHRDPGNKAGMTSLPGGPQSRGRREARRTRKYLELYIVADHTL
FLTRHRNLNHTKQRLLEVANYVDQLLRTLDIQVALTGLEVWTERDRSRVTQDANATLWAFLQWRRGLWAQRPHD
SAQLLTGRAFQGATVGLAPVEGMCRAESSGGVSTDHSELPIGAAATMAHEIGHSLGLSHDPDGCCVEAAAESGG
CVMAAATGHPFPRVFSACSRRQLRAFFRKGGGACLSNAPDPGLPVPPALCGNGFVEAGEECDCGPGQECRDLCC
FAHNCSLRPGAQCAHGDCCVRCLLKPAGALCRQAMGDCDLPEFCTGTSSHCPPDVYLLDGSPCAKGSGYCWDGA
CPTLEQQCQQLWGPGSHPAPEACFQVVNSAGDAHGNCGQDSEGHFLPCAGRDALCGKLQCQGGKPSLLAPHMVP
VDSTVHLDGQEVTCRGALALPSAQLDLLGLGLVEPGTQCGPRMVCQSRRCRKNAFQELQRCLTACHSHGVCNSN
HNCHCAPGWAPPFCDKPGFGGSMDSGPVQAENHDTFLLAMLLSVLLPLLPGAGLAWCCYRLPGAHLQRCSWGCR
RDPACSGPKDGPHRDHPLGGVHPMELGPTATGQPWPLDPENSHEPSSHPEKPLPAVSPDPQGGSLAAWGPSPLG
DNISSSFSADQVQMPRSCLCGEPWGGHVGRKEGSKRGGPRLGERPVWSPGSPGCAAELEKRSSAERSWHGANSR
KAGGTQK

NOV7c

In an alternative embodiment, a NOV7 variant is NOV7c (alternatively referred to herein as CG50367-03), which includes the 2642 nucleotide sequence (SEQ ID NO: 25) shown in Table 7E. An open reading frame for the mature protein was identified beginning with an ATG codon at nucleotides 3-5 and ending with a TGA codon at nucleotides 2625-2627. The start and stop codons of the open reading frame are highlighted in bold type. Putative untranslated regions are underlined and found upstream from the initiation codon and downstream from the termination codon.

TABLE 7E
NOV7c Nucleotide Sequence

(SEQ ID NO:25)

CTATGGGCTGGAGGCCCCGGAGAGCTCGGGGGACCCCGTTGCTGCTGCTGCTACTACTGCTGCTGCTCTGGCCAG
TGCCAGGCGCCGGGGTGCTTCAAGGACATATCCCTGGGCAGCCAGTCACCCCGCACTGGGTCCTGGATGGACAAC
CCTGGCGCACCGTCAGCCTGGAGGAGCCGGTCTCGAAGCCAGACATGGGGCTGGTGGCCCTGGAGGCTGAAGGCC
AGGAGCTCCTGCTTGAGCTGGAGAAGAACCACAGGCTGCTGGCCCCAGGATACATAGAAACCCACTACGGCCCAG
ATGGGCAGCCAGTGGTGCTGGCCCCCAACCACACGGATCATTGCCACTACCAAGGGCGAGTAAGGGGCTTCCCCG
ACTCCTGGGTAGTCCTCTGCACCTGCTCTGGGATGAGTGGCCTGATCACCCTCAGCAGGAATGCCAGCTATTATC
TGCGTCCCTGGCCACCCCGGGGCTCCAAGGACTTCTCAACCCACGAGATCTTTCGGATGGAGCAGCTGCTCACCT
GGAAAGGAACCTGTGGCCACAGGGATCCTGGGAACAAAGCGGGCATGACCAGCCTTCCTGGTGGTCCCCAGAGCA
GGGGCAGGCGAGAAGCGCGCAGGACCCGGAAGTACCTGGAACTGTACATTGTGGCAGACCACACCCTGTTCTTGA
CTCGGCACCGAAACTTGAACCACACCAAACAGCGTCTCCTGGAAGTCGCCAACTACGTGGACCAGCTTCTCAGGA
CTCTGGACATTCAGGTGGCGCTGACCGGCCTGGAGGTGTGGACCGAGCGGGACCGCAGCCGCGTCACGCAGGACG
CCAACGCCACGCTCTGGGCCTTCCTGCAGTGGCGCCGGGGGCTGTGGGCGCAGCGGCCCCACGACTCCGCGCAGC
TGCTCACGGGCCGCGCCTTCCAGGGCGCCACAGTGGGCCTGGCGCCCGTCGAGGGCATGTGCCGCGCCGAGAGCT
CGGGAGGCGTGAGCACGGACCACTCGGAGCTCCCCATCGGCGCCGCAGCCACCATGGCCCATGAGATCGGCCACA
GCCTCGGCCTCAGCCACGACCCCGACGGCTGCTGCGTGGAGGCTGCGGCCGAGTCCGGAGGCTGCGTCATGGCTG
CGGCCACCGGGCACCCGTTTCCGCGCGTGTTCAGCGCCTGCAGCCGCCGCCAGCTGCGCGCCTTCTTCCGCAAGG
GGGGCGGCGCTTGCCTCTCCAATGCCCCGGACCCCGGACTCCCGGTGCCGCCGGCGCTCTGCGGGAACGGCTTCG
TGGAAGCGGGCGAGGAGTGTGACTGCGGCCCTGGCCAGGAGTGCCGCGACCTCTGCTGCTTTGCTCACAACTGCT
CGCTGCGCCCGGGGGCCCAGTGCGCCCACGGGGACTGCTGCGTGCGCTGCCTGCTGAAGCCGGCTGGAGCGCTGT
GCCGCCAGGCCATGGGTGACTGTGACCTCCCTGAGTTTTGCACGGGCACCTCCTCCCACTGTCCCCCAGACGTTT
ACCTACTGGACGGCTCACCCTGTGCCAAGGGCAGTGGCTACTGCTGGGATGGCGCATGTCCCACGCTGGAGCAGC
AGTGCCAGCAGCTCTGGGGGCCTGGCTCCCACCCAGCTCCCGAGGCCTGTTTCCAGGTGGTGAACTCTGCGGGAG
ATGCTCATGGAAACTGCGGCCAGGACAGCGAGGGCCACTTCCTGCCCTGTGCAGGGAGGGATGCCCTGTGTGGGA
AGCTGCAGTGCCAGGGTGGAAAGCCCAGCCTGCTCGCACCGCACATGGTGCCAGTGGACTCTACCGTTCACCTAG
ATGGCCAGGAAGTGACTTGTCGGGGAGCCTTGGCACTCCCCAGTGCCCAGCTGGACCTGCTTGGCCTGGGCCTGG
TAGAGCCAGGCACCCAGTGTGGACCTAGAATGGTGTGCCAGAGCAGGCGCTGCAGGAAGAATGCCTTCCAGGAGC
TTCAGCGCTGCCTGACTGCCTGCCACAGCCACGGGGTTTGCAATAGCAACCATAACTGCCACTGTGCTCCAGGCT
GGGCTCCACCCTTCTGTGACAAGCCAGGCTTTGGTGGCAGCATGGACAGTGGCCCTGTGCAGGCTGAAAACCATG
ACACCTTCCTGCTGGCCATGCTCCTCAGCGTCCTGCTGCCTCTGCTCCCAGGCGCCGGCCTGGCCTGGTGTTGCT
ACCGACTCCCAGGAGCCCATCTGCAGCGATGCAGCTGGGGCTGCAGAAGGGACCCTGCGTGCAGTGGCCCCAAAG
ATGGCCCACACAGAGACCACCCCCTGGGCGGCGTTCACCCCATGGAGTTGGGCCCCACAGCCACTGGACAGCCCT
GGCCCCTGGACCCTGAGAACTCTCATGAGCCCAGCAGCCACCCTGAGAAGCCTCTGCCAGCAGTCTCGCCTGACC
CCCAAGCAGATCAAGTCCAGATGCCAAGATCCTGCCTCTGTGGCGAACCCTGGGGAGGCCACGTGGGAAGGAAAG
AGGGCTCTAAGAGGGGAGGCCCCAGACTGGGGGAGAGGCCTGTCTGGAGCCCAGGATCACCTGGCTGTGCTGCAG
AACTGGAGAAGAGAAGCTCAGCAGAAAGGAGCTGGCATGGGGCCAACAGCAGAAAAGCAGGAGGCACGCAGAAGT
GACTGGGAAGCAGGA
GG

The sequence of NOV7c was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel stabilin-like gene were obtained by SeqCallingTM Technology and are reported here as NOV7c. These methods used to amplify NOV7c cDNA are described in Example 2.

The NOV7c polypeptide (SEQ ID NO: 26) encoded by SEQ ID NO: 25 is 874 amino acid residues in length and is presented using the one-letter amino acid code in Table 7F. The SignalP, Psort and/or Hydropathy results predict that NOV7c has a signal peptide and is likely to be localized extracellularly at the plasma membrane with a certainty of 0.4600. In alternative embodiments, a NOV7c polypeptide is located to the microbody (peroxisome) with a certainty of 0.1000, the endoplasmic reticulum (membrane) with a certainty of 0.1000, or the endoplasmic reticulum (lumen) with a certainty of 0.1000. The SignalP predicts a likely cleavage site for a NOV7c peptide between amino acid positions 29 and 30, i.e. at the dash in the sequence GAG-VL.

TABLE 7F
Encoded NOV7b Protein Sequence

(SEQ ID NO:26)

MGWRPRRARGTPLLLLLLLLLLWPVPGAGVLQGHIPGQPVTPHWVLDGQPWRTVSLEEPVSKPDMGLVALEAEGQE
LLLELEKNHRLLAPGYIETHYGPDGQPVVLAPNHTDHCHYQGRVRGFPDSWVVLCTCSGMSGLITLSRNASYYLRP
WPPRGSKDFSTHEIFRMEQLLTWKGTCGHRDPGNKAGMTSLPGGPQSRGRREARRTRKYLELYIVADHTLFLTRHR
NLNHTKQRLLEVANYVDQLLRTLDIQVALTGLEVWTERDRSRVTQDANATLWAFLQWRRGLWAQRPHDSAQLLTGR
AFQGATVGLAPVEGMCRAESSGGVSTDHSELPIGAAATMAHEIGHSLGLSHDPDGCCVEAAAESGGCVMAAATGHP
FPRVFSACSRRQLRAFFRKGGGACLSNAPDPGLPVPPALCGNGFVEAGEECDCGPGQECRDLCCFAHNCSLRPGAQ
CAHGDCCVRCLLKPAGALCRQAMGDCDLPEFCTGTSSHCPPDVYLLDGSPCAKGSGYCWDGACPTLEQQCQQLWGP
GSHPAPEACFQVVNSAGDAHGNCGQDSEGHFLPCAGRDALCGKLQCQGGKPSLLAPHMVPVDSTVHLDGQEVTCRG
ALALPSAQLDLLGLGLVEPGTQCGPRMVCQSRRCRKNAFQELQRCLTACHSHGVCNSNHNCHCAPGWAPPFCDKPG
FGGSMDSGPVQAENHDTFLLAMLLSVLLPLLPGAGLAWCCYRLPGAHLQRCSWGCRRDPACSGPKDGPHRDHPLGG
VHPMELGPTATGQPWPLDPENSHEPSSHPEKPLPAVSPDPQADQVQMPRSCLCGEPWGGHVGRKEGSKRGGPRLGE
RPVWSPGSPGCAAELEKRSSAERSWHGANSRKAGGTQK

SNP variants of NOV7 are disclosed in Example 3.

NOV7 Clones

Unless specifically addressed as NOV7a, NOV7b, or NOV7c, any reference to NOV7 is assumed to encompass all variants.

The amino acid sequence of NOV7 has high homolgy to other proteins as shown in Table 7G.

TABLE 7G
BLASTX Results from Patp Database for NOV7

| | | High | Smallest Sum |
|---|---|---|---|
| Sequences Producing High-Scoring Segment Pairs: | | Score | Prob P (N) |
| patp: AAB47106 | Second splice variant of MAPP - Homo sapiens | 4372 | 0.0 |
| patp: AAB47105 | First splice variant of MAPP - Homo sapiens | 3666 | 0.0 |
| patp: AAB50935 | ADAM protein #1 - Homo sapiens | 1790 | 1.6e−188 |
| patp: AAB50942 | ADAM gene #1 peptide #1 - Homo sapiens | 1790 | 6.6e−186 |
| patp: AAW25716 | Mouse beta meltrin protein | 1753 | 2.1e−180 |

In a search of sequence databases, it was found, for example, that the NOV7a nucleic acid sequence of this invention has 811 of 840 bases (96%) identical to a gb:GENBANK-ID:HSM801104|acc:AL117415.1 mRNA from Homo sapiens (Homo sapiens mRNA; cDNA DKFZp434K0521 (from clone DKFZp434KO521)). Further, the fill amino acid sequence of the disclosed NOV7a protein of the invention has 553 of 554 amino acid residues (99%) identical to, and 553 of 554 amino acid residues (99%) similar to, the 702 amino acid residue ptnr:TREMBLNEW-ACC:CAC16509 protein from Homo sapiens (Human) (DJ964F7.1 (NOVEL PROTEIN (DISINTEGRIN AND METALLOPROTEINASE))).

In a similar search of sequence databases, it was found, for example, that the NOV7b and NOV7c nucleic acid sequences have 1409 of 2252 bases (62%) identical to a gb:GENBANK-ID:XLU66003|acc:U66003.1 mRNA from Xenopus laevis (Xenopus laevis ADAM 13 mRNA, complete cds). Further, the fill amino acid sequence of the disclosed NOV7b and NOV7c proteins of the invention have 388 of 746 amino acid residues (52%) identical to, and 507 of 746 amino acid residues (67%) similar to, the 914 amino acid residue ptnr:SPTREMBL-ACC:O12960 protein from Xenopus laevis (African clawed frog) (ADAM 13).

Additional BLASTP results are shown in Table 7H.

TABLE 7H
NOV7 BLASTP Results

| Gene Index/ | | Length of | | | Expect |
|---|---|---|---|---|---|
| Identifier | Protein/Organism | aa | Identity (%) | Positives (%) | Value |
| CAC33154 | SEQUENCE 3 FROM | 812 | 785/811 | 789/811 | 0.0 |
| | PATENT WO0109293 - | | (96%) | (97%) | |
| | Homo sapiens (Human) | | | | |
| Q9BZ11 | DJ964F7.1 (NOVEL | 728 | 699/716 | 701/716 | 0.0 |
| | DISINTEGRIN AND | | (97%) | (97%) | |
| | REPROLYSIN | | | | |
| | METALLOPROTEINASE | | | | |
| | FAMILY PROTEIN) - | | | | |
| | Homo sapiens (Human) | | | | |
| CAC33153 | SEQUENCE 1 FROM | 802 | 661/661 | 661/661 | 0.0 |
| | PATENT WO0109293 - | | (100%) | (100%) | |
| | Homo sapiens (Human) | | | | |
| AAK67164 | ADAM33 - Mus musculus | 685 | 498/690 | 543/690 | 3.8e−280 |
| | (Mouse) | | (72%) | (78%) | |
| O12960 | ADAM 13 - Xenopus laevis | 914 | 388/746 | 507/746 | 9.4e−211 |
| | (African clawed frog) | | (52%) | (67%) | |

A multiple sequence alignment is given in Table 7I, with the NOV7 protein of the invention being shown in lines 1, 2, and 3 in a ClustalW analysis comparing NOV7 with related protein sequences of Table 7H.

[image]

[image]

[image]

Domain results for NOV7 were collected from the Pfam database, and then identified by the Interpro domain accession number. The results are listed in Table 7J with the statistics and domain description. These results indicate that the NOV7 polypeptides have properties similar to those of other proteins known to contain these domains.

TABLE 7J
Domain Analysis of NOV7

| | Score | E |
|---|---|---|
| PSSMs Producing Significant Alignments | (bits) | Value |
| Reprolysin (M12B) family zinc metalloprotease: domain 1 of 1, | 306.6 | 3.1e−88 |
| from 210 to 409 | | |

| | | |
|---|---|---|
| Reprolysin | kYiELvIVvDhgmytkygsdlnkirqrVhqivNlvNeiYrpqLNIrV | (SEQ ID NO:84) |
| | +\|+\|\|+\|\|+\|+ ++ ++++++++++ ++++\|+++ +++ \| \|+\| | |
| NOV7a | KYLELYIVADHTLFLTRHRNLNHTKQRLLEVANYVDQLLRT-LDIQV | (SEQ ID NO:22) |
| | vLvgLEIWsdgDkInvqsdandTLhsFgeWRetdLlkrksHDnAqLLtgi | |
| | +\|++\|\|+\|+++\|+ ++++++ \|\| \|++\|\|+ \| +++\|\| \|+\|\|+++ | |
| NOV7a | ALTGLEVWTERDRSRVTQDANATLWAFLQWRRG-LWAQRPHDSAQLLTGR | |
| | dfdgntiGaAyvggmCspkrSvGVvqdhspivllvAvtMAHELGHNLGmt | |
| | +++ ++\|+\|++ ++\| +++\|+\|\| ++++ +++ \|++\|\|\|\|+\|\|+\|\|+ | |
| NOV7a | AFQGATVGLAPVEGMCRAESSGGVSTDHSELPIGAAATMAHEIGHSLGLS | |
| | HDdknkdgCtCe...gggsCIMnpvassspskKkFSnCSkddyqkFltkq | |
| | \|\|++ +\|+ + ++++\|+\|++++++++++ \|\| \|\|+ +++ \| ++ | |
| NOV7a | HDPD---GCCVEaaaESGGCVMAAATGHPFPR-VFSACSRRQLRAFFRKG | |
| | kpqCLlNkP | |
| | +++\|\| \|+\| | |
| NOV7a | GGACLSNAP | |

| | | |
|---|---|---|
| Pep_M12B_propep (Reprolysin family propeptide): domain 1 of 1, | 112.3 | 9e−30 |
| from 80 to 198 | | |

| | | |
|---|---|---|
| M12B Propep | hLeknrsllapdftvttYdedGtlvteepliqddHCyYqGyVeGypn | (SEQ ID NO:85) |
| | \|+++++++++++ +++\|+++\|+ +++ ++++ +\|\|+\|+\|+\|+\|+++ | |
| NOV7a | ELEKNHRLLAPGYIETHYGPDGQPVVLAPNHT-DHCHYQGRVRGFPD | (SEQ ID NO:22) |
| | SaVslSTCsGgLRGilqlenlsYgIEPle..ssdgf.eHiiYqiendkte | |
| | \|+\| ++\|\|+\| +++ + +\|++ \| +++++ ++++\| +++++++ + | |
| NOV7a | SWVVLCTCSGMSGLITLSRNASYYLRPWPprGSKDFsTHEIFRMEQLLTW | |
| | pspcgecgslststdssygirsasp | |
| | +++++++++ + + + + + ++ | |
| NOV7a | KGTCGHRDPGN-KAGMTSLPGGPQ | |

The NOV7 disclosed in this invention is expressed in at least the following tissues: Ascending Colon, Cervix, Heart, Liver, Lymph node, Mammary gland/Breast, Ovary, Peripheral Blood, Placenta, Retina, Skin, Stomach, Testis, Uterus, and Whole Organism. This information was derived by determining the tissue sources of the sequences that were included in the invention including but not limited to SeqCalling sources, Public EST sources, Literature sources, and/or RACE sources.

The protein similarity information, expression pattern, and map location for the ADAM13-like protein and nucleic acid disclosed herein suggest that this protein may have important structural and/or physiological functions characteristic of the ADAM protein family. Therefore, the nucleic acids and proteins of the invention are useful in potential diagnostic and therapeutic applications and as a research tool. For example, the compositions of the present invention will have efficacy for treatment of patients suffering from: Xerostomia, Scleroderma, Hypercalceimia, Ulcers, Von Hippel-Lindau (VHL) syndrome, Cirrhosis,Transplantation, Cirrhosis, Inflammatory bowel disease, Diverticular disease, Hirschsprung's disease, Crohn's Disease, Appendicitis, Endometriosis,Fertility, Cardiomyopathy, Atherosclerosis, Hypertension, Congenital heart defects, Aortic stenosis ,Atrial septal defect (ASD),Atrioventricular (A-V) canal defect, Ductus arteriosus, Pulmonary stenosis, Subaortic stenosis, Ventricular septal defect (VSD), valve diseases,Tuberous sclerosis, Scleroderma, Obesity, Aneurysm, Fibromuscular dysplasia, Stroke, Bleeding disorders, Hemophilia, hypercoagulation, Idiopathic thrombocytopenic purpura, autoimmume disease,allergies, immunodeficiencies, Graft vesus host, Anemia, Ataxia-telangiectasia, Lymphedema, Allergies, and Tonsilitis and other diseases, disorders and conditions of the like.

The novel nucleic acid encoding the ADAM13-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV7 protein has multiple hydrophilic regions, each of which can be used as an immunogen. In one embodiment, a contemplated NOV7 epitope is from about amino acids 40 to 60. In another embodiment, a contemplated NOV7 epitope is from about amino acids 70 to 125. In alternative embodiments, contemplated NOV7 epitopes include from about amino acids 140 to 210, 220 to 250, 260 to 310, 320 to 360, 370 to 410, 420 to 460, 470 to 610, 620 to 700, and 710 to 910.

NOV8

Yet a further NOVX protein of the invention, referred to herein as NOV8 (alternatively referred to as CG50321-01), is a leucine-rich repeat containing an F-box protein-like protein.

F-box proteins are an expanding family of eukaryotic proteins characterized by an approximately 40 amino acid motif, the F box (so named because cyclin F was one of the first proteins in which this motif was identified). Some F-box proteins are known to be critical for the controlled degradation of cellular regulatory proteins. In fact, F-box proteins are one of the four subunits of ubiquitin protein ligases called SCFs. SCF ligases bring ubiquitin conjugating enzymes (either Ubc3 or Ubc4) to substrates that are specifically recruited by the different F-box proteins. The need for high substrate specificity and the large number of known F-box proteins in yeast and worms suggest the existence of a large family of mammalian F-box proteins. Some of these proteins contain WD-40 domains or leucine-rich repeats; others contain either different protein-protein interaction modules or no recognizable motifs. They named the F-box proteins that contain WD-40 domains Fbws, those containing leucine-rich repeats, Fbls, and the remaining ones Fbxs. The marked differences in F-box gene expression in human tissues is exemplar of their distinct role in ubiquitin-dependent protein degradation.

The NOV8 protein predicted here is localized extracellularly at the plasma membrane. Therefore, it is likely that this leucine-rich containing F-box protein-like protein is accessible to a diagnostic probe, and for the various therapeutic applications described herein.

The NOV8 protein disclosed in this invention maps to chromosome 17. This information was assigned using OMIM, the electronic northern bioinformatic tool implemented by CuraGen Corporation, public ESTs, public literature references and/or genomic clone homologies.

The NOV8 nucleic acid (SEQ ID NO: 27) of 1307 nucleotides encodes a novel leucine-rich containing F-box protein-like protein and is shown in Table 8A. An open reading frame for the mature protein was identified beginning with a ATG initiation codon at nucleotides 17-19 and ending with a TGA codon at nucleotides 1283-1285. Putative untranslated regions upstream from the start codon and downstream from the termination codon are underlined in Table 8A. The start and stop codons are in bold letters.

TABLE 8A
NOV8 Nucleotide Sequence

(SEQ ID NO:27)

CAAGAGCAGGTTTGAGATGTTCTCAAATAGTGATGAAGCTGTAATCAATAAAAAACTTCCCAAAGAACTCCTGTT
ACGGATATTTTCTTTTCTAGATGTTGTTACCCTGTGCCGCTGTGCTCAGGTCTCCAGGGCCTGGAATGTTCTGGC
TCTGGATGGCAGTAACTGGCAGCGAATTGACCTATTTGATTTCCAGAGGGATATTGAGGGCCGAGTAGTGGAGAA
TATTTCAAAACGATGTGGGGGCTTTTTACGAAAGTTAAGTCTTCGTGGATGTCTTGGAGTGGGAGACAATGCATT
AAGAACCTTTGCACAAAACTGCAGGAACATTGAAGTACTGAATCTAAATGGGTGTACAAAGACAATAGACGCTAC
ATGTACTAGCCTTAGCAAGTTCTGTTCCAAACTCAGGCACCTTGACTTGGCTTCCTGTACATCAATAACAAACAT
GCCTCTAAAAGCTCTGAGTGAGGGATGTCCACTGTTGGAGCAGTTGAACATTTCCTGGTGTGACCAAGTAACCAA
GGATGGCATTCAAGCACTAGTGAGGGGCTGTGGGGGTCTCAAGGCCTTATTCTTAAAAGGCTGCACGCAGCTAGA
AGATGAAGCTCTCAAGTACATAGGTGCACACTGCCCTGAACTGGTGACTTTGAACTTGCAGACTTGCTTGCAAAT
CACAGATGAAGGTCTCATTACTATATGCAGAGGGTGCCATAAGTTACAATCCCTTTGTGCCTCTGGCTGCTCCAA
CATCACAGATGCCATCCTGAATGCTCTAGGTCAGAACTGCCCACGGCTTAGAATATTGGAAGTGGCAAGATGTTC
TCAATTAACAGATGTGGGCTTTACCACTCTAGCCAGGAATTGCCATGAACTTGAAAAGATGGACCTGGAAGAGTG
TGTTCAGATAACAGATAGCACATTAATCCAACTTTCTATACACTGTCCTCGACTTCAAGTATTGAGTCTGTCTCA
CTGTGAGCTGATCACAGATGATGGAATTCGTCACCTGGGGAATGGGGCCTGCGCCCATGACCAGCTGGAGGTGAT
TGAGCTGGACAACTGCCCACTAATCACAGATGCATCCCTGGAGCACTTGAAGAGCTGTCATAGCCTTGAGCGGAT
AGAACTCTATGACTGCCAGCAAATCACACGGGCTGGAATCAAGAGACTCAGGACCCATTTACCCAATATTAAAGT
CCACGCCTACTTCGCACCTGTCACTCCACCCCCATCAGTAGGGGGCAGCAGACAGCGCTTCTGCAGATGCTGCAT
CATCCTATGACAATGGAGGTGGTCAACCTTGG

The sequence of NOV8 was derived by laboratory cloning of cDNA fragments covering the full length and/or part of the DNA sequence of the invention, and/or by in silico prediction of the full length and/or part of the DNA sequence of the invention from public human sequence databases.

The cDNA coding for the NOV8 sequence was cloned by the polymerase chain reaction (PCR). PCR primers were designed based on in silico predictions of the full length or some portion (one or more exons) of the cDNA/protein sequence of the invention. The DNA sequence and protein sequence for a novel leucine-rich containing F-Box protein-like gene were obtained by exon linking, or SeqCalling™ Technology and are reported here as NOV8. These primers and methods used to amplify NOV8 cDNA are described in Example 2.

The NOV8 polypeptide (SEQ ID NO: 28) encoded by SEQ ID NO: 27 is 422 amino acid residues in length and is presented using the one-letter amino acid code in Table 8B. The SignalP, Psort and/or Hydropathy results predict that NOV8 has a signal peptide and is likely to be localized extracellularly at the plasma membrane with a certainty of 0.6500. In alternative embodiments, a NOV8 polypeptide is located to the cytoplasm with a certainty of 0.4500, the microbody (peroxisome) with a certainty of 0.3000, or the mitochondrial matrix space with a certainty of 0.1000.

TABLE 8B
Encoded NOV8 Protein Sequence

(SEQ ID NO:28)

MFSNSDEAVINKKLPKELLLRIFSFLDVVTLCRCAQVSRAWNVLALDGSNWQRIDLFDFQRDIEGRVVENISKR
CGGFLRKLSLRGCLGVGDNALRTFAQNCRNIEVLNLNGCTKTIDATCTSLSKFCSKLRHLDLASCTSITNMPLK
ALSEGCPLLEQLNISWCDQVTKDGIQALVRGCGGLKALFLKGCTQLEDEALKYIGAHCPELVTLNLQTCLQITD
EGLITICRGCHKLQSLCASGCSNITDAILNALGQNCPRLRILEVARCSQLTDVGFTTLARNCHELEKMDLEECV
QITDSTLIQLSIHCPRLQVLSLSHCELITDDGIRHLGNGACAHDQLEVIELDNCPLITDASLEHLKSCHSLERI
ELYDCQQITRAGIKRLRTHLPNIKVHAYFAPVTPPPSVGGSRQRFCRCCI
IL

SNP variants of NOV8 are disclosed in Example 3.

The amino acid sequence of NOV8 has high homology to other proteins as shown in Table 8C.

TABLE 8C
BLASTX Results from Patp Database for NOV8

| | | | Smallest Sum |
|---|---|---|---|
| Sequences Producing High-Scoring Segment Pairs: | | High Score | Prob P (N) |
| patp: AAB48290 | Human ZF1 protein | 1819 | 2.1e−187 |
| patp: AAB92961 | Human protein sequence | 1818 | 2.7e−187 |
| patp: AAB92791 | Human protein sequence | 1817 | 3.4e−187 |
| patp: AAY83090 | F-box protein FBP-22 - Homo sapiens | 1786 | 6.5e−184 |
| patp: AAY02274 A | F-box protein sequence - Homo sapiens | 1562 | 3.6e−160 |

In a search of sequence databases, it was found, for example, that the NOV8 nucleic acid sequence of this invention has 737 of 801 bases (92%) identical to a gb:GENBANK-ID:AF182443|acc:AF182443.1 mRNA from Rattus norvegicus (Rattus norvegicus F-box protein FBL2 (FBL2) mRNA, complete cds). Further, the full amino acid sequence of the disclosed NOV8 protein of the invention has 328 of 422 amino acid residues (77%) identical to, and 375 of 422 amino acid residues (88%) similar to, the 423 amino acid residue ptnr:SPTREMBL-ACC:Q9UK27 protein from Homo sapiens (Human) (LEUCINE-RICH REPEATS CONTAINING F-BOX PROTEIN FBL3).

Additional BLASTP results are shown in Table 8D.

TABLE 8D
NOV8 BLASTP Results

| Gene Index/ | | Length of | | | Expect |
|---|---|---|---|---|---|
| Identifier | Protein/Organism | aa | Identity (%) | Positives (%) | Value |
| AAH07557 | RIKEN CDNA 2610511F20 | 422 | 420/422 | 420/422 | 2.0e−230 |
| | GENE - Homo sapiens | | (99%) | (99%) | |
| | (Human) | | | | |
| Q9CZV8 | 2610511F20RIK PROTEIN - | 422 | 416/422 | 417/422 | 1.1e−227 |
| | Mus musculus (Mouse) | | (98%) | (98%) | |
| Q9UK27 | LEUCINE-RICH | 423 | 328/422 | 375/422 | 1.6e−187 |
| | REPEATS CONTAINING | | (77%) | (88%) | |
| | F-BOX PROTEIN FBL3 - | | | | |
| | Homo sapiens (Human) | | | | |
| Q9UKA5 | F-BOX PROTEIN FBL2 - | 425 | 328/422 | 375/422 | 2.7e−187 |
| | Homo sapiens (Human) | | (77%) | (88%) | |
| Q9NVQ8 | CDNA FLJ10576 FIS, | 423 | 327/422 | 375/422 | 3.4e−187 |
| | CLONE NT2RP2003329, | | (77%) | (88%) | |
| | WEAKLY SIMILAR TO | | | | |
| | PUTATIVE ADENYLATE | | | | |
| | CYCLASE | | | | |
| | REGULATORY PROTEIN - | | | | |
| | Homo sapiens (Human) | | | | |

A multiple sequence alignment is given in Table 8E in a ClustalW analysis comparing NOV8 with related protein sequences disclosed in Table 8D.

[image]

[image]

Domain results for NOV8 were collected from BLAST sample domains found in the Smart and Pfam collections, and then identified by the Interpro domain accession number. The results are listed in Table 8F with the statistics and domain description. These results indicate that the NOV8 polypeptide has properties similar to those of other proteins known to contain these domains and similar to the properties of these domains.

TABLE 8F
Domain Analysis of NOV8

| | Score | E |
|---|---|---|
| PSSMs Producing Significant Alignments | (bits) | Value |
| F-box: domain 1 of 2, from 9 to 56 | 29.3 | 9e−05 |

| | | |
|---|---|---|
| F-box | fsllrLPddllekilsrLplkdllslskvskkfrslvdsl.ldv.kl | (SEQ ID NO:91) |
| | \|\| ++++ ++++\|+ ++++++++++ ++ ++ +++++ ++ | |
| NOV8 | VINKKLPKELLLRIFSFLDVVTLCRCAQVSRAWNVLALDGsNWQrID | (SEQ ID NO:28) |
| | l | |
| | + | |
| NOV8 | L | |

The Leucine-rich containing F-Box protein-like protein disclosed in this invention is expressed in at least the following tissues: Adrenal Gland, Bladder, Bone marrow, Brain (fetal), Brain (whole), Brain (amygdala), Brain (cerebellum), Brain (hippocampus), Brain (thalamus), Cerebral Cortex, Colorectal, Endothelial cells, Heart, Kidney, Kidney (fetal), Liver, Liver (fetal), Lymph node, Lung, Lung (fetal), Mammary gland, Ovary, Pancreas, Pituitary gland, Placenta, Prostate, Salivary gland, Skeletal Muscle, Small intestine, Spinal cord, Spleen, Stomach, Testis, Trachea, Thymus, Thyroid, Uterus, and several cancer cell lines including Breast ca. (except Breast ca. MDA-N), CNS ca, Colon ca., Gastric ca., Liver ca., Melanoma, Ovarian ca., Pancreatic ca., Prostate ca, and Renal ca. at a measurably higher level than the following tissues: Adipose and one cancer cell line Breast ca. MDA-N. Furthermore, the expression level is even higher in two particular cancer cell lines: Lung ca. (non-s.cl) NCI-H522 and Gastric ca. (liver met) NCI-N87.

The protein similarity information, expression pattern, and map location for the leucine-rich repeats containing F-Box protein-like protein and nucleic acid disclosed herein suggest that this protein may have important structural and/or physiological functions characteristic of the F-Box protein family. Therefore, the NOV8 nucleic acids and proteins of the invention are useful in potential diagnostic and therapeutic applications and as a research tool. For example, since the protein of the invention is ubiquitously expressed in many tissues, the compositions of the present invention will have efficacy for treatment of patients suffering from diseases associated with these tissues. Also since the expression level of the invention is much higher in two particular cancer cell lines: Lung ca. (non-s.cl) NCI-H522 and Gastric ca. (liver met) NCI-N87, the invention may be useful in diagnosis and treatment of these cancers.

The novel nucleic acid encoding the leucine-rich repeats containing F-Box protein-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 10 to 15. In another embodiment, a contemplated NOV8 epitope is from about amino acids 40 to 80. In other specific embodiments, contemplated NOV8 epitopes are from about amino acids 85 to 110, 120 to 140, 148 to 150, 155 to 180, 190 to 210, 225 to 230, 240 to 250, 253 to 260, 262 to 270, 275 to 300, 325 to 345, 350 to 400, and 405 to 420.

NOV9

Still yet a further NOVX protein of the invention, referred to herein as NOV9 (alternatively referred to as CG55902-01), is a steroid binding-like protein.

Steroid binding proteins are involved in reproductive behavior, cell cycle progression and various important physiologic pathologies.

The NOV9 protein disclosed herein is predicted to localize extracellularly. Therefore, it is likely that this steroid binding protein-like protein is accessible to a diagnostic probe, and for the various therapeutic applications described herein.

The NOV9 protein disclosed in this invention maps to chromosome 12. This information was assigned using OMIM, the electronic northern bioinformatic tool implemented by CuraGen Corporation, public ESTs, public literature references and/or genomic clone homologies.

The NOV9 nucleic acid (SEQ ID NO: 29) of 499 nucleotides encodes a novel steroid binding protein-like protein and is shown in Table 9A. An open reading frame for the mature protein was identified beginning with a ATG initiation codon at nucleotides 19-21 and ending with a TGA codon at nucleotides 442-444. Putative untranslated regions upstream from the start codon and downstream from the termination codon are underlined in Table 9A. The start and stop codons are in bold letters.

TABLE 9A
NOV9 Nucleotide Sequence

(SEQ ID NO:29)

TTCACTGTGGTGGGCCCCATGCCAGGGCAGTGGCTGCAGCAGCTGGCAGTGCTAGTCCTGATTCTGGTGCTAGCCT
GGGGGGCTGGTCTACTATGGCAGGAGAAGGATCAGCCCATCTATTTGGCAGTGAAGGGAGTGGGGCTTGATGTCAC
CTCTGGAAAGGGGTTTTATGGACAAAGAGCCCCCTACAATGCCTTGACCAGGAAGGACTCTGCTAGAGGGGTAGCC
AAGGTGTCCTTGGATCATGTAGACCTTACCTGTGACACAACAGGTCTCATAGCCAAGAAGTTGGAGTCCATGGATG
ATGTCTTCACCAGTGTGTACAAAGCCAAACACCCAATTGTCAGCTACAGGGCTCAGACAATTCTCAATGAGTTTGG
CAGCCCCAACCTGGACTTCAAGGCTGAAGACCAGCCCCTTTTTGACAAGAAGGAGGGGTTCTGAGGTTTCATCTGC
AGGAGCAGGTTTTTGGGAGAGTGAGGTAGGAAGACATTCCAGC

The sequence of NOV9 was derived by laboratory cloning of cDNA fragments covering the full length and/or part of the DNA sequence of the invention, and/or by in silico prediction of the full length and/or part of the DNA sequence of the invention from public human sequence databases.

The NOV9 polypeptide (SEQ ID NO: 30) encoded by SEQ ID NO: 29 is 141 amino acid residues in length and is presented using the one-letter amino acid code in Table 9B. The SignalP, Psort and/or Hydropathy results predict that NOV9 has a signal peptide and is likely to be localized extracellularly with a certainty of 0.8200. In alternative embodiments, a NOV9 polypeptide is located to the microbody peroxisome) with a certainty of 0.1274, the endoplasmic reticulum (membrane) with a certainty of 0.1000, or the endoplasmic reticulum (lumen) with a certainty of 0.1000.

TABLE 9B
Encoded NOV9 Protein Sequence

(SEQ ID NO:30)

MPGQWLQQLAVLVLILVLAWGAGLLWQEKDQPIYLAVKGVGLDVTSGKGFYGQRAPYNALTRKDSARGVAKV
SLDHVDLTCDTTGLIAKKLESMDDVFTSVYKAKHPIVSYRAQTILNEFGSPNLDFKAEDQPLFDKKEGF

The amino acid sequence of NOV9 has high homology to other proteins as shown in Table 9C.

TABLE 9C
BLASTX Results from Patp Database for NOV9

| | | | Smallest |
|---|---|---|---|
| | | High | Sum |
| Sequences Producing High-Scoring Segment Pairs: | | Score | Prob P (N) |
| patp: AAY94866 | Human protein clone HP10557 | 427 | 2.2e−42 |
| patp: AAB98322 | Human PA27 protein | 427 | 4.6e−42 |
| patp: AAY76019 | Rat dermal papilla protein DP3 | 412 | 6.6e−41 |
| patp: AAB55958 | Skin cell protein | 412 | 6.6e−41 |
| patp: AAB98325 | Human ortholog of r0v0-176.7A (PA27) protein sequence | 240 | 8.7e−23 |

In a search of sequence databases, it was found, for example, that the NOV9 nucleic acid sequence of this invention has 392 of 484 bases (80%) identical to a gb:GENBANK-ID:AF173937|acc:AF173937.1 mRNA from Homo sapiens (Homo sapiens secreted protein of unknown function (SPUF), mRNA, complete cds). Further, the full amino acid sequence of the disclosed protein of the invention has 85 of 115 amino acid residues (73%) identical to, and 96 of 115 amino acid residues (83%) similar to, the 172 amino acid residue ptnr:SPTREMBL-ACC:Q9UMX5 protein from Homo sapiens (Human) (SECRETED PROTEIN OF UNKNOWN FUNCTION).

Additional BLASTP results are shown in Table 9D.

TABLE 9D
NOV9 BLASTP Results

| Gene Index/ | | Length of | | Positives | Expect |
|---|---|---|---|---|---|
| Identifier | Protein/Organism | aa | Identity (%) | (%) | Value |
| Q9UMX5 | SECRETED PROTEIN OF | 172 | 85/115 | 96/115 | 2.8e−42 |
| | UNKNOWN FUNCTION - | | (73%) | (83%) | |
| | Homo sapiens (Human) | | | | |
| Q9CQ45 | 1110060M21RIK | 171 | 84/115 | 96/115 | 2.3e−39 |
| | PROTEIN - Mus musculus | | (73%) | (83%) | |
| | (Mouse) | | | | |
| Q9SK39 | PUTATIVE STEROID | 100 | 30/82 | 53/82 | 3.5e−11 |
| | BINDING PROTEIN - | | (36%) | (64%) | |
| | Arabidopsis thaliana | | | | |
| | (Mouse-ear cress) | | | | |
| Q9FVZ7 | PUTATIVE STEROID | 232 | 32/94 | 54/94 | 6.4e−09 |
| | MEMBRANE BINDING | | (34%) | (57%) | |
| | PROTEIN - Oryza sativa | | | | |
| | (Rice) | | | | |

A multiple sequence alignment is given in Table 9E in a ClustalW analysis comparing NOV9 with related protein sequences disclosed in Table 9D.

[image]

[image]

Domain results for NOV9 were collected from BLAST sample domains found in the Smart and Pfam collections, and then identified by the Interpro domain accession number. The results are listed in Table 9F with the statistics and domain description. These results indicate that the NOV9 polypeptide has properties similar to those of other proteins known to contain these domains and similar to the properties of these domains.

TABLE 9F
Domain Analysis of NOV9

| | Score | E |
|---|---|---|
| PSSMs Producing Significant Alignments | (bits) | Value |
| Steroid Binding Domain (SBD): domain 1 of 1, from 28 to | 52.2 | 1.2e−11 |
| 113 | | |

| | | |
|---|---|---|
| SBD | DFTpeELrkYDGsdedkpIylAikGkVYDVtrGrkFYGPgGPYslFA | (SEQ ID NO:96) |
| | ++ +++\|++\|++\| \|\|++\|+ \|\|\| +\|\|++++ | |
| NOV9 | ------------EK-DQPIYLAVKGVGLDVTSGKGFYGQRAPYNALT | (SEQ ID NO:30) |
| | GrDASRaLatmsfDeedlkdsDeEidDlsdLsadeleaLreWetk.FkaK | |
| | +\|+ \|++++ ++\| +++ \|+++\| ++ +++ ++++++ +++\| | |
| NOV9 | RKDSARGVAKVSLDHVDLT------CDTTGLIAKKLESMDDVFTSvYKAK | |
| | YpvVGrLi | |
| | ++\| + | |
| NOV9 | HPIVSYRA | |

The steroid binding protein-like protein disclosed in this invention is expressed in a variety of tssues.

The protein similarity information, expression pattern, and map location for the steroid binding protein-like protein and nucleic acid disclosed herein suggest that this protein may have important structural and/or physiological functions characteristic of the steroid binding protein family. Therefore, the NOV9 nucleic acids and proteins of the invention are useful in potential diagnostic and therapeutic applications and as a research tool. For example, the compositions of the present invention will have efficacy for treatment of patients suffering from: cancer, cataracts, obesity, diabetes, hyperlipidemia, infertility, inflammation, CNS disorders and other diseases, disorders and conditions of the like.

The novel nucleic acid encoding the steroid binding protein-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. In one embodiment, a contemplated NOV9 epitope is from about amino acids 25 to 37. In another embodiment, a contemplated NOV9 epitope is from about amino acids 42 to 78. In other specific embodiments, contemplated NOV9 epitopes are from about amino acids 81 to 92, and 95 to 135.

NOV10

Another NOVX protein of the invention, referred to herein as NOV10, includes two novel steroid dehydrogenase-like proteins. The disclosed proteins have been named NOV10a and NOV10b.

Steroid dehydrogenase enzymes influence mammalian reproduction, hypertension, neoplasia, and digestion. The three-dimensional structures of steroid dehydrogenase enzymes reveal the position of the catalytic triad, a possible mechanism of keto-hydroxyl interconversion, a molecular mechanism of inhibition, and the basis for selectivity.

The NOV10 proteins disclosed here are predicted to localize at the plasma membrane. Therefore, it is likely that these proteins are accessible to a diagnostic probe, and for the various therapeutic applications described herein.

The NOV10 proteins in this invention map to chromosome 16. This information was assigned using OMIM, the electronic northern bioinformatic tool implemented by CuraGen Corporation, public ESTs, public literature references and/or genomic clone homologies.

NOV10a

In one embodiment, a NOV10 variant is NOV10a (alternatively referred to herein as CG50307-01), which encodes a novel steroid dehydrogenase-like protein and includes the 1831 nucleotide sequence (SEQ ID NO: 31) shown in Table 10A. An open reading frame for the mature protein was identified beginning with an ATG codon at nucleotides 183-185 and ending with a TGA codon at nucleotides 1173-1175. Putative untranslated regions downstream from the termination codon and upstream from the initiation codon are underlined in Table 10A, and the start and stop codons are in bold letters.

TABLE 10A
NOV10a Nucleotide Sequence

(SEQ ID NO:31)

ACCGGTTTGGAAGACTTTGCCGGCCTGCAGGACACATGATGACATTGGAC
CCACCCTCCCCAGCTCGGAGTCTTTAACTCAGTCACATCTACGGAGTCCC
TTTGGCCACATAAGATTGGCCTTAAGAGAAGGACGGAGCCACATACTGCT
GACGGCCCAGAACTGGCAGAGAGAAGGTTGCCATGGCTGCTGTTGACAGT
TTCTACCTCTTGTACAGGGAAATCGCCAGGTCTTGCAATTGCTATATGGA
AGCTCTAGCTTTGGTTGGAGCCTGGTATACGGCCAGAAAAAGCATCACTG
TCATCTGTGACTTTTACAGCCTGATCAGGCTGCATTTTATCCCCCGCCTG
GGGAGCAGAGCAGACTTGATCAAGCAGTATGGAAGATGGGCCGTTGTCAG
CGGTGCAACAGATGGGATTGGAAAAGCCTACGCTGAAGAGTTAGCAAGCC
GAGGTCTCAATATAATCCTGATTAGTCGGAACGAGGAGAAGTTGCAGGTT
GTTGCTAAAGACATAGCCGACACGTACAAAGTGGAAACTGATATTATAGT
TGCGGACTTCAGCAGCGGTCGTGAGATCTACCTTCCAATTCGAGAAGCCC
TGAAGGACAAAGACGTTGGCATCTTGGTAAATAACGTGGGTGTGTTTTAT
CCCTACCCGCAGTATTTCACTCAGCTGTCCGAGGACAAGCTCTGGGACAT
CATAAATGTGAACATTGCCGCCGCTAGTTTGATGGTCCATGTTGTGTTAC
CGGGAATGGTGGAGAGAAAGAAAGGTGCCATCGTCACGATCTCTTCTGGC
TCCTGCTGCAAACCCACTCCTCAGCTGGCTGCATTTTCTGCTTCTAAGGC
TTATTTAGACCACTTCAGCAGAGCCTTGCAATATGAATATGCCTCTAAAG
GAATCTTTGTACAGAGTCTAATCCCTTTCTATGTAGCCACCAGCATGACA
GCACCCAGCAACTTTCTGCACAGGTGCTCGTGGTTGGTGCCTTCGCCAAA
AGTCTATGCACATCATGCTGTTTCTACTCTTGGGATTTCCAAAAGGACCA
CAGGATATTGGTCCCATTCTATTCAGTTTCTTTTTGCACAGTATATGCCT
GAATGGCTCTGGGTGTGGGGAGCAAATATTCTCAACCGTTCACTACGTAA
GGAAGCCTTATCCTGCACAGCCTGAGTCTGGATGGCCACTTGAGAAGTTT
TGCCAACTCCTGGGAACCTCGATATTCTGACATTTGGAAAAACACATTTA
ATTTATCTCCTGTGTTTCATTGCTGATTATTCAGCATACTGTTGATTCGT
CATTTGCAAAACACACATAATACCGTCAGAGTGCTGTGAAAAACCTTAAG
GGTGTGTGGATGGCACAGGATCAATAATGCCTGAGGCTGATTGACGACAT
CTACATTTCAGTGCTTTTTCCCTAAGCTGTTTGAAAGTTACGCTTTTCTG
TTGTTCTAGAGCCACAGCAGTCTAATATTGAAATATAATATGATTGTCAG
GTCTTATAATTTCAGATGTTGTTTTTTAAGGGAAATTGACCATTTCACTA
GAGGAGTTGTGCTGGTTTTTACATGTGCATCAAGGAAAGACTACTGGAAA
AGTATTTATTTTGGTAACTAAGATTGCTGGCTACTATTAGGGACACACTC
CGGGCTGTTTGGTATAGCTCTACCTGGTTTGACTATCTGTCATGGAAATG
CTGCCTTCCACTGGTTTTTCCTTTGAGACGGGGTGTGTGCCTGGGTTGTG
GGGCCCTTGGGCCCCTTTTTTTTGGTGCCCCTTCTTCCACCCACTTTCGG
CCCGCGGGCCCCCTGGCGCTCTGGGTTTCCC

The sequence of NOV10a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel transmembrane-like gene were obtained by SeqCallingTM Technology and are reported here as NOV10a. These methods used to amplify NOV10a cDNA are described in Example 2.

The NOV10a polypeptide (SEQ ID NO: 32) encoded by SEQ ID NO: 31 is 330 amino acid residues in length and is presented using the one-letter amino acid code in Table 10B. The SignalP, Psort and/or Hydropathy results predict that NOV10a has no known signal peptide and is likely to be localized at the plasma membrane with a certainty of 0.7000. In alternative embodiments, a NOV10a polypeptide is located to the mitochondrial inner membrane with a certainty of 0.6577, the microbody (peroxisome) with a certainty of 0.4556, or the mitochondrial matrix space with a certainty of 0.2792.

TABLE 10B
Encoded NOV10a Protein Sequence

(SEQ ID NO:32)

MAAVDSFYLLYREIARSCNCYMEALALVGAWYTARKSTTVICDFYSLIRL
HFIPRLGSRADLIKQYGRWAVVSGATDGIGKAYAEELASRGLNIILISRN
EEKLQVVAKDIADTYKVETDIIVADFSSGREIYLPIREALKDKDVGILVN
NVGVFYPYPQYFTQLSEDKLWDIINVNIAAASLMVHVVLPGMVERKKGAI
VTISSGSCCKPTPQLAAFSASKAYLDHFSRALQYEYASKGIFVQSLIPFY
VATSMTAPSNFLHRCSWLVPSPKVYAHHAVSTLGISKRTTGYWSHSIQFL
FAQYMPEWLWVWGANILNRSLRKEALSCTA

NOV10b

In an alternative embodiment, a NOV10 variant is NOV10b (alternatively referred to herein as CG50307-02), which includes the 1152 nucleotide sequence (SEQ ID NO: 33) shown in Table 10C. An open reading frame for the mature protein was identified beginning with an ATG codon at nucleotides 97-99 and ending with a TGA codon at nucleotides 1087-1089. The start and stop codons of the open reading frame are highlighted in bold type. Putative untranslated regions are underlined and found upstream from the initiation codon and downstream from the termination codon.

TABLE 10C
NOV10b Nucleotide Sequence

(SEQ ID NO:33)

ATCTACGGAGTCCCTTTGGCCACATAAGATTGGCCTTAAGAGAAGGACGG
AGCCACATACTGCTGACGGCCCAGAACTGGCAGAGAGAAGGTTGCCATGG
CTGCTGTTGACAGTTTCTACCTCTTGTACAGGGAAATCGCCAGGTCTTGC
AATTGCTATATGGAAGCTCTAGCTTTGGTTGGAGCCTGGTATACGGCCAG
AAAAAGCATCACTGTCATCTGTGACTTTTACAGCCTGATCAGGCTGCATT
TTATCCCCCGCCTGGGGAGCAGAGCAGACTTGATCAAGCAGTATGGAAGA
TGGGCCGTTGTCAGCGGTGCAACAGATGGGATTGGAAAAGCCTACGCTGA
AGAGTTAGCAAGCCGAGGTCTCAATATAATCCTGATTAGTCGGAACGAGG
AGAAGTTGCAGGTTGTTGCTAAAGACATAGCCGACACGTACAAAGTGGAA
ACTGATATTATAGTTGCGGACTTCAGCAGCGGTCGTGAGATCTACCTTCC
AATTCGAGAAGCCCTGAAGGACAAAGACGTTGGCATCTTGGTAAATAACG
TGGGTGTGTTTTATCCCTACCCGCAGTATTTCACTCAGCTGTCCGAGGAC
AAGCTCTGGGACATCATAAATGTGAACATTGCCGCCGCTAGTTTGATGGT
CCATGTTGTGTTACCGGGAATGGTGGAGAGAAAGAAAGGTGCCATCGTCA
CGATCTCTTCTGGCTCCTGCTGCAAACCCACTCCTCAGCTGGCTGCATTT
TCTGCTTCTAAGGCTTATTTAGACCACTTCAGCAGAGCCTTGCAATATGA
ATATGCCTCTAAAGGAATCTTTGTACAGAGTCTAATCCCTTTCTATGTAG
CCACCAGCATGACAGCACCCAGCAACTTTCTGCACAGGTGCTCGTGGTTG
GTGCCTTCGCCAAAAGTCTATGCACATCATGCTGTTTCTACTCTTGGGAT
TTCCAAAAGGACCACAGGATATTGGTCCCATTCTATTCAGTTTCTTTTTG
CACAGTATATGCCTGAATGGCTCTGGGTGTGGGGAGCAAATATTCTCAAC
CGTTCACTACGTAAGGAAGCCTTATGCTGCACAGCCTGAGTCTGGATGGC
CACTTGAGAAGTTTTGCCAACTCCTGGGAACCTCGATATTCTGACATTTG
GA

The sequence of NOV10b was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The cDNA coding for the NOV10b sequence was cloned by the polymerase chain reaction (PCR). Primers were designed based on in silico predictions of the full length or some portion (one or more exons) of the cDNA/protein sequence of the invention, or by translated homology of the predicted exons to closely related human sequences or to sequences from other species. The DNA sequence and protein sequence for a novel transmembrane-like gene were obtained by exon linking and are reported here as NOV10b. These primers and methods used to amplify NOV10b cDNA are described in Example 2.

The NOV10b polypeptide (SEQ ID NO: 34) encoded by SEQ ID NO: 33 is 330 amino acid residues in length and is presented using the one-letter amino acid code in Table 10D. The SignalP, Psort and/or Hydropathy results predict that NOV10b has no known signal peptide and is likely to be localized at the plasma membrane with a certainty of 0.7000. In alternative embodiments, a NOV10b polypeptide is located to the mitochondrial inner membrane with a certainty of 0.6577, the microbody (peroxisome) with a certainty of 0.4320, or the mitochondrial matrix space with a certainty of 0.2792.

TABLE 10D
Encoded NOV10b Protein Sequence

(SEQ ID NO:34)

MAAVDSFYLLYREIARSCNCYMEALALVGAWYTARKSITVICDFYSLIRL
HFIPRLGSRADLIKQYGRWAVVSGATDGIGKAYAEELASRGLNIILISRN
EEKLQVVAKDIADTYKVETDIIVADFSSGREIYLPIREALKDKDVGILVN
NVGVFYPYPQYFTQLSEDKLWDIINVNIAAASLMVHVVLPGMVERKKGAI
VTISSGSCCKPTPQLAAFSASKAYLDHFSRALQYEYASKGIFVQSLIPFY
VATSMTAPSNFLHRCSWLVPSPKVYAHHAVSTLGISKRTTGYWSHSIQFL
FAQYMPEWLWVWGANILNRSLRKEALCCTA

SNP variants of NOV10 are disclosed in Example 3.

NOV10 Clones

Unless specifically addressed as NOV10a or NOV10b, any reference to NOV10 is assumed to encompass all variants.

The amino acid sequence of NOV10 has high homolgy to other proteins as shown in Table 10E.

TABLE 10E
BLASTX Results from Patp Database for NOV10

| | | | Smallest |
|---|---|---|---|
| | | High | Sum |
| Sequences Producing High-Scoring Segment Pairs: | | Score | Prob P (N) |
| patp: AAM39603 | Human polypeptide | 1715 | 2.2e−176 |
| patp: AAM41389 | Human polypeptide | 1715 | 2.2e−176 |
| patp: AAM93392 | Human polypeptide | 1710 | 7.4e−176 |
| patp: AAU18335 | Human endocrine polypeptide | 1449 | 3.4e−148 |
| patp: AAM42370 | Human polypeptide | 1264 | 1.4e−128 |

In a search of sequence databases, it was found, for example, that the NOV10a nucleic acid sequence of this invention has 859 of 899 bases (95%) identical to a gb:GENBANK-ID:AK025626|acc:AK025626.1 mRNA from Homo sapiens (Homo sapiens cDNA: FLJ21973 fis, clone HEP05846). Further, the fill amino acid sequence of the disclosed NOV10a protein of the invention has 123 of 302 amino acid residues (40%) identical to, and 188 of 302 amino acid residues (62%) similar to, the 312 amino acid residue ptnr:SPTREMBL-ACC:Q9Y6G8 protein from Homo sapiens (Human) (STEROID DEHYDROGENASE HOMOLOG).

In a similar search of sequence databases, it was found, for example, that the NOV10b nucleic acid sequence of this invention has 350 of 351 bases (99%) identical to a gb:GENBANK-ID:AK025626|acc:AK025626.1 mRNA from Homo sapiens (Homo sapiens cDNA: FLJ21973 fis, clone HEP05846). Further, the full amino acid sequence of the disclosed protein of the invention has 122 of 299 amino acid residues (40%) identical to, and 187 of 299 amino acid residues (62%) similar to, the 312 amino acid residue ptnr:SPTREMBL-ACC:Q9Y6G8 protein from Homo sapiens (Human) (STEROID DEHYDROGENASE HOMOLOG).

Additional BLASTP results are shown in Table 10F.

TABLE 10F
NOV10 BLASTP Results

| Gene Index/ | | Length of | | | Expect |
|---|---|---|---|---|---|
| Identifier | Protein/Organism | aa | Identity (%) | Positives (%) | Value |
| Q9BY22 | STEROID | 309 | 309/309 | 309/309 | 2.6e−164 |
| | DEHYDROGENASE-LIKE | | (100%) | (100%) | |
| | PROTEIN - Homo sapiens | | | | |
| | (Human) | | | | |
| Q9VJG9 | CG13284 PROTEIN - | 339 | 125/310 | 191/310 | 9.5e−57 |
| | Drosophila melanogaster | | (40%) | (61%) | |
| | (Fruit fly) | | | | |
| Q9Y6G8 | STEROID | 312 | 123/302 | 188/302 | 2.5e−56 |
| | DEHYDROGENASE | | (40%) | (62%) | |
| | HOMOLOG - | | | | |
| | Homo sapiens (Human) | | | | |
| O57314 | Putative steroid | 312 | 121/228 | 163/238 | 5.3e−56 |
| | dehydrogenase SPM2 (EC | | (50%) | (68%) | |
| | 1.1.1.-) - | | | | |
| | Anas platyrhynchos | | | | |
| | (Domestic duck) | | | | |
| O70503 | Putative steroid | 312 | 122/281 | 180/281 | 3.7e−55 |
| | dehydrogenase KIK-I (EC | | (43%) | (64%) | |
| | 1.1.1.-) - Mus musculus | | | | |
| | (Mouse) | | | | |

A multiple sequence alignment is given in Table 10G, with the NOV10 protein of the invention being shown in lines 1 and 2, in a ClustalW analysis comparing NOV10 with related protien sequences of Table 10F.

[image]

[image]

Domain results for NOV10 were collected from the Pfam database, and then identified by the Interpro domain accession number. The results are listed in Table 10H with the statistics and domain description. These results indicate that the NOV10 polypeptides have properties similar to those of other proteins known to contain these domains.

TABLE 10H
Domain Analysis of NOV10

| | Score | E |
|---|---|---|
| PSSMs Producing Significant Alignments | (bits) | Value |
| Short Chain Alcohol Dehydrogenase (adh_short): domain 1 of 1, | 95.6 | 9.8e-25 |
| from 66 to 306 | | |

| | | |
|---|---|---|
| ADH Short | tgKvaLvTGassGIGlaiAkrLakeGakVvvvdrreekaegvaaelk | (SEQ ID NO:102) |
| | ++++++ \|++ \|\|\|+++\|++\|+++\| +++++ +++++++ +++ ++ | |
| NOV10a | YGRWAVVSGATDGIGKAYAEELASRGLNIILISRNEEKLQVVAKDIA | (SEQ ID NO:32) |
| NOV10a | aelGdralfiqlDvtdeeqvkaavaqaverlGd.rlDvLVNNAGilgpgp | |
| | + ++ +++\| ++ + + +++++ ++ + +\|\|\|\|+\|+ + + | |
| NOV10a | DTYKVETDIIVADFSSGRE---IYLPIREALKDkDVGILVNNVGVFYPYP | |
| | pfe.elseedwervidvNltGvflltqavlpamdhmlkrkgGrIvNisSv | |
| | + ++++ +++++\|+ ++ +++ +++ + ++++\| \|+ ++\| | |
| NOV10a | QYFtQLSEDKLWDIINVNIAAASLMVHVVLP---GMVERKKGAIVTTSSG | |
| | aGlnvgvpglsaYsASKaavigltrsLAlElaphgtgIrVnavaPGgvdT | |
| | ++ ++ +++++ +\|\|\|+ + +++++\|+ \|++ ++ \| \| + \| +++\| | |
| NOV10a | SCC-KPTPQLAAFSASKAYLDHFSRALQYEYASKG--IFVQSLIPFYVAT | |
| | dmtkalrsrlieakkkvrevadiadpeleerits.titplgrygv.tpee | |
| | ++++ ++ + + ++ ++ + + + + | |
| NOV10a | SMTAPSN---------------------FLHRCSwLV-PSPKVYAhHAVS | |
| | ianavlfLasdgasysvtgqtlnvdggl | |
| | ++ + ++ + ++++ + | |
| NOV10a | TLGISKRTTGYWSHS---IQFLFAQYMP | |

The NOV10 proteins disclosed in this invention is expressed in at least the following tissues: adrenal gland/suprarenal gland, bone, bone marrow, brain—whole, brain—hippocampus, brain—hypothalamus, dermis, epidermis, hair follicles, lymph node, t-cell, eye, ovary and testis. This information was derived by determining the tissue sources of the sequences that were included in the invention including but not limited to SeqCalling sources, Public EST sources, Literature sources, and/or RACE sources.

The protein similarity information, expression pattern, and map location for the steroid dehydrogenase-like protein and nucleic acid disclosed herein suggest that this protein may have important structural and/or physiological functions characteristic of the steroid dehydrogenase family. Therefore, the nucleic acids and proteins of the invention are useful in potential diagnostic and therapeutic applications and as a research tool. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, adrenoleukodystrophy, congenital adrenal hyperplasia, neoplasia, diabetes, digestion, Von Hippel-Lindau (VHL) syndrome, cirrhosis, pancreatitis, endometriosis, fertility, hemophilia, hypercoagulation, idiopathic thrombocytopenic purpura, autoimmume disease, allergies, immunodeficiencies, transplantation, graft versus host disease, osteoporosis, hypercalceimia, arthritis, ankylosing spondylitis, scoliosis, muscular dystrophy, Lesch-Nyhan syndrome, myasthenia gravis, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, psoriasis, actinic keratosis, acne, hair growth/loss, allopecia, pigmentation disorders, endocrine disorders, and other diseases, disorders and conditions of the like.

The novel nucleic acid encoding the steroid dehydrogenase-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV10 protein has multiple hydrophilic regions, each of which can be used as an immunogen. In one embodiment, a contemplated NOV10 epitope is from about amino acids 10 to 15. In another embodiment, a contemplated NOV10 epitope is from about amino acids 50 to 70. In other specific embodiments, contemplated NOV10 epitopes are from about amino acids 75 to 80, 80 to 85, 85 to 95, 100 to 110, 120 to 125, 125 to 140, 155 to 175, 200 to 205, 210 to 215, 215 to 225, 225 to 240, 260 to 275, 275 to 300, and 310 to 325.

NOV11

Yet a further NOVX protein of the invention, referred to herein as NOV11 (alternatively referred to as CG50311-01), is a myosin heavy chain-like protein.

Myosins are molecular motors that upon interaction with actin filaments convert energy from ATP hydrolysis into mechanical force. Myosins can be divided into at least three main classes, with two types of unconventional myosin being no more related to each other than they are to conventional myosin. Myosins have traditionally been classified as conventional or unconventional, with many of the unconventional myosin proteins thought to be distributed in a narrow range of organisms. Members of all three of these main classes are likely to be present in most or all eukaryotes.

Although SignalP, Psort and/or hydropathy suggest that the myosin heavy chain-like protein may be localized in the nucleus, the NOVI I protein predicted here is similar to the myosin heavy chain family, some members of which are expected to have intracellular sub-cellular localization. Therefore it is likely that this novel myosin heavy chain-like protein is available at the same sub-cellular localization and hence accessible to a diagnostic probe and for various therapeutic applications.

The NOV11 protein disclosed in this invention maps to chromosome 22. This information was assigned using OMIM, the electronic northern bioinformatic tool implemented by CuraGen Corporation, public ESTs, public literature references and/or genomic clone homologies.

The NOV11 nucleic acid (SEQ ID NO: 35) of 7396 nucleotides encodes a novel myosin heavy chain-like protein and is shown in Table 11A. An open reading frame for the mature protein was identified beginning with a ATG initiation codon at nucleotides 140-142 and ending with a TAA codon at nucleotides 6017-6019. Putative untranslated regions upstream from the start codon and downstream from the termination codon are underlined in Table 11A. The start and stop codons are in bold letters.

TABLE 11A
NOV11 Nucleotide Sequence

| | |
|---|---|
| CAAGGCTGACCTGCTGCAGCTCCCGCCTCGTGCGCTCGCCCCACCCGGCCGCCGCCCGAGCGCTCGAGAAAGTC | (SEQ ID NO:35) |
| CTCTCGGGAGAAGCAGCGCCTGTTCCCGGGGCAGATCCAGGTTCAGGTCCTGGCTATAAGTCACCATGGCACAG | |
| CAAGCTGCCGATAAGTATCTCTATGTGGATAAAAACTTCATCAACAATCCGCTGGCCCAGGCCGACTGGGCTGC | |
| CAAGAAGCTGGTATGGGTGCCTTCCGACAAGAGTGGCTTTGAGCCAGCCAGCCTCAAGGAGGAGGTGGGCGAAG | |
| AGGCCATCGTGGAGCTGGTGGAGAATGGGAAGAAGGTGAAGGTGAACAAGGATGACATCCAGAAGATGAACCCG | |
| CCCAAGTTCTCCAAGGTGGAGGACATGGCAGAGCTCACGTGCCTCAACGAAGCCTCGGTGCTGCACAACCTCAA | |
| GGAGCGTTACTACTCAGGGCTCATCTACACCTATTCAGGCCTGTTCTGTGTGGTCATCAATCCTTACAAGAACC | |
| TGCCCATCTACTCTGAAGAGATTGTGGAAATGTACAAGGGCAAGAAGAGGCACGAGATGCCCCCTCACATCTAT | |
| GCCATCACAGACACCGCCTACAGGAGTATGATGCAAGACCGAGAAGATCAATCCATCTTGTGCACTGGTGAATC | |
| TGGAGCTGGCAAGACGGAGAACACCAAGAAGGTCATCCAGTATCTGGCGTACGTGGCGTCCTCGCACAAGAGCA | |
| AGAAGGACCAGGGCGAGCTGGAGCGGCAGCTGCTGCAGGCCAACCCCATCCTGGAGGCCTTCGGGAACGCCAAG | |
| ACCGTGAAGAATGACAACTCCTCCCGCTTCGGCAAATTCATTCGCATCAACTTTGATGTCAATGGCTACATTGT | |
| TGGAGCCAACATTGAGACTTATCTTTTGGAGAAATCTCGTGCTATCCGCCAAGCCAAGGAAGAACGGACCTTCC | |
| ACATCTTCTATTATCTCCTGTCTGGGGCTGGAGAGCACCTGAAGACCGATCTCCTGTTGGAGCCGTACAACAAA | |
| TACCGCTTCCTGTCCAATGGACACGTCACCATCCCCGGGCAGCAGGACAAGGACATGTTCCAGGAGACCATGGA | |
| GGCCATGAGGATTATGGGCATCCCAGAAGAGGAGCAAATGGGCCTGCTGCGGGTCATCTCAGGGGTTCTTCAGC | |
| TCGGCAACATCGTCTTCAAGAAGGAGCGGAACACTGACCAGGCGTCCATGCCCGACAACACAGCTGCCCAAAAG | |
| GTGTCCCATCTCTTGGGTATCAATGTGACCGATTTCACCAGAGGAATCCTCACCCCGCGCATCAAGGTGGGACG | |
| GGATTACGTCCAGAAGGCGCAGACTAAAGAGCAGGCTGACTTTGCCATCGAGGCCTTGGCCAAGGCGACCTATG | |
| AGCGGATGTTCCGCTGGCTGGTGCTGCGCATCAACAAGGCTCTGGACAAGACCAAGAGGCAGGGCGCCTCCTTC | |
| ATCGGGATCCTGGACATTGCCGGCTTCGAGATCTTTGATCTGAACTCGTTTGAGCAGCTGTGCATCAATTACAC | |
| CAATGAGAAGCTGCAGCAGCTCTTCAACCACACCATGTTCATCCTGGAGCAGGAGGAGTACCAGCGCGAGGGCA | |
| TCGAGTGGAACTTCATCGACTTTGGCCTCGACCTGCAGCCCTGCATCGACCTCATTGAGAAGCCAGCAGGCCCC | |
| CCGGGCATTCTGGCCCTGCTGGACGAGGAGTGCTGGTTCCCCAAAGCCACCGACAAGAGCTTCGTGGAGAAGGT | |
| GATGCAGGAGCAGGGCACCCACCCCAAGTTCCAGAAGCCCAAGCAGCTGAAGGACAAAGCTGATTTCTGCATTA | |
| TCCACTATGCCGGCAAGGTGGATTACAAAGCTGACGAGTGGCTGATGAAGAACATGGATCCCCTGAATGACAAC | |
| ATCGCCACACTGCTCCACCAGTCCTCTGACAAGTTTGTCTCGGAGCTGTGGAAGGATGTGGACCGCATCATCGG | |
| CCTGGACCAGGTGGCCGGCATGTCGGAGACCGCACTGCCCGGGGCCTTCAAGACGCGGAAGGGCATGTTCCGCA | |
| CTGTGGGGCAGCTTTACAAGGAGCAGCTGGCCAAGCTGATGGCTACGCTGAGGAACACGAACCCCAACTTTGTC | |
| CGCTGCATCATCCCCAACCACGAGAAGAAGGCCGGCAAGCTGGACCCGCATCTCGTGCTGGACCAGCTGCGCTG | |
| CAACGGTGTTCTCGAGGGCATCCGTATCTGCCGCCAGGGCTTCCCCAACAGGGTGGTCTTCCAGGAGTTTCGGC | |
| AGAGATATGAGATCCTGACTCCAAACTCCATTCCCAAGGGTTTCATGGACGGGAAGCAGGCGTGCGTGCTCATG | |
| ATAAAAGCCCTGGAGCTCGACAGCAATCTGTACCGCATTGGCCAGAGCAAAGTCTTCTTCCGTGCCGGTGTGCT | |
| GGCCCACCTGGAGGAGGAGCGAGACCTGAAGATCACCGACGTCATCATAGGGTTCCAGGCCTGCTGCAGGGGCT | |
| ACCTGGCCAGGAAAGCATTTGCCAAGCGGCAGCAGCAGCTTACCGCCATGAAGGTCCTCCAGCGGAACTGCGCT | |
| GCCTACCTGAAGCTGCGGAACTGGCAGTGGTGGCGGCTCTTCACCAAGGTCAAGCCGCTGCTGCAGGTGAGCCG | |
| GCAGGAGGAGGAGATGATGGCCAAGGAGGAGGAGCTGGTGAAGGTCAGAGAGAAGCAGCTGGCTGCGGAGAACA | |
| GGCTCATGGAGATGGAGACGCTGCAGTCTCAGCTCATGGCAGAGAAATTGCAGCTGCAGGAGCAGCTCCAGGCA | |
| GAAACCGAGCTGTGTGCCGAGGCTGAGGAGCTCCGGGCCCGCCTGACCGCCAAGAAGCAGGAATTAGAAGAGAT | |
| CTGCCATGACCTAGAGGCCAGGGTGGAGGAGGAGGAGGAGCGCTACCAGCACCTGCAGGCGGAGAAGAAGAAGA | |
| TGCAGCAGAACATCCAGGAGCTTGAGGAGCAGCTGGAGGAGGAGGAGAGCGCCCGGCAGAAGCTGCAGCTGGAG | |
| AAGGTGACCACCGAGGCGAAGCTGAAAAAGCTGGAGGAGGAGCAGATCATCCTGGAGGACCAGAACTGCAAGCT | |
| GGCCAAGGAAAAGAAACTGCTGGAAGACAGAATAGCTGAGTTCACCACCAACCTCACAGAAGAGGAGGAGAAAT | |
| CTAAGAGCCTCGCCAAGCTCAAGAACAAGCATGAGGCAATGATCACTGACTTGGAAGAGCGCCTCCGCAGGGAG | |
| GAGAAGCAGCGACAGGAGCTGGAGAAGACCCGCCGGAAGCTGGAGGGAGACTCCACAGACCTCAGCGACCAGAT | |
| CGCCGAGCTCCAGGCCCAGATCGCGGAGCTCAAGATGCAGCTGGCCAAGAAAGAGGAGGAGCTCCAGGCCGCCC | |
| TGGCCAGAGTGGAAGAGGAAGCTGCCCAGAAGAACATGGCCCTCAAGAAGATCCGGGAGCTGGAATCTCAGATC | |
| TCTGAACTCCAGGAAGACCTGGAGTCTGAGCGTGCTTCCAGGAATAAAGCTGAGAAGCAGAAACGGGACCTTGG | |
| GGAAGAGCTAGAGGCGCTGAAAACAGAGTTGGAGGACACGCTGGATTCCACAGCTGCCCAGCAGGAGCTCAGGT | |
| CAAAACGTGAGCAGGAGGTGAACATCCTGAAGAAGACCCTGGAGGAGGAGGCCAAGACCCACGAGGCCCAGATC | |
| CAGGAGATGAGGCAGAAGCACTCACAGGCCGTGGAGGAGCTGGCGGAGCAGCTGGAGCAGACGAAGCGGGTGAA | |
| AGCAAACCTCGAGAAGGCAAAGCAGACTCTGGAGAACGAGCGGGGGGAGCTGGCCAACGAGGTGAAGGTGCTGC | |
| TGCAGGGCGGAAGGGACTCGGAGCACAAGCGCAAGAAAGTGGAGGCGCAGCTGCAGGAGCTGCAGGTCAAGTTC | |
| AACGAGGGAGAGCGGGTGCGCACAGAGCTGGCCGACAAGGTCACCAAGCTGCAGGTGGAGCTGGACAACGTGAC | |
| CGGGCTTCTCAGCCAGTCCGACAGCAAGTCCAGCAAGCTCACCAAGGACTTCTCCGCGCTGGAGTCCCAGCTGC | |
| AGGACACTCAGGAGCTGCTGCAGGAGGAGAACCGGCAGAAGCTGAGCCTGAGCACCAAGCTCAAGCAGGTGGAG | |
| GACGAGAAGAATTCCTTCCGGGAGCAGCTGGAGGAGGAGGAGGCCAAGCACAACCTGGAGAAGCAGATCGCCAC | |
| CCTCCATGCCCAGGTGGCCGACATGAAAAAGAAGATGGAGGACAGTGTGGGGTGCCTGGAAACTGCTGAGGAGG | |
| TGAAGAGGAAGCTCCAGAAGGACCTGGAGGGCCTGAGCCAGCGGCACGAGGAGAAGGTGGCCGCCTACGACAAG | |
| CTGGAGAAGACCAAGACGCGGCTGCAGGAGGAGCTGGACGACCTGCTGGTGGACCTGGACCACCAGCGCCAGAG | |
| CGCGTGCAACCTGGAGAAGAAGCAGAAGAAGTTTGACCAGCTCCTGGCGGAGGAGAAGACCATCTCTGCCAAGT | |
| ATGCAGAGGAGCGCGACCGGGCTGAGGCGGAGGCCCGAGAGAAGGAGACCAAGGCTCTGTCGCTGGCCCGGGCC | |
| CTGGAGGAAGCCATGGAGCAGAAGGCGGAGCTGGAGCGGCTCAACAAGCAGTTCCGCACGGAGATGGAGGACCT | |
| TATGAGCTCCAAGGATGATGTGGGCAAGAGTGTCCACGAGCTGGAGAAGTCCAAGCGGGCCCTAGAGCAGCAGG | |
| TGGAGGAGATGAAGACGCAGCTGGAAGAGCTGGAGGACGAGCTGCAGGCCACCGAAGATGCCAAGCTGCGGTTG | |
| GAGGTCAACCTGCAGGCCATGAAGGCCCAGTTCGAGCGGGACCTGCAGGGCCGGGACGAGCAGAGCGAGGAGAA | |
| GAAGAAGCAGCTGGTCAGACAGGTGCGGGAGATGGAGGCAGAGCTGGAGGACGAGAGGAAGCAGCGCTCGATGG | |
| CAGTGGCGGCGCGGAAGAAGCTGGAGATGGACCTGAAGGACCTGGAGGCGCACATCGACTCGGCCAACAAGAAC | |
| CGGGACGAAGCCATCAAACAGCTGCGGAAGCTGCAGGCCCAGATGAAGGACTGCATGCGCGAGCTGGATGACAC | |
| CCGCGCCTCTCGTGAGGAGATCCTGGCCCAGGCCAAAGAGAACGAGAAGAAGCTGAAGAGCATGGAGGCCGAGA | |
| TGATCCAGTTGCAGGAGGAACTGGCAGCCGCGGAGCGTGCCAAGCGCCAGGCCCAGCAGGAGCGGGATGAGCTG | |
| GCTGACGAGATCGCCAACAGCAGCGGCAAAGGAGCCCTGGCGTTAGAGGAGAAGCGGCGTCTGGAGGCCCGCAT | |
| CGCCCAGCTGGAGGAGGAGCTGGAGGAGGAGCAGGGCAACACGGAGCTGATCAACGACCGGCTGAAGAAGGCCA | |
| ACCTGCAGATCGACCAGATCAACGCCGACCTGAACCTGGAGCGCGGGCACGCCCAGAAGAACGAGAATGCTCGG | |
| CAGCAGCTGGAACGCCAGAACAAGGAGCTTAAGGTCAAGCTGCAGGAGATGGAGGGCACTGTCAAGTCCAAGTA | |
| CAAGGCCTCCATCACCGCCCTCGAGGCCAAGATTGCACAGCTGGAGGAGCAGCTGGACAACGAGACCAAGGAGC | |
| GCCAGGCAGCCTGCAAACAGGTGCGTCGGACCGAGAAGAAGCTGAAGGATGTGCTGCTGCAGGTGGATGACGAG | |
| CGGAGGAACGCCGAGCAGTACAAGGACCAGGCCGACAAGGCATCTACCCGCCTGAAGCAGCTCAAGCGGCAGCT | |
| GGAGGAGGCCGAAGAGGAGGCCCAGCGGGCCAACGCCTCCCGCCGGAAACTGCAGCGCGAGCTGGAGGACGCCA | |
| CTGAGACGGCCGATGCCATGAACCGCGAAGTCAGCTCCCTAAAGAACAAGCTCAGGCGCGGGGACCTGCCGTTT | |
| GTCGTGCCCCGCCGAATGGCCCGGAAAGGCGCCGGGGATGGCTCCGACGAAGAGGTAGATGGCAAAGCGGATGG | |
| GGCTGAGGCCAAACCTGCCGAATAAGCCTCTTCTCCTGCAGCCTGAGATGGATGGACAGACAGACACCACAGCC | |
| TCCCCTTCCCAGACCCCGCAGCACGCCTCTCCCCACCTTCTTGGGACTGCTGTGAACATGCCTCCTCCTGCCCT | |
| CCGCCCCGTCCCCCCATCCCGTTTCCCTCCAGGTGTTGTTGAGGGCATTTGGCTTCCTCTGCTGCATCCCCTTC | |
| CAGCTCCCTCCCCTGCTCAGAATCTGATACCAAAGAGACAGGGCCCGGGCCAGGCAGAGAGCGACCAGCAGGCT | |
| CCTCAGCCCTCTCTTGCCAAAAAGCACAAGATGTTGAGGCGAGCAGGGCAGGCCCCCGGGGAGGGCAGAGTTTT | |
| CTATGAATCTATTTTTCTTCAGACTGAGGCCTTTTGGTAGTCGGAGCTCCCCCAGTCGTCAGCCTCCCTGACGT | |
| CTGCCACCAGCGCCCCCCACTCCTCCTCCTTTCTTTGCTGTTTGCAATCACACGTGGTGACCTCACACACCTCT | |
| GCCCCTTGGGCCTCCCACTCCATGGCTCTGGGCGGTCAGAAGGAGCAGGCCTGGGCTCCACCTCTGTGCAGGGC | |
| ACAGAAGGCTGGGGTGGGGGGAGGAGTGGATTCCTCCTACCTGTCCCAGCAGCGCCACTGTCGCTGTCTCCTCT | |
| GATTCTAAAATGTCTCAAGTGCAATGCCCCCTCCCCTCCTTTACCGAGGACAGCCTGCCTCTGCCACAGCAAGG | |
| CTGTCGGGGTCAAGCTGGAAAGGCCAGCAGCCTTCCAGTGGCTTCTCCCGAACACTCTTGGGGACCAAATATAC | |
| TTAATGGTTAAGGGACTTGTCCCAAGTCTGACAGCCAGAGCGTTAGAGGGGCCAGCGGCTCCCCAGGCGATCTT | |
| GTGTCTACTCTAGGACTGGGCCCGAGGGTGGTTTACCTGCACCGTTGACTCAGTATAGTTTAAAAATCTGCCAC | |
| CTGCACAGGTATTTTTGAAAGCAAAATAAGGTTTTCTTTTTTCCCCTTTCTTGTAATAAATGATAAAATTCCGA | |
| GTCTTTCTCACTGCCTTTGTTTAGAAGAGAGTACTCGTCCTCACTGGTCTACACTGGTTGCCGAATTTACTTGT | |
| ATTCCTAACTGTTTTGTATATGCTGCATTGAGACTTACGGGCAAGAAGGGCATTTTTTTTTTTTAAAGGAAACA | |
| AACTCTCAAATCATGAAGTGATATAAAAGCTGCATATGCCTACAAAGCTCTGAATTCAGGTCCCAGTTGCTGTC | |
| ACAAAGGAGTGAGTGAAAACACCCACCCTACCCCCTTTTTTATATAATAAAAGTGCCTTAGCATGTGTTGCAGC | |
| TGTCACCACTACAGTAAGCTGGTTTACAGATGTTTTCCACTGAGCATCACAATAAAGAGAACCATGTGCT | |

The sequence of NOV11 was derived by laboratory cloning of cDNA fragments covering the full length and/or part of the DNA sequence of the invention, and/or by in silico prediction of the full length and/or part of the DNA sequence of the invention from public human sequence databases.

The cDNA coding for the NOV11 sequence was cloned by the polymerase chain reaction (PCR). PCR primers were designed based on in silico predictions of the full length or some portion (one or more exons) of the cDNA/protein sequence of the invention. The DNA sequence and protein sequence for a novel myosin heavy chain-like gene were obtained by exon linking, or SeqCalling™ Technology and are reported here as NOV11. These primers and methods used to amplify NOV11 cDNA are described in Example 2.

The NOV11 polypeptide (SEQ ID NO: 36) encoded by SEQ ID NO: 35 is 1959 amino acid residues in length and is presented using the one-letter amino acid code in Table 11B. The SignalP, Psort and/or Hydropathy results predict that NOV11 has no known signal peptide and is likely to be localized at the nucleus with a certainty of 0.9600. In alternative embodiments, a NOV11 polypeptide is located to the microbody (peroxisome) with a certainty of 0.3000, the mitochondrial matrix space with a certainty of 0.1000, or the lysosome (lumen) with a certainty of 0.1000.

TABLE 11B
Encoded NOV11 Protein Sequence

| | |
|---|---|
| MAQQAADKYLYVDKNFINNPLAQADWAAKKLVWVPSDKSGFEPASLKEEVGEEAIVELVENGKKVKVNKDDIQKM | (SEQ ID NO:36) |
| NPPKFSKVEDMAELTCLNEASVLHNLKERYYSGLIYTYSGLFCVVINPYKNLPIYSEEIVEMYKGKKRHEMPPHI | |
| YAITDTAYRSMMQDREDQSILCTGESGAGKTENTKKVIQYLAYVASSHKSKKDQGELERQLLQANPILEAFGNAK | |
| TVKNDNSSRFGKFIRINFDVNGYIVGANIETYLLEKSRAIRQAKEERTFHIFYYLLSGAGEHLKTDLLLEPYNKY | |
| RFLSNGHVTIPGQQDKDMFQETMEAMRIMGIPEEEQMGLLRVISGVLQLGNIVFKKERNTDQASMPDNTAAQKVS | |
| HLLGINVTDFTRGILTPRIKVGRDYVQKAQTKEQADFAIEALAKATYERMFRWLVLRINKALDKTKRQGASFIGI | |
| LDIAGFEIFDLNSFEQLCINYTNEKLQQLFNHTMFILEQEEYQREGIEWNFIDFGLDLQPCIDLIEKPAGPPGIL | |
| ALLDEECWFPKATDKSFVEKVMQEQGTHPKFQKPKQLKDKADFCIIHYAGKVDYKADEWLMKNMDPLNDNIATLL | |
| HQSSDKFVSELWKDVDRIIGLDQVAGMSETALPGAFKTRKGMFRTVGQLYKEQLAKLMATLRNTNPNFVRCIIPN | |
| HEKKAGKLDPHLVLDQLRCNGVLEGIRICRQGFPNRVVFQEFRQRYEILTPNSIPKGFMDGKQACVLMIKALELD | |
| SNLYRIGQSKVFFRAGVLAHLEEERDLKITDVIIGFQACCRGYLARKAFAKRQQQLTAMKVLQRNCAAYLKLRNW | |
| QWWRLFTKVKPLLQVSRQEEEMMAKEEELVKVREKQLAAENRLMEMETLQSQLMAEKLQLQEQLQAETELCAEAE | |
| ELRARLTAKKQELEEICHDLEARVEEEEERYQHLQAEKKKMQQNIQELEEQLEEEESARQKLQLEKVTTEAKLKK | |
| LEEEQIILEDQNCKLAKEKKLLEDRIAEFTTNLTEEEEKSKSLAKLKNKHEAMITDLEERLRREEKQRQELEKTR | |
| RKLEGDSTDLSDQIAELQAQIAELKMQLAKKEEELQAALARVEEEAAQKNMALKKIRELESQISELQEDLESERA | |
| SRNKAEKQKRDLGEELEALKTELEDTLDSTAAQQELRSKREQEVNILKKTLEEEAKTHEAQIQEMRQKHSQAVEE | |
| LAEQLEQTKRVKANLEKAKQTLENERGELANEVKVLLQGGRDSEHKRKKVEAQLQELQVKFNEGERVRTELADKV | |
| TKLQVELDNVTGLLSQSDSKSSKLTKDFSALESQLQDTQELLQEENRQKLSLSTKLKQVEDEKNSFREQLEEEEA | |
| KHNLEKQIATLHAQVADMKKKMEDSVGCLETAEEVKRKLQKDLEGLSQRHEEKVAAYDKLEKTKTRLQQELDDLL | |
| VDLDHQRQSACNLEKKQKKFDQLLAEEKTISAKYAEERDRAEAEAREKETKALSLARALEEAMEQKAELERLNKQ | |
| FRTEMEDLMSSKDDVGKSVHELEKSKRALEQQVEEMKTQLEELEDELQATEDAKLRLEVNLQAMKAQFERDLQGR | |
| DEQSEEKKKQLVRQVREMEAELEDERKQRSMAVAARKKLEMDLKDLEAHIDSANKNRDEAIKQLRKLQAQMKDCM | |
| RELDDTRASREEILAQAKENEKKLKSMEAEMIQLQEELAAAERAKRQAQQERDELADEIANSSGKGALALEEKRR | |
| LEARIAQLEEELEEEQGNTELINDRLKKANLQIDQINADLNLERGHAQKNENARQQLERQNKELKVKLQEMEGTV | |
| KSKYKASITALEAKIAQLEEQLDNETKERQAACKQVRRTEKKLKDVLLQVDDERRNAEQYKDQADKASTRLKQLK | |
| RQLEEAEEEAQRANASRRKLQRELEDATETADAMNREVSSLKNKLRRGDLPFVVPRRMARKGAGDGSDEEVDGKA | |
| DGAEAKPAE | |

SNP variants of NOV11 are disclosed in Example 3.

The amino acid sequence of NOV11 has high homology to other proteins as shown in Table 11C.

TABLE 11C
BLASTX Results from Patp Database for NOV11

| | | | Smallest |
|---|---|---|---|
| | | High | Sum |
| Sequences Producing High-Scoring Segment Pairs: | | Score | Prob P (N) |
| patp: AAM78854 | Human protein | 9773 | 0.0 |
| patp: AAM79838 | Human protein | 9760 | 0.0 |
| patp: AAM40999 | Human polypeptide | 7760 | 0.0 |
| patp: AAM41000 | Human polypeptide | 7760 | 0.0 |
| patp: AAW00024 | Smooth muscle myosin heavy | 7619 | 0.0 |
| | chain SM1 isoform protein - | | |
| | Mus musculus | | |

In a search of sequence databases, it was found, for example, that the NOV11 nucleic acid sequence of this invention has 5116 of 5122 bases (99%) identical to a gb:GENBANK-ID:HUMMYONM|acc:M31013.1 mRNA from Homo sapiens (Human nonmuscle myosin heavy chain (NMHC) mRNA, 3′ end). Further, the fill amino acid sequence of the disclosed protein of the invention was found to have 1953 of 1960 amino acid residues (99%) identical to, and 1953 of 1960 amino acid residues (99%) similar to, the 1960 amino acid residue ptnr:SWISSPROT-ACC:P35579 protein from Homo sapiens (Human) (MYOSIN HEAVY CHAIN, NONMUSCLE TYPE A (CELLULAR MYOSIN HEAVY CHAIN, TYPE A) (NMMHC-A)).

Additional BLASTP results are shown in Table 11D.

TABLE 11D
NOV11 BLASTP Results

| Gene Index/ | | Length of | | | Expect |
|---|---|---|---|---|---|
| Identifier | Protein/Organism | aa | Identity (%) | Positives (%) | Value |
| A61231 | myosin heavy chain | 1961 | 1955/1961 | 1956/1961 | 0.0 |
| | nonmuscle form A - human | | (99%) | (99%) | |
| P35579 | Myosin heavy chain, | 1960 | 1953/1960 | 1953/1960 | 0.0 |
| | nonmuscle type A (Cellular | | (99%) | (99%) | |
| | myosin heavy chain, type | | | | |
| | A) (Nonmuscle myosin | | | | |
| | heavy chain-A) (NMMHC- | | | | |
| | A) - Homo sapiens (Human) | | | | |
| Q62812 | Myosin heavy chain, | 1961 | 1879/1961 | 1916/1961 | 0.0 |
| | nonmuscle type A (Cellular | | (95%) | (97%) | |
| | myosin heavy chain, type | | | | |
| | A) (Nonmuscle myosin | | | | |
| | heavy chain-A) (NMMHC- | | | | |
| | A) - Rattus norvegicus (Rat) | | | | |
| P14105 | Myosin heavy chain, | 1959 | 1813/1959 | 1892/1959 | 0.0 |
| | nonmuscle (Cellular myosin | | (92%) | (96%) | |
| | heavychain) (NMMHC) - | | | | |
| | Gallus gallus (Chicken) | | | | |
| Q63731 | NEURONAL MYOSIN | 1999 | 1781/1951 | 1838/1951 | 0.0 |
| | HEAVY CHAIN - Rattus | | (91%) | (94%) | |
| | norvegicus (Rat) | | | | |

A multiple sequence alignment is given in Table 11E in a ClustalW analysis comparing NOV11 with related protein sequences disclosed in Table 11D.

[image]

[image]

[image]

[image]

[image]

Domain results for NOV11were collected from the Pfam database, and then identified by the Interpro domain accession number. The results are listed in Table 11F with the statistics and domain description. These results indicate that the NOV11 polypeptide has properties similar to those of other proteins known to contain these domains.

TABLE 11F
Domain Analysis of NOV11

| | Score | E |
|---|---|---|
| PSSMs Producing Significant Alignments | (bits) | Value |
| myosin_head (Motor domain): domain 1 of 1, from 83 to 764 | 1494.5 | 0.0 |

| | | |
|---|---|---|
| Myosin Head | vEDmveLtyLnEpsvlhNLKkRyksdlIYTYsGlvLvsvNPYkrLpq | (SEQ ID NO:108) |
| | +\|\|+++\|++\|+\| +++++\|\|\|+\|\| ++++\|\|\|\|+\|++++++\|\|\|++\|+ | |
| NOV11 | VEDMAELTCLNEASVLHNLKERYYSGLIYTYSGLFCVVINPYKNLP- | (SEQ ID NO:36) |
| | iYteeiiakYrGKrryElPPHiFAiADeAYRsMlsdkeNQsilISGESGA | |
| | +\|+++++++ \|+\|\|+++\|+\|\|\|++\|+ \| \|\|\|+\| +++++\|+++++\|\|\|\|\| | |
| NOV11 | IYSEEIVEMYKGKKRHEMPPHIYAITDTAYRSMMQDREDQSILCTGESGA | |
| | GKTEntKkvmqYlAaVsggnsgngeevpsvkvgrvEdqILqsNpiLEAFG | |
| | \|\|\|\|++\| ++++\|+\|+\|+++++++ + +++\| ++\|++\|\|+\|\|\|\|\| | |
| NOV11 | GKTENTKKVIQYLAYVASSHKSK------KDQGELERQLLQANPILEAFG | |
| | NAKTtRNNNSSRFGKyieIqFdktGkivGakIenYLLEKSRVvyQtegER | |
| | \|\|\|\|++\|+\|\|\|\|\|\|\|+++ \|+\|+ +\|+\|\|\|++\|++ \|\|\|\|\|\|\|+++\|+++\|\| | |
| NOV11 | NAKTVKNDNSSRFGKFIRINFDVNGYIVGANIETYLLEKSRAIRQAKEER | |
| | NFHIFYQLLaGasqqnlkkeLkLtndpedYhYLnqggevkpcytvdGiDD | |
| | +\|\|\|\|\|+\|\|+\|+ + ++ +\|+\|+ ++\|++\|++++ ++++\| +\| | |
| NOV11 | TFHIFYYLLSGAGEH-LKTDLLLE-PYNKYRFLSNGH-----VTIPGQQD | |
| | segnveeFketrkAmdilGftdeeqrsIFrivAaILhlGNikFkqrrkee | |
| | + +\|+++ +\|++++\|+ ++++++ +++++++\|++\|\|+ \|+++++ + | |
| NOV11 | K----DMFQETMEAMRIMGIPEEEQMGLLRVISGVLQLGNIVFKKERNTD | |
| | aaipddnnadtkalekaaeLlGvdatelekALlsrriktGtegrkStvtk | |
| | +++++++ ++++++++\|+\|+++++++++++++++++ \|+++ +++ | |
| NOV11 | QASMPDN----TAAQKVSHLLGINVTDFTRGILTPRIKVGRDY----VQK | |
| | pqnveQAsyARDALAKalYsRLFdWIVnrINktLdfkakegqdasfIGVL | |
| | +++++\|\| + \|++\|\|\|\|+ \|+\|+\| \|+\| +\|\|++\|+++++++ +++\|\|+\| | |
| NOV11 | AQTKEQADFAIEALAKATYERMFRWLVLRINKALDKTKRQG--ASFIGIL | |
| | DIyGFEIFekNSFEQLCINYvNEKLQQfFNhhmFklEQEEYkrEGIeWtf | |
| | \|\|+\|\|\|\|\|+ \|\|\|\|\|\|\|\|\|\|+\|\|\|\|\|\|+\|\|+++\|++\|\|\|\|\| ++\|\|\|+\|++ | |
| NOV11 | DIAGFEIFDLNSFEQLCINYTNEKLQQLFNHTMFILEQEEYQREGIEWNF | |
| | IdFgdNLQpcIDLIEkKs.PpGILsLLDEeClfPkaqSGtDqtFldKLys | |
| | \|+\|+++\|\|++\|\|\|\|\|++ +\|+\|\|\|+\|\|\|\|+\|++\|++ +\|++\|++\|+ + | |
| NOV11 | IDFGLDLQPCIDLIEKPAgPPGILALLDEECWFPKA---TDKSFVEKVMQ | |
| | tfskhpahfekfsPrfrqkksgahFiikHYAGdVeYnvegFleKNKDpLf | |
| | + ++ +++ ++++ ++++ \|++ \|\|\|\|+\|+\| ++ +++\|\|+\|+\|+ | |
| NOV11 | EQGTHP-KFQ----KPKQLKDKADFCIIHYAGVKDYKADEWLMKNMDPLN | |
| | ddlisllksSsnpllaeLFpdeetlagpfeadpsslskkrksgskNkstg | |
| | +++ ++ +\|+++++ +\|+++ +++ + ++ ++ +++ + | |
| NOV11 | DNIATLLHQSSDKFVSELWKDVDRIIGLDQVAGMSETALPGAF------- | |
| | kktkksnfiTvGaqfKeslneLMktLsstnLPHFvRCIkPNekKkagvfD | |
| | ++++++++\|+\|+++\|+++++\|\|++\|++++ \|+\|+\|\|\|+\|\|++\|+++++ \| | |
| NOV11 | -KTRKGMFRTVGQLYKEQLAKLMATLRNTN-PNFVRCIIPNHEKKAGKLD | |
| | aslVlhQLrclGVLEgiRIrRaGFPnRitfdeFlqRYriLapktwPkwsg | |
| | ++\|++\|\|+++ \|\|\|\|++\|\|+\|+\|\|\|+\|+ +++\|++\|\|++\|+++ +\|++++ | |
| NOV11 | PHLVLDQLRCNGVLEGIRICRQGFPNPVVFQEFRQRYEILTPNSIPKGFM | |
| | dakkgeknEIvaceklLqsLnlDkgeeyrfGkTKIFFR | |
| | ++++ ++ +++++\|++\|+ + +++ \|++\|+\|\|\| | |
| NOV11 | DGKQ-------ACVLMIKALELDS-NLYRIGQSKVFFR | |

The myosin heavy chain-like protein disclosed in this invention is expressed in at least the following tissues: Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Bone, Cervix, Chorionic Villus, Cochlea, Cornea, Coronary Artery, Dermis, Epidermis, Foreskin, Hair Follicles, Hypothalamus, Kidney Cortex, Liver, Lung, Lymph node, Lymphoid tissue, Oesophagus, Ovary, Parathyroid Gland, Peripheral Blood, Tonsils, Umbilical Vein, Whole Organism. This information was derived by determining the tissue sources of the sequences that were included in the invention including but not limited to SeqCalling sources, Public EST sources, Literature sources, and/or RACE sources.

The protein similarity information, expression pattern, and map location for the myosin heavy chain-like protein and nucleic acid disclosed herein suggest that this protein may have important structural and/or physiological functions characteristic of the nonmuscle myosins family. Therefore, the NOV11 nucleic acids and proteins of the invention are useful in potential diagnostic and therapeutic applications and as a research tool. For example, the compositions of the present invention may have efficacy for treatment of patients suffering from: restenosis, neurological, glomerular diseases and other diseases, disorders and conditions of the like.

The novel nucleic acid encoding the myosin heavy chain-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV11 protein has multiple hydrophilic regions, each of which can be used as an immunogen. In one embodiment, a contemplated NOV11 epitope is from about amino acids 1 to 150. In another embodiment, a contemplated NOV11 epitope is from about amino acids 150 to 225. In other specific embodiments, contemplated NOV11 epitopes are from about amino acids 300 through 1950.

NOV12

Another NOVX protein of the invention, referred to herein as NOV12, includes three novel pancreatitis-associated protein (PAP)-like protein. The disclosed proteins have been named NOV12a, NOV12b, and NOV12c.

PAP is synthesized as a preprotein with a molecular weight of 16.6 kDa. A search of protein databases reveals marked homolgy with the carbohydrate binding region of animal lectins. Although PAP has no hemagglutination activity, it does induce extensive bacterial aggregation. Further, the pattern of expression for PAP reveals that it is not found in the liver, stomach, salivary glands, brain, kidney, or testis. Such an expression pattern correlates to a stress protein involved in the control of bacterial proliferation.

At least the NOV12a protein disclosed herein is predicted to localize extracellularly. Therefore, it is likely that this protein is accessible to a diagnostic probe, and for the various therapeutic applications described herein.

NOV12a

In one embodiment, a NOV12 variant is NOV12a (alternatively referred to herein as CG50323-01), which encodes a novel pancreatitis-associated protein (PAP)-like protein and includes the 530 nucleotide sequence (SEQ ID NO: 37) shown in Table 12A. An open reading frame for the mature protein was identified beginning with an ATG codon at nucleotides 3-5 and ending with a TAA codon at nucleotides 528-530. Putative untranslated regions downstream from the termination codon and upstream from the initiation codon are underlined in Table 12A, and the start and stop codons are in bold letters.

TABLE 12A

NOV12a Nucleotide Sequence

(SEQ ID NO:37)

CCATGGCCCTGCCAAGTGTATCTTGGATGCTGCTTTCCTGCCTCATGCTG
CTGTCTCAGGTTCAAGGTGAAGAACCCCAGAGGGAACTGCCCTCTGCACG
GATCCGCTGTCCCAAAGGCTCCAAGGCCTATGGCTCCCACTGCTATGCCT
TGTTTTTGTCACCAAAATCCTGGACAGATGCAGATCTGGCCTGCCAGAAG
CGGCCCTCTGGAAACCTGGTGTCTGTGCTCAGTGGGGCTGAGGGATCCTT
CGTGTCCTCCCTGGTGAAGAGCATTGGTAACAGCTACTCATACGTCTGGA
TTGGGCTCCATGACCCCACACAGGGCACCGAGCCCAATGGAGAAGGTTGG
GAGTGGAGTAGCAGTGATGTGATGAATTACTTTGCATGGGAGAGAAATCC
CTCCACCATCTCAAGCCCCGGCCACTGTGCGAGCCTGTCGAGAAGCACAG
CATTTCTGAGGTGGAAAGATTATAACTGTAATGTGAGGTTACCCTATGTC
TGCAAGTTCAAATACTGGAGGCAATTGTAA

The sequence of NOV12a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The cDNA coding for the NOV12a sequence was cloned by the polymerase chain reaction (PCR). PCR primers were designed based on in silico predictions of the full length or some portion (one or more exons) of the cDNA/protein sequence of the invention. The DNA sequence and protein sequence for a novel PAP-like gene were obtained by exon linking, or SeqCalling™ Technology and are reported here as NOV12a. These primers and methods used to amplify NOV12a cDNA are described in Example 2.

The NOV12a polypeptide (SEQ ID NO: 38) encoded by SEQ ID NO: 37 is 175 amino acid residues in length and is presented using the one-letter amino acid code in Table 12B. The SignalP, Psort and/or Hydropathy results predict that NOV12a has a signal peptide and is likely to be localized extracellularly with a certainty of 0.4896. In alternative embodiments, a NOV12a polypeptide is located to the microbody (peroxisome) with a certainty of 0.1669, the endoplasmic reticulum (membrane) with a certainty of 0.1000, or the endoplasmic reticulum (lumen) with a certainty of 0.1000.

TABLE 12B
Encoded NOV12a Protein Sequence

(SEQ ID NO:38)

MALPSVSWMLLSCLMLLSQVQGEEPQRELPSARIRCPKGSKAYGSHCYAL
FLSPKSWTDADLACQKRPSGNLVSVLSGAEGSFVSSLVKSIGNSYSYVWI
GLHDPTQGTEPNGEGWEWSSSDVMNYFAWERNPSTISSPGHCASLSRSTA
FLRWKDYNCNVRLPYVCKFKYWRQL

NOV12b -NOV12c

In alternative embodiments, a NOV12 variant is NOV12b or NOV12c (alternatively referred to herein as 169475472 and 169475476, respectively), which include a 471 nucleotide sequence. NOV12b and NOV12c are insert assemblies that encode an open reading frame of NOV12a between residues 23 and 173. Table 12C notes the minor nucleotide and amino acid changes in NOV12b and NOV12c from the parent clone, NOV12a.

| Nov | Alternate | Change in DNA | Change in Protein Seq. |
|---|---|---|---|
| No. | Reference | Seq. from NOV12a | from NOV12a |
| 12b | 169475472 | A → G at bp 395 | No change |
| 12c | 169475476 | T → C at bp 479 | No change |

The sequences of NOV12b and NOV12c were derived by laboratory cloning of cDNA fragments coding for a domain of the full length form of CG50323-01 (NOV12a), between residues 23 to 173. The cDNA coding for the NOV12b and NOV12c sequences was cloned by the polymerase chain reaction (PCR). The PCR template is the previoisly identified plasma (NOV12a), when available, or human cDNA. These primers and methods used to amplify NOV12b and NOV12c cDNA are described in Example 2.

SNP variants of NOV12 are disclosed in Example 3.

NOV12 Clones

Unless specifically addressed as NOV12a, NOV12b, or NOV12c, any reference to NOV12 is assumed to encompass all variants.

The amino acid sequence of NOV12 has high homolgy to other proteins as shown in Table 12D.

TABLE 12D
BLASTX Results from Patp Database for NOV12

| | | | Smallest |
|---|---|---|---|
| | | High | Sum |
| Sequences Producing High-Scoring Segment Pairs: | | Score | Prob P (N) |
| patp: AAR54098 | Mouse PAP | 921 | 3.0e−92 |
| patp: AAR57117 | Human Pancreatitis-Associated Protein | 921 | 3.0e−92 |
| patp: AAB43568 | Human cancer associated protein | 921 | 3.0e−92 |
| patp: AAR14795 | Fragment A3 from human pancreatitis associated protein | 915 | 1.3e−91 |
| patp: AAW71682 | Human pancreatitis-associated protein | 813 | 8.4e−81 |

In a search of sequence databases, it was found, for example, that the NOV12a nucleic acid sequence of the invention has 514 of 520 bases (98%) identical to a gb:GENBANK-ID:S51768|acc:S51768.1 mRNA from Homo sapiens (PAP-H=pancreatitis-associated protein [human, pancreas, mRNA, 797 nt]). Further, the full amino acid sequence of the disclosed protein of the invention has 169 of 169 amino acid residues (100%) identical to, and 169 of 169 amino acid residues (100%) similar to, the 175 amino acid residue ptnr:SWISSPROT-ACC:Q06141 protein from Homo sapiens (Human) (PANCREATITIS-ASSOCIATED PROTEIN 1 PRECURSOR).

Additional BLASTP results are shown in Table 12E.

TABLE 12E
NOV12 BLASTP Results

| Gene Index/ | | Length of | | | |
|---|---|---|---|---|---|
| Identifier | Protein/Organism | aa | Identity (%) | Positives (%) | Expect Value |
| Q06141 | Pancreatitis-associated | 175 | 169/169 | 169/169 | 3.8e−92 |
| | protein 1 precursor - Homo | | (100%) | (100%) | |
| | sapiens (Human) | | | | |
| P23132 | Lithostathine precursor | 175 | 118/169 | 144/169 | 2.8e−66 |
| | (Pancreatic stone protein) | | (69%) | (85%) | |
| | (PSP) (Pancreatic thread | | | | |
| | protein) (PTP) (Islet of | | | | |
| | langerhans regenerating | | | | |
| | protein) (REG) (Islet cells | | | | |
| | regeneration factor) | | | | |
| | (ICRF) - Bos taurus | | | | |
| | (Bovine) | | | | |
| P25031 | Pancreatitis-associated | 175 | 117/169 | 140/169 | 1.9e−65 |
| | protein 1 precursor (Peptide | | (69%) | (82%) | |
| | 23) (REG-2) - Rattus | | | | |
| | norvegicus (Rat) | | | | |
| P35230 | Pancreatitis-associated | 175 | 115/164 | 135/164 | 2.2e−64 |
| | protein 1 precursor (REG | | (70%) | (82%) | |
| | III-beta) - Mus musculus | | | | |
| | (Mouse) | | | | |
| P42854 | Pancreatitis-associated | 174 | 117/170 | 141/170 | 5.3e−63 |
| | protein 3 precursor - Rattus | | (68%) | (82%) | |
| | norvegicus (Rat) | | | | |

A multiple sequence alignment is given in Table 12F, with the NOV12 protein of the invention being shown in lines 1, 2, and 3, in a ClustalW analysis comparing NOV12 with related protien sequences of Table 12E.

[image]

Domain results for NOV12 were collected from the Pfam database, and then identified by the Interpro domain accession number. The results are listed in Table 12G with the statistics and domain description. These results indicate that the NOV12 polypeptides have properties similar to those of other proteins known to contain these domains.

TABLE 12G
Domain Analysis of NOV12

| | Score | E |
|---|---|---|
| PSSMSs Producing Significant Alignments | (bits) | Value |
| lectin_c type domain: domain 1 of 1, from 53 to 169 | 146.5 | 4.5e−40 |

| | | |
|---|---|---|
| Lectin-C | esktWaeAelaCqkegghAHLvsIqsaeEqsfvvafltsltkksnty | (SEQ ID NO:114) |
| | ++++\|++\|+++\|++++++ +\|+++ + +\| ++++++++++ + ++++ | |
| NOV12a | SPKSWTDADLACQKRPSG-NLVSVLSGAEGSFVSSLVKSIGN-SYSY | (SEQ ID NO:38) |
| | aWIGLtdintegtwvwegwetdgspvnytenWapgePnnrgnhGgnEdCv | |
| | +\|\|\|\|+++++ ++++++++++ +++++++ \| +++++ ++\|+ | |
| NOV12a | VWIGLHDPTQGTEPNGEGWEWSSSDVMNYFAWERNPSTISS----PGHCA | |
| | eiytdtdflaGkWnDepCdsklpyvCef | |
| | +++++++++ +\|+\| +\|++ ++++\|++ | |
| NOV12a | SLSRSTAFL-RWKDYNCNVRLPYVCKF | |

The NOV12 proteins disclosed in this invention are expressed in at least the following tissues: at very low expression level in healthy pancreas and at much higher level during the acute phase of pancreatitis; it is also expressed at high level in normal small intestine. This information was derived by determining the tissue sources of the sequences that were included in the invention including but not limited to SeqCalling sources, Public EST sources, Literature sources, and/or RACE sources.

The protein similarity information, expression pattern, and map location for the PAP-like protein and nucleic acid disclosed herein suggest that this protein may have important structural and/or physiological functions characteristic of the Lectin C family. Therefore, the NOV12 nucleic acids and proteins of the invention are useful in potential diagnostic and therapeutic applications and as a research tool. For example, the compositions of the present invention will have efficacy for treatment of patients suffering from: acute pancreatitis and chronic pancreatitis, and other diseases, disorders and conditions of the like.

The novel NOV12 proteins of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV12 protein has multiple hydrophilic regions, each of which can be used as an immunogen. In one embodiment, a contemplated NOV12 epitope is from about amino acids 20 to 45. In another embodiment, a contemplated NOV12 epitope is from about amino acids 45 to 57. In other specific embodiments, contemplated NOV12 epitopes are from about amino acids 55 to 70, 72 to 77, 95 to 143, and 145 to 170.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, 25, 27, 29, 31, 33, 35, and 37, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, 27, 29, 31, 33, 35, and 37 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.) A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25, 27, 29, 31, 33, 35, and 37, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27, 29, 31, 33, 35, and 37, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS: 1, 3, 5, 7, 9, 11, 13, 15, 17, 19, 21, 23, 25, 27, 29, 31, 33, 35, and 37 is one that is sufficiently complementary to the nucleotide sequence shown SEQ ID NOS: 1, 3, 5, 7, 9, 11, 13, 15, 17, 19, 21, 23, 25, 27,29, 31, 33, 35, and 37 that it can hydrogen bond with little or no mismatches to the nucleotide sequence shown SEQ ID NOS: 1, 3, 5, 7, 9, 11, 13, 15, 17, 19, 21, 23, 25, 27, 29, 31, 33, 35, and 37 thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 29, 31, 33, 35, and 37, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, 27, 29, 31, 33, 35, and 37; or an anti-sense strand nucleotide sequence of SEQ ID NOS: 1, 3, 5, 7, 9, 11, 13, 15, 17, 19, 21, 23, 25, 27, 29, 31, 33, 35, and 37; or of a naturally occurring mutant of SEQ ID NOS: 1, 3, 5, 7, 9, 11, 13, 15, 17, 19, 21, 23, 25, 27, 29, 31, 33, 35, and 37.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27, 29, 31, 33, 35, and 37, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, 25, 27, 29, 31, 33, 35, and 37 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, 25, 27, 29, 31, 33, 35, and 37. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, 32, 34, 36, and 38.

In addition to the human NOVX nucleotide sequences shown in SEQ ID NOS: 1, 3, 5, 7, 9, 11, 13, 15, 17, 19, 21, 23, 25, 27, 29, 31, 33, 35, and 37, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, 25, 27, 29, 31, 33, 35, and 37 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, 31, 33, 35, and 37. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, 25, 27, 29, 31, 33, 35, and 37, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, 31, 33, 35, and 37, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27, 29, 31, 33, 35, and 37, or fragments, analogs or derivatives thereof, under conditions of low stringency, is provided. A non-limiting example of low stringency hybridization conditions are hybridization in 35% formrnamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, 25, 27, 29, 31, 33, 35, and 37, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, 18, 20, 22, 24, 26, 28, 30, 32, 34, 36, and 38. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, 23, 25, 27, 29, 31, 33, 35, and 37 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, 18, 20, 22, 24, 26, 28, 30, 32, 34, 36, and 38. Preferably, the protein encoded by the nucleic acid molecule is at least about 60% homologous to SEQ ID NOS: 2, 4, 6, 8, 10, 12, 14, 16, 18, 20, 22, 24, 26, 28, 30, 32, 34, 36, and 38; more preferably at least about 70% homologous SEQ ID NOS: 2, 4, 6, 8, 10, 12, 14, 16, 18, 20, 22, 24, 26, 28, 30, 32, 34, 36, and 38; still more preferably at least about 80% homologous to SEQ ID NOS: 2, 4, 6, 8, 10, 12, 14, 16, 18, 20, 22, 24, 26, 28, 30, 32, 34, 36, and 38; even more preferably at least about 90% homologous to SEQ ID NOS: 2, 4, 6, 8, 10, 12, 14, 16, 18, 20, 22, 24, 26, 28, 30, 32, 34, 36, and 38; and most preferably at least about 95% homologous to SEQ ID NOS: 2, 4, 6, 8, 10, 12, 14, 16, 18, 20, 22, 24, 26, 28, 30, 32, 34, 36, and 38.

An isolated nucleic acid molecule encoding an NOVX protein homologous to the protein of SEQ ID NOS: 2, 4, 6, 8, 10, 12, 14, 16, 18, 20, 22, 24, 26, 28, 30, 32, 34, 36, and 38 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 29, 31, 33, 35, and 37, such that one or more amino acid substitutions, additions or deletions are introduced into the encoded protein.

Mutations can be introduced into SEQ ID NOS: 1, 3, 5, 7, 9, 11, 13, 15, 17, 19, 21, 23, 25, 27, 29, 31, 33, 35, and 37 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27, 29, 31, 33, 35, and 37, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 29, 31, 33, 35,and 37, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, 18, 20, 22, 24, 26, 29, and 31, or antisense nucleic acids complementary to an NOVX nucleic acid sequence of SEQ ID NOS: 1, 3, 5, 7, 9, 11, 13, 15, 17, 19, 21, 23, 25, 27, 29, 31, 33, 35, and 37, are additionally provided.

In one embodiment, an antisense nucleic acid molecule is antisense to a “coding region” of the coding strand of a nucleotide sequence encoding an NOVX protein. The term coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-metbylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (erg., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330).

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, 27, 29, 31, 33, 35, and 37). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g, to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/098 10) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, 18, 20, 22, 24, 26, 28, 30, 32, 34, 36, and 38. The invention also includes a mutant or variant protein any of whose residues may be changed from the corresponding residues shown in SEQ ID NOS: 2, 4, 6, 8, 10, 12, 14, 16, 18, 20, 22, 24, 26, 28, 30, 32, 34, 36, and 38 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein” ), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i. e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, 18, 20, 22, 24, 26, 28, 30, 32, 34, 36, and 38) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24, 26, 28, 30, 32, 34, 36, and 38. In other embodiments, the NOVX protein is substantially homologous to SEQ ID NOS: 2, 4, 6, 8, 10, 12, 14, 16, 18, 20, 22, 24, 26, 28, 30, 32, 34, 36, and 38, and retains the functional activity of the protein of SEQ ID NOS: 2, 4, 6, 8, 10, 12, 14, 16, 18, 20, 22, 24, 26, 28, 30, 32, 34, 36, and 38, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24, 26, 28, 30, 32, 34, 36, and 38, and retains the functional activity of the NOVX proteins of SEQ ID NOS: 2, 4, 6, 8, 10, 12, 14, 16, 18, 20, 22, 24, 26, 28, 30, 32, 34, 36, and 38.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31, 33, 35, and 37. The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24, 26, 28, 30, 32, 34, 36, and 38, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol, 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May, 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the ¤-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i. e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, 25, 27, 29, 31, 33, 35, and 37 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g. the cDNA of SEQ ID NOS: 1, 3, 5, 7, 9, 11, 13, 15, 17, 19, 21, 23, 25, 27, 29, 31, 33, 35, and 37), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, 25, 27, 29, 31, 33, 35, and 37 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. USA. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e g stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glyco ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, 25, 27, 29, 31, 33, 35, and 37, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 29, 31, 33, 35, and 37 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, 25, 27, 29, 31, 33, 35, and 37, or a port such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al, 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immnunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff pl=effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1. 1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10-5M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10-5M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×106 cells/ml in DMEM 5% FCS (Hyclone), 100 μM non essential amino acids (Gibco), 1 mM sodium pyruvate (Gibco), mercaptoethanol (5.5×10-5M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10-5M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10-5M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10-5M (Gibco), and 10 mM Hepes (Gibco) and plated at 106 cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10-5M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10-5M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 106 cells/ml in DMEM 5% FCS (Hyclone), 100 μM non essential amino acids (Gibco), 1 mM sodium pyruvate (Gibco), mercaptoethanol 5.5×10-5M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 105-106 cells/ml in DMEM 5% FCS (Hyclone), 100 μM non essential amino acids (Gibco), 1 mM sodium pyruvate (Gibco), mercaptoethanol 5.5×10-5M (Gibco), 10 mM Hepes (Gibco) and IL-2 (4 ng/ml). IL-12 (5ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10-5M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×105 cells/ml for 8 days, changing the media every 3 days and adjusting the cell concentration to 5×105 cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10-5M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10-5M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 107 cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22 /Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD =Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample.

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

NOV1a and NOV1b (AC084364.5/cg-AC084364.5 and 11400078/CG50736-10: Stabilin_Like)

Expression of gene AC084364.5 and variant CG50736-10 was assessed using the primer-probe sets Ag03, Ag068, Ag812, Ag2742, Ag2743, Ag2744, Ag2745 and Ag2746, described in Tables AA, AB, AC, AD, AE, AF, AG, AH and AI. Results of the RTQ-PCR runs are shown in Tables AJ, AK, AL, AM and AN.

TABLE AA

Probe Name Ag03

| | | | |
|---|---|---|---|
| | | | Start |
| Primers | Sequences | Length | Position |

| | | | |
|---|---|---|---|
| Forward | 5′-ctggttgtaggttgccatggt-3′ (SEQ ID NO:115) | 21 | 7156 |
| Probe | TET-5′-cagcttcgttggcacaggcctctc-3′-TAMRA (SEQ ID | 24 | 7130 |
| | NO:116) | | |
| Reverse | 5′-ccagtataagctgacctttgacaaag-3′ (SEQ ID NO:117) | 26 | 7101 |

TABLE AB

Probe Name Ag068

| | | | |
|---|---|---|---|
| | | | Start |
| Primers | Sequences | Length | Position |

| | | | |
|---|---|---|---|
| Forward | 5′-ctggttgtaggttgccatggt-3′ (SEQ ID NO:118) | 21 | 7156 |
| Probe | TET-5′-cagcttcgttggcacaggcctctc-3′-TAMRA (SEQ ID | 24 | 7130 |
| | NO:119) | | |
| Reverse | 5′-ccagtataagctgacctttgacaaag-3′ (SEQ ID NO:120) | 26 | 7101 |

TABLE AC

Probe Name Ag793

| | | | |
|---|---|---|---|
| | | | Start |
| Primers | Sequences | Length | Position |

| | | | |
|---|---|---|---|
| Forward | 5′-ccaaggttttagctgtggatct-3′ (SEQ ID NO:121) | 22 | 5936 |
| Probe | TET-5′-acatccactgcctggaagaccctg-3′-TAMRA (SEQ ID | 24 | 5962 |
| | NO:122) | | |
| Reverse | 5′-cacatttcacactcagctctga-3′ (SEQ ID NO:123) | 22 | 5992 |

TABLE AD

Probe Name Ag812

| | | | |
|---|---|---|---|
| | | | Start |
| Primers | Sequences | Length | Position |

| | | | |
|---|---|---|---|
| Forward | 5′-caggagcatttcgtgaaaga-3′ (SEQ ID NO:124) | 20 | 5329 |
| Probe | TET-5′-ttttgcaoctttatctgcagcctttg-3′-TAMRA (SEQ ID | 26 | 5376 |
| | NO:125) | | |
| Reverse | 5′-tttaacccgagcttcctcat-3′ (SEQ ID NO:126) | 20 | 5402 |

TABLE AE

Probe Name Ag2742

| | | | |
|---|---|---|---|
| | | | Start |
| Primers | Sequences | Length | Position |

| | | | |
|---|---|---|---|
| Forward | 5′-ctgcaaaatcttacgactttgg-3′ (SEQ ID NO:127) | 22 | 5701 |
| Probe | TET-5′-caacaaacaatggctacatcaaatttagca-3′-TAMRA (SEQ | 30 | 5723 |
| | ID NO:128) | | |
| Reverse | 5′-atgacactcagcaaacctgagt-3′ (SEQ ID NO:129) | 22 | 5765 |

TABLE AF

Probe Name Ag2743

| | | | |
|---|---|---|---|
| | | | Start |
| Primers | Sequences | Length | Position |

| | | | |
|---|---|---|---|
| Forward | 5′-ctgcaaaatcttacgactttgg-3′ (SEQ ID NO:130) | 22 | 5701 |
| Probe | TET-5═-caacaaacaatggctacatcaaatttagca-3′-TAMRA (SEQ | 30 | 5723 |
| | ID NO:131) | | |
| Reverse | 5′-atgacactcagcaaacctgagt-3′ (SEQ ID NO:132) | 22 | 5765 |

TABLE AG

Probe Name Ag2744

| | | | |
|---|---|---|---|
| | | | Start |
| Primers | Sequences | Length | Position |

| | | | |
|---|---|---|---|
| Forward | 5′-ctgcaaaatcttacgactttgg-3′ (SEQ ID NO:133) | 22 | 5701 |
| Probe | TET-5′-caacaaacaatggctacatcaaatttagca-3′-TAMRA (SEQ | 30 | 5723 |
| | ID NO:134) | | |
| Reverse | 5′-tcagcaaacctgagtcctgta-3′ (SEQ ID NO:135) | 21 | 5759 |

TABLE AH

Probe Name Ag2745

| | | | |
|---|---|---|---|
| | | | Start |
| Primers | Sequences | Length | Position |

| | | | |
|---|---|---|---|
| Forward | 5′-ctgcaaaatcttacgactttgg-3′ (SEQ ID NO:136) | 22 | 5701 |
| Probe | TET-5′-caacaaacaatggctacatcaaatttagca-3′-TAMRA (SEQ | 30 | 5723 |
| | ID NO:137) | | |
| Reverse | 5′-atgacactcagcaaacctgagt-3′ (SEQ ID NO:138) | 22 | 5765 |

TABLE AI

Probe Name Ag2746

| | | | |
|---|---|---|---|
| | | | Start |
| Primers | Sequences | Length | Position |

| | | | |
|---|---|---|---|
| Forward | 5′-ctgcaaaatcttacgactttgg-3′ (SEQ ID NO:139) | 22 | 5701 |
| Probe | TET-5′-caacaaacaatggctacatcaaatttagca-3′-TAMRA (SEQ | 30 | 5723 |
| | ID NO:140) | | |
| Reverse | 5′-atgacactcagcaaacctgagt-3′ (SEQ ID NO:141) | 22 | 5765 |

TABLE AJ
Panel 1

| | Rel. Exp. (%) | Rel. Exp. (%) | | Rel. Exp. (%) | Rel. Exp. (%) |
|---|---|---|---|---|---|
| | Ag03, Run | Ag068, Run | | Ag03, Run | Ag068, Run |
| Tissue Name | 87353672 | 87361479 | Tissue Name | 87353672 | 87361479 |

| | | | | | |
|---|---|---|---|---|---|
| Endothelial cells | 0.0 | 0.0 | Renal ca. 786-0 | 0.0 | 0.0 |
| Endothelial cells | 0.0 | 0.0 | Renal ca. A498 | 0.1 | 0.5 |
| (treated) | | | | | |
| Pancreas | 0.0 | 2.3 | Renal ca. RXF | 0.0 | 0.0 |
| | | | 393 | | |
| Pancreatic ca. | 0.0 | 0.0 | Renal ca. | 0.0 | 0.0 |
| CAPAN 2 | | | ACHN | | |
| Adrenal gland | 0.5 | 1.3 | Renal ca. UO- | 0.0 | 0.1 |
| | | | 31 | | |
| Thyroid | 1.8 | 2.1 | Renal ca. TK-10 | 0.2 | 0.9 |
| Salivary gland | 2.5 | 3.0 | Liver | 14.2 | 12.9 |
| Pituitary gland | 0.8 | 0.8 | Liver (fetal) | 25.7 | 15.6 |
| Brain (fetal) | 0.1 | 0.4 | Liver ca. | 0.0 | 0.1 |
| | | | (hepatoblast) | | |
| | | | HepG2 | | |
| Brain (whole) | 0.3 | 0.6 | Lung | 0.5 | 0.1 |
| Brain (amygdala) | 0.1 | 0.4 | Lung (fetal) | 4.4 | 6.2 |
| Brain | 0.4 | 0.7 | Lung ca. (small | 0.0 | 0.0 |
| (cerebellum) | | | cell) LX-1 | | |
| Brain | 0.3 | 0.9 | Lung ca. (small | 0.1 | 0.4 |
| (hippocampus) | | | cell) NCI-H69 | | |
| Brain (substantia | 0.1 | 0.3 | Lung ca. (s.cell | 0.0 | 0.0 |
| nigra) | | | var.) SHP-77 | | |
| Brain (thalamus) | 0.1 | 0.2 | Lung ca. (large | 0.0 | 0.0 |
| | | | cell)NCI-H460 | | |
| Brain | 0.4 | 0.5 | Lung ca. (non- | 0.0 | 0.1 |
| (hypothalamus) | | | sm. cell) A549 | | |
| Spinal cord | 0.0 | 0.3 | Lung ca. (non- | 0.0 | 0.3 |
| | | | s.cell) NCI-H23 | | |
| glio/astro U87- | 0.0 | 0.1 | Lung ca. (non- | 0.0 | 0.1 |
| MG | | | s.cell) HOP-62 | | |
| glio/astro U-118- | 0.1 | 0.2 | Lung ca. (non- | 0.0 | 0.0 |
| MG | | | s.cl) NCI-H522 | | |
| astrocytoma | 0.0 | 0.0 | Lung ca. | 0.0 | 0.1 |
| SW1783 | | | (squam.) SW | | |
| | | | 900 | | |
| neuro*; met SK- | 0.0 | 0.0 | Lung ca. | 0.1 | 0.3 |
| N-AS | | | (squam.) NCI- | | |
| | | | H596 | | |
| astrocytoma SF- | 0.0 | 0.1 | Mammary gland | 4.7 | 5.1 |
| 539 | | | | | |
| astrocytoma SNB- | 0.1 | 0.2 | Breast ca.* | 0.0 | 0.2 |
| 75 | | | (pl.ef) MCF-7 | | |
| glioma SNB-19 | 0.6 | 1.6 | Breast ca.* | 0.1 | 0.4 |
| | | | (pl.ef) MDA- | | |
| | | | MB-231 | | |
| glioma U251 | 0.2 | 0.9 | Breast ca.* (pl. | 0.1 | 0.4 |
| | | | ef) T47D | | |
| glioma SF-295 | 0.1 | 0.2 | Breast ca. BT- | 0.0 | 0.0 |
| | | | 549 | | |
| Heart | 0.3 | 0.3 | Breast ca. | 0.1 | 0.6 |
| | | | MDA-N | | |
| Skeletal muscle | 0.3 | 0.4 | Ovary | 3.6 | 1.3 |
| Bone marrow | 3.5 | 3.0 | Ovarian ca. | 0.0 | 0.1 |
| | | | OVCAR-3 | | |
| Thymus | 0.3 | 0.2 | Ovarian ca. | 0.0 | 0.0 |
| | | | OVCAR-4 | | |
| Spleen | 100.0 | 100.0 | Ovarian ca. | 0.1 | 0.6 |
| | | | OVCAR-5 | | |
| Lymph node | 29.3 | 81.2 | Ovarian ca. | 0.2 | 0.7 |
| | | | OVCAR-8 | | |
| Colon (ascending) | 0.8 | 1.0 | Ovarian ca. | 0.0 | 0.3 |
| | | | IGROV-1 | | |
| Stomach | 1.2 | 1.5 | Ovarian ca. | 0.0 | 0.2 |
| | | | (ascites) SK- | | |
| | | | OV-3 | | |
| Small intestine | 1.6 | 1.7 | Uterus | 0.2 | 0.4 |
| Colon ca. SW480 | 0.0 | 0.0 | Placenta | 1.9 | 1.6 |
| Colon ca.* | 0.0 | 0.1 | Prostate | 0.7 | 1.0 |
| SW620 (SW480 | | | | | |
| met) | | | | | |
| Colon ca. HT29 | 0.0 | 0.1 | Prostate ca.* | 0.0 | 0.0 |
| | | | (bone met) PC-3 | | |
| Colon ca. HCT- | 0.0 | 0.0 | Testis | 23.5 | 22.1 |
| 116 | | | | | |
| Colon ca. CaCo-2 | 0.1 | 0.1 | Melanoma | 0.0 | 0.1 |
| | | | Hs688(A).T | | |
| Colon ca. HCT-15 | 0.1 | 0.7 | Melanoma* | 0.0 | 0.1 |
| | | | (met) | | |
| | | | Hs688(B).T | | |
| Colon ca. HCC- | 0.0 | 0.3 | Melanoma | 0.0 | 0.0 |
| 2998 | | | UACC-62 | | |
| Gastric ca. (liver | 0.1 | 0.2 | Melanoma M14 | 0.1 | 0.5 |
| met) NCI-N87 | | | | | |
| Bladder | 1.1 | 0.2 | Melanoma LOX | 0.1 | 0.5 |
| | | | IMVI | | |
| Trachea | 2.4 | 2.2 | Melanoma* | 0.0 | 0.0 |
| | | | (met) SK-MEL-5 | | |
| Kidney | 0.1 | 0.4 | Melanoma SK- | 0.3 | 1.3 |
| | | | MEL-28 | | |
| Kidney (fetal) | 1.0 | 1.3 | | | |

TABLE AK
Panel 1.2

| | Rel. Exp. (%) | Rel. Exp. (%) | | Rel. Exp. (%) | Rel. Exp. (%) |
|---|---|---|---|---|---|
| | Ag812, Run | Ag812, Run | | Ag812, Run | Ag812, Run |
| Tissue Name | 118348259 | 121953945 | Tissue Name | 118348259 | 121953945 |

| | | | | | |
|---|---|---|---|---|---|
| Endothelial cells | 0.0 | 0.0 | Renal ca. 786-0 | 0.0 | 0.0 |
| Heart (Fetal) | 0.3 | 5.6 | Renal ca. A498 | 0.3 | 0.1 |
| Pancreas | 2.0 | 0.6 | Renal ca. RXF | 0.0 | 0.0 |
| | | | 393 | | |
| Pancreatic ca. | 0.0 | 0.0 | Renal ca. | 0.0 | 0.0 |
| CAPAN 2 | | | ACHN | | |
| Adrenal gland | 0.3 | 0.9 | Renal ca. UO- | 0.0 | 0.0 |
| | | | 31 | | |
| Thyroid | 2.0 | 0.7 | Renal ca. TK- | 0.0 | 0.0 |
| | | | 10 | | |
| Salivary gland | 6.3 | 6.7 | Liver | 100.0 | 100.0 |
| Pituitary gland | 0.1 | 0.4 | Liver (fetal) | 37.9 | 58.2 |
| Brain (fetal) | 0.1 | 0.0 | Liver ca. | 0.0 | 0.0 |
| | | | (hepatoblast) | | |
| | | | HepG2 | | |
| Brain (whole) | 1.2 | 0.1 | Lung | 0.4 | 0.8 |
| Brain (amygdala) | 0.0 | 0.1 | Lung (fetal) | 2.4 | 2.5 |
| Brain | 7.6 | 0.0 | Lung ca. (small | 0.0 | 0.0 |
| (cerebellum) | | | cell) LX-1 | | |
| Brain | 0.1 | 0.2 | Lung ca. (small | 0.1 | 0.6 |
| (hippocampus) | | | cell) NCI-H69 | | |
| Brain (thalamus) | 0.0 | 0.0 | Lung ca. (s.cell | 0.0 | 0.0 |
| | | | var.) SHP-77 | | |
| Cerebral Cortex | 0.1 | 0.1 | Lung ca. (large | 0.0 | 0.2 |
| | | | cell)NCI-H460 | | |
| Spinal cord | 0.0 | 0.1 | Lung ca. (non- | 0.1 | 0.2 |
| | | | sm. cell) A549 | | |
| glio/astro U87- | 0.0 | 0.0 | Lung ca. (non- | 0.0 | 0.1 |
| MG | | | s.cell) NCI- | | |
| | | | H23 | | |
| glio/astro U-118- | 0.0 | 0.0 | Lung ca. (non- | 0.0 | 0.0 |
| MG | | | s.cell) HOP-62 | | |
| astrocytoma | 0.0 | 0.0 | Lung ca. (non- | 0.0 | 0.0 |
| SW1783 | | | s.cl) NCI-H522 | | |
| neuro*; met SK- | 0.0 | 0.0 | Lung ca. | 0.0 | 0.0 |
| N-AS | | | (squam.) SW | | |
| | | | 900 | | |
| astrocytoma SF- | 0.0 | 0.1 | Lung ca. | 0.1 | 0.2 |
| 539 | | | (squam.) NCI- | | |
| | | | H596 | | |
| astrocytoma | 0.0 | 0.0 | Mammary | 2.9 | 2.3 |
| SNB-75 | | | gland | | |
| glioma SNB-19 | 0.0 | 0.1 | Breast ca.* | 0.0 | 0.0 |
| | | | (pl.ef) MCF-7 | | |
| glioma U251 | 0.0 | 0.1 | Breast ca.* | 0.0 | 0.0 |
| | | | (pl.ef) MDA- | | |
| | | | MB-231 | | |
| glioma SF-295 | 0.0 | 0.0 | Breast ca.* (pl. | 0.1 | 0.3 |
| | | | ef) T47D | | |
| Heart | 0.8 | 1.8 | Breast ca. BT- | 0.0 | 0.0 |
| | | | 549 | | |
| Skeletal muscle | 2.4 | 1.5 | Breast ca. | 0.0 | 0.1 |
| | | | MDA-N | | |
| Bone marrow | 2.7 | 3.5 | Ovary | 1.4 | 4.2 |
| Thymus | 0.2 | 0.3 | Ovarian ca. | 2.9 | 0.0 |
| | | | OVCAR-3 | | |
| Spleen | 44.8 | 44.4 | Ovarian ca. | 4.1 | 0.0 |
| | | | OVCAR-4 | | |
| Lymph node | 39.2 | 51.8 | Ovarian ca. | 0.2 | 0.3 |
| | | | OVCAR-5 | | |
| Colorectal | 0.0 | 0.2 | Ovarian ca. | 0.0 | 0.1 |
| | | | OVCAR-8 | | |
| Stomach | 1.0 | 2.9 | Ovarian ca. | 0.0 | 0.0 |
| | | | IGROV-1 | | |
| Samll intestine | 1.2 | 2.7 | Ovarian ca. | 0.0 | 0.0 |
| | | | (ascites) SK- | | |
| | | | OV-3 | | |
| Colon ca. SW480 | 0.0 | 0.0 | Uterus | 0.2 | 0.7 |
| Colon ca.* | 0.0 | 0.0 | Placenta | 0.8 | 0.9 |
| SW620 (SW480 | | | | | |
| met) | | | | | |
| Colon ca. HT29 | 0.0 | 0.1 | Prostate | 0.3 | 0.5 |
| Colon ca. HCT- | 0.0 | 0.0 | Prostate ca.* | 0.0 | 0.0 |
| 116 | | | (bone met) PC-3 | | |
| Colon ca. CaCo-2 | 0.0 | 0.0 | Testis | 12.2 | 8.4 |
| CC Well to Mod | 0.1 | 0.4 | Melanoma | 0.0 | 0.0 |
| Diff (ODO3866) | | | Hs688(A).T | | |
| Colon ca. HCC- | 0.0 | 0.0 | Melanoma* | 0.0 | 0.1 |
| 2998 | | | (met) | | |
| | | | Hs688(B).T | | |
| Gastric ca. (liver | 0.0 | 0.1 | Melanoma | 0.0 | 0.0 |
| met) NCI-N87 | | | UACC-62 | | |
| Bladder | 3.7 | 3.8 | Melanoma | 0.1 | 0.2 |
| | | | M14 | | |
| Trachea | 1.1 | 1.9 | Melanoma | 0.0 | 0.0 |
| | | | LOX IMVI | | |
| Kidney | 0.1 | 0.4 | Melanoma* | 0.1 | 0.0 |
| | | | (met) SK- | | |
| | | | MEL-5 | | |
| Kidney (fetal) | 0.8 | 2.1 | | | |

TABLE AL
Panel 1.3D

| | Rel. Exp. (%) | Rel. Exp. (%) | Rel. Exp. (%) | Rel. Exp. (%) | Rel. Exp. (%) |
|---|---|---|---|---|---|
| | Ag2742, Run | Ag2743, Run | Ag2744, Run | Ag2745, Run | Ag2746, Run |
| Tissue Name | 153641674 | 153658349 | 153670718 | 153664738 | 153675151 |
| Liver | 0.0 | 0.0 | 0.0 | 0.0 | 0.0 |
| adenocarcinoma | | | | | |
| Pancreas | 0.2 | 0.4 | 0.3 | 0.2 | 0.2 |
| Pancreatic ca. | 0.0 | 0.0 | 0.0 | 0.0 | 0.0 |
| CAPAN 2 | | | | | |
| Adrenal gland | 0.2 | 0.0 | 0.0 | 0.4 | 0.2 |
| Thyroid | 0.5 | 0.6 | 1.1 | 1.4 | 0.6 |
| Salivary gland | 1.0 | 1.0 | 0.7 | 0.7 | 0.1 |
| Pituitary gland | 0.0 | 0.0 | 0.1 | 0.0 | 0.0 |
| Brain (fetal) | 0.0 | 0.0 | 0.0 | 0.0 | 0.0 |
| Brain (whole) | 0.0 | 0.0 | 0.1 | 0.0 | 0.0 |
| Brain (amygdala) | 0.0 | 0.3 | 0.1 | 0.0 | 0.0 |
| Brain | 0.0 | 0.0 | 0.0 | 0.0 | 0.0 |
| (cerebellum) | | | | | |
| Brain | 0.3 | 0.0 | 0.1 | 0.0 | 0.0 |
| (hippocampus) | | | | | |
| Brain (substantia | 0.0 | 0.0 | 0.0 | 0.0 | 0.0 |
| nigra) | | | | | |
| Brain (thalamus) | 0.0 | 0.0 | 0.0 | 0.0 | 0.0 |
| Cerebral Cortex | 0.0 | 0.0 | 0.0 | 0.1 | 0.0 |
| Spinal cord | 0.0 | 0.0 | 0.0 | 0.0 | 0.0 |
| glio/astro U87- | 0.0 | 0.0 | 0.0 | 0.0 | 0.0 |
| MG | | | | | |
| glio/astro U-118- | 0.0 | 0.0 | 0.0 | 0.0 | 0.0 |
| MG | | | | | |
| astrocytoma | 0.0 | 0.0 | 0.0 | 0.0 | 0.0 |
| SW1783 | | | | | |
| neuro*; met SK- | 0.0 | 0.0 | 0.0 | 0.0 | 0.0 |
| N-AS | | | | | |
| astrocytoma SF- | 0.0 | 0.0 | 0.0 | 0.0 | 0.0 |
| 539 | | | | | |
| astrocytoma SNB- | 0.0 | 0.0 | 0.0 | 0.0 | 0.0 |
| 75 | | | | | |
| glioma SNB-19 | 0.0 | 0.0 | 0.0 | 0.0 | 0.0 |
| glioma U251 | 0.0 | 0.0 | 0.0 | 0.0 | 0.0 |
| glioma SF-295 | 0.0 | 0.0 | 0.0 | 0.0 | 0.0 |
| Heart (Fetal) | 1.8 | 0.7 | 2.1 | 2.1 | 1.5 |
| Heart | 0.1 | 0.2 | 0.0 | 0.1 | 0.2 |
| Skeletal muscle | 8.5 | 6.9 | 9.3 | 9.5 | 6.8 |
| (Fetal) | | | | | |
| Skeletal muscle | 0.0 | 0.0 | 0.0 | 0.1 | 0.0 |
| Bone marrow | 2.6 | 2.2 | 3.3 | 2.4 | 3.1 |
| Thymus | 0.1 | 0.0 | 0.1 | 0.1 | 0.2 |
| Spleen | 100.0 | 100.0 | 100.0 | 100.0 | 100.0 |
| Lymph node | 20.7 | 17.9 | 25.5 | 26.4 | 32.8 |
| Colorectal | 1.6 | 1.1 | 0.6 | 0.7 | 0.4 |
| Stomach | 1.3 | 0.5 | 1.2 | 0.7 | 0.9 |
| Small intestine | 1.3 | 1.0 | 1.1 | 1.1 | 1.2 |
| Colon ca. SW480 | 0.0 | 0.0 | 0.0 | 0.0 | 0.0 |
| Colon ca.* | 0.0 | 0.0 | 0.0 | 0.0 | 0.0 |
| SW620 (SW480 | | | | | |
| met) | | | | | |
| Colon ca. HT29 | 0.0 | 0.0 | 0.0 | 0.0 | 0.0 |
| Colon ca. HCT- | 0.0 | 0.0 | 0.0 | 0.0 | 0.0 |
| 116 | | | | | |
| Colon ca. CaCo-2 | 0.0 | 0.0 | 0.0 | 0.0 | 0.0 |
| CC Well to Mod | 0.1 | 0.1 | 0.0 | 0.2 | 0.1 |
| Diff (ODO3866) | | | | | |
| Colon ca. HCC- | 0.0 | 0.0 | 0.0 | 0.0 | 0.0 |
| 2998 | | | | | |
| Gastric ca. (liver | 0.0 | 0.0 | 0.0 | 0.0 | 0.1 |
| met) NCI-N87 | | | | | |
| Bladder | 0.3 | 0.4 | 0.6 | 0.6 | 0.4 |
| Trachea | 1.3 | 0.7 | 1.4 | 1.2 | 1.3 |
| Kidney | 0.0 | 0.0 | 0.1 | 0.0 | 0.0 |
| Kidney (fetal) | 2.4 | 3.4 | 3.8 | 2.1 | 3.0 |
| Renal ca. 786-0 | 0.0 | 0.0 | 0.0 | 0.0 | 0.0 |
| Renal ca. A498 | 0.0 | 0.0 | 0.0 | 0.0 | 0.0 |
| Renal ca. RXF | 0.0 | 0.0 | 0.0 | 0.0 | 0.0 |
| 393 | | | | | |
| Renal ca. ACHN | 0.0 | 0.0 | 0.0 | 0.0 | 0.0 |
| Renal ca. UO-31 | 0.0 | 0.0 | 0.0 | 0.0 | 0.0 |
| Renal ca. TK-10 | 0.0 | 0.0 | 0.0 | 0.0 | 0.0 |
| Liver | 5.6 | 8.8 | 6.1 | 6.4 | 10.3 |
| Liver (fetal) | 33.4 | 33.2 | 33.9 | 33.4 | 36.6 |
| Liver ca. | 0.0 | 0.0 | 0.0 | 0.0 | 0.0 |
| (hepatoblast) | | | | | |
| HepG2 | | | | | |
| Lung | 0.6 | 0.6 | 0.6 | 0.7 | 0.2 |
| Lung (fetal) | 2.0 | 1.9 | 2.4 | 1.0 | 3.1 |
| Lung ca. (small | 0.0 | 0.0 | 0.0 | 0.0 | 0.0 |
| cell) LX-1 | | | | | |
| Lung ca. (small | 0.0 | 0.0 | 0.0 | 0.0 | 0.0 |
| cell) NCI-H69 | | | | | |
| Lung ca. (s.cell | 0.0 | 0.0 | 0.0 | 0.0 | 0.0 |
| var.) SHP-77 | | | | | |
| Lung ca. (large | 0.0 | 0.0 | 0.0 | 0.0 | 0.0 |
| cell)NCI-H460 | | | | | |
| Lung ca. (non-sm. | 0.0 | 0.0 | 0.0 | 0.0 | 0.0 |
| cell) A549 | | | | | |
| Lung ca. (non- | 0.0 | 0.0 | 0.0 | 0.0 | 0.0 |
| s.cell) NCI-H23 | | | | | |
| Lung ca. (non- | 0.0 | 0.0 | 0.0 | 0.0 | 0.0 |
| s.cell) HOP-62 | | | | | |
| Lung ca. (non- | 0.0 | 0.0 | 0.0 | 0.0 | 0.0 |
| s.cl) NCI-H522 | | | | | |
| Lung ca. (squam.) | 0.0 | 0.0 | 0.0 | 0.0 | 0.0 |
| SW 900 | | | | | |
| Lung ca. (squam.) | 0.0 | 0.0 | 0.0 | 0.0 | 0.0 |
| NCI-H596 | | | | | |
| Mammary gland | 1.6 | 1.5 | 1.1 | 1.7 | 1.5 |
| Breast ca.* (pl.ef) | 0.0 | 0.0 | 0.0 | 0.0 | 0.0 |
| MCF-7 | | | | | |
| Breast ca.* (pl.ef) | 0.0 | 0.0 | 0.0 | 0.0 | 0.0 |
| MDA-MB-231 | | | | | |
| Breast ca.* (pl. ef) | 0.0 | 0.0 | 0.0 | 0.0 | 0.0 |
| T47D | | | | | |
| Breast ca. BT-549 | 0.0 | 0.0 | 0.0 | 0.0 | 0.0 |
| Breast ca. MDA-N | 0.0 | 0.0 | 0.0 | 0.0 | 0.0 |
| Ovary | 5.0 | 5.4 | 4.5 | 6.0 | 4.6 |
| Ovarian ca. | 0.0 | 0.0 | 0.0 | 0.0 | 0.0 |
| OVCAR-3 | | | | | |
| Ovarian ca. | 0.0 | 0.0 | 0.0 | 0.0 | 0.0 |
| OVCAR-4 | | | | | |
| Ovarian ca. | 0.0 | 0.0 | 0.0 | 0.0 | 0.0 |
| OVCAR-5 | | | | | |
| Ovarian ca. | 0.0 | 0.0 | 0.0 | 0.0 | 0.0 |
| OVCAR-8 | | | | | |
| Ovarian ca. | 0.0 | 0.0 | 0.0 | 0.0 | 0.0 |
| IGROV-1 | | | | | |
| Ovarian ca. | 0.0 | 0.0 | 0.0 | 0.0 | 0.0 |
| (ascites) SK-OV-3 | | | | | |
| Uterus | 0.2 | 0.2 | 0.5 | 0.3 | 0.1 |
| Placenta | 0.4 | 0.0 | 0.4 | 0.1 | 0.2 |
| Prostate | 0.2 | 0.1 | 0.1 | 0.1 | 0.1 |
| Prostate ca.* | 0.0 | 0.0 | 0.0 | 0.0 | 0.0 |
| (bone met) PC-3 | | | | | |
| Testis | 7.5 | 5.9 | 4.7 | 6.5 | 5.6 |
| Melanoma | 0.0 | 0.0 | 0.0 | 0.0 | 0.0 |
| Hs688(A).T | | | | | |
| Melanoma* (met) | 0.0 | 0.0 | 0.0 | 0.0 | 0.0 |
| Hs688(B).T | | | | | |
| Melanoma | 0.0 | 0.0 | 0.0 | 0.0 | 0.0 |
| UACC-62 | | | | | |
| Melanoma M14 | 0.0 | 0.0 | 0.0 | 0.0 | 0.0 |
| Melanoma LOX | 0.0 | 0.0 | 0.0 | 0.0 | 0.0 |
| IMVI | | | | | |
| Melanoma* (met) | 0.0 | 0.0 | 0.0 | 0.0 | 0.0 |
| SK-MEL-5 | | | | | |
| Adipose | 1.0 | 1.5 | 0.6 | 0.5 | 1.1 |

TABLE AM
Panel 2D

| | Rel. Exp. (%) | Rel. Exp. (%) | Rel. Exp. (%) | Rel. Exp. (%) | Rel. Exp. (%) |
|---|---|---|---|---|---|
| | Ag2742, Run | Ag2743, Run | Ag2744, Run | Ag2745, Run | Ag2746, Run |
| Tissue Name | 153641758 | 153658357 | 153670751 | 153664739 | 153675220 |
| Normal Colon | 3.2 | 3.8 | 4.4 | 4.8 | 4.5 |
| CC Well to Mod | 0.1 | 0.1 | 0.1 | 0.0 | 0.0 |
| Diff (ODO3866) | | | | | |
| CC Margin | 0.7 | 0.5 | 0.9 | 1.2 | 0.3 |
| (ODO3866) | | | | | |
| CC Gr.2 | 0.2 | 0.4 | 0.2 | 0.2 | 0.1 |
| rectosigmoid | | | | | |
| (ODO3868) | | | | | |
| CC Margin | 0.1 | 0.1 | 0.0 | 0.2 | 0.1 |
| (ODO3868) | | | | | |
| CC Mod Diff | 0.0 | 0.1 | 0.1 | 0.0 | 0.0 |
| (ODO3920) | | | | | |
| CC Margin | 1.1 | 2.0 | 3.1 | 1.0 | 1.4 |
| (ODO3920) | | | | | |
| CC Gr.2 ascend | 0.2 | 0.3 | 0.9 | 0.5 | 0.7 |
| colon | | | | | |
| (ODO3921) | | | | | |
| CC Margin | 0.7 | 0.3 | 0.9 | 1.0 | 0.2 |
| (ODO3921) | | | | | |
| CC from Partial | 7.6 | 8.4 | 10.2 | 9.5 | 8.8 |
| Hepatectomy | | | | | |
| (ODO4309) Mets | | | | | |
| Liver Margin | 100.0 | 100.0 | 100.0 | 100.0 | 100.0 |
| (ODO4309) | | | | | |
| Colon mets to | 0.4 | 0.3 | 0.3 | 1.5 | 0.4 |
| lung (OD04451- | | | | | |
| 01) | | | | | |
| Lung Margin | 0.2 | 0.1 | 0.8 | 0.2 | 0.2 |
| (OD04451-02) | | | | | |
| Normal Prostate | 0.4 | 0.0 | 0.2 | 0.1 | 0.1 |
| 6546-1 | | | | | |
| Prostate Cancer | 0.2 | 0.2 | 0.0 | 0.3 | 0.0 |
| (OD04410) | | | | | |
| Prostate Margin | 0.0 | 0.2 | 0.3 | 0.0 | 0.6 |
| (OD04410) | | | | | |
| Prostate Cancer | 0.6 | 0.1 | 0.2 | 0.2 | 0.3 |
| (OD04720-01) | | | | | |
| Prostate Margin | 0.5 | 0.5 | 0.6 | 0.3 | 0.2 |
| (OD04720-02) | | | | | |
| Normal Lung | 5.6 | 5.2 | 6.3 | 8.1 | 5.9 |
| Lung Met to | 0.0 | 0.0 | 0.0 | 0.0 | 0.0 |
| Muscle | | | | | |
| (ODO4286) | | | | | |
| Muscle Margin | 0.5 | 0.0 | 0.1 | 0.2 | 0.0 |
| (ODO4286) | | | | | |
| Lung Malignant | 1.0 | 1.0 | 1.3 | 0.8 | 1.0 |
| Cancer | | | | | |
| (OD03126) | | | | | |
| Lung Margin | 0.9 | 1.1 | 0.9 | 1.4 | 1.4 |
| (OD03126) | | | | | |
| Lung Cancer | 1.3 | 0.8 | 1.6 | 1.5 | 1.4 |
| (OD04404) | | | | | |
| Lung Margin | 2.0 | 2.5 | 4.3 | 3.4 | 3.2 |
| (OD04404) | | | | | |
| Lung Cancer | 0.2 | 0.1 | 0.5 | 0.0 | 0.2 |
| (OD04565) | | | | | |
| Lung Margin | 0.3 | 0.0 | 0.3 | 0.4 | 0.7 |
| (OD04565) | | | | | |
| Lung Cancer | 0.4 | 0.9 | 1.2 | 1.5 | 1.2 |
| (OD04237-01) | | | | | |
| Lung Margin | 5.6 | 5.4 | 7.9 | 6.3 | 5.6 |
| (OD04237-02) | | | | | |
| Ocular Mel Met | 0.2 | 0.0 | 0.0 | 0.1 | 0.4 |
| to Liver | | | | | |
| (ODO4310) | | | | | |
| Liver Margin | 52.9 | 64.6 | 79.6 | 81.8 | 63.3 |
| (ODO4310) | | | | | |
| Melanoma | 0.0 | 0.0 | 0.0 | 0.1 | 0.3 |
| Metastasis | | | | | |
| Lung Margin | 0.2 | 2.0 | 0.9 | 1.5 | 0.5 |
| (OD04321) | | | | | |
| Normal Kidney | 0.5 | 0.3 | 0.3 | 0.7 | 0.3 |
| Kidney Ca, | 0.0 | 0.1 | 0.0 | 0.0 | 0.1 |
| Nuclear grade 2 | | | | | |
| (OD04338) | | | | | |
| Kidney Margin | 0.0 | 0.2 | 0.5 | 0.1 | 0.3 |
| (OD04338) | | | | | |
| Kidney Ca | 0.0 | 0.0 | 0.0 | 0.0 | 0.0 |
| Nuclear grade 1/2 | | | | | |
| (OD04339) | | | | | |
| Kidney Margin | 0.0 | 0.1 | 0.1 | 0.3 | 0.1 |
| (OD04339) | | | | | |
| Kidney Ca, Clear | 0.0 | 0.1 | 0.0 | 0.0 | 0.1 |
| cell type | | | | | |
| (OD04340) | | | | | |
| Kidney Margin | 0.2 | 0.1 | 0.8 | 0.0 | 0.0 |
| (OD04340) | | | | | |
| Kidney Ca, | 0.0 | 0.0 | 0.2 | 0.0 | 0.1 |
| Nuclear grade 3 | | | | | |
| (OD04348) | | | | | |
| Kidney Margin | 0.0 | 0.1 | 0.2 | 0.1 | 0.1 |
| (OD04348) | | | | | |
| Kidney Cancer | 0.2 | 0.1 | 0.1 | 1.2 | 0.2 |
| (OD04622-01) | | | | | |
| Kidney Margin | 0.0 | 0.1 | 0.0 | 0.0 | 0.0 |
| (OD04622-03) | | | | | |
| Kidney Cancer | 0.0 | 0.0 | 0.0 | 0.0 | 0.0 |
| (OD04450-01) | | | | | |
| Kidney Margin | 0.1 | 0.0 | 0.1 | 0.1 | 0.1 |
| (OD04450-03) | | | | | |
| Kidney Cancer | 0.0 | 0.0 | 0.0 | 0.0 | 0.0 |
| 8120607 | | | | | |
| Kidney Margin | 0.1 | 0.0 | 0.2 | 0.0 | 0.0 |
| 8120608 | | | | | |
| Kidney Cancer | 0.3 | 0.2 | 0.2 | 0.1 | 0.5 |
| 8120613 | | | | | |
| Kidney Margin | 0.3 | 0.1 | 0.4 | 0.3 | 0.0 |
| 8120614 | | | | | |
| Kidney Cancer | 0.0 | 0.1 | 0.1 | 0.1 | 0.2 |
| 9010320 | | | | | |
| Kidney Margin | 0.1 | 0.0 | 0.0 | 0.1 | 0.2 |
| 9010321 | | | | | |
| Normal Uterus | 0.5 | 0.1 | 0.8 | 0.1 | 0.5 |
| Uterine Cancer | 0.8 | 0.7 | 1.3 | 0.4 | 0.6 |
| 064011 | | | | | |
| Normal Thyroid | 1.8 | 1.1 | 1.5 | 0.9 | 2.3 |
| Thyroid Cancer | 0.0 | 0.0 | 0.0 | 0.0 | 0.0 |
| Thyroid Cancer | 0.5 | 1.0 | 1.3 | 0.6 | 0.8 |
| A302152 | | | | | |
| Thyroid Margin | 2.3 | 2.4 | 3.5 | 3.9 | 2.4 |
| A302153 | | | | | |
| Normal Breast | 9.0 | 6.8 | 9.0 | 9.9 | 5.5 |
| Breast Cancer | 0.1 | 0.0 | 0.1 | 0.0 | 0.0 |
| Breast Cancer | 0.3 | 0.4 | 0.2 | 0.7 | 0.2 |
| (OD04590-01) | | | | | |
| Breast Cancer | 1.7 | 2.4 | 2.5 | 2.5 | 1.4 |
| Mets (OD04590- | | | | | |
| 03) | | | | | |
| Breast Cancer | 10.7 | 13.2 | 22.1 | 15.6 | 12.8 |
| Metastasis | | | | | |
| Breast Cancer | 0.5 | 0.8 | 0.8 | 0.7 | 0.7 |
| Breast Cancer | 4.1 | 2.5 | 5.1 | 3.1 | 3.6 |
| Breast Cancer | 0.9 | 0.2 | 0.2 | 0.2 | 0.2 |
| 9100266 | | | | | |
| Breast Margin | 0.7 | 0.4 | 1.0 | 0.7 | 1.2 |
| 9100265 | | | | | |
| Breast Cancer | 1.2 | 1.2 | 1.1 | 1.9 | 0.8 |
| A209073 | | | | | |
| Breast Margin | 1.5 | 1.0 | 2.1 | 1.5 | 0.6 |
| A2090734 | | | | | |
| Normal Liver | 27.5 | 27.9 | 37.4 | 42.3 | 27.2 |
| Liver Cancer | 0.8 | 0.5 | 0.3 | 0.6 | 0.5 |
| Liver Cancer | 33.7 | 36.6 | 36.6 | 39.0 | 27.5 |
| 1025 | | | | | |
| Liver Cancer | 4.6 | 3.6 | 5.7 | 5.8 | 5.2 |
| 1026 | | | | | |
| Liver Cancer | 36.9 | 38.7 | 50.3 | 46.7 | 38.7 |
| 6004-T | | | | | |
| Liver Tissue | 1.5 | 1.0 | 1.3 | 1.3 | 1.6 |
| 6004-N | | | | | |
| Liver Cancer | 4.5 | 3.9 | 4.2 | 4.3 | 2.9 |
| 6005-T | | | | | |
| Liver Tissue | 22.2 | 24.5 | 32.8 | 27.5 | 28.7 |
| 6005-N | | | | | |
| Normal Bladder | 5.3 | 3.4 | 4.8 | 4.2 | 5.5 |
| Bladder Cancer | 1.6 | 1.3 | 1.5 | 1.6 | 1.7 |
| Bladder Cancer | 0.9 | 0.4 | 1.5 | 1.0 | 0.3 |
| Bladder Cancer | 0.0 | 0.0 | 0.0 | 0.0 | 0.0 |
| (OD04718-01) | | | | | |
| Bladder Normal | 5.2 | 6.2 | 7.8 | 6.0 | 4.1 |
| Adjacent | | | | | |
| (OD04718-03) | | | | | |
| Normal Ovary | 1.6 | 3.0 | 5.4 | 3.6 | 3.6 |
| Ovarian Cancer | 0.0 | 0.1 | 0.1 | 0.6 | 0.5 |
| Ovarian Cancer | 0.0 | 0.0 | 0.0 | 0.1 | 0.0 |
| (OD04768-07) | | | | | |
| Ovary Margin | 2.3 | 1.7 | 3.1 | 2.4 | 2.6 |
| (OD04768-08) | | | | | |
| Normal Stomach | 0.9 | 0.8 | 1.7 | 0.8 | 0.8 |
| Gastric Cancer | 0.0 | 0.4 | 0.8 | 0.4 | 0.2 |
| 9060358 | | | | | |
| Stomach Margin | 1.2 | 0.8 | 1.3 | 1.0 | 0.9 |
| 9060359 | | | | | |
| Gastric Cancer | 0.1 | 0.2 | 0.3 | 0.5 | 0.6 |
| 9060395 | | | | | |
| Stomach Margin | 0.8 | 0.6 | 1.7 | 0.7 | 0.6 |
| 9060394 | | | | | |
| Gastric Cancer | 0.1 | 0.1 | 0.1 | 0.0 | 0.0 |
| 9060397 | | | | | |
| Stomach Margin | 0.3 | 0.1 | 0.6 | 0.1 | 0.3 |
| 9060396 | | | | | |
| Gastric Cancer | 0.7 | 0.6 | 0.4 | 1.1 | 0.3 |
| 064005 | | | | | |

TABLE AN
Panel 4D

| | Rel. | Rel. | Rel. | Rel. | Rel. | Rel. |
|---|---|---|---|---|---|---|
| | Exp. (%) | Exp. (%) | Exp. (%) | Exp. (%) | Exp. (%) | Exp. (%) |
| | Ag2742, | Ag2743, | Ag2744, | Ag2745, | Ag2746, | Ag812, |
| | Run | Run | Run | Run | Run | Run |
| Tissue Name | 153641803 | 153658360 | 153670759 | 153664740 | 153675321 | 138175358 |
| Secondary Th1 act | 0.0 | 0.0 | 0.0 | 0.0 | 0.0 | 0.0 |
| Secondary Th2 act | 0.0 | 0.7 | 0.0 | 0.0 | 0.0 | 0.0 |
| Secondary Tr1 act | 1.7 | 0.0 | 1.9 | 0.0 | 7.5 | 2.8 |
| Secondary Th1 rest | 0.0 | 0.0 | 0.0 | 0.0 | 0.0 | 0.0 |
| Secondary Th2 rest | 0.0 | 0.0 | 0.0 | 0.0 | 0.0 | 0.0 |
| Secondary Tr1 rest | 0.0 | 0.0 | 0.0 | 0.0 | 0.0 | 2.8 |
| Primary Th1 act | 0.0 | 0.0 | 0.0 | 0.0 | 0.0 | 0.0 |
| Primary Th2 act | 1.6 | 0.0 | 0.0 | 0.0 | 0.0 | 4.8 |
| Primary Tr1 act | 0.0 | 0.0 | 0.0 | 0.0 | 0.0 | 0.0 |
| Primary Th1 rest | 0.0 | 0.0 | 0.0 | 0.0 | 0.0 | 0.0 |
| Primary Th2 rest | 0.0 | 0.0 | 0.0 | 0.0 | 0.0 | 0.0 |
| Primary Tr1 rest | 0.0 | 0.0 | 0.0 | 0.0 | 0.0 | 0.0 |
| CD45RA CD4 | 0.0 | 0.0 | 0.0 | 0.0 | 0.0 | 0.0 |
| lymphocyte act | | | | | | |
| CD45RO CD4 | 0.0 | 0.0 | 0.0 | 0.0 | 0.0 | 0.0 |
| lymphocyte act | | | | | | |
| CD8 lymphocyte | 0.0 | 0.0 | 0.0 | 0.0 | 0.0 | 0.0 |
| act | | | | | | |
| Secondary CD8 | 0.0 | 0.0 | 0.0 | 0.0 | 0.0 | 0.0 |
| lymphocyte rest | | | | | | |
| Secondary CD8 | 0.7 | 0.0 | 0.0 | 7.8 | 0.0 | 1.8 |
| lymphocyte act | | | | | | |
| CD4 lymphocyte | 0.0 | 0.0 | 0.0 | 0.0 | 0.0 | 0.0 |
| none | | | | | | |
| 2ry | 0.0 | 0.0 | 0.0 | 0.0 | 0.0 | 0.0 |
| Th1/Th2/Tr1_anti- | | | | | | |
| CD95 CH11 | | | | | | |
| LAK cells rest | 0.0 | 0.0 | 0.0 | 0.0 | 0.0 | 0.0 |
| LAK cells IL-2 | 0.0 | 0.0 | 0.0 | 0.0 | 0.0 | 4.7 |
| LAK cells IL- | 0.0 | 0.0 | 0.0 | 0.0 | 1.9 | 0.0 |
| 2+IL-12 | | | | | | |
| LAK cells IL- | 0.0 | 1.5 | 3.3 | 5.0 | 0.0 | 3.0 |
| 2+IFN gamma | | | | | | |
| LAK cells IL-2+ | 0.0 | 0.0 | 2.1 | 0.0 | 0.0 | 0.0 |
| IL-18 | | | | | | |
| LAK cells | 0.0 | 0.0 | 0.0 | 0.0 | 0.0 | 0.0 |
| PMA/ionomycin | | | | | | |
| NK Cells IL-2 rest | 0.0 | 0.0 | 0.0 | 0.0 | 0.0 | 0.0 |
| Two Way MLR 3 | 0.0 | 0.0 | 0.0 | 0.0 | 0.0 | 0.0 |
| day | | | | | | |
| Two Way MLR 5 | 0.0 | 0.0 | 0.0 | 0.0 | 0.0 | 0.0 |
| day | | | | | | |
| Two Way MLR 7 | 2.0 | 0.0 | 6.4 | 0.0 | 0.0 | 11.3 |
| day | | | | | | |
| PBMC rest | 0.0 | 0.0 | 0.0 | 0.0 | 0.0 | 5.1 |
| PBMC PWM | 0.0 | 2.3 | 0.0 | 0.0 | 0.0 | 2.7 |
| PBMC PHA-L | 0.0 | 1.5 | 2.5 | 0.0 | 0.0 | 2.2 |
| Ramos (B cell) | 0.0 | 0.0 | 0.0 | 0.0 | 0.0 | 0.0 |
| none | | | | | | |
| Ramos (B cell) | 0.0 | 0.0 | 0.0 | 0.0 | 0.0 | 0.0 |
| ionomycin | | | | | | |
| B lymphocytes | 0.0 | 0.0 | 0.0 | 0.0 | 0.0 | 2.0 |
| PWM | | | | | | |
| B lymphocytes | 0.0 | 0.0 | 0.0 | 0.0 | 0.0 | 6.0 |
| CD40L and IL-4 | | | | | | |
| EOL-1 dbcAMP | 0.0 | 0.0 | 0.0 | 0.0 | 0.0 | 0.0 |
| EOL-1 dbcAMP | 0.0 | 0.0 | 0.0 | 0.0 | 0.0 | 0.0 |
| PMA/ionomycin | | | | | | |
| Dendritic cells | 1.4 | 2.9 | 0.0 | 0.0 | 4.9 | 0.0 |
| none | | | | | | |
| Dendritic cells LPS | 0.0 | 0.0 | 0.0 | 0.0 | 0.0 | 0.0 |
| Dendritic cells anti- | 0.0 | 0.0 | 3.3 | 0.0 | 0.0 | 0.0 |
| CD40 | | | | | | |
| Monocytes rest | 0.0 | 0.0 | 0.0 | 0.0 | 0.0 | 0.0 |
| Monocytes LPS | 0.0 | 0.0 | 0.0 | 0.0 | 0.0 | 0.0 |
| Macrophages rest | 0.0 | 0.0 | 0.0 | 0.0 | 0.0 | 0.0 |
| Macrophages LPS | 0.0 | 0.0 | 0.0 | 2.4 | 0.0 | 0.0 |
| HUVEC none | 0.0 | 0.0 | 0.0 | 0.0 | 0.0 | 0.0 |
| HUVEC starved | 0.0 | 0.0 | 0.0 | 0.0 | 0.0 | 0.0 |
| HUVEC IL-1beta | 0.0 | 0.0 | 0.0 | 0.0 | 0.0 | 0.0 |
| HUVEC IFN | 3.7 | 0.0 | 0.0 | 0.0 | 2.7 | 0.0 |
| gamma | | | | | | |
| HUVEC TNF | 0.0 | 0.0 | 0.0 | 0.0 | 0.0 | 0.0 |
| alpha + IFN | | | | | | |
| gamma | | | | | | |
| HUVEC TNF | 0.0 | 0.0 | 0.0 | 0.0 | 0.0 | 0.0 |
| alpha + IL4 | | | | | | |
| HUVEC IL-11 | 1.4 | 0.0 | 0.0 | 0.0 | 0.0 | 2.6 |
| Lung | 1.6 | 0.0 | 2.2 | 0.0 | 0.0 | 0.0 |
| Microvascular EC | | | | | | |
| none | | | | | | |
| Lung | 3.1 | 2.9 | 0.0 | 0.0 | 0.0 | 0.0 |
| Microvascular EC | | | | | | |
| TNFalpha + IL- | | | | | | |
| 1beta | | | | | | |
| Microvascular | 0.0 | 3.1 | 5.0 | 0.0 | 0.0 | 12.6 |
| Dermal EC none | | | | | | |
| Microsvasular | 1.4 | 1.1 | 0.0 | 0.0 | 1.6 | 0.0 |
| Dermal EC | | | | | | |
| TNFalpha + IL- | | | | | | |
| 1beta | | | | | | |
| Bronchial | 0.0 | 0.0 | 0.0 | 0.0 | 0.0 | 0.0 |
| epithelium | | | | | | |
| TNFalpha + | | | | | | |
| IL1beta | | | | | | |
| Small airway | 0.0 | 0.0 | 0.0 | 0.0 | 0.0 | 0.0 |
| epithelium none | | | | | | |
| Small airway | 0.0 | 0.0 | 0.0 | 0.0 | 0.0 | 0.0 |
| epithelium | | | | | | |
| TNFalpha + IL- | | | | | | |
| 1beta | | | | | | |
| Coronery artery | 0.0 | 0.0 | 0.0 | 0.0 | 0.0 | 0.0 |
| SMC rest | | | | | | |
| Coronery artery | 0.0 | 0.0 | 0.0 | 0.0 | 0.0 | 0.0 |
| SMC TNFalpha + | | | | | | |
| IL-1beta | | | | | | |
| Astrocytes rest | 0.0 | 0.0 | 0.0 | 0.0 | 0.0 | 0.0 |
| Astrocytes | 0.0 | 0.0 | 0.0 | 0.0 | 0.0 | 0.0 |
| TNFalpha + IL- | | | | | | |
| 1beta | | | | | | |
| KU-812 (Basophil) | 0.0 | 0.0 | 0.0 | 0.0 | 0.0 | 0.0 |
| rest | | | | | | |
| KU-812 (Basophil) | 0.0 | 0.0 | 0.0 | 0.0 | 0.0 | 0.0 |
| PMA/ionomycin | | | | | | |
| CCD1106 | 0.0 | 0.0 | 0.0 | 0.0 | 0.0 | 0.0 |
| (Keratinocytes) | | | | | | |
| none | | | | | | |
| CCD1106 | 0.0 | 0.0 | 0.0 | 0.0 | 0.0 | |
| (Keratinocytes) | | | | | | |
| TNFalpha + IL- | | | | | | |
| 1beta | | | | | | |
| Liver cirrhosis | 100.0 | 100.0 | 100.0 | 100.0 | 100.0 | 100.0 |
| Lupus kidney | 0.0 | 1.4 | 0.0 | 0.0 | 0.0 | 0.0 |
| NCI-H292 none | 0.0 | 0.0 | 0.0 | 0.0 | 0.0 | 0.0 |
| NCI-H292 IL-4 | 0.0 | 0.0 | 0.0 | 0.0 | 2.3 | 0.0 |
| NCI-H292 IL-9 | 0.0 | 0.0 | 0.0 | 0.0 | 0.0 | 0.0 |
| NCI-H292 IL-13 | 0.0 | 0.0 | 0.0 | 0.0 | 0.0 | 0.0 |
| NCI-H292 IFN | 0.0 | 0.0 | 0.0 | 0.0 | 0.0 | 0.0 |
| gamma | | | | | | |
| HPAEC none | 4.8 | 1.4 | 0.0 | 0.0 | 0.0 | 3.0 |
| HPAEC TNF alpha | 0.0 | 0.0 | 0.0 | 0.0 | 0.0 | 0.0 |
| + IL-1 beta | | | | | | |
| Lung fibroblast | 0.0 | 0.0 | 0.0 | 0.0 | 0.0 | 0.0 |
| none | | | | | | |
| Lung fibroblast | 0.0 | 0.0 | 0.0 | 0.0 | 0.0 | 0.0 |
| TNF alpha + IL-1 | | | | | | |
| beta | | | | | | |
| Lung fibroblast IL-4 | 0.0 | 0.0 | 0.0 | 0.0 | 0.0 | 0.0 |
| Lung fibroblast IL-9 | 0.0 | 0.0 | 0.0 | 0.0 | 0.0 | 0.0 |
| Lung fibroblast IL- | 0.0 | 0.0 | 0.0 | 0.0 | 0.0 | 0.0 |
| 13 | | | | | | |
| Lung fibroblast | 0.0 | 0.0 | 0.0 | 0.0 | 0.0 | 0.0 |
| IFN gamma | | | | | | |
| Dermal fibroblast | 0.0 | 0.0 | 0.0 | 0.0 | 0.0 | 0.0 |
| CCD1070 rest | | | | | | |
| Dermal fibroblast | 0.0 | 0.0 | 0.0 | 0.0 | 0.0 | 0.0 |
| CCD1070 TNF | | | | | | |
| alpha | | | | | | |
| Dermal fibroblast | 0.0 | 0.0 | 0.0 | 0.0 | 0.0 | 0.0 |
| CCD1070 IL-1 | | | | | | |
| beta | | | | | | |
| Dermal fibroblast | 0.0 | 0.0 | 0.0 | 0.0 | 0.0 | 5.3 |
| IFN gamma | | | | | | |
| Dermal fibroblast | 0.0 | 0.0 | 0.0 | 0.0 | 0.0 | 0.0 |
| IL-4 | | | | | | |
| IBD Colitis 2 | 1.7 | 0.0 | 0.0 | 0.0 | 0.0 | 0.0 |
| IBD Crohn's | 4.5 | 1.6 | 6.7 | 6.1 | 3.3 | 3.0 |
| Colon | 5.5 | 2.1 | 23.0 | 8.3 | 4.7 | 11.1 |
| Lung | 41.5 | 26.2 | 27.7 | 39.2 | 37.1 | 49.7 |
| Thymus | 4.2 | 0.0 | 4.1 | 0.0 | 1.4 | 20.6 |
| Kidney | 4.7 | 5.2 | 8.7 | 5.9 | 6.3 | 6.7 |

Panel 1 Summary: Ag03/Ag068

Two experiments with the same probe and primer set produce results that are in excellent agreement, with highest expression of the AC084364.5 gene in the spleen (CTs=21-25). Overall, this gene appears to be more highly expressed in normal tissue than in cancer cell lines. There are however detectable levels of expression in cell lines derived from melanoma, breast, renal, ovarian, lung, gastric and colon cancers. Thus, the difference in levels of expression of this gene could potentially be used to differentiate between these cancer cell line samples and other samples on this panel and between normal tissues and malignancies from those cancers.

There are also higher levels of expression in lung, and kidney tissue from fetal sources (CTs=25-28) when compared to levels of expression in the adult (CTs=38-31). Thus, expression of this gene could also be used to differentiate between adult and fetal lung and kidney tissue.

Among tissues with metabolic function, this gene is expressed in the liver, pituitary, thyroid, heart, skeletal muscle and adrenal gland. This suggests that the protein encoded by this gene may be invovled in the homeostasis of these tissues. Therefore, therapeutic modulation of the expression or function of this gene product may be effective in the treatment of metabolic disorders, including obesity and diabetes.

This gene is a homolog of Stabilin-1, and is also expressed at moderate levels in all brain regions examined. Because stabilin is involved in angiogenesis, the therapeutic modulation of this gene or its protein product may be of benefit in the treatment of stroke/cerebral ischemia/cerebral infarct.

Panel 1.2 Summary: Ag812

Two experiments with the same probe and primer set show highest expression of the AC084364.5 gene in the liver (CTs=25). Significant expression is also found in other metabolic tissues including fetal and adult heart, skeletal muscle, pancreas, thyroid, pituitary and adrenal gland. The high expression of this gene in the liver suggests that this gene may be involved in the normal homeostasis of that organ. Therapeutic modulation of the expression or function of this gene may be effective in the treatment of disease that involve the liver.

This gene also shows low to moderate expression in the brain. Please see Panel 1 for discussion of potential utility of this gene in the central nervous system.

While this gene shows a greater association for normal tissue, there are significant levels of expression in a cluster of ovarian cancer cell lines. Thus, expression of this gene could be used to differentiate between those samples and other samples on this panel, and between normal and malignant ovarian tissue. Furthermore, therapeutic modulation of the expression or function of this protein may be effective in the treatment of ovarian cancer. Please note that data from a third experiment with the probe and primer set Ag793 is not included, because the controls indicate that the experiment failed.

Panel 1.3D Summary: Ag2742, Ag2743, Ag2744, Ag2745, Ag2746

Multiple experiments with the same probe and primer set produce results that are in excellent agreement, with all experiments showing highest expression of the AC084364.5 gene in the liver (CTs=25). Significant expression is also found in the spleen (CTs=28-29). This result is in concordance with the results from Panel 1.

This gene appears to be expressed at higher levels in the fetal kidney and skeletal muscle (CTs=32-34) than in the comparable adult tissues (CTs=40). Thus, expression of this gene could be used to differentiate between kidney and skeletal muscle tissue from adult and fetal sources. Furthermore, the higher levels of expression of this gene in the fetal tissues suggest that this gene product may be involved in the development of these organs. Thus, therapeutic modulation of the expression or function of these genes may be effective in treating disease of these organs in the adult.

In this panel, this gene appears to exclusively associate with normal tissue samples, a preference that is also observed in panels 1 and 1.2. Thus, absence of expression of this gene may be useful in differentiating between the cancerous cell lines on this panel, and their corresponding normal tissues, specifically cancers of the ovary, breast and colon.

Panel 2D Summary: Ag2742/Ag2743/Ag2744/Ag2745/Ag2746

Multiple experiments with the same probe and primer set show expression of the AC084364.5 gene to be highest and almost exclusive in the liver (CTs=27-29). Furthermore, there is higher expression in liver tissue when compared to colon cancer or melanoma that have metastasized to the liver. This liver specific expression is in concordance with the results from previous panels. The low/undetectable levels of expression in cancer samples are also in agreement with the results observed in the preceding experiments. Thus, the expression profile of this gene suggests that expression of this gene could be used to differentiate between liver tissue and other samples on this panel and as a marker for liver tissue. Furthermore, therapeutic modulation of the expression or function of the protein encoded by this gene could be effective in the treatment of liver cancer or other disease that involve the liver. Additionally, slightly higher expression of this gene is seen in normal bladder, ovary and stomach compared to the adjacent tumor tissue. Hence, expression of this gene might be used as a marker to identify normal tissue from cancerous tissue in these organs. In addition, polypeptide molecules could potentially be used to therapeutically inhibit bladder, ovary and stomach cancer.

Panel 4D Summary: Ag812/Ag2742/Ag2743/Ag2744/Ag2745/Ag2746

The expression of the AC084364.5 gene appears to be highest in samples from cirrhotic liver, (CTs=32-33). Low level expression is also detected in samples derived from normal lung. The presence of this gene in liver cirrhosis (a component of which involves liver inflammation and fibrosis) suggests that therapeutic agents involving this gene may be useful in reducing or inhibiting the inflammation associated with fibrotic and other inflammatory diseases.

NOV2a and NOV2b (CG50646-04/cg142106342 and CG50646-05: Polydom Protein)

Expression of gene CG50646-04 and variant CG50646-05 was assessed using the primer-probe set Ag768, described in Table BA. Results of the RTQ-PCR runs are shown in Tables BB and BC.

TABLE BA
Probe Name Ag768

| | | | | Start |
|---|---|---|---|---|
| Primers | Sequences | | Length | Position |
| Forward | 5′-gggctataagtcagtcggaagt-3′ | (SEQ ID NO:142) | 22 | 6772 |
| Probe | TET-5′-cctgtatttgtctgccaagccaatcg-3′-TAMRA | (SEQ ID NO:143) | 26 | 6794 |
| Reverse | 5′-acagtcgagaggaacacacatc-3′ | (SEQ ID NO:144) | 22 | 6844 |

TABLE BB
Panel 1.2

| | Rel. Exp.(%) Ag768, | | Rel. Exp.(%) Ag768, |
|---|---|---|---|
| Tissue Name | Run 116422776 | Tissue Name | Run 116422776 |
| Endothelial cells | 0.0 | Renal ca. 786-0 | 0.0 |
| Heart (Fetal) | 0.0 | Renal ca. A498 | 0.0 |
| Pancreas | 0.0 | Renal ca. RXF 393 | 0.0 |
| Pancreatic ca. CAPAN 2 | 0.0 | Renal ca. ACHN | 0.0 |
| Adrenal gland | 0.1 | Renal ca. UO-31 | 0.0 |
| Thyroid | 0.0 | Renal ca. TK-10 | 0.0 |
| Salivary gland | 0.0 | Liver | 0.0 |
| Pituitary gland | 0.0 | Liver (fetal) | 0.0 |
| Brain (fetal) | 0.0 | Liver ca. (hepatoblast) | 0.0 |
| | | HepG2 | |
| Brain (whole) | 0.0 | Lung | 0.6 |
| Brain (amygdala) | 0.0 | Lung (fetal) | 0.0 |
| Brain (cerebellum) | 0.0 | Lung ca. (small cell) | 0.0 |
| | | LX-1 | |
| Brain (hippocampus) | 0.0 | (Lung ca. (small cell) | 0.0 |
| | | NCI-H69 | |
| Brain (thalamus) | 0.0 | Lung ca. (s. cell var.) | 0.0 |
| | | SHP-77 | |
| Cerebral Cortex | 0.0 | Lung ca. (large | 0.0 |
| | | cell)NCI-H460 | |
| Spinal cord | 0.0 | Lung ca. (non-sm. | 0.0 |
| | | cell) A549 | |
| glio/astro U87-MG | 0.0 | Lung ca. (non-s. cell) | 0.0 |
| | | NCI-H23 | |
| glio/astro U-118-MG | 0.0 | Lung ca. (non-s. cell) | 0.0 |
| | | HOP-62 | |
| astrocytoma SW1783 | 0.0 | Lung ca. (non-s. cl) | 0.0 |
| | | NCI-H522 | |
| neuro*; met SK-N-AS | 0.0 | Lung ca. (squam.) | 0.0 |
| | | SW 900 | |
| astrocytoma SF-539 | 0.0 | Lung ca. (squam.) | 0.0 |
| | | NCI-H596 | |
| astrocytoma SNB-75 | 0.0 | Mammary gland | 4.4 |
| glioma SNB-19 | 0.0 | Breast ca.* (pl. ef) | 0.0 |
| | | MCF-7 | |
| glioma U251 | 0.0 | Breast ca.* (pl. ef) | 0.0 |
| | | MDA-MB-231 | |
| glioma SF-295 | 0.0 | Breast ca.* (pl. ef) | 0.0 |
| | | T47D | |
| Heart | 0.1 | Breast ca. BT-549 | 0.0 |
| Skeletal muscle | 0.2 | Breast ca. MDA-N | 0.0 |
| Bone marrow | 0.0 | Ovary | 0.0 |
| Thymus | 0.0 | Ovarian ca. OVCAR-3 | 0.0 |
| Spleen | 0.0 | Ovarian ca. OVCAR-4 | 0.0 |
| Lymph node | 0.7 | Ovarian ca. OVCAR-5 | 0.0 |
| Colorectal | 0.0 | Ovarian ca. OVCAR-8 | 0.0 |
| Stomach | 0.0 | Ovarian ca. IGROV-1 | 0.0 |
| Small intestine | 0.8 | Ovarian ca. (ascites) | 0.0 |
| | | SK-OV-3 | |
| Colon ca. SW480 | 0.0 | Uterus | 0.2 |
| Colon ca.* SW620 | 0.0 | Placenta | 100.0 |
| (SW480 met) | | | |
| Colon ca. HT29 | 0.0 | Prostate | 0.0 |
| Colon ca. HCT-116 | 0.0 | Prostate ca.* | 0.0 |
| | | (bonemet) PC-3 | |
| Colon ca. CaCo-2 | 0.0 | Testis | 0.0 |
| CC Well to Mod Diff | 0.0 | Melanoma | 0.0 |
| (ODO3866) | | Hs688(A).T | |
| Colon ca. HCC-2998 | 0.0 | Melanoma* (met) | 0.0 |
| | | Hs688(B).T | |
| Gastric ca. (liver met) | 0.0 | Melanoma UACC-62 | 0.0 |
| NCI-N87 | | | |
| Bladder | 0.0 | Melanoma M14 | 0.0 |
| Trachea | 0.0 | Melanoma LOX IMVI | 0.0 |
| Kidney | 0.0 | Melanoma* (met) | 0.0 |
| | | SK-MEL-5 | |
| Kidney (fetal) | 0.0 | | |

TABLE BC
Panel 4D

| | Rel. Exp.(%) | | Rel. Exp.(%) |
|---|---|---|---|
| | Ag768, Run | | Ag768, Run |
| Tissue Name | 138175130 | Tissue Name | 138175130 |
| Secondary Th1 act | 0.0 | HUVEC IL-1beta | 0.2 |
| Secondary Th2 act | 0.1 | HUVEC IFN gamma | 0.3 |
| Secondary Tr1 act | 0.0 | HUVEC TNF alpha + IFN | 0.1 |
| | | gamma | |
| Secondary Th1 rest | 0.1 | HUVEC TNF alpha + IL4 | 0.1 |
| Secondary Th2 rest | 0.1 | HUVEC IL-11 | 0.1 |
| Secondary Tr1 rest | 0.2 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.1 |
| | | none | |
| Primary Tr1 act | 0.0 | Microsvasular Dermal EC | 0.2 |
| | | TNFalpha + IL-1beta | |
| Primary Th1 rest | 0.2 | Bronchial epithelium | 0.2 |
| | | TNFalpha + IL1beta | |
| Primary Th2 rest | 0.0 | Small airway epithelium none | 2.3 |
| Primary Tr1 rest | 0.0 | Small airway epithelium | 1.3 |
| | | TNFalpha + IL-1beta | |
| CD45RA CD4 | 6.4 | Coronery artery SMC rest | 15.1 |
| lymphocyte act | | | |
| CD45RO CD4 | 0.1 | Coronery artery SMC | 8.2 |
| lymphocyte act | | TNFalpha + IL-1beta | |
| CD8 lymphocyte act | 0.0 | Astrocytes rest | 1.8 |
| Secondary CD8 | 0.1 | Astrocytes TNFalpha + IL- | 2.4 |
| lymphocyte rest | | 1beta | |
| Secondary CD8 | 0.1 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.4 |
| CD95 CH11 | | none | |
| LAK cells rest | 0.2 | 93580_CCD1106 | 1.4 |
| | | (Keratinocytes)_TNFa and | |
| | | IFNg | |
| LAK cells IL-2 | 0.0 | Liver cirrhosis | 10.6 |
| LAK cells IL-2 + IL-12 | 0.1 | Lupus kidney | 7.0 |
| LAK cells IL-2 + IFN | 0.3 | NCI-H292 none | 24.1 |
| gamma | | | |
| LAK cells IL-2 + IL-18 | 0.2 | NCI-H292 IL-4 | 21.6 |
| LAK cells | 3.5 | NCI-H292 IL-9 | 30.4 |
| PMA/ionomycin | | | |
| NK Cells IL-2 rest | 0.0 | NCI-H292 IL-13 | 15.3 |
| Two Way MLR 3 day | 0.0 | NCI-H292 IFN gamma | 14.6 |
| Two Way MLR 5 day | 0.0 | HPAEC none | 0.4 |
| Two Way MLR 7 day | 0.0 | HPAEC TNF alpha + IL-1 beta | 0.0 |
| PBMC rest | 0.0 | Lung fibroblast none | 31.0 |
| PBMC PWM | 2.5 | Lung fibroblast TNF alpha + | 7.7 |
| | | IL-1 beta | |
| PBMC PHA-L | 0.3 | Lung fibroblast IL-4 | 55.5 |
| Ramos (B cell) none | 0.0 | Lung fibroblast IL-9 | 37.4 |
| Ramos (B cell) | 0.1 | Lung fibroblast IL-13 | 86.5 |
| ionomycin | | | |
| B lymphocytes PWM | 0.1 | Lung fibroblast IFN gamma | 100.0 |
| B lymphocytes CD40L | 0.0 | Dermal fibroblast CCD1070 | 28.9 |
| and IL-4 | | rest | |
| EOL-1 dbcAMP | 0.0 | Dermal fibroblast CCD1070 | 23.7 |
| | | TNF alpha | |
| EOL-1 dbcAMP | 0.0 | Dermal fibroblast CCD1070 | 20.2 |
| PMA/ionomycin | | IL-1 beta | |
| Dendritic cells none | 0.0 | Dermal fibroblast IFN gamma | 22.5 |
| Dendritic cells LPS | 0.2 | Dermal fibroblast IL-4 | 47.0 |
| Dendritic cells anti- | 0.1 | IBD Colitis 2 | 0.5 |
| CD40 | | | |
| Monocytes rest | 0.1 | IBD Crohn's | 2.0 |
| Monocytes LPS | 3.1 | Colon | 9.2 |
| Macrophages rest | 0.1 | Lung | 20.3 |
| Macrophages LPS | 3.3 | Thymus | 13.0 |
| HUVEC none | 0.0 | Kidney | 6.4 |
| HUVEC starved | 0.2 | | |

Panel 1.2 Summary: Ag768

Highest expression of the CG50646-04 (NOV2a) gene is seen in placenta (CT=21). This gene encodes a polydom-like protein and is also highly expressed in mammary gland, skeletal muscle. This gene may be involved in cellular adhesion (ref. 1). Thus, expression of this gene may be used to differentiate between placental tissues and other tissues on this panel. Modulation of this gene or its protein product may be useful in reproductive and skeletal muscle physiology.

This gene is more highly expressed in fetal kidney (CT=33) than in adult kidney (CT=40). Conversely, this gene is more highly expressed in adult lung and liver (CTs=28-32) than in fetal lung and liver (CTs=38-40). Thus, expression of this gene could be used to differentiate between the adult and fetal sources of these tissues.

References:

Gilges D, Vinit M A, Callebaut I, Coulombel L, Cacheux V, Romeo P H, Vigon I. Polydom: a secreted protein with pentraxin, complement control protein, epidermal growth factor and von Willebrand factor A domains. Biochem J Nov. 15, 2000;352 Pt 1:49-59

To identify extracellular proteins with epidermal growth factor (EGF) domains that are potentially involved in the control of haemopoiesis, we performed degenerate reverse-transcriptase-mediated PCR on the murine bone-marrow stromal cell line MS-5 and isolated a new partial cDNA encoding EGF-like domains related to those in the Notch proteins. Cloning and sequencing of the full-length cDNA showed that it encoded a new extracellular multidomain protein that we named polydom. This 387 kDa mosaic protein contained a signal peptide followed by a new association of eight different protein domains, including a pentraxin domain and a von Willebrand factor type A domain, ten EGF domains, and 34 complement control protein modules. The human polydom mRNA is strongly expressed in placenta, its expression in the other tissues being weak or undetectable. The particular multidomain structure of the encoded protein suggests an important biological role in cellular adhesion and/or in the immune system.

PMID: 11062057

Panel 4D Summary: Ag768

Highest expression of the CG50646-04 gene is seen in lung fibroblasts stimulated with IFN-gamma (CT=27.4). Significant expression is seen in many samples derived from the lung including lung fibroblasts stimulated with different cytokines, the pulmonary mucoepidermoid cell line H292 stimulated with the same cytokines, and normal lung tissue. The expression of this gene in lung cells and lung tissue suggests that this gene may be involved in normal homeostasis of the lung, as well as pathological and inflammatory lung disorders, including chronic obstructive pulmonary disease, asthma, allergy and emphysema.

Significant levels of expression of this gene in dermal fibroblasts suggests that this gene may be involved in skin disorders, including psoriasis.

Moderate to low expression of this gene is also seen in many other cells with important immune function, including stimulated macrophages and monocytes, coronary artery smooth muscle cells, stimulated peripheral blood mononuclear cells, lymphocyte activated killer cells (LAK), astrocytes, activated CD45RA cells, and normal colon, thymus and kidney. This widespread expression suggests that this protein encoded by this gene may be involved in other inflammatory and autoimmune conditions, including inflammatory bowel disease, rheumatoid arthritis and osteoarthritis.

NOV3a and NOV3b (CG50273-01 and CG50273-02/152792120: Novel Transmembrane Protein)

Expression of gene CG50273-01 and variant CG50273-02 was assessed using the primer-probe set Ag2556, described in Table CA. Results of the RTQ-PCR runs are shown in Tables CB, CC, CD, CE, CF and CG.

TABLE CA
Probe Name Ag2556

| | | | | Start |
|---|---|---|---|---|
| Primers | Sequences | | Length | Position |
| Forward | 5′-gaggacagctttgatttcattg-3′ | (SEQ ID NO:145) | 22 | 526 |
| Probe | TET-5′-tggatttgatccatttcctctctacca-3′-TAMRA | (SEQ ID NO:146) | 27 | 549 |
| Reverse | 5′-aagagactggatggcttttcat-3′ | (SEQ ID NO:147) | 22 | 581 |

TABLE CB
CNS_neurodegeneration_v1.0

| | Rel. Exp.(%) Ag2556, | | Rel. Exp.(%) Ag2556, |
|---|---|---|---|
| Tissue Name | Run 206974724 | Tissue Name | Run 206974724 |
| AD 1 Hippo | 22.4 | Control (Path) 3 | 6.9 |
| | | Temporal Ctx | |
| AD 2 Hippo | 100.0 | Control (Path) 4 | 21.8 |
| | | Temporal Ctx | |
| AD 3 Hippo | 3.7 | AD 1 Occipital Ctx | 6.1 |
| AD 4 Hippo | 33.0 | AD 2 Occipital Ctx | 0.0 |
| | | (Missing) | |
| AD 5 Hippo | 21.9 | AD 3 Occipital Ctx | 2.1 |
| AD 6 Hippo | 71.2 | AD 4 Occipital Ctx | 21.9 |
| Control 2 Hippo | 55.5 | AD 5 Occipital Ctx | 18.2 |
| Control 4 Hippo | 55.5 | AD 5 Occipital Ctx | 3.0 |
| Control (Path) 3 | 14.4 | Control 1 Occipital | 2.3 |
| Hippo | | Ctx | |
| AD 1 Temporal Ctx | 14.1 | Control 2 Occipital | 19.5 |
| | | Ctx | |
| AD 2 Temporal Ctx | 57.4 | Control 3 Occipital | 9.3 |
| | | Ctx | |
| AD 3 Temporal Ctx | 5.3 | Control 4 Occipital | 13.6 |
| | | Ctx | |
| AD 4 Temporal Ctx | 39.5 | Control (Path) 1 | 41.5 |
| | | Occipital Ctx | |
| AD 5 Inf Temporal | 42.0 | Control (Path) 2 | 6.1 |
| Ctx | | Occipital Ctx | |
| AD 5 Sup Temporal | 66.0 | Control (Path) 3 | 1.8 |
| Ctx | | Occipital Ctx | |
| AD 6 Inf Temporal | 26.1 | Control (Path) 4 | 9.0 |
| Ctx | | Occipital Ctx | |
| AD 6 Sup Temporal | 14.1 | Control 1 Parietal | 12.0 |
| Ctx | | Ctx | |
| Control 1 Temporal | 18.8 | Control 2 Parietal | 41.5 |
| Ctx | | Ctx | |
| Control 2 Temporal | 29.3 | Control 3 Parietal | 12.6 |
| Ctx | | Ctx | |
| Control 3 Temporal | 14.0 | Control (Path) 1 | 27.9 |
| Ctx | | Parietal Ctx | |
| Control 3 Temporal | 26.1 | Control (Path) 2 | 16.6 |
| Ctx | | Parietal Ctx | |
| Control (Path) 1 | 43.8 | Control (Path) 3 | 2.9 |
| Temporal Ctx | | Parietal Ctx | |
| Control (Path) 2 | 42.0 | Control (Path) 4 | 17.6 |
| Temporal Ctx | | Parietal Ctx | |

TABLE CC
Panel 1.3D

| | Rel. Exp. (%) Ag2556, | | Rel. Exp. (%) Ag2556, |
|---|---|---|---|
| Tissue Name | Run 162292610 | Tissue Name | Run 162292610 |
| Liver adenocarcinoma | 0.0 | Kidney (fetal) | 5.6 |
| Pancreas | 0.0 | Renal ca. 786-0 | 6.3 |
| Pancreatic ca. CAPAN 2 | 0.0 | Renal ca. A498 | 0.5 |
| Adrenal gland | 0.7 | Renal ca. RXF 393 | 2.0 |
| Thyroid | 0.4 | Renal ca. ACHN | 3.3 |
| Salivary gland | 0.6 | Renal ca. UO-31 | 0.9 |
| Pituitary gland | 2.8 | Renal ca. TK-10 | 24.3 |
| Brain (fetal) | 3.7 | Liver | 0.0 |
| Brain (whole) | 22.7 | Liver (fetal) | 0.0 |
| Brain (amygdala) | 59.9 | Liver ca. | 0.0 |
| | | (hepatoblast) HepG2 | |
| Brain (cerebellum) | 39.0 | Lung | 0.0 |
| Brain (hippocampus) | 83.5 | Lung (fetal) | 0.0 |
| Brain (substantia nigra) | 30.8 | Lung ca. (small cell) | 0.0 |
| | | LX-1 | |
| Brain (thalamus) | 40.3 | Lung ca. (small cell) | 0.0 |
| | | NCI-H69 | |
| Cerebral Cortex | 55.9 | Lung ca. (s.cell var.) | 0.0 |
| | | SHP-77 | |
| Spinal cord | 72.2 | Lung ca. (large | 0.0 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) | 0.5 |
| | | SW 900 | |
| astrocytoma SNB-75 | 0.0 | Lung ca. (squam.) | 0.0 |
| | | NCI-H596 | |
| glioma SNB-19 | 0.4 | Mammary gland | 0.0 |
| glioma U251 | 0.0 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (Fetal) | 4.5 | Breast ca.* (pl. ef) | 0.0 |
| | | T47D | |
| Heart | 0.0 | Breast ca. BT-549 | 0.0 |
| Skeletal muscle (Fetal) | 100.0 | Breast ca. MDA-N | 0.0 |
| Skeletal muscle | 0.0 | Ovary | 0.4 |
| Bone marrow | 0.0 | Ovarian ca. OVCAR-3 | 0.0 |
| Thymus | 5.2 | Ovarian ca. OVCAR-4 | 0.7 |
| Spleen | 0.0 | Ovarian ca. OVCAR-5 | 2.0 |
| Lymph node | 0.0 | Ovarian ca. OVCAR-8 | 0.9 |
| Colorectal | 2.7 | Ovarian ca. IGROV-1 | 0.0 |
| Stomach | 0.0 | Ovarian ca. (ascites) | 20.7 |
| | | SK-OV-3 | |
| Small intenstine | 0.8 | Uterus | 0.5 |
| Colon ca. SW480 | 0.0 | Placenta | 11.0 |
| Colon ca.* SW620 | 0.0 | Prostate | 0.0 |
| (SW480 met) | | | |
| Colon ca. HT29 | 0.0 | Prostate ca.* | 0.0 |
| | | (bonemet) PC-3 | |
| Colon ca. HCT-116 | 0.0 | Testis | 5.6 |
| Colon ca. CaCo-2 | 7.3 | Melanoma | 0.0 |
| | | Hs688(A).T | |
| CC Well to Mod Diff | 0.5 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX | 0.0 |
| | | IMVI | |
| Trachea | 1.1 | Melanoma* (met) | 0.0 |
| | | SK-MEL-5 | |
| Kidney | 20.4 | Adipose | 0.8 |

TABLE CD
Panel 2D

| | Rel. Exp. (%) | | Rel. Exp. (%) |
|---|---|---|---|
| | Ag2556, | | Ag2556, |
| Tissue Name | Run 161921170 | Tissue Name | Run 161921170 |
| Normal Colon | 1.0 | Kidney Margin | 30.6 |
| | | 8120608 | |
| CC Well to Mod Diff | 0.0 | Kidney Cancer | 0.3 |
| (ODO3866) | | 8120613 | |
| CC Margin (ODO3866) | 0.1 | Kidney Margin | 4.1 |
| | | 8120614 | |
| CC Gr.2 rectosigmoid | 0.0 | Kidney Cancer | 3.7 |
| (ODO3868) | | 9010320 | |
| CC Margin (ODO3868) | 0.1 | Kidney Margin | 100.0 |
| | | 9010321 | |
| CC Mod Diff (ODO3920) | 0.3 | Normal Uterus | 0.0 |
| CC Margin (ODO3920) | 0.0 | Uterine Cancer | 0.0 |
| | | 064011 | |
| CC Gr.2 ascend colon | 0.3 | Normal Thyroid | 0.8 |
| (ODO3921) | | | |
| CC Margin (ODO3921) | 0.0 | Thyroid Cancer | 0.4 |
| CC from Partial | 0.3 | Thyroid Cancer | 1.5 |
| Hepatectomy (ODO4309) | | A302152 | |
| Mets | | | |
| Liver Margin (ODO4309) | 0.0 | Thyroid Margin | 1.4 |
| | | A302153 | |
| Colon mets to lung | 0.0 | Normal Breast | 0.0 |
| (OD04451-01) | | | |
| Lung Margin (OD04451- | 0.1 | Breast Cancer | 0.0 |
| 02) | | | |
| Normal Prostate 6546-1 | 0.2 | Breast Cancer | 0.0 |
| | | (OD04590-01) | |
| Prostate Cancer | 0.1 | Breast Cancer Mets | 0.0 |
| (OD04410) | | (OD04590-03) | |
| Prostate Margin | 0.3 | Breast Cancer | 0.0 |
| (OD04410) | | Metastasis | |
| Prostate Cancer | 0.3 | Breast Cancer | 0.2 |
| (OD04720-01) | | | |
| Prostate Margin | 0.0 | Breast Cancer | 0.1 |
| (OD04720-02) | | | |
| Normal Lung | 0.1 | Breast Cancer | 0.0 |
| | | 9100266 | |
| Lung Met to Muscle | 0.0 | Breast Margin | 0.1 |
| (ODO4286) | | 9100265 | |
| Muscle Margin | 0.0 | Breast Cancer | 0.0 |
| (ODO4286) | | A209073 | |
| Lung Malignant Cancer | 1.0 | Breast Margin | 0.0 |
| (OD03126) | | A2090734 | |
| Lung Margin (OD03126) | 0.1 | Normal Liver | 0.0 |
| Lung Cancer (OD04404) | 0.0 | Liver Cancer | 0.0 |
| Lung Margin (OD04404) | 0.0 | Liver Cancer 1025 | 0.0 |
| Lung Cancer (OD04565) | 0.2 | Liver Cancer 1026 | 0.0 |
| Lung Margin (OD04565) | 0.0 | Liver Cancer 6004-T | 0.0 |
| Lung Cancer (OD04237- | 0.1 | Liver Tissue 6004-N | 0.0 |
| 01) | | | |
| Lung Margin (OD04237- | 0.1 | Liver Cancer 6005-T | 0.0 |
| 02) | | | |
| Ocular Mel Met to Liver | 1.0 | Liver Tissue 6005-N | 0.0 |
| (ODO4310) | | | |
| Liver Margin (ODO4310) | 0.0 | Normal Bladder | 0.0 |
| Melanoma Metastasis | 0.3 | Bladder Cancer | 0.0 |
| Lung Margin (OD04321) | 0.0 | Bladder Cancer | 0.0 |
| Normal Kidney | 2.4 | Bladder Cancer | 0.9 |
| | | (OD04718-01) | |
| Kidney Ca, Nuclear grade | 15.1 | Bladder Normal | 0.0 |
| 2 (OD04338) | | Adjacent (OD04718- | |
| | | 03) | |
| Kidney Margin | 16.0 | Normal Ovary | 0.0 |
| (OD04338) | | | |
| Kidney Ca Nuclear grade | 3.7 | Ovarian Cancer | 0.1 |
| 1/2 (OD04339) | | | |
| Kidney Margin | 15.9 | Ovarian Cancer | 0.2 |
| (OD04339) | | (OD04768-07) | |
| Kidney Ca, Clear cell type | 1.6 | Ovary Margin | 0.1 |
| (OD04340) | | (OD04768-08) | |
| Kidney Margin | 3.3 | Normal Stomach | 0.3 |
| (OD04340) | | | |
| Kidney Ca, Nuclear grade | 0.1 | Gastric Cancer | 0.2 |
| 3 (OD04348) | | 9060358 | |
| Kidney Margin | 20.3 | Stomach Margin | 0.0 |
| (OD04348) | | 9060359 | |
| Kidney Cancer | 8.8 | Gastric Cancer | 0.2 |
| (OD04622-01) | | 9060395 | |
| Kidney Margin | 6.0 | Stomach Margin | 0.1 |
| (OD04622-03) | | 9060394 | |
| Kidney Cancer | 1.9 | Gastric Cancer | 0.3 |
| (OD04450-01) | | 9060397 | |
| Kidney Margin | 1.9 | Stomach Margin | 0.0 |
| (OD04450-03) | | 9060396 | |
| Kidney Cancer 8120607 | 0.2 | Gastric Cancer 064005 | 0.3 |

TABLE CE
Panel 3D

| | Rel. Exp. (%) | | Rel. Exp. (%) |
|---|---|---|---|
| | Ag2556, Run | | Ag2556, Run |
| Tissue Name | 164827571 | Tissue Name | 164827571 |
| Daoy-Medulloblastoma | 1.8 | Ca Ski-Cervical epidermoid | 0.0 |
| | | carcinoma (metastasis) | |
| TE671-Medulloblastoma | 100.0 | ES-2-Ovarian clear cell | 0.0 |
| | | carcinoma | |
| D283 Med- | 0.0 | Ramos-Stimulated with | 0.0 |
| Medulloblastoma | | PMA/ionomycin 6h | |
| PFSK-1-Primitive | 0.0 | Ramos-Stimulated with | 0.0 |
| Neuroectodermal | | PMA/ionomycin 14h | |
| XF-498-CNS | 0.5 | MEG-01-Chronic | 0.0 |
| | | myelogenous leukemia | |
| | | (megokaryoblast) | |
| SNB-78-Glioma | 0.4 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH- | 0.0 | CA46-Burkitt's lymphoma | 0.0 |
| Neuroblastoma | | | |
| (metastasis) | | | |
| SF-295-Glioblastoma | 0.0 | RL-non-Hodgkin's B-cell | 0.0 |
| | | lymphoma | |
| Cerebellum | 30.8 | JM1-pre-B-cell lymphoma | 0.3 |
| Cerebellum | 49.3 | Jurkat-T cell leukemia | 0.0 |
| NCI-H292- | 0.0 | TF-1-Erythroleukemia | 0.0 |
| Mucoepidermoid lung | | | |
| carcinoma | | | |
| DMS-114-Small cell | 1.1 | HUT 78-T-cell lymphoma | 0.3 |
| lung cancer | | | |
| DMS-79-Small cell lung | 32.5 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 0.0 | 769-P-Clear cell renal | 2.4 |
| lung cancer | | carcinoma | |
| NCI-N417-Small cell | 0.0 | Caki-2-Clear cell renal | 0.6 |
| lung cancer | | carcinoma | |
| NCI-H82-Small cell lung | 0.0 | SW 839-Clear cell renal | 0.0 |
| cancer | | carcinoma | |
| NCI-H157-Squamous | 0.0 | G401-Wilms' tumor | 9.5 |
| cell lung cancer | | | |
| (metastasis) | | | |
| NCI-H1155-Large cell | 0.0 | Hs766T-Pancreatic carcinoma | 0.0 |
| lung cancer | | (LN metastasis) | |
| NCI-H1299-Large cell | 0.0 | CAPAN-1-Pancreatic | 0.0 |
| lung cancer | | adenocarcinoma (liver | |
| | | metastasis) | |
| NCI-H727-Lung | 4.1 | SU86.86-Pancreatic | 0.6 |
| carcinoid | | carcinoma (liver metastasis) | |
| NCI-UMC-11-Lung | 0.0 | BxPC-3-Pancreatic | 0.0 |
| carcinoid | | adenocarcinoma | |
| LX-1-Small cell lung | 0.4 | HPAC-Pancreatic | 1.0 |
| cancer | | adenocarcinoma | |
| Colo-205-Colon cancer | 0.0 | MIA PaCa-2-Pancreatic | 1.9 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic | 7.7 |
| | | epithelioid ductal carcinoma | |
| NCI-H716-Colon cancer | 0.0 | T24-Bladder carcinma | 0.3 |
| | | (transitional cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 3.7 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 0.0 | MG-63-Osteosarcoma | 0.4 |
| carcinoma | | | |
| KATO III-Gastric | 0.0 | SK-LMS-1-Leiomyosarcoma | 0.0 |
| carcinoma | | (vulva) | |
| NCI-SNU-16-Gastric | 0.0 | SJRH30-Rhabdomyosarcoma | 3.0 |
| carcinoma | | (met to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 0.0 |
| carcinoma | | adenocarcinoma | |
| NCI-N87-Gastric | 0.0 | SCC-4-Squamous cell | 0.0 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.0 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE CF
Panel 4D

| | Rel. Exp. (%) | | Rel. Exp. (%) |
|---|---|---|---|
| | Ag2556, Run | | Ag2556, Run |
| Tissue Name | 164035630 | Tissue Name | 164035630 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.7 |
| | | TNFalpha + IL-1beta | |
| Primary Th2 rest | 0.0 | Small airway epithelium | 1.4 |
| | | none | |
| Primary Tr1 rest | 0.0 | Small airway epithelium | 5.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 0.0 | Lupus kidney | 3.2 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 5.2 |
| Macrophages rest | 0.0 | Lung | 1.3 |
| Macrophages LPS | 0.0 | Thymus | 100.0 |
| HUVEC none | 0.0 | Kidney | 12.9 |
| HUVEC starved | 0.0 | | |

TABLE CG
Panel CNS_1

| | Rel. Exp. (%) Ag2556, | | Rel. Exp. (%) Ag2556, |
|---|---|---|---|
| Tissue Name | Run 171656437 | Tissue Name | Run 171656437 |
| BA4 Control | 15.2 | BA17 PSP | 8.5 |
| BA4 Control2 | 21.9 | BA17 PSP2 | 0.0 |
| BA4 | 4.4 | Sub Nigra Control | 66.9 |
| Alzheimer's2 | | | |
| BA4 Parkinson's | 34.9 | Sub Nigra Control2 | 41.2 |
| BA4 | 12.9 | Sub Nigra | 33.4 |
| Parkinson's2 | | Alzheimer's2 | |
| BA4 | 28.9 | Sub Nigra | 71.7 |
| Huntington's | | Parkinson's2 | |
| BA4 | 16.5 | Sub Nigra | 92.7 |
| Huntington's2 | | Huntington's | |
| BA4 PSP | 7.0 | Sub Nigra | 12.9 |
| | | Huntington's2 | |
| BA4 PSP2 | 18.3 | Sub Nigra PSP2 | 20.6 |
| BA4 Depression | 12.2 | Sub Nigra | 10.2 |
| | | Depression | |
| BA4 | 3.5 | Sub Nigra | 5.9 |
| Depression2 | | Depression2 | |
| BA7 Control | 5.8 | Glob Palladus | 40.9 |
| | | Control | |
| BA7 Control2 | 17.0 | Glob Palladus | 34.6 |
| | | Control2 | |
| BA7 | 10.9 | Glob Palladus | 42.9 |
| Alzheimer's2 | | Alzheimer's | |
| BA7 Parkinson's | 30.4 | Glob Palladus | 14.8 |
| | | Alzheimer's2 | |
| BA7 | 8.2 | Glob Palladus | 100.0 |
| Parkinson's2 | | Parkinson's | |
| BA7 | 19.8 | Glob Palladus | 20.6 |
| Huntington's | | Parkinson's2 | |
| BA7 | 32.8 | Glob Palladus PSP | 16.5 |
| Huntington's2 | | | |
| BA7 PSP | 16.3 | Glob Palladus PSP2 | 23.0 |
| BA7 PSP2 | 12.1 | Glob Palladus | 13.4 |
| | | Depression | |
| BA7 Depression | 4.9 | Temp Pole Control | 10.8 |
| BA9 Control | 17.0 | Temp Pole Control2 | 58.6 |
| BA9 Control2 | 54.0 | Temp Pole | 27.5 |
| | | Alzheimer's | |
| BA9 Alzheimer's | 10.3 | Temp Pole | 13.4 |
| | | Alzheimer's2 | |
| BA9 | 26.8 | Temp Pole | 34.4 |
| Alzheimer's2 | | Parkinson's | |
| BA9 Parkinson's | 33.0 | Temp Pole | 35.4 |
| | | Parkinson's2 | |
| BA9 | 28.7 | Temp Pole | 57.8 |
| Parkinson's2 | | Huntington's | |
| BA9 | 58.2 | Temp Pole PSP | 8.8 |
| Huntington's | | | |
| BA9 | 27.9 | Temp Pole PSP2 | 8.9 |
| Huntington's2 | | | |
| BA9 PSP | 17.1 | Temp Pole | 8.4 |
| | | Depression2 | |
| BA9 PSP2 | 3.6 | Cing Gyr Control | 51.1 |
| BA9 Depression | 6.8 | Cing Gyr Control2 | 33.7 |
| BA9 | 5.8 | Cing Gyr Alzheimer's | 49.0 |
| Depression2 | | | |
| BA17 Control | 9.4 | Cing Gyr | 11.1 |
| | | Alzheimer's2 | |
| BA17 Control2 | 14.2 | Cing Gyr Parkinson's | 61.1 |
| BA17 | 4.4 | Cing Gyr | 42.0 |
| Alzheimer's2 | | Parkinson's2 | |
| BA17 | 22.8 | Cing Gyr | 77.4 |
| Parkinson's | | Huntington's | |
| BA17 | 4.9 | Cing Gyr | 38.2 |
| Parkinson's2 | | Huntington's2 | |
| BA17 | 14.6 | Cing Gyr PSP | 20.0 |
| Huntington's | | | |
| BA17 | 8.8 | Cing Gyr PSP2 | 25.0 |
| Huntington's2 | | | |
| BA17 | 7.2 | Cing Gyr Depression | 23.7 |
| Depression | | | |
| BA17 | 4.6 | Cing Gyr | 11.3 |
| Depression2 | | Depression2 | |

CNS_Neurodegeneration_{—v}1.0 Summary: Ag2556

No difference was detected in the expression of the CG50273-01 gene in the postmortem brains of Alzheimer's patients when compared normal controls; however this panel demonstrates the expression of this gene in the CNS of an independent group of patients. See panel 1.3d for discussion of utility of this gene in the central nervous system.

Panel 1.3D Summary: Ag2556

Highest expression of the CG50273-01 gene is seen in fetal skeletal muscle (CT=31.4). Furthermore, this gene appears to be expressed at much higher levels in fetal skeletal muscle than in the adult (CT=40). This expression pattern suggests that the protein encoded by this gene may be involved in the development of this tissue. Furthermore, therapeutic application of the protein product may help in restoring muscle mass or function to weak or dystrophic muscle in the adult.

This gene also shows highly brain preferential expression. The CG50273-01 gene encodes a novel transmembrane protein. The combination of brain and skeletal muscle-preferential expression is consistent with a protein present in cholinergic synapses. Indeed, this gene shows homology to the cholinergic receptor CHRNA4 subunit. Therefore, this gene may be useful in the treatment of multiple sclerosis, ALS, or any disease in which the cholinergic system has been implicated (Alzheimer's disease).

Low but significant levels of expression are seen in renal and ovarian cancer cell lines. Thus, expression of this gene could potentially be used to differentiate between these samples and other samples on this panel or as a marker to detect the presence of these cancers.

Panel 2D Summary: Ag2556

Highest expression of the CG50273-01 gene is seen in normal kidney (CT=28.4). Furthermore, this gene appears to be more highly expressed in normal kidney tissue adjacent to a kidney cancer, than in the cancer itself. Thus, expression of this gene could potentially be used as a marker to differentiate between normal and cancerous kidney tissue. Moreover, therapeutic modulation of the expression or function of this gene could potentially be useful in the treatment of kidney cancer.

Panel 3D Summary: Ag2556

Expression of the CG50273-01 gene is restricted to a few cell lines on this panel including two lung cancer cell lines, medulloblastoma, two renal and three pancreatic cancer cell lines as well as the cerebellum samples which reflect the brain expression seen in Panel 1.3D.

Panel 4D Summary: Ag2556

The CG50273-01 gene appears to be preferentially expressed in normal thymus (CT=32.1). Since the thymus is involved in the development of the immune system, the transcript encoded by this gene could be used for detection of thymus/thymic cells as well as play a role in the homeostasis of the tissue and/or thymic/immune cells.

Panel CNS_{—}1 Summary: Ag2556

The widespread expression of the CG50273-01 gene in this panel confirms that it is expressed in the brain. Please see Panel 1.3D for discussion of potential utility of this gene in the central nervous system.

NOV4 (CG50289-01: Serine Protease)

Expression of gene CG50289-01 was assessed using the primer-probe sets Ag3600, Ag792 and Ag2555, described in Tables DA, DB and DC. Results of the RTQ-PCR runs are shown in Tables DD, DE, and DF.

TABLE DA
Probe Name Ag3600

| | | | | Start |
|---|---|---|---|---|
| Primers | Sequences | | Length | Position |
| Forward | 5′-agccaagcagcagtgactac-3′ | (SEQ ID NO:148) | 20 | 507 |
| Probe | TET-5′-accatccacgaggacatgctgtg-3′-TAMRA | (SEQ ID NO:149) | 23 | 527 |
| Reverse | 5′-aaatggcctttcctgttatgag-3′ | (SEQ ID NO:150) | 22 | 560 |

TABLE DB
Probe Name Ag792

| | | | | Start |
|---|---|---|---|---|
| Primers | Sequences | | Length | Position |
| Forward | 5′-agccaagcagcagtgactac-3′ | (SEQ ID NO:151) | 20 | 507 |
| Probe | TET-5′-accatccacgaggacatgctgtg-3′-TAMRA | (SEQ ID NO:152) | 23 | 527 |
| Reverse | 5′-aaatggcctttcctgttatgag-3′ | (SEQ ID NO:153) | 22 | 560 |

TABLE DC
Probe Name Ag2555

| | | | | Start |
|---|---|---|---|---|
| Primers | Sequences | | Length | Position |
| Forward | 5′-ctcataacaggaaaggccattt-3′ | (SEQ ID NO:154) | 22 | 560 |
| Probe | TET-5′-agactccaggggtcccctcgtct-3′-TAMRA | (SEQ ID NO:155) | 23 | 589 |
| Reverse | 5′-aggaaccaggtgccatttaat-3′ | (SEQ ID NO:156) | 21 | 616 |

TABLE DD
General_screening_panel_v1.4

| | Rel. Exp. (%) Ag3600, | | Rel. Exp. (%) Ag3600, |
|---|---|---|---|
| Tissue Name | Run 217676536 | Tissue Name | Run 217676536 |
| Adipose | 0.0 | Renal ca. TK-10 | 0.0 |
| Melanoma* | 0.0 | Bladder | 1.2 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 100.0 | Colon ca. HT29 | 0.0 |
| Prostate ca.* | 1.3 | Colon ca. HCT-116 | 0.0 |
| (bonemet) PC-3 | | | |
| Prostate Pool | 0.0 | Colon ca. CaCo-2 | 3.7 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 1.3 | Colon Pool | 2.6 |
| Ovarian ca. OVCAR-5 | 0.0 | Small Intestine Pool | 0.6 |
| Ovarian ca. IGROV-1 | 0.0 | Stomach Pool | 4.0 |
| Ovarian ca. OVCAR-8 | 0.0 | Bone Marrow Pool | 3.0 |
| Ovary | 0.0 | Fetal Heart | 0.0 |
| Breast ca. MCF-7 | 0.0 | Heart Pool | 0.4 |
| Breast ca. MDA- | 0.0 | Lymph Node Pool | 2.5 |
| MB-231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 1.7 | Spleen Pool | 0.0 |
| Breast Pool | 1.3 | Thymus Pool | 2.6 |
| Trachea | 0.0 | CNS cancer (glio/astro) | 0.0 |
| | | U87-MG | |
| Lung | 0.0 | CNS cancer (glio/astro) | 0.0 |
| | | U-118-MG | |
| Fetal Lung | 0.0 | CNS cancer (neuro;met) | 0.0 |
| | | SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) | 0.0 |
| | | SNB-75 | |
| Lung ca. NCI-H146 | 0.0 | CNS cancer (glio) SNB- | 0.0 |
| | | 19 | |
| Lung ca. SHP-77 | 2.4 | CNS cancer (glio) SF- | 0.0 |
| | | 295 | |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 1.3 | Adrenal Gland | 0.0 |
| Fetal Kidney | 1.3 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 1.2 | Pancreatic ca. CAPAN2 | 0.0 |
| Renal ca. UO-31 | 0.0 | Pancreas Pool | 1.8 |

TABLE DE
Panel 1.2

| | Rel. Exp. (%) Ag792, | | Rel. Exp. (%) Ag792, |
|---|---|---|---|
| Tissue Name | Run 118335897 | Tissue Name | Run 118335897 |
| Endothelial cells | 0.0 | Renal ca. 786-0 | 0.0 |
| Heart (Fetal) | 0.0 | Renal ca. A498 | 0.2 |
| Pancreas | 2.4 | Renal ca. RXF 393 | 0.0 |
| Pancreatic ca. CAPAN2 | 0.0 | Renal ca. ACHN | 0.0 |
| Adrenal gland | 0.0 | Renal ca. UO-31 | 0.1 |
| Thyroid | 0.0 | Renal ca. TK-10 | 0.0 |
| Salivary gland | 0.1 | Liver | 0.2 |
| Pituitary gland | 0.0 | Liver (fetal) | 0.1 |
| Brain (fetal) | 0.0 | Liver ca. (hepatoblast) | 0.0 |
| | | HepG2 | |
| Brain (whole) | 0.0 | Lung | 0.1 |
| Brain (amygdala) | 0.0 | Lung (fetal) | 0.0 |
| Brain (cerebellum) | 0.0 | Lung ca. (small cell) | 0.0 |
| | | LX-1 | |
| Brain (hippocampus) | 0.0 | Lung ca. (small cell) | 2.3 |
| | | NCI-H69 | |
| Brain (thalamus) | 0.0 | Lung ca. (s.cell var.) | 0.2 |
| | | SHP-77 | |
| Cerebral Cortex | 0.0 | Lung ca. (large | 0.1 |
| | | cell)NCI-H460 | |
| Spinal cord | 0.0 | Lung ca. (non-sm. | 1.0 |
| | | cell) A549 | |
| glio/astro U87-MG | 0.0 | Lung ca. (non-s.cell) | 0.0 |
| | | NCI-H23 | |
| glio/astro U-118-MG | 0.0 | Lung ca. (non-s.cell) | 0.2 |
| | | HOP-62 | |
| astrocytoma SW1783 | 0.0 | Lung ca. (non-s.cl) | 0.0 |
| | | NCI-H522 | |
| neuro*; met SK-N-AS | 0.0 | Lung ca. (squam.) SW | 0.1 |
| | | 900 | |
| astrocytoma SF-539 | 0.0 | Lung ca. (squam.) | 1.2 |
| | | NCI-H596 | |
| astrocytoma SNB-75 | 0.0 | Mammary gland | 0.0 |
| glioma SNB-19 | 0.3 | Breast ca.* (pl.ef) | 0.0 |
| | | MCF-7 | |
| glioma U251 | 0.0 | Breast ca.* (pl.ef) | 0.0 |
| | | MDA-MB-231 | |
| glioma SF-295 | 0.0 | Breast ca.* (pl. ef) | 0.8 |
| | | T47D | |
| Heart | 0.0 | Breast ca. BT-549 | 0.2 |
| Skeletal muscle | 0.0 | Breast ca. MDA-N | 0.2 |
| Bone marrow | 0.0 | Ovary | 0.0 |
| Thymus | 0.0 | Ovarian ca. OVCAR-3 | 0.0 |
| Spleen | 0.5 | Ovarian ca. OVCAR-4 | 0.0 |
| Lymph node | 0.0 | Ovarian ca. OVCAR-5 | 2.7 |
| Colorectal | 0.0 | Ovarian ca. OVCAR-8 | 0.0 |
| Stomach | 0.1 | Ovarian ca. IGROV-1 | 0.3 |
| Small intestine | 0.0 | Ovarian ca. (ascites) | 0.0 |
| | | SK-OV-3 | |
| Colon ca. SW480 | 0.0 | Uterus | 0.0 |
| Colon ca.* SW620 | 0.0 | Placenta | 0.0 |
| (SW480 met) | | | |
| Colon ca. HT29 | 0.1 | Prostate | 0.1 |
| Colon ca. HCT-116 | 0.0 | Prostate ca.* (bone | 0.0 |
| | | met) PC-3 | |
| Colon ca. CaCo-2 | 0.0 | Testis | 100.0 |
| CC Well to Mod Diff | 0.7 | Melanoma | 0.0 |
| (ODO3866) | | Hs688(A).T | |
| Colon ca. HCC-2998 | 0.0 | Melanoma* (met) | 0.2 |
| | | Hs688(B).T | |
| Gastric ca. (liver met) | 0.2 | Melanoma UACC-62 | 0.0 |
| NCI-N87 | | | |
| Bladder | 2.0 | Melanoma M14 | 1.6 |
| Trachea | 0.0 | Melanoma LOX IMVI | 0.0 |
| Kidney | 0.0 | Melanoma* (met) SK- | 0.0 |
| | | MEL-5 | |
| Kidney (fetal) | 0.0 | | |

TABLE DF
Panel 1.3D

| | Rel. Exp. (%) | Rel. Exp. (%) | | Rel. Exp. (%) | Rel. Exp. (%) |
|---|---|---|---|---|---|
| | Ag2555, Run | Ag792, Run | | Ag2555, Run | Ag792, Run |
| Tissue Name | 162292287 | 165693644 | Tissue Name | 162292287 | 165693644 |

| | | | | | |
|---|---|---|---|---|---|
| Liver | 0.0 | 0.0 | Kidney (fetal) | 0.0 | 0.0 |
| adenocarcinoma | | | | | |
| Pancreas | 0.0 | 0.0 | Renal ca. 786-0 | 0.0 | 0.0 |
| Pancreatic ca. | 0.0 | 0.0 | Renal ca. A498 | 0.0 | 0.0 |
| CAPAN 2 | | | | | |
| Adrenal gland | 0.0 | 0.0 | Renal ca. RXF | 0.0 | 0.0 |
| | | | 393 | | |
| Thyroid | 0.0 | 0.0 | Renal ca. | 0.0 | 0.0 |
| | | | ACHN | | |
| Salivary gland | 0.0 | 0.0 | Renal ca. UO- | 0.0 | 0.0 |
| | | | 31 | | |
| Pituitary gland | 0.0 | 0.0 | Renal ca. TK- | 0.0 | 0.0 |
| | | | 10 | | |
| Brain (fetal) | 0.0 | 0.0 | Liver | 0.0 | 0.0 |
| Brain (whole) | 0.0 | 0.0 | Liver (fetal) | 0.0 | 0.0 |
| Brain (amygdala) | 0.0 | 0.0 | Liver ca. | 0.0 | 0.0 |
| | | | (hepatoblast) | | |
| | | | HepG2 | | |
| Brain | 0.0 | 0.0 | Lung | 0.0 | 0.0 |
| (cerebellum) | | | | | |
| Brain | 0.0 | 0.0 | Lung (fetal) | 0.0 | 0.0 |
| (hippocampus) | | | | | |
| Brain (substantia | 0.0 | 0.0 | Lung ca. (small | 0.0 | 0.0 |
| nigra) | | | cell) LX-1 | | |
| Brain (thalamus) | 0.0 | 0.0 | Lung ca. (small | 0.0 | 0.0 |
| | | | cell) NCI-H69 | | |
| Cerebral Cortex | 0.0 | 0.0 | Lung ca. (s.cell | 0.0 | 0.0 |
| | | | var.) SHP-77 | | |
| Spinal cord | 0.0 | 0.0 | Lung ca. (large | 0.0 | 0.0 |
| | | | cell)NCI-H460 | | |
| glio/astro U87- | 0.0 | 0.0 | Lung ca. (non- | 0.0 | 4.5 |
| MG | | | sm. cell) A549 | | |
| glio/astro U-118- | 0.0 | 0.0 | Lung ca. (non- | 0.0 | 0.0 |
| MG | | | s.cell) NCI- | | |
| | | | H23 | | |
| astrocytoma | 0.0 | 0.0 | Lung ca. (non- | 0.0 | 0.0 |
| SW1783 | | | s.cell) HOP-62 | | |
| neuro*; met SK- | 0.0 | 0.0 | Lung ca. (non- | 0.0 | 0.0 |
| N-AS | | | s.cl) NCI-H522 | | |
| astrocytoma SF- | 0.0 | 2.4 | Lung ca. | 0.0 | 0.0 |
| 539 | | | (squam.) SW | | |
| | | | 900 | | |
| astrocytoma SNB- | 0.0 | 0.0 | Lung ca. | 0.0 | 0.0 |
| 75 | | | (squam.) NCI- | | |
| | | | H596 | | |
| glioma SNB-19 | 0.0 | 0.0 | Mammary | 0.0 | 0.0 |
| | | | gland | | |
| glioma U251 | 0.0 | 2.5 | Breast ca.* | 0.0 | 0.0 |
| | | | (pl.ef) MCF-7 | | |
| glioma SF-295 | 0.0 | 0.0 | Breast ca.* | 0.0 | 0.0 |
| | | | (pl.ef) MDA- | | |
| | | | MB-231 | | |
| Heart (Fetal) | 0.0 | 0.0 | Breast ca.* (pl. | 0.0 | 0.0 |
| | | | ef) T47D | | |
| Heart | 0.0 | 0.0 | Breast ca. BT- | 0.0 | 0.0 |
| | | | 549 | | |
| Skeletal muscle | 0.0 | 4.2 | Breast ca. | 0.0 | 0.0 |
| (Fetal) | | | MDA-N | | |
| Skeletal muscle | 0.0 | 0.0 | Ovary | 0.0 | 0.0 |
| Bone marrow | 0.0 | 1.9 | Ovarian ca. | 0.0 | 2.0 |
| | | | OVCAR-3 | | |
| Thymus | 0.0 | 0.0 | Ovarian ca. | 0.0 | 0.0 |
| | | | OVCAR-4 | | |
| Spleen | 0.0 | 0.0 | Ovarian ca. | 0.0 | 0.0 |
| | | | OVCAR-5 | | |
| Lymph node | 0.0 | 0.0 | Ovarian ca. | 0.0 | 0.0 |
| | | | OVCAR-8 | | |
| Colorectal | 0.0 | 3.4 | Ovarian ca. | 0.0 | 0.0 |
| | | | IGROV-1 | | |
| Stomach | 0.0 | 3.7 | Ovarian ca. | 0.0 | 0.0 |
| | | | (ascites) SK- | | |
| | | | OV-3 | | |
| Small intestine | 0.0 | 2.2 | Uterus | 0.0 | 0.0 |
| Colon ca. SW480 | 0.0 | 0.0 | Placenta | 0.0 | 0.0 |
| Colon ca.* | 0.0 | 0.0 | Prostate | 0.0 | 0.0 |
| SW620 (SW480 | | | | | |
| met) | | | | | |
| Colon ca. HT29 | 0.0 | 0.0 | Prostate ca.* | 0.0 | 0.0 |
| | | | (bone met) PC-3 | | |
| Colon ca. HCT- | 0.0 | 0.0 | Testis | 100.0 | 100.0 |
| 116 | | | | | |
| Colon ca. CaCo-2 | 0.0 | 0.0 | Melanoma | 0.0 | 0.0 |
| | | | Hs688(A).T | | |
| CC Well to Mod | 0.0 | 0.0 | Melanoma* | 0.0 | 0.0 |
| Diff (ODO3866) | | | (met) | | |
| | | | Hs688(B).T | | |
| Colon ca. HCC- | 0.0 | 0.0 | Melanoma | 0.0 | 0.0 |
| 2998 | | | UACC-62 | | |
| Gastric ca. (liver | 0.0 | 0.0 | Melanoma | 0.0 | 4.0 |
| met) NCI-N87 | | | M14 | | |
| Bladder | 0.0 | 1.0 | Melanoma | 0.0 | 0.0 |
| | | | LOX IMVI | | |
| Trachea | 0.0 | 0.0 | Melanoma* | 0.0 | 0.0 |
| | | | (met) SK- | | |
| | | | MEL-5 | | |
| Kidney | 0.0 | 0.0 | Adipose | 0.0 | 0.0 |

CNS_Neurodegeneration_v1.0 Summary: Ag3600

Expression of the CG50289-01 gene is low/undetectable in all samples on this panel (CT>35).

General_Screening_Panel_v1.4 Summary: Ag3600

Expression of the CG50289-01 gene is exclusive to the testis (CT=31.8). This gene encodes a serine protease homolog. Serine proteases are important in many aspects of cellular physiology including post-translational processing, protein degradation and cellular signalling. The exclusive expression of this gene in the testis suggests that the protein encoded by this gene may be an excellent target for modulating male reproduction.

Panel 1.2 Summary: Ag792

Highest expression of the CG50289-01 gene is seen in the testis (CT=27.5), a result that is concordant with the results in General_screening_panel_v1.4. Low but significant expression is also seen in the pancreas. This expression profile suggests that the protein encoded by this gene may be an excellent target for modulation of male reproduction and/or hormone release from the pancreas.

Panel 1.3D Summary: Ag792/Ag2555

Two experiments with the same probe and primer set show expression of the CG50289-01 gene to be exclusive to the testis (CTs=32-33). This result is in excellent agreement with the results from Panel 1.2 and General_screening_panel_v1.4. Thus, this exclusive expression of this gene in the testis suggests that the protein encoded by this gene may be an excellent target for modulating male reproduction.

Panel 2D Summary: Ag2555

Expression of the CG50289-01 gene is low/undetectable in all samples on this panel (CT>35).

Panel 4.1D Summary: Ag3600

Expression of the CG50289-01 gene is low/undetectable in all samples on this panel (CT>35).

Panel 4D Summary: Ag2555

Expression of the CG50289-01 gene is low/undetectable in all samples on this panel (CT>35).

NOV5a (CG50353-01: Wnt7a-like)

Expression of gene CG50353-01 was assessed using the primer-probe set Ag3093, described in Table EA. Results of the RTQ-PCR runs are shown in Tables EB, and EC.

TABLE EA
Probe Name Ag3093

| | | | | Start |
|---|---|---|---|---|
| Primers | Sequences | | Length | Position |
| Forward | 5′-ctgtgacctcatgtgctgtg-3′ | (SEQ ID NO:157) | 20 | 909 |
| Probe | TET-5′-gtggctacaacacccaccagtacgc-3′-TAMRA | (SEQ ID NO:158) | 25 | 932 |
| Reverse | 5′-acatagcagcaccagtggaa-3′ | (SEQ ID NO:159) | 20 | 982 |

TABLE EB
Panel 1.3D

| | Rel. Exp. (%) Ag3093, | | Rel. Exp. (%) Ag3093, |
|---|---|---|---|
| Tissue Name | Run 167985246 | Tissue Name | Run 167985246 |
| Liver adenocarcinoma | 2.8 | Kidney (fetal) | 0.1 |
| Pancreas | 0.0 | Renal ca. 786-0 | 0.2 |
| Pancreatic ca. CAPAN 2 | 1.7 | Renal ca. A498 | 0.0 |
| Adrenal gland | 0.0 | Renal ca. RXF 393 | 0.4 |
| Thyroid | 0.0 | Renal ca. ACHN | 0.0 |
| Salivary gland | 0.0 | Renal ca. UO-31 | 0.5 |
| Pituitary gland | 0.0 | Renal ca. TK-10 | 0.0 |
| Brain (fetal) | 3.6 | Liver | 0.0 |
| Brain (whole) | 1.5 | Liver (fetal) | 0.0 |
| Brain (amygdala) | 1.8 | Liver ca. | 0.0 |
| | | (hepatoblast) HepG2 | |
| Brain (cerebellum) | 0.9 | Lung | 0.2 |
| Brain (hippocampus) | 1.4 | Lung (fetal) | 0.9 |
| Brain (substantia nigra) | 0.9 | Lung ca. (small cell) | 0.0 |
| | | LX-1 | |
| Brain (thalamus) | 0.0 | Lung ca. (small cell) | 0.2 |
| | | NCI-H69 | |
| Cerebral Cortex | 3.5 | Lung ca. (s.cell var.) | 0.0 |
| | | SHP-77 | |
| Spinal cord | 0.6 | Lung ca. (large | 0.0 |
| | | cell)NCI-H460 | |
| glio/astro U87-MG | 0.6 | Lung ca. (non-sm. | 0.2 |
| | | Cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.2 | Lung ca. (squam.) | 0.0 |
| | | SW 900 | |
| astrocytoma SNB-75 | 0.1 | Lung ca. (squam.) | 0.3 |
| | | NCI-H596 | |
| glioma SNB-19 | 0.0 | Mammary gland | 0.0 |
| glioma U251 | 0.0 | Breast ca.* (pl.ef) | 0.2 |
| | | MCF-7 | |
| glioma SF-295 | 0.0 | Breast ca.* (pl.ef) | 0.0 |
| | | MDA-MB-231 | |
| Heart (Fetal) | 0.0 | Breast ca.* (pl. ef) | 0.0 |
| | | T47D | |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.1 |
| Thymus | 0.0 | Ovarian ca. OVCAR-4 | 37.1 |
| Spleen | 0.3 | Ovarian ca. OVCAR-5 | 0.7 |
| Lymph node | 0.0 | Ovarian ca. OVCAR-8 | 0.0 |
| Colorectal | 0.0 | Ovarian ca. IGROV-1 | 6.8 |
| Stomach | 0.0 | Ovarian ca. (ascites) | 100.0 |
| | | SK-OV-3 | |
| Small intestine | 0.0 | Uterus | 0.0 |
| Colon ca. SW480 | 0.4 | Placenta | 0.0 |
| Colon ca.* SW620 | 1.4 | Prostate | 0.0 |
| (SW480 met) | | | |
| Colon ca. HT29 | 0.0 | Prostate ca.* (bone | 2.0 |
| | | met) PC-3 | |
| Colon ca. HCT-116 | 0.0 | Testis | 0.3 |
| Colon ca. CaCo-2 | 0.2 | Melanoma | 0.0 |
| | | Hs688(A).T | |
| CC Well to Mod Diff | 0.0 | Melanoma* (met) | 0.0 |
| (ODO3866) | | Hs688(B).T | |
| Colon ca. HCC-2998 | 0.1 | Melanoma UACC-62 | 0.0 |
| Gastric ca. (liver met) | 0.5 | Melanoma M14 | 0.0 |
| NCI-N87 | | | |
| Bladder | 0.0 | Melanoma LOX | 0.0 |
| | | IMVI | |
| Trachea | 0.1 | Melanoma* (met) | 0.0 |
| | | SK-MEL-5 | |
| Kidney | 0.0 | Adipose | 0.2 |

TABLE EC
Panel 4D

| | Rel. Exp. (%) | | Rel. Exp. (%) |
|---|---|---|---|
| | Ag3093, Run | | Ag3093, Run |
| Tissue Name | 164392077 | Tissue Name | 164392077 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| PrimaryTr1 act | 0.0 | Microsvasular Dermal EC | 0.0 |
| | | TNFalpha + IL-1beta | |
| Primary Th1 rest | 0.0 | Bronchial epithelium | 57.4 |
| | | TNFalpha + IL 1beta | |
| Primary Th2 rest | 0.0 | Small airway epithelium | 17.7 |
| | | none | |
| Primary Tr1 rest | 0.0 | Small airway epithelium | 100.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 4.9 | KU-812 (Basophil) | 1.2 |
| | | PMA/ionomycin | |
| 2ry Th1/Th2/Tr1_anti- | 0.0 | CCD1106 (Keratinocytes) | 47.6 |
| CD95 CH11 | | none | |
| LAK cells rest | 0.0 | CCD1106 (Keratinocytes) | 33.7 |
| | | TNFalpha + IL-1beta | |
| LAK cells IL-2 | 0 0 | Liver cirrhosis | 1.4 |
| LAK cells IL-2 + IL-12 | 0.0 | Lupus kidney | 0.0 |
| LAK cells IL-2 + IFN | 0.0 | NCI-H292 none | 4.1 |
| gamma | | | |
| LAK cells IL-2 + IL-18 | 0.0 | NCI-H292 IL-4 | 4.8 |
| LAK cells | 0.0 | NCI-H292 IL-9 | 1.8 |
| PMA/ionomycin | | | |
| NK Cells IL-2 rest | 0.0 | NCI-H292 IL-13 | 2.5 |
| Two Way MLR 3 day | 0.0 | NCI-H292 IFN gamma | 1.6 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 3.5 | Lung fibroblast none | 0.0 |
| PBMC PWM | 0.8 | Lung fibroblast TNF alpha + | 0.0 |
| | | IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 1.0 |
| Macrophages rest | 0.0 | Lung | 2.0 |
| Macrophages LPS | 0.0 | Thymus | 0.0 |
| HUVEC none | 0.0 | Kidney | 0.0 |
| HUVEC starved | 0.0 | | |

Panel 1.3D Summary: Ag3093

The CG50353-01 gene is expressed exclusively in two ovarian cancer cell lines, with highest expression in the SK-OV-3 cell line (CT=30.28). This cell line is unusual because it is derived from ascites. Thus, this gene could potentially be used as a marker for ovarian cancer, particularly ascites derived cancer or as a marker for ascites. Furthermore, antibodies or small molecule drugs could potentially be used in a therapeutic manner to modulate the activity of this gene in ovarian cancer.

Panel 2.2 Summary: Ag3093

Expression of the CG50353-01 gene is low/undetectable in all samples on this panel (CTs>35).

Panel 4D Summary: Ag3093

The CG50353-01 gene is expressed at the highest level in TNF alpha+IL-1 beta treated small airway epithelial cells (CT=32.6) as well as TNF alpha+IL-1 beta treated bronchial epithelial cells and CCD1106 keratinocytes (treated and non-treated). The presence of this transcript in keratinocytes suggests that this gene may be important in skin disorders including psoriasis. Expression in airway/bronchial cell types suggests that this gene may also be involved in inflammatory lung disorders that include chronic obstructive pulmonary disease, asthma, allergy and emphysema. Therefore, therapeutic modalities that involve this gene or gene product may be beneficial in the treatment of these conditions.

NOV6a (CG50221-01: Apical Endosomal Glycoprotein)

Expression of gene CG5022 1-01 was assessed using the primer-probe sets Ag2495 and Ag4806, described in Tables FA and FB. Results of the RTQ-PCR runs are shown in Table FC.

TABLE FA
Probe Name Ag2495

| | | | | Start |
|---|---|---|---|---|
| Primers | Sequences | | Length | Position |
| Forward | 5′-ctggcacccctgctatactc-3′ | (SEQ ID NO:160) | 20 | 1003 |
| Probe | TET-5′-attccaagcctcaggcacctccaact-3′-TAMRA | (SEQ ID NO:161) | 26 | 1034 |
| Reverse | 5′-tgatagaagaccagccatctca-3′ | (SEQ ID NO:162) | 22 | 1066 |

TABLE FB
Probe Name Ag4806

| | | | | Start |
|---|---|---|---|---|
| Primers | Sequences | | Length | Position |
| Forward | 5′-ctacgtggctctggatgatct-3′ | (SEQ ID NO:163) | 21 | 2909 |
| Probe | TET-5′-cctgccctcagccaggttcctgt-3′-TAMRA | (SEQ ID NO:164) | 23 | 2947 |
| Reverse | 5′-acacaggccagactcaaaatc-3′ | (SEQ ID NO:165) | 21 | 2970 |

TABLE FC
General_screening_panel_v1.4

| | Rel. Exp. (%) Ag4806, | | Rel. Exp. (%) Ag4806, |
|---|---|---|---|
| Tissue Name | Run 223204110 | Tissue Name | Run 223204110 |
| Adipose | 7.6 | Renal ca. TK-10 | 4.4 |
| Melanoma* | 4.6 | Bladder | 13.7 |
| Hs688(A).T | | | |
| Melanoma* | 12.7 | Gastric ca. (liver met.) | 6.6 |
| Hs688(B).T | | NCI-N87 | |
| Melanoma* M14 | 27.7 | Gastric ca. KATO III | 0.0 |
| Melanoma* | 0.0 | Colon ca. SW-948 | 7.7 |
| LOXIMVI | | | |
| Melanoma* SK- | 0.0 | Colon ca. SW480 | 9.6 |
| MEL-5 | | | |
| Squamous cell | 0.0 | Colon ca.* (SW480 | 7.2 |
| carcinoma SCC-4 | | met) SW620 | |
| Testis Pool | 0.0 | Colon ca. HT29 | 15.0 |
| Prostate ca.* (bone | 0.0 | Colon ca. HCT-116 | 15.2 |
| met) PC-3 | | | |
| Prostate Pool | 3.6 | Colon ca. CaCo-2 | 12.5 |
| Placenta | 4.7 | Colon cancer tissue | 15.8 |
| Uterus Pool | 0.0 | Colon ca.SW1116 | 16.8 |
| Ovarian ca. OVCAR-3 | 0.3 | Colon ca. Colo-205 | 13.9 |
| Ovarian ca. SK-OV-3 | 19.9 | Colon ca. SW-48 | 0.0 |
| Ovarian ca. OVCAR-4 | 0.0 | Colon Pool | 0.0 |
| Ovarian ca. OVCAR-5 | 14.2 | Small Intestine Pool | 0.0 |
| Ovarian ca. IGROV-1 | 24.1 | Stomach Pool | 1.3 |
| Ovarian ca. OVCAR-8 | 23.2 | Bone Marrow Pool | 0.0 |
| Ovary | 0.7 | Fetal Heart | 17.0 |
| Breast ca. MCF-7 | 6.7 | Heart Pool | 4.2 |
| Breast ca. MDA- | 65.1 | Lymph Node Pool | 3.5 |
| MB-231 | | | |
| Breast ca. BT 549 | 18.8 | Fetal Skeletal Muscle | 6.5 |
| Breast ca. T47D | 100.0 | Skeletal Muscle Pool | 11.8 |
| Breast ca. MDA-N | 8.7 | Spleen Pool | 18.7 |
| Breast Pool | 1.9 | Thymus Pool | 12.9 |
| Trachea | 0.0 | CNS cancer (glio/astro) | 25.9 |
| | | U87-MG | |
| Lung | 0.0 | CNS cancer (glio/astro) | 37.9 |
| | | U-118-MG | |
| Fetal Lung | 19.9 | CNS cancer (neuro; met) | 9.3 |
| | | SK-N-AS | |
| Lung ca. NCI-N417 | 0.0 | CNS cancer (astro) SF- | 3.2 |
| | | 539 | |
| Lung ca. LX-1 | 11.0 | CNS cancer (astro) | 33.4 |
| | | SNB-75 | |
| Lung ca. NCI-H146 | 0.0 | CNS cancer (glio) SNB- | 11.0 |
| | | 19 | |
| Lung ca. SHP-77 | 11.4 | CNS cancer (glio) SF- | 9.8 |
| | | 295 | |
| Lung ca. A549 | 33.7 | Brain (Amygdala) Pool | 9.0 |
| Lung ca. NCI-H526 | 0.0 | Brain (cerebellum) | 22.4 |
| Lung ca. NCI-H23 | 7.0 | Brain (fetal) | 14.4 |
| Lung ca. NCI-H460 | 9.7 | Brain (Hippocampus) | 8.4 |
| | | Pool | |
| Lung ca. HOP-62 | 7.9 | Cerebral Cortex Pool | 0.0 |
| Lung ca. NCI-H522 | 0.0 | Brain (Substantia nigra) | 18.9 |
| | | Pool | |
| Liver | 40.1 | Brain (Thalamus) Pool | 8.2 |
| Fetal Liver | 5.1 | Brain (whole) | 3.5 |
| Liver ca. HepG2 | 16.6 | Spinal Cord Pool | 7.8 |
| Kidney Pool | 11.5 | Adrenal Gland | 4.0 |
| Fetal Kidney | 16.7 | Pituitary gland Pool | 13.9 |
| Renal ca. 786-0 | 25.7 | Salivary Gland | 0.8 |
| Renal ca. A498 | 0.0 | Thyroid (female) | 1.8 |
| Renal ca. ACHN | 0.0 | Pancreatic ca. CAPAN2 | 0.5 |
| Renal ca. UO-31 | 8.8 | Pancreas Pool | 11.7 |

CNS_Neurodegeneration_v1.0 Summary: Ag2495

Expression of the CG50221-01 gene is low/undetectable in all samples on this panel (CT>35).

General_Screening_Panel_v1.4 Summary: Ag4806

Expression of the CG50221-01 gene is highest in a breast cancer cell line (CT=31.5). This gene is also expressed in breast, ovarian and colon cancer cell lines at higher levels when compared to normal tissue samples. Hence, expression of this gene might be used as a marker to identify normal tissue from cancerous tissue in these organs.

There is relatively low level of expression in most endocrine (metabolic)-related tissues except for liver. Modulation of this gene or gene-product may therefore be beneficial in treating various abnormalities related to liver function. The higher levels of expression in adult liver (CT=32.7) when compared to fetal liver suggest that expression of this gene can also be used to differentiate fetal vs adult liver tissue. Conversely, higher levels of expression in fetal lung (CT=33) when compared to adult lung (CT=40) suggest involvement of this gene in the development of the lung. Expression of this gene could also therefore be used to differentiate between fetal and adult lung tissue.

Panel 1.3D Summary: Ag2495

Expression of the CG50221-01 gene is low/undetectable in all samples on this panel (CT>35).

Panel 2D Summary: Ag2495

Expression of the CG50221-01 gene is low/undetectable in all samples on this panel (CT>35).

Panel 4D Summary: Ag2495

Expression of the CG5022 1-01 gene is low/undetectable in all samples on this panel (CT>35).

NOV7a (CG50367-01: ADAM13-like)

Expression of gene CG50367-01 was assessed using the primer-probe set Ag2425, described in Table GA. Results of the RTQ-PCR runs are shown in Tables GB, GC, and GD.

TABLE GA
Probe Name Ag2425

| | | | | Start |
|---|---|---|---|---|
| Primers | Sequences | | Length | Position |
| Forward | 5′-ggctcctgctgaccatattc-3′ | (SEQ ID NO:166) | 20 | 2342 |
| Probe | TET-5′-catttaccctccaccatttctcccag-3′-TAMRA | (SEQ ID NO:167) | 26 | 2366 |
| Reverse | 5′-gctgggctcatgagagttct-3′ | (SEQ ID NO:168) | 20 | 2398 |

TABLE GB
Panel 1.3D

| | Rel. Exp. (%) | | Rel. Exp. (%) |
|---|---|---|---|
| | Ag2425, | | Ag2425, |
| Tissue Name | Run 155561580 | Tissue Name | Run 155561580 |
| Liver adenocarcinoma | 0.0 | Kidney (fetal) | 3.9 |
| Pancreas | 1.8 | Renal ca. 786-0 | 0.0 |
| Pancreatic ca. CAPAN 2 | 0.0 | Renal ca. A498 | 0.0 |
| Adrenal gland | 0.9 | Renal ca. RXF 393 | 0.0 |
| Thyroid | 2.7 | Renal ca. ACHN | 1.6 |
| Salivary gland | 1.1 | Renal ca. UO-31 | 0.0 |
| Pituitary gland | 0.5 | Renal ca. TK-10 | 0.0 |
| Brain (fetal) | 4.6 | Liver | 0.0 |
| Brain (whole) | 2.3 | Liver (fetal) | 1.2 |
| Brain (amygdala) | 4.2 | Liver ca. | 0.0 |
| | | (hepatoblast) HepG2 | |
| Brain (cerebellum) | 0.0 | Lung | 2.8 |
| Brain (hippocampus) | 25.3 | Lung (fetal) | 17.9 |
| Brain (substantia nigra) | 2.4 | Lung ca. (small cell) | 0.0 |
| | | LX-1 | |
| Brain (thalamus) | 9.4 | Lung ca. (small cell) | 0.0 |
| | | NCI-H69 | |
| Cerebral Cortex | 1.5 | Lung ca. (s. cell var.) | 1.0 |
| | | SHP-77 | |
| Spinal cord | 3.9 | Lung ca. (large | 0.0 |
| | | cell) NCI-H460 | |
| glio/astro U87-MG | 0.0 | Lung ca. (non-sm. | 1.7 |
| | | cell) A549 | |
| glio/astro U-118-MG | 1.1 | Lung ca. (non-s. cell) | 1.8 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.8 | Mammary gland | 13.5 |
| glioma U251 | 0.0 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (Fetal) | 3.4 | Breast ca.* (pl. ef) | 0.0 |
| | | T47D | |
| Heart | 1.1 | Breast ca. BT-549 | 1.6 |
| Skeletal muscle (Fetal) | 100.0 | Breast ca. MDA-N | 0.0 |
| Skeletal muscle | 0.9 | Ovary | 1.9 |
| Bone marrow | 3.3 | Ovarian ca. OVCAR-3 | 0.0 |
| Thymus | 4.1 | Ovarian ca. OVCAR-4 | 0.0 |
| Spleen | 2.7 | Ovarian ca. OVCAR-5 | 0.0 |
| Lymph node | 4.6 | Ovarian ca. OVCAR-8 | 0.0 |
| Colorectal | 5.9 | Ovarian ca. IGROV-1 | 0.0 |
| Stomach | 7.3 | Ovarian ca. (ascites) | 0.0 |
| | | SK-OV-3 | |
| Small intestine | 18.4 | Uterus | 37.4 |
| Colon ca. SW480 | 0.0 | Placenta | 1.8 |
| Colon ca.* SW620 | 0.0 | Prostate | 8.8 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 7.5 |
| Colon ca. CaCo-2 | 0.0 | Melanoma | 5.0 |
| | | Hs688(A).T | |
| CC Well to Mod Diff | 0.0 | Melanoma* (met) | 3.3 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX | 0.0 |
| | | IMVI | |
| Trachea | 15.8 | Melanoma* (met) | 0.0 |
| | | SK-MEL-5 | |
| Kidney | 1.8 | Adipose | 1.6 |

TABLE GC
Panel 2D

| | Rel. Exp. (%) | | Rel. Exp. (%) |
|---|---|---|---|
| | Ag2425, Run | | Ag2425, Run |
| Tissue Name | 155562155 | Tissue Name | 155562155 |
| Normal Colon | 100.0 | Kidney Margin | 0.0 |
| | | 8120608 | |
| CC Well to Mod Diff | 5.6 | Kidney Cancer | 0.0 |
| (ODO3866) | | 8120613 | |
| CC Margin (ODO3866) | 4.5 | Kidney Margin | 2.1 |
| | | 8120614 | |
| CC Gr.2 rectosigmoid | 20.7 | Kidney Cancer | 2.2 |
| (ODO3868) | | 9010320 | |
| CC Margin (ODO3868) | 21.9 | Kidney Margin | 6.1 |
| | | 9010321 | |
| CC Mod Diff (ODO3920) | 6.7 | Normal Uterus | 49.3 |
| CC Margin (ODO3920) | 61.6 | Uterine Cancer | 92.7 |
| | | 064011 | |
| CC Gr.2 ascend colon | 1.0 | Normal Thyroid | 18.3 |
| (ODO3921) | | | |
| CC Margin (ODO3921) | 6.2 | Thyroid Cancer | 0.0 |
| CC from Partial | 0.0 | Thyroid Cancer | 2.8 |
| Hepatectomy (ODO4309) | | A302152 | |
| Mets | | | |
| Liver Margin (ODO4309) | 0.0 | Thyroid Margin | 20.4 |
| | | A302153 | |
| Colon mets to lung | 0.0 | Normal Breast | 53.2 |
| (OD04451-01) | | | |
| Lung Margin (OD04451-02) | 0.0 | Breast Cancer | 0.0 |
| Normal Prostate 6546-1 | 66.4 | Breast Cancer | 2.6 |
| | | (OD04590-01) | |
| Prostate Cancer | 25.9 | Breast Cancer Mets | 11.5 |
| (OD04410) | | (OD04590-03) | |
| Prostate Margin | 72.7 | Breast Cancer | 0.0 |
| (OD04410) | | Metastasis | |
| Prostate Cancer | 45.4 | Breast Cancer | 24.7 |
| (OD04720-01) | | | |
| Prostate Margin | 33.7 | Breast Cancer | 51.8 |
| (OD04720-02) | | | |
| Normal Lung | 84.1 | Breast Cancer | 3.1 |
| | | 9100266 | |
| Lung Met to Muscle | 9.4 | Breast Margin | 12.9 |
| (ODO4286) | | 9100265 | |
| Muscle Margin | 0.0 | Breast Cancer | 17.3 |
| (ODO4286) | | A209073 | |
| Lung Malignant Cancer | 0.0 | Breast Margin | 99.3 |
| (OD03126) | | A2090734 | |
| Lung Margin (OD03126) | 10.6 | Normal Liver | 0.0 |
| Lung Cancer (OD04404) | 0.0 | Liver Cancer | 4.0 |
| Lung Margin (OD04404) | 0.0 | Liver Cancer 1025 | 4.9 |
| Lung Cancer (OD04565) | 13.7 | Liver Cancer 1026 | 0.0 |
| Lung Margin (OD04565) | 10.6 | Liver Cancer 6004-T | 0.0 |
| Lung Cancer (OD04237- | 0.0 | Liver Tissue 6004-N | 8.5 |
| 01) | | | |
| Lung Margin (OD04237- | 0.0 | Liver Cancer 6005-T | 0.0 |
| 02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 11.1 |
| Melanoma Metastasis | 0.0 | Bladder Cancer | 3.1 |
| Lung Margin (OD04321) | 2.9 | Bladder Cancer | 14.6 |
| Normal Kidney | 9.7 | Bladder Cancer | 2.4 |
| | | (OD04718-01) | |
| Kidney Ca, Nuclear grade 2 | 0.0 | Bladder Normal | 10.6 |
| (OD04338) | | Adjacent (OD04718- | |
| | | 03) | |
| Kidney Margin | 6.0 | Normal Ovary | 0.0 |
| (OD04338) | | | |
| Kidney Ca Nuclear grade | 0.0 | Ovarian Cancer | 0.0 |
| 1/2 (OD04339) | | | |
| Kidney Margin | 0.0 | Ovarian Cancer | 3.2 |
| (OD04339) | | (OD04768-07) | |
| Kidney Ca, Clear cell type | 0.0 | Ovary Margin | 6.0 |
| (OD04340) | | (OD04768-08) | |
| Kidney Margin | 4.0 | Normal Stomach | 12.7 |
| (OD04340) | | | |
| Kidney Ca, Nuclear grade | 0.0 | Gastric Cancer | 9.9 |
| 3 (OD04348) | | 9060358 | |
| Kidney Margin | 6.7 | Stomach Margin | 0.0 |
| (OD04348) | | 9060359 | |
| Kidney Cancer | 0.0 | Gastric Cancer | 39.2 |
| (OD04622-01) | | 9060395 | |
| Kidney Margin | 7.4 | Stomach Margin | 26.4 |
| (OD04622-03) | | 9060394 | |
| Kidney Cancer | 0.0 | Gastric Cancer | 6.2 |
| (OD04450-01) | | 9060397 | |
| Kidney Margin | 0.0 | Stomach Margin | 3.3 |
| (OD04450-03) | | 9060396 | |
| Kidney Cancer 8120607 | 0.0 | Gastric Cancer 064005 | 25.3 |

TABLE GD
Panel 4D

| | Rel. Exp. (%) | | Rel. Exp. (%) |
|---|---|---|---|
| | Ag2425, Run | | Ag2425, Run |
| Tissue Name | 155562267 | Tissue Name | 155562267 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 1.3 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 1.8 | Coronery artery SMC rest | 0.0 |
| lymphocyte act | | | |
| CD45RO CD4 | 1.4 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 1.3 | KU-812 (Basophil) | 0.0 |
| | | PMA/ionomycin | |
| 2ry Th1/Th2/Tr1_anti- | 3.9 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 4.9 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + | 0.0 |
| | | IL-1beta | |
| PBMC rest | 0.0 | Lung fibroblast none | 13.0 |
| PBMC PWM | 0.0 | Lung fibroblast TNF alpha + | 2.7 |
| | | IL-1beta | |
| PBMC PHA-L | 0.0 | Lung fibroblast IL-4 | 3.4 |
| Ramos (B cell) none | 0.0 | Lung fibroblast IL-9 | 10.7 |
| Ramos (B cell) | 0.0 | Lung fibroblast IL-13 | 5.9 |
| ionomycin | | | |
| B lymphocytes PWM | 0.0 | Lung fibroblast IFN | 3.3 |
| | | gamma | |
| B lymphocytes CD40L | 0.0 | Dermal fibroblast | 23.7 |
| and IL-4 | | CCD1070 rest | |
| EOL-1 dbcAMP | 0.0 | Dermal fibroblast | 5.6 |
| | | CCD1070 TNF alpha | |
| EOL-1 dbcAMP | 0.0 | Dermal fibroblast | 12.5 |
| PMA/ionomycin | | CCD1070 IL-1beta | |
| Dendritic cells none | 0.0 | Dermal fibroblast IFN | 64.6 |
| | | gamma | |
| Dendritic cells LPS | 0.0 | Dermal fibroblast IL-4 | 100.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 28.3 |
| Macrophages rest | 0.0 | Lung | 21.6 |
| Macrophages LPS | 0.0 | Thymus | 0.0 |
| HUVEC none | 0.0 | Kidney | 0.0 |
| HUVEC starved | 0.0 | | |

CNS_Neurodegeneration_v1.0 Summary: Ag2425

Expression of the CG50367-01 gene is low/undetectable in all samples on this panel (CT>34.5).

Panel 1.3D Summary: Ag2425

Highest expression of the CG50367-01 gene is seen in fetal skeletal muscle (CT=31.1). This gene appears to be more highly expressed in fetal skeletal muscle when compared to expression in adult skeletal muscle (CT=40). Thus expression of this gene could be used to differentiate between fetal and adult skeletal muscle. Furthermore, the higher levels of expression in the fetal source of the tissue suggest that the protein encoded by this gene may be involved in the development of the skeletal muscle in the fetus. Thus, therapeutic modulation of the expression or function of this gene may restore muscle mass or function to weak or dystrophic muscle in the adult.

This gene is expressed at a very low level in all the cancer cell lines used in this panel. The absence of expression of this gene in the cancer cell lines suggests that modulation of the function of the gene product through the use of peptides, polypeptides, chimeric molecules or small molecule drugs, may be useful in the therapy of cancer.

This gene is a cell-surface metalloprotease expressed at low levels in the hippocampus. It may be useful in the treatment of diseases in which the hippocampus is involved, such as Alzheimer's disease, Parkinson's disease, schizophrenia, bipolar disorder, or temporal lobe epilepsy.

Panel 2D Summary: Ag2425

The CG50367-01 gene is expressed at low levels in this panel, with highest expression in the colon (CT=32.2). Moderately higher levels of expression are seen in normal breast, uterine and thyroid tissues compared to the adjacent cancers. Hence, expression of this gene might be used as a marker to identify normal tissue from cancerous tissue in these organs. Therapeutic modulation of the activity of the product of this gene, through the use of peptides, polypeptides, chimeric molecules or small molecule drugs, may be useful in the therapy of these cancers.

Panel 4D Summary: Ag2425

The CG50367-01 transcript is most highly expressed in dermal fibroblast upon treatment with either Il-4 or Ifn gamma (CTs=31-32) and at lower levels in resting dermal fibroblasts. This transcript is also expressed in lung fibroblasts and normal lung and thymus. This transcript encodes for a ADAM like protein, a member of membrane-anchored glycoproteins that have been implicated in diverse cellular processes from cell cell interaction to shedding of cell surface proteases. The expression of this transcript in dermal and lung fibroblasts suggests that the protein encoded by this transcript might be involved in disease associated with fibrosis or fibroplasia. Modulation of the expression or the function of this molecule might be useful for the treatment of psoriasis, chronic obstructive pulmonary diseases and potentially for osteoarthritis and rheumatoid arthritis.

NOV8 (CG50321-01: Leucine Rich Containing F Box Protein)

Expression of gene CG50321-01 was assessed using the primer-probe set Ag2557, described in Table HA. Results of the RTQ-PCR runs are shown in Tables HB, HC and HD.

TABLE HA
Probe Name Ag2557

| | | | | Start |
|---|---|---|---|---|
| Primers | Sequences | | Length | Position |
| Forward | 5′-tgactttgaacttgcagacttg-3′ | (SEQ ID NO:169) | 22 | 646 |
| Probe | TET-5′-cttgcaaatcacagatgaaggtctca-3′-TAMRA | (SEQ ID NO:170) | 26 | 668 |
| Reverse | 5′-aggcacaaagggattgtaactt-3′ | (SEQ ID NO:171) | 22 | 717 |

TABLE HB
CNS_neurodegeneration_v1.0

| | Rel. Exp. | | |
|---|---|---|---|
| | (%) | | Rel. Exp. (%) |
| | Ag2557, | | Ag2557, |
| | Run | | Run |
| Tissue Name | 206974725 | Tissue Name | 206974725 |
| AD 1 Hippo | 14.9 | Control (Path) 3 | 6.7 |
| | | Temporal Ctx | |
| AD 2 Hippo | 27.4 | Control (Path) 4 | 24.8 |
| | | Temporal Ctx | |
| AD 3 Hippo | 6.1 | AD 1 Occipital Ctx | 12.5 |
| AD 4 Hippo | 4.9 | AD 2 Occipital Ctx | 0.0 |
| | | (Missing) | |
| AD 5 Hippo | 92.7 | AD 3 Occipital Ctx | 5.0 |
| AD 6 Hippo | 49.0 | AD 4 Occipital Ctx | 16.7 |
| Control 2 Hippo | 28.5 | AD 5 Occipital Ctx | 15.6 |
| Control 4 Hippo | 8.4 | AD 5 Occipital Ctx | 48.0 |
| Control (Path) 3 | 6.9 | Control 1 Occipital | 3.6 |
| Hippo | | Ctx | |
| AD 1 Temporal Ctx | 15.8 | Control 2 Occipital | 71.2 |
| | | Ctx | |
| AD 2 Temporal Ctx | 29.9 | Control 3 Occipital | 12.4 |
| | | Ctx | |
| AD 3 Temporal Ctx | 3.8 | Control 4 Occipital | 5.7 |
| | | Ctx | |
| AD 4 Temporal Ctx | 18.7 | Control (Path) 1 | 100.0 |
| | | Occipital Ctx | |
| AD 5 Inf Temporal | 92.7 | Control (Path) 2 | 8.4 |
| Ctx | | Occipital Ctx | |
| AD 5 Sup Temporal | 32.1 | Control (Path) 3 | 2.1 |
| Ctx | | Occipital Ctx | |
| AD 6 Inf Temporal | 36.9 | Control (Path) 4 | 8.4 |
| Ctx | | Occipital Ctx | |
| AD 6 Sup Temporal | 37.1 | Control 1 Parietal | 7.2 |
| Ctx | | Ctx | |
| Control 1 Temporal | 6.6 | Control 2 Parietal | 33.7 |
| Ctx | | Ctx | |
| Control 2 Temporal | 51.1 | Control 3 Parietal | 17.7 |
| Ctx | | Ctx | |
| Control 3 Temporal | 13.2 | Control (Path) 1 | 95.3 |
| Ctx | | Parietal Ctx | |
| Control 3 Temporal | 6.8 | Control (Path) 2 | 17.6 |
| Ctx | | Parietal Ctx | |
| Control (Path) 1 | 66.0 | Control (Path) 3 | 5.1 |
| Temporal Ctx | | Parietal Ctx | |
| Control (Path) 2 | 29.3 | Control (Path) 4 | 39.8 |
| Temporal Ctx | | Parietal Ctx | |

TABLE HC
Panel 1.3D

| | Rel. Exp. (%) | | Rel. Exp. (%) |
|---|---|---|---|
| | Ag2557, | | Ag2557, |
| Tissue Name | Run 165640108 | Tissue Name | Run 165640108 |
| Liver adenocarcinoma | 23.8 | Kidney (fetal) | 12.7 |
| Pancreas | 5.4 | Renal ca. 786-0 | 5.9 |
| Pancreatic ca. CAPAN 2 | 20.7 | Renal ca. A498 | 21.0 |
| Adrenal gland | 12.2 | Renal ca. RXF 393 | 13.5 |
| Thyroid | 5.8 | Renal ca. ACHN | 10.5 |
| Salivary gland | 7.2 | Renal ca. UO-31 | 6.3 |
| Pituitary gland | 8.5 | Renal ca. TK-10 | 23.2 |
| Brain (fetal) | 31.4 | Liver | 4.0 |
| Brain (whole) | 55.1 | Liver (fetal) | 13.1 |
| Brain (amygdala) | 43.5 | Liver ca. | 7.5 |
| | | (hepatoblast) HepG2 | |
| Brain (cerebellum) | 44.8 | Lung | 7.7 |
| Brain (hippocampus) | 42.3 | Lung (fetal) | 12.4 |
| Brain (substantia nigra) | 14.8 | Lung ca. (small cell) | 12.2 |
| | | LX-1 | |
| Brain (thalamus) | 30.6 | Lung ca. (small cell) | 14.2 |
| | | NCI-H69 | |
| Cerebral Cortex | 18.7 | Lung ca. (s. cell var.) | 15.4 |
| | | SHP-77 | |
| Spinal cord | 11.2 | Lung ca. (large | 54.0 |
| | | cell) NCI-H460 | |
| glio/astro U87-MG | 15.9 | Lung ca. (non-sm. | 42.3 |
| | | cell) A549 | |
| glio/astro U-118-MG | 22.4 | Lung ca. (non-s. cell) | 17.3 |
| | | NCI-H23 | |
| astrocytoma SW1783 | 24.7 | Lung ca. (non-s. cell) | 32.1 |
| | | HOP-62 | |
| neuro*; met SK-N-AS | 16.4 | Lung ca. (non-s. cl) | 14.2 |
| | | NCI-H522 | |
| astrocytoma SF-539 | 6.3 | Lung ca. (squam.) | 8.6 |
| | | SW 900 | |
| astrocytoma SNB-75 | 18.7 | Lung ca. (squam.) | 10.7 |
| | | NCI-H596 | |
| glioma SNB-19 | 22.7 | Mammary gland | 15.8 |
| glioma U251 | 32.1 | Breast ca.* (pl. ef) | 9.3 |
| | | MCF-7 | |
| glioma SF-295 | 30.4 | Breast ca.* (pl. ef) | 16.3 |
| | | MDA-MB-231 | |
| Heart (Fetal) | 3.7 | Breast ca.* (pl. ef) | 24.5 |
| | | T47D | |
| Heart | 7.9 | Breast ca. BT-549 | 12.6 |
| Skeletal muscle (Fetal) | 2.3 | Breast ca. MDA-N | 4.4 |
| Skeletal muscle | 15.7 | Ovary | 5.8 |
| Bone marrow | 10.8 | Ovarian ca. OVCAR-3 | 10.4 |
| Thymus | 14.6 | Ovarian ca. OVCAR-4 | 6.2 |
| Spleen | 15.1 | Ovarian ca. OVCAR-5 | 15.5 |
| Lymph node | 21.3 | Ovarian ca. OVCAR-8 | 3.3 |
| Colorectal | 8.1 | Ovarian ca. IGROV-1 | 2.7 |
| Stomach | 15.7 | Ovarian ca. (ascites) | 28.9 |
| | | SK-OV-3 | |
| Small intestine | 20.9 | Uterus | 22.4 |
| Colon ca. SW480 | 10.2 | Placenta | 8.5 |
| Colon ca.* SW620 | 6.7 | Prostate | 6.8 |
| (SW480 met) | | | |
| Colon ca. HT29 | 1.0 | Prostate ca.* (bone | 17.6 |
| | | met) PC-3 | |
| Colon ca. HCT-116 | 10.7 | Testis | 27.0 |
| Colon ca. CaCo-2 | 9.0 | Melanoma | 4.2 |
| | | Hs688(A).T | |
| CC Well to Mod Diff | 7.7 | Melanoma* (met) | 5.9 |
| (ODO3866) | | Hs688(B).T | |
| Colon ca. HCC-2998 | 7.7 | Melanoma UACC-62 | 9.7 |
| Gastric ca. (liver met) | 100.0 | Melanoma M14 | 36.1 |
| NCI-N87 | | | |
| Bladder | 14.9 | Melanoma LOX | 2.2 |
| | | IMVI | |
| Trachea | 9.9 | Melanoma* (met) | 4.1 |
| | | SK-MEL-5 | |
| Kidney | 3.5 | Adipose | 7.4 |

TABLE HD
Panel 4D

| | Rel. Exp. (%) | | Rel. Exp. (%) |
|---|---|---|---|
| | Ag2557, Run | | Ag2557, Run |
| Tissue Name | 164393419 | Tissue Name | 164393419 |
| Secondary Th1 act | 27.4 | HUVEC IL-1beta | 4.8 |
| Secondary Th2 act | 19.8 | HUVEC IFN gamma | 19.8 |
| Secondary Tr1 act | 28.7 | HUVEC TNF alpha + IFN | 3.9 |
| | | gamma | |
| Secondary Th1 rest | 11.4 | HUVEC TNF alpha + IL4 | 6.9 |
| Secondary Th2 rest | 17.9 | HUVEC IL-11 | 8.0 |
| Secondary Tr1 rest | 19.3 | Lung Microvascular EC | 19.5 |
| | | none | |
| Primary Th1 act | 26.8 | Lung Microvascular EC | 13.7 |
| | | TNF alpha + IL-1beta | |
| Primary Th2 act | 23.5 | Microvascular Dermal EC | 35.6 |
| | | none | |
| Primary Tr1 act | 36.9 | Microsvasular Dermal EC | 12.2 |
| | | TNF alpha + IL-1beta | |
| Primary Th1 rest | 75.8 | Bronchial epithelium | 29.9 |
| | | TNF alpha + IL1beta | |
| Primary Th2 rest | 43.8 | Small airway epithelium | 17.2 |
| | | none | |
| Primary Tr1 rest | 34.2 | Small airway epithelium | 47.6 |
| | | TNF alpha + IL-1beta | |
| CD45RA CD4 | 16.6 | Coronery artery SMC rest | 19.9 |
| lymphocyte act | | | |
| CD45RO CD4 | 36.3 | Coronery artery SMC | 17.2 |
| lymphocyte act | | TNF alpha + IL-1beta | |
| CD8 lymphocyte act | 23.3 | Astrocytes rest | 21.8 |
| Secondary CD8 | 22.1 | Astrocytes TNF alpha + IL- | 21.5 |
| lymphocyte rest | | 1beta | |
| Secondary CD8 | 17.4 | KU-812 (Basophil) rest | 16.2 |
| lymphocyte act | | | |
| CD4 lymphocyte none | 15.9 | KU-812 (Basophil) | 56.6 |
| | | PMA/ionomycin | |
| 2ry Th1/Th2/Tr1_anti- | 36.6 | CCD1106 (Keratinocytes) | 14.6 |
| CD95 CH11 | | none | |
| LAK cells rest | 21.5 | CCD1106 (Keratinocytes) | 6.0 |
| | | TNF alpha + IL-1beta | |
| LAK cells IL-2 | 33.9 | Liver cirrhosis | 3.9 |
| LAK cells IL-2 + IL-12 | 23.2 | Lupus kidney | 3.9 |
| LAK cells IL-2 + IFN | 33.0 | NCI-H292 none | 46.3 |
| gamma | | | |
| LAK cells IL-2 + IL-18 | 35.4 | NCI-H292 IL-4 | 57.8 |
| LAK cells | 8.7 | NCI-H292 IL-9 | 47.0 |
| PMA/ionomycin | | | |
| NK Cells IL-2 rest | 23.3 | NCI-H292 IL-13 | 28.5 |
| Two Way MLR 3 day | 20.3 | NCI-H292 IFN gamma | 24.3 |
| Two Way MLR 5 day | 16.7 | HPAEC none | 18.3 |
| Two Way MLR 7 day | 10.7 | HPAEC TNF alpha + IL-1 | 12.8 |
| | | beta | |
| PBMC rest | 26.6 | Lung fibroblast none | 12.8 |
| PBMC PWM | 85.3 | Lung fibroblast TNF alpha + | 16.5 |
| | | IL-1beta | |
| PBMC PHA-L | 43.5 | Lung fibroblast IL-4 | 13.6 |
| Ramos (B cell) none | 15.1 | Lung fibroblast IL-9 | 13.7 |
| Ramos (B cell) | 92.7 | Lung fibroblast IL-13 | 9.6 |
| ionomycin | | | |
| B lymphocytes PWM | 93.3 | Lung fibroblast IFN | 11.2 |
| | | gamma | |
| B lymphocytes CD40L | 39.5 | Dermal fibroblast | 21.9 |
| and IL-4 | | CCD1070 rest | |
| EOL-1 dbcAMP | 65.5 | Dermal fibroblast | 52.9 |
| | | CCD1070 TNF alpha | |
| EOL-1 dbcAMP | 58.2 | Dermal fibroblast | 19.5 |
| PMA/ionomycin | | CCD1070 IL-1beta | |
| Dendritic cells none | 18.9 | Dermal fibroblast IFN | 13.8 |
| | | gamma | |
| Dendritic cells LPS | 17.7 | Dermal fibroblast IL-4 | 23.0 |
| Dendritic cells anti- | 23.7 | IBD Colitis 2 | 1.3 |
| CD40 | | | |
| Monocytes rest | 33.9 | IBD Crohn's | 2.8 |
| Monocytes LPS | 46.7 | Colon | 39.2 |
| Macrophages rest | 42.6 | Lung | 22.4 |
| Macrophages LPS | 26.6 | Thymus | 47.0 |
| HUVEC none | 12.9 | Kidney | 100.0 |
| HUVEC starved | 37.6 | | |

CNS_Neurodegeneration_v1.0 Summary: Ag2557

A small decrease is detected in the expression of the CG5032 1-01 gene in the postmortem brains of Alzheimer's patients when compared normal controls. This protein is an F-Box protein containing leucine-rich repeats; these proteins are involved in ubiquitination and proteosomal degradation of proteins. This gene is therefore an excellent drug target for the treatment of diseases involving protein precipitation including Alzheimer's disease, Huntington's disease, Parkinson's disease, progressive supranuclear palsy, or spinocerebellar ataxia.

Reference:

Ilyin G P, Rialland M, Pigeon C, Guguen-Guillouzo C. cDNA cloning and expression analysis of new members of the mammalian F-box protein family. Genomics Jul. 1, 2000;67(1):40-7

F-box proteins are critical components of the SCF ubiquitin-protein ligase complex and are involved in substrate recognition and recruitment for ubiquitination and consequent degradation by the proteasome. We have isolated cDNAs encoding a further 10 mammalian F-box proteins. Five of them (FBL3 to FBL7) share structural similarities with Skp2 and contain C-terminal leucine-rich repeats. The other 5 proteins have different putative protein-protein interaction motifs. Specifically, FBS and FBWD4 proteins contain Sec7 and WD40-repeat domains, respectively. The C-terminal region of FBA shares similarity with bacterial protein ApaG while FBG2 shows homology with the F-box protein NFB42. The marked differences in F-box gene expression in human tissues suggest their distinct role in ubiquitin-dependent protein degradation.

Panel 1.3D Summary: Ag2557

The CG50321-01 gene is expressed at a moderate to low level in most of the cell lines and tissues on this panel, with highest expression in a gastric cancer cell line (CT=30.4). This ubiquitous expression suggests a role in cell prolferation and survival.

There is a broad range of expression of this gene in endocrine (metabolic)-related tissues including adrenal, brain, GI tract, liver and skeletal muscle. Targeting this gene and/or gene-product may aid in the treatment of any number of endocrine or metabolically-related diseases, including obesity and diabetes.

This panel demonstrates the expression of this gene in the CNS in an independent group of patients. See panel CNS_Neurodegeneration for a discussion of utility of this gene in the central nervous system.

Panel 4D Summary: Ag 2557

Highest expression of the CG50321-01 transcript is found in kidney (CT=29.1). High levels of expression are also detected in activated B cells (primary B cells and B cell lymphoma), effector Th1 and the eosinphili cell line (EOL-1). At lower levels this transcript is expressed in a wide range of cell types of significance in the immune response in health and disease. This transcrpit encodes for leucine rich protein with a F-box domain. F-box proteins have been described as components of ubiquitin-ligase complexes, in which they bind substrates for ubiquitin-mediated proteolysis. It is therefore theorized that they participate in the regulation of many processes, including cell division, transcription, signal transduction and development (ref 1). Targeting this gene and/or gene-product by small molecules may aid in the treatment of diseases associated with T and B cell or eosinophil involvement and lead to improvement of the symptoms of patients suffering from autoimmune, inflammatory and atopic diseases such as asthma, allergies, inflammatory bowel diseases, lupus erythematosus, rheumatoid arthritis, psoriasis and atopic skin diseases.

Reference:

1. Patton E E, Willems A R, Tyers M. Combinatorial control in ubiquitin-dependent proteolysis: don't Skp the F-box hypothesis.

Trends Genet June 1998;14(6):236-43

The ubiquitin-dependent proteolytic pathway targets many key regulatory proteins for rapid intracellular degradation. Specificity in protein ubiquitination derives from E3 ubiquitin protein ligases, which recognize substrate proteins. Recently, analysis of the E3s that regulate cell division has revealed common themes in structure and function. One particularly versatile class of E3s, referred to as Skp1p-Cdc53p-F-box protein (SCF) complexes, utilizes substrate-specific adaptor subunits called F-box proteins to recruit various substrates to a core ubiquitination complex. A vast array of F-box proteins have been revealed by genome sequencing projects, and the early returns from genetic analysis in several organisms promise that F-box proteins will participate in the regulation of many processes, including cell division, transcription, signal transduction and development.

NOV9 (CG55902-01/AC079907.6: Steroid Binding Protein)

Expression of gene CG55902-01 was assessed using the primer-probe set Ag2626, described in Table JA. Please note that results from Panels 1.3D, 2.2 and 4D have been filed previously.

TABLE JA

Probe Name Ag2626

| | | | |
|---|---|---|---|
| | | | Start |
| Primers | Sequences | Length | Position |

| | | | | |
|---|---|---|---|---|
| Forward | 5′-ttctcaatgagtttggcagc-3′ | (SEQ ID NO:172) | 20 | 365 |
| Probe | TET-5′-aacctggacttcaaggctgaagacca-3′-TAMRA | (SEQ ID NO:173) | 26 | 388 |
| Reverse | 5′-aaacctcagaacccctcctt-3′ | (SEQ ID NO:174) | 20 | 430 |

TABLE JB
CNS_neurodegeneration_v1.0

CNS_neurodegeneration_v1.0 Summary: Ag2626
Expression of the CG55902-01 gene is low/undetectable
in all samples on this panel (CT > 34.5).
NOV10a and NOV10b (CG50307-01 and CG50307-02:
Steroid Dehydogenase-like)

Expression of gene CG50307-01 and variant CG50307-02 was assessed using the primer-probe sets Ag2248 and Ag2548, described in Tables KA and KB. Results of the RTQ-PCR runs are shown in Tables KC, KD, KE, KF, KG, KH, KI and KJ.

TABLE KA

Probe Name Ag2248

| | | | |
|---|---|---|---|
| | | | Start |
| Primers | Sequences | Length | Position |

| | | | | |
|---|---|---|---|---|
| Forward | 5′-agcctacgctgaagagttagc-3′ | (SEQ ID NO:175) | 21 | 425 |
| Probe | TET-5′-aagccgaggtctcaatataatcctga-3′-TMARA | (SEQ ID NO:176) | 26 | 446 |
| Reverse | 5′-acctgcaacttctcctcgtt-3′ | (SEQ ID NO:177) | 20 | 480 |

TABLE KB

Probe Name Ag2548

| | | | |
|---|---|---|---|
| | | | Start |
| Primers | Sequences | Length | Position |

| | | | | |
|---|---|---|---|---|
| Forward | 5′-gacgttggcatcttggtaaata-3′ | (SEQ ID NO:178) | 22 | 612 |
| Probe | TET-5′-cgcagtatttcactcagctgtccgag-3′-TAMRA | (SEQ ID NO:179) | 26 | 658 |
| Reverse | 5′-ttatgatgtcccagagcttgtc-3′ | (SEQ ID NO:180) | 22 | 684 |

TABLE KC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag2248, Run | Ag2548, Run | | Ag2248, Run | Ag2548, Run |
| Tissue Name | 207928610 | 208300028 | Tissue Name | 207928610 | 208300028 |
| AD 1 Hippo | 20.2 | 14.7 | Control | 4.4 | 4.1 |
| | | | (Path) 3 | | |
| | | | Temporal | | |
| | | | Ctx | | |
| AD 2 Hippo | 27.2 | 46.7 | Control | 37.6 | 37.9 |
| | | | (Path) 4 | | |
| | | | Temporal | | |
| | | | Ctx | | |
| AD 3 Hippo | 5.6 | 6.9 | AD 1 | 11.0 | 20.0 |
| | | | Occipital Ctx | | |
| AD 4 Hippo | 10.8 | 11.4 | AD 2 | 0.0 | 0.0 |
| | | | Occipital Ctx | | |
| | | | (Missing) | | |
| AD 5 Hippo | 85.3 | 2.6 | AD 3 | 6.1 | 6.2 |
| | | | Occipital Ctx | | |
| AD 6 Hippo | 69.7 | 50.0 | AD 4 | 21.6 | 22.7 |
| | | | Occipital Ctx | | |
| Control 2 | 42.9 | 45.7 | AD 5 | 14.0 | 11.8 |
| Hippo | | | Occipital Ctx | | |
| Control 4 | 11.3 | 12.5 | AD 5 | 47.3 | 49.0 |
| Hippo | | | Occipital Ctx | | |
| Control | 7.6 | 7.2 | Control 1 | 1.4 | 1.8 |
| (Path) 3 | | | Occipital Ctx | | |
| Hippo | | | | | |
| AD 1 | 18.2 | 20.4 | Control 2 | 81.8 | 83.5 |
| Temporal | | | Occipital Ctx | | |
| Ctx | | | | | |
| AD 2 | 27.4 | 42.6 | Control 3 | 13.1 | 15.9 |
| Temporal | | | Occipital Ctx | | |
| Ctx | | | | | |
| AD 3 | 5.5 | 6.8 | Control 4 | 6.7 | 6.5 |
| Temporal | | | Occipital Ctx | | |
| Ctx | | | | | |
| AD 4 | 17.4 | 22.5 | Control | 91.4 | 100.0 |
| Temporal | | | (Path) 1 | | |
| Ctx | | | Occipital Ctx | | |
| AD 5 Inf | 89.5 | 99.3 | Control | 11.8 | 9.7 |
| Temporal | | | (Path) 2 | | |
| Ctx | | | Occipital Ctx | | |
| AD 5 Sup | 34.6 | 50.7 | Control | 1.6 | 2.0 |
| Temporal | | | (Path) 3 | | |
| Ctx | | | Occipital Ctx | | |
| AD 6 Inf | 42.9 | 42.0 | Control | 15.5 | 15.1 |
| Temporal | | | (Path) 4 | | |
| Ctx | | | Occipital Ctx | | |
| AD 6 Sup | 50.3 | 45.1 | Control 1 | 8.1 | 4.6 |
| Temporal | | | Parietal Ctx | | |
| Ctx | | | | | |
| Control 1 | 3.7 | 3.5 | Control 2 | 30.1 | 33.4 |
| Temporal | | | Parietal Ctx | | |
| Ctx | | | | | |
| Control 2 | 56.6 | 42.6 | Control 3 | 24.0 | 21.9 |
| Temporal | | | Parietal Ctx | | |
| Ctx | | | | | |
| Control 3 | 19.1 | 12.3 | Control | 100.0 | 84.7 |
| Temporal | | | (Path) 1 | | |
| Ctx | | | Parietal Ctx | | |
| Control 3 | 7.1 | 8.0 | Control | 26.8 | 20.3 |
| Temporal | | | (Path) 2 | | |
| Ctx | | | Parietal Ctx | | |
| Control | 64.2 | 74.2 | Control | 4.7 | 3.7 |
| (Path) 1 | | | (Path) 3 | | |
| Temporal | | | Parietal Ctx | | |
| Ctx | | | | | |
| Control | 23.5 | 30.1 | Control | 44.4 | 66.4 |
| (Path) 2 | | | (Path) 4 | | |
| Temporal | | | Parietal Ctx | | |
| Ctx | | | | | |

TABLE KD
Panel 1.3D

| | Rel. Exp. (%) | Rel. Exp. (%) | | Rel. Exp. (%) | Rel. Exp. (%) |
|---|---|---|---|---|---|
| | Ag2248, Run | Ag2548, Run | | Ag2248, Run | Ag2548, Run |
| Tissue Name | 159035206 | 162292266 | Tissue Name | 159035206 | 162292266 |
| Liver | 8.7 | 25.5 | Kidney (fetal) | 5.0 | 14.1 |
| adenocarcinoma | | | | | |
| Pancreas | 1.1 | 1.7 | Renal ca. 7860 | 5.3 | 7.5 |
| Pancreatic Ca. | 2.2 | 6.3 | Renal ca. A498 | 10.2 | 9.0 |
| CAPAN 2 | | | | | |
| Adrenal gland | 6.6 | 6.0 | Renal ca. RXF | 1.5 | 9.0 |
| | | | 393 | | |
| Thyroid | 9.0 | 19.3 | Renal ca. ACHN | 1.7 | 12.8 |
| Salivary gland | 2.9 | 3.7 | Renal ca. UO31 | 5.4 | 15.1 |
| Pituitary gland | 19.8 | 16.2 | Renal ca. TK10 | 1.5 | 6.7 |
| Brain (fetal) | 28.3 | 14.3 | Liver | 1.3 | 0.4 |
| Brain (whole) | 22.7 | 25.2 | Liver (fetal) | 3.1 | 2.3 |
| Brain (amygdala) | 24.7 | 24.3 | Liver ca. | 8.1 | 18.7 |
| | | | (hepatoblast) | | |
| | | | HepG2 | | |
| Brain | 11.6 | 14.6 | Lung | 10.2 | 7.4 |
| (cerebellum) | | | | | |
| Brain | 100.0 | 45.1 | Lung (fetal) | 9.5 | 12.5 |
| (hippocampus) | | | | | |
| Brain (substantia | 5.1 | 7.2 | Lung ca. (small | 11.0 | 16.0 |
| nigra) | | | cell) LX-1 | | |
| Brain (thalamus) | 19.2 | 25.2 | Lung ca. (small | 7.5 | 6.3 |
| | | | cell) NCI-H69 | | |
| Cerebral Cortex | 44.8 | 100.0 | Lung ca. (s. cell | 42.9 | 73.7 |
| | | | var.) SHP-77 | | |
| Spinal cord | 4.8 | 14.9 | Lung ca. (large | 2.7 | 10.1 |
| | | | cell) NCI-H460 | | |
| glio/astro U87MG | 11.7 | 42.3 | Lung ca. (non- | 1.8 | 4.1 |
| | | | sm. cell) A549 | | |
| glio/astro U-118- | 20.7 | 12.0 | Lung Ca. (non- | 11.7 | 28.5 |
| MG | | | s. cell) NCI-H23 | | |
| astrocytoma | 8.1 | 38.2 | Lung ca. (non- | 5.3 | 24.0 |
| SW1783 | | | s. cell) HOP-62 | | |
| neuro*; met SK- | 14.2 | 6.5 | Lung ca. (non- | 5.0 | 15.1 |
| N-AS | | | s. cl) NCI-H522 | | |
| astrocytoma SF- | 3.9 | 15.2 | Lung ca. (squam.) | 3.4 | 12.6 |
| 539 | | | SW 900 | | |
| astrocytoma SNB- | 8.8 | 11.3 | Lung ca. (squam.) | 1.5 | 1.9 |
| 75 | | | NCI-H596 | | |
| glioma SNB-19 | 4.1 | 20.0 | Mammary | 7.5 | 9.6 |
| | | | gland | | |
| glioma U251 | 2.5 | 5.8 | Breast ca.* | 25.3 | 88.9 |
| | | | (pl. ef) MCF-7 | | |
| glioma SF-295 | 3.4 | 24.0 | Breast ca.* | 21.8 | 6.4 |
| | | | (pl. ef) MDA- | | |
| | | | MB-231 | | |
| Heart (Fetal) | 9.7 | 35.1 | Breast ca.* (pl. | 13.6 | 29.3 |
| | | | ef) T47D | | |
| Heart | 3.6 | 11.4 | Breast ca. BT- | 22.1 | 7.4 |
| | | | 549 | | |
| Skeletal muscle | 8.2 | 44.1 | Breast ca. | 5.7 | 11.1 |
| (Fetal) | | | MDA-N | | |
| Skeletal muscle | 5.6 | 47.6 | Ovary | 5.4 | 26.6 |
| Bone marrow | 3.2 | 1.7 | Ovarian ca. | 2.5 | 4.8 |
| | | | OVCAR-3 | | |
| Thymus | 3.5 | 40.6 | Ovarian ca. | 0.6 | 3.6 |
| | | | OVCAR-4 | | |
| Spleen | 5.4 | 10.9 | Ovarian ca. | 3.8 | 13.1 |
| | | | OVCAR-5 | | |
| Lymph node | 2.8 | 4.4 | Ovarian Ca. | 5.9 | 21.2 |
| | | | OVCAR-8 | | |
| Colorectal | 1.9 | 9.4 | Ovarian ca. | 1.4 | 3.1 |
| | | | IGROV-1 | | |
| Stomach | 2.2 | 2.7 | Ovarian ca | 5.3 | 13.1 |
| | | | (ascites) SK- | | |
| | | | OV-3 | | |
| Small intestine | 5.0 | 7.3 | Uterus | 3.9 | 6.0 |
| Colon ca. SW480 | 6.0 | 12.6 | Placenta | 5.2 | 8.8 |
| Colon ca.* | 4.7 | 11.1 | Prostate | 2.0 | 6.7 |
| SW620 (SW480 | | | | | |
| met) | | | | | |
| Colon ca. HT29 | 2.6 | 7.1 | Prostate ca.* | 5.4 | 9.7 |
| | | | (bone met) PC-3 | | |
| Colon ca. HCT- | 9.5 | 22.4 | Testis | 7.7 | 24.8 |
| 116 | | | | | |
| Colon ca. CaCo-2 | 6.7 | 18.0 | Melanoma | 3.3 | 7.7 |
| | | | Hs688(A).T | | |
| CC Well to Mod | 4.8 | 13.2 | Melanoma | 1.2 | 6.9 |
| Diff(ODO3866) | | | (met) | | |
| | | | Hs688(B).T | | |
| Colon ca. HCC- | 17.2 | 10.2 | Melanoma | 1.5 | 5.6 |
| 2998 | | | UACC-62 | | |
| Gastric ca. (liver | 10.8 | 14.7 | Melanoma | 4.3 | 8.1 |
| met) NCI-N87 | | | M14 | | |
| Bladder | 2.6 | 11.0 | Melanoma | 4.8 | 2.9 |
| | | | LOX IMVI | | |
| Trachea | 6.4 | 13.8 | Melanoma* | 6.9 | 10.4 |
| | | | (met) SK- | | |
| | | | MEL-5 | | |
| Kidney | 1.7 | 14.1 | Adipose | 2.3 | 6.0 |

TABLE KB
Panel 2D

| | Rel. Exp. (%) | Rel. Exp. (%) | | Rel. Exp. (%) | Rel. Exp. (%) |
|---|---|---|---|---|---|
| | Ag2248, Run | Ag2548, Run | | Ag2248, Run | Ag2548, Run |
| Tissue Name | 159035545 | 162326203 | Tissue Name | 159035545 | 162326203 |
| Normal Colon | 49.3 | 39.5 | Kidney Margin | 4.7 | 6.3 |
| | | | 8120608 | | |
| CC Well to Mod | 14.2 | 10.7 | Kidney Cancer | 7.1 | 14.0 |
| Diff (ODO3866) | | | 8120613 | | |
| CC Margin | 10.7 | 8.9 | Kidney Margin | 8.8 | 10.7 |
| (ODO3866) | | | 8120614 | | |
| CC Gr.2 | 6.6 | 5.9 | Kidney Cancer | 10.9 | 13.7 |
| rectosigmoid | | | 9010320 | | |
| (ODO3868) | | | | | |
| CC Margin | 5.8 | 6.9 | Kidney Margin | 9.7 | 18.4 |
| (ODO3868) | | | 9010321 | | |
| CC Mod Diff | 38.4 | 21.5 | Normal Uterus | 6.5 | 8.0 |
| (0D03920) | | | | | |
| CC Margin | 14.5 | 9.5 | Uterine Cancer | 42.9 | 24.1 |
| (ODO3920) | | | 064011 | | |
| CC Gr.2 ascend | 25.5 | 15.8 | Normal | 40.3 | 31.0 |
| colon | | | Thyroid | | |
| (ODO3921) | | | | | |
| CC Margin | 7.7 | 5.9 | Thyroid | 21.0 | 21.0 |
| (ODO3921) | | | Cancer | | |
| CC from Partial | 32.5 | 28.5 | Thyroid | 21.9 | 18.4 |
| Hepatectomy | | | Cancer | | |
| (ODO4309) Mets | | | A302152 | | |
| Liver Margin | 12.2 | 9.0 | Thyroid | 37.9 | 39.0 |
| (ODO4309) | | | Margin | | |
| | | | A302153 | | |
| Colon mets to | 15.6 | 8.5 | Normal Breast | 18.9 | 23.8 |
| lung (OD04451- | | | | | |
| 01) | | | | | |
| Lung Margin | 12.6 | 9.2 | Breast Cancer | 14.2 | 20.2 |
| (OD04451-02) | | | | | |
| Normal Prostate | 6.6 | 57.4 | Breast Cancer | 100.0 | 100.0 |
| 6546-1 | | | (OD04590-01) | | |
| Prostate Cancer | 40.3 | 31.0 | Breast Cancer | 87.1 | 90.1 |
| (OD04410) | | | Mets | | |
| | | | (OD04590-03) | | |
| Prostate Margin | 27.0 | 21.8 | Breast Cancer | 37.6 | 37.4 |
| (OD04410) | | | Metastasis | | |
| Prostate Cancer | 28.5 | 18.3 | Breast Cancer | 14.6 | 14.1 |
| (OD04720-01) | | | | | |
| Prostate Margin | 35.8 | 25.0 | Breast Cancer | 27.4 | 28.9 |
| (OD04720-02) | | | | | |
| Normal Lung | 56.6 | 39.0 | Breast Cancer | 46.7 | 41.5 |
| | | | 9100266 | | |
| Lung Met to | 33.4 | 22.7 | Breast Margin | 15.5 | 16.7 |
| Muscle | | | 9100265 | | |
| (ODO4286) | | | | | |
| Muscle Margin | 22.1 | 12.3 | Breast Cancer | 42.3 | 42.9 |
| (ODO4286) | | | A209073 | | |
| Lung Malignant | 33.4 | 27.0 | Breast Margin | 21.3 | 17.2 |
| Cancer | | | A2090734 | | |
| (OD03126) | | | | | |
| Lung Margin | 27.5 | 21.9 | Normal Liver | 5.4 | 4.6 |
| (OD03126) | | | | | |
| Lung Cancer | 13.3 | 14.9 | Liver Cancer | 3.8 | 3.0 |
| (OD04404) | | | | | |
| Lung Margin | 12.0 | 11.6 | Liver Cancer | 4.2 | 2.3 |
| (OD04404) | | | 1025 | | |
| Lung Cancer | 14.1 | 14.3 | Liver Cancer | 3.0 | 1.3 |
| (OD04565) | | | 1026 | | |
| Lung Margin | 6.9 | 11.0 | Liver Cancer | 3.6 | 1.6 |
| (OD04565) | | | 6004-T | | |
| Lung Cancer | 95.9 | 82.4 | Liver Tissue | 11.7 | 9.0 |
| (OD04237-01) | | | 6004-N | | |
| Lung Margin | 15.5 | 13.7 | Liver Cancer | 2.2 | 2.9 |
| (OD04237-02) | | | 6005-T | | |
| Ocular Mel Met | 27.4 | 19.9 | Liver Tissue | 4.4 | 3.8 |
| to Liver | | | 6005-N | | |
| (ODO4310) | | | | | |
| Liver Margin | 5.1 | 3.4 | Normal | 26.6 | 16.0 |
| (ODO4310) | | | Bladder | | |
| Melanoma | 24.8 | 18.8 | Bladder | 5.0 | 3.0 |
| Metastasis | | | Cancer | | |
| Lung Margin | 23.8 | 20.0 | Bladder | 17.1 | 8.8 |
| (OD04321) | | | Cancer | | |
| Normal Kidney | 40.1 | 48.0 | Bladder | 22.2 | 15.5 |
| | | | Cancer | | |
| | | | (OD04718-01) | | |
| Kidney Ca, | 30.6 | 41.8 | Bladder | 21.2 | 15.6 |
| Nuclear grade 2 | | | Normal | | |
| (OD04338) | | | Adjacent | | |
| | | | (OD04718-03) | | |
| Kidney Margin | 16.4 | 15.9 | Normal Ovary | 12.6 | 8.8 |
| (OD04338) | | | | | |
| Kidney Ca | 11.3 | 15.8 | Ovarian | 21.6 | 16.5 |
| Nuclear grade ½ | | | Cancer | | |
| (OD04339) | | | | | |
| Kidney Margin | 19.6 | 24.5 | Ovarian | 40.1 | 33.9 |
| (OD04339) | | | Cancer | | |
| | | | (OD04768-07) | | |
| Kidney Ca, Clear | 20.4 | 30.8 | Ovary Margin | 11.3 | 4.0 |
| cell type | | | (OD04768-08) | | |
| (OD04340) | | | | | |
| Kidney Margin | 18.4 | 13.9 | Normal | 12.2 | 8.8 |
| (OD04348) | | | Stomach | | |
| Kidney Ca, | 13.3 | 6.1 | Gastric Cancer | 5.0 | 3.0 |
| Nuclear grade 3 | | | 9060358 | | |
| (OD04348) | | | | | |
| Kidney Margin | 21.2 | 19.3 | Stomach | 16.0 | 11.0 |
| (OD04348) | | | Margin | | |
| | | | 9060359 | | |
| Kidney Cancer | 19.3 | 19.2 | Gastric Cancer | 16.3 | 12.6 |
| (OD04622-01) | | | 9060395 | | |
| Kidney Margin | 4.4 | 5.3 | Stomach | 13.9 | 10.6 |
| (OD04622-03) | | | Margin | | |
| | | | 9060394 | | |
| Kidney Cancer | 23.8 | 27.0 | Gastric Cancer | 24.0 | 12.1 |
| (OD04450-01) | | | 9060397 | | |
| Kidney Margin | 15.2 | 20.0 | Stomach | 6.4 | 7.1 |
| (OD04450-03) | | | Margin | | |
| | | | 9060396 | | |
| Kidney Cancer | 5.6 | 4.2 | Gastric Cancer | 37.1 | 20.3 |
| 8120607 | | | 064005 | | |

TABLE KF
Panel 3D

| | Rel. Exp. (%) | | Rel. Exp. (%) |
|---|---|---|---|
| | Ag2548, Run | | Ag2548, Run |
| Tissue Name | 164886193 | Tissue Name | 164886193 |
| Daoy-Medulloblastoma | 8.7 | Ca Ski-Cervical epidermoid | 10.6 |
| | | carcinoma (metastasis) | |
| TE671-Medulloblastoma | 10.7 | ES-2-Ovarian clear cell | 11.3 |
| | | carcinoma | |
| D283 Med- | 40.6 | Ramos-Stimulated with | 2.0 |
| Medulloblastoma | | PMA/ionomycin 6 h | |
| PFSK-1-Primitive | 9.0 | Ramos-Stimulated with | 8.8 |
| Neuroectodermal | | PMA/ionomycin 14 h | |
| XF-498-CNS | 9.3 | MEG-01-Chronic | 11.5 |
| | | myelogenous leukemia | |
| | | (megokaryoblast) | |
| SNB-78-Glioma | 12.9 | Raji-Burkitt's lymphoma | 4.5 |
| SF-268-Glioblastoma | 9.4 | Daudi-Burkitt's lymphoma | 12.0 |
| T98G-Glioblastoma | 13.7 | U266-B-cell plasmacytoma | 28.1 |
| SK-N-SH- | 14.9 | CA46-Burkitt's lymphoma | 9.2 |
| Neuroblastoma | | | |
| (metastasis) | | | |
| SF-295-Glioblastoma | 9.9 | RL-non-Hodgkin's B-cell | 2.2 |
| | | lymphoma | |
| Cerebellum | 21.5 | JM1-pre-B-cell lymphoma | 6.3 |
| Cerebellum | 6.0 | Jurkat-T cell leukemia | 18.7 |
| NCI-H292- | 25.7 | TF-1-Erythroleukemia | 9.7 |
| Mucoepidermoid lung | | | |
| carcinoma | | | |
| DMS-114-Small cell | 16.3 | HUT 78-T-cell lymphoma | 17.1 |
| lung cancer | | | |
| DMS-79-Small cell lung | 100.0 | U937-Histiocytic lymphoma | 11.2 |
| cancer | | | |
| NCI-H146-Small cell | 20.9 | KU-812-Myelogenous | 5.3 |
| lung cancer | | leukemia | |
| NCI-H526-Small cell | 36.6 | 769-P-Clear cell renal | 6.2 |
| lung cancer | | carcinoma | |
| NCI-N417-Small cell | 9.7 | Caki-2-Clear cell renal | 8.1 |
| lung cancer | | carcinoma | |
| NCI-H82-Small cell lung | 14.2 | SW 839-Clear cell renal | 2.9 |
| cancer | | carcinoma | |
| NCI-H157-Squamous | 19.6 | G401-Wilms' tumor | 8.8 |
| cell lung cancer | | | |
| (metastasis) | | | |
| NCI-H1155-Large cell | 34.6 | Hs766T-Pancreatic carcinoma | 13.3 |
| lung cancer | | (LN metastasis) | |
| NCI-H1299-Large cell | 19.9 | CAPAN-1-Pancreatic | 7.7 |
| lung cancer | | adenocarcinoma (liver | |
| | | metastasis) | |
| NCI-H727-Lung | 14.2 | SU86.86-Pancreatic | 10.0 |
| carcinoid | | carcinoma (liver metastasis) | |
| NCI-UMC-11-Lung | 12.6 | BxPC-3-Pancreatic | 4.3 |
| carcinoid | | adenocarcinoma | |
| LX-1-Small cell lung | 20.0 | HPAC-Pancreatic | 6.6 |
| cancer | | adenocarcinoma | |
| Colo-205-Colon cancer | 15.8 | MIA PaCa-2-Pancreatic | 4.6 |
| | | carcinoma | |
| KM12-Colon cancer | 9.3 | CFPAC-1-Pancreatic ductal | 19.5 |
| | | adenocarcinoma | |
| KM20L2-Colon cancer | 3.0 | PANC-1-Pancreatic | 9.5 |
| | | epithelioid ductal carcinoma | |
| NCI-H716-Colon cancer | 19.1 | T24-Bladder carcinma | 9.9 |
| | | (transitional cell) | |
| SW-48-Colon | 7.9 | 5637-Bladder carcinoma | 4.7 |
| adenocarcinoma | | | |
| SW1116-Colon | 7.4 | HT-1197-Bladder carcinoma | 6.1 |
| adenocarcinoma | | | |
| LS 174T-Colon | 4.6 | UM-UC-3-Bladder carcinma | 2.8 |
| adenocarcinoma | | (transitional cell) | |
| SW-948-Colon | 1.1 | A204-Rhabdomyosarcoma | 3.4 |
| adenocarcinoma | | | |
| SW-480-Colon | 2.7 | HT-1080-Fibrosarcoma | 10.7 |
| adenocarcinoma | | | |
| NCI-SNU-5-Gastric | 9.3 | MG-63-Osteosarcoma | 1.3 |
| carcinoma | | | |
| KATO III-Gastric | 24.0 | SK-LMS-1-Leiomyosarcoma | 9.5 |
| carcinoma | | (vulva) | |
| NCI-SNU-16-Gastric | 9.5 | SJRH30-Rhabdomyosarcoma | 10.2 |
| carcinoma | | (met to bone marrow) | |
| NCI-SNU-1-Gastric | 12.2 | A431-Epidermoid carcinoma | 5.0 |
| carcinoma | | | |
| RF-1-Gastric | 5.1 | WM266-4-Melanoma | 10.5 |
| adenocarcinoma | | | |
| RF-48-Gastric | 8.1 | DU 145-Prostate carcinoma | 0.0 |
| adenocarcinoma | | (brain metastasis) | |
| MKN-45-Gastric | 5.3 | MDA-MB-468-Breast | 20.7 |
| carcinoma | | adenocarcinoma | |
| NCI-N87-Gastric | 7.4 | SCC-4-Squamous cell | 0.0 |
| carcinoma | | carcinoma of tongue | |
| OVCAR-5-Ovarian | 2.7 | SCC-9-Squamous cell | 0.0 |
| carcinoma | | carcinoma of tongue | |
| RL95-2-Uterine | 3.8 | SCC-15-Squamous cell | 0.0 |
| carcinoma | | carcinoma of tongue | |
| HelaS3-Cervical | 10.7 | CAL 27-Squamous cell | 5.5 |
| adenocarcinoma | | carcinoma of tongue | |

TABLE KG
Panel 4D

| | Rel. Exp. (%) | | Rel. Exp. (%) |
|---|---|---|---|
| | Ag2248, Run | | Ag2248, Run |
| Tissue Name | 159034717 | Tissue Name | 159034717 |
| Secondary Th1 act | 27.2 | HUVEC IL-1beta | 8.4 |
| Secondary Th2 act | 33.4 | HUVEC IFN gamma | 14.4 |
| Secondary Tr1 act | 37.4 | HUVEC TNF alpha + IFN | 7.4 |
| | | gamma | |
| Secondary Th1 rest | 11.7 | HUVEC TNF alpha + IL4 | 6.6 |
| Secondary Th2 rest | 10.4 | HUVEC IL-11 | 11.8 |
| Secondary Tr1 rest | 12.5 | Lung Microvascular EC | 9.9 |
| | | none | |
| Primary Th1 act | 28.5 | Lung Microvascular EC | 18.2 |
| | | TNF alpha + IL-1beta | |
| Primary Th2 act | 29.3 | Microvascular Dermal EC | 28.9 |
| | | none | |
| Primary Tr1 act | 29.3 | Microsvasular Dermal EC | 20.2 |
| | | TNF alpha + IL-1beta | |
| Primary Th1 rest | 62.4 | Bronchial epithelium | 20.7 |
| | | TNF alpha + IL1beta | |
| Primary Th2 rest | 39.8 | Small airway epithelium | 6.9 |
| | | none | |
| Primary Tr1 rest | 15.3 | Small airway epithelium | 40.3 |
| | | TNF alpha + IL-1beta | |
| CD45RA CD4 | 18.6 | Coronery artery SMC rest | 15.4 |
| lymphocyte act | | | |
| CD45RO CD4 | 20.9 | Coronery artery SMC | 6.8 |
| lymphocyte act | | TNF alpha + IL-1beta | |
| CD8 lymphocyte act | 14.7 | Astrocytes rest | 20.0 |
| Secondary CD8 | 11.9 | Astrocytes TNF alpha + IL- | 15.8 |
| lymphocyte rest | | 1beta | |
| Secondary CD8 | 19.9 | KU-812 (Basophil) rest | 8.1 |
| lymphocyte act | | | |
| CD4 lymphocyte none | 8.2 | KU-812 (Basophil) | 20.2 |
| | | PMA/ionomycin | |
| 2ry Th1/Th2/Tr1_anti- | 17.4 | CCD1106 (Keratinocytes) | 11.5 |
| CD95 CH11 | | none | |
| LAK cells rest | 19.2 | CCD1106 (Keratinocytes) | 5.3 |
| | | TNF alpha + IL-1beta | |
| LAK cells IL-2 | 18.2 | Liver cirrhosis | 2.4 |
| LAK cells IL-2 + IL-12 | 11.0 | Lupus kidney | 1.8 |
| LAK cells IL-2 + IFN | 19.5 | NCI-H292 none | 39.5 |
| gamma | | | |
| LAK cells IL-2 + IL-18 | 17.7 | NCI-H292 IL-4 | 38.2 |
| LAK cells | 3.6 | NCI-H292 IL-9 | 40.1 |
| PMA/ionomycin | | | |
| NK Cells IL-2 rest | 11.0 | NCI-H292 IL-13 | 18.2 |
| Two Way MLR 3 day | 19.2 | NCI-H292 IFN gamma | 14.7 |
| Two Way MLR 5 day | 8.9 | HPAEC none | 19.2 |
| Two Way MLR 7 day | 6.7 | HPAEC TNF alpha + | 28.5 |
| | | IL-1beta | |
| PBMC rest | 5.8 | Lung fibroblast none | 17.4 |
| PBMC PWM | 40.6 | Lung fibroblast TNF alpha + | 17.1 |
| | | IL-1beta | |
| PBMC PHA-L | 25.9 | Lung fibroblast IL-4 | 30.4 |
| Ramos (B cell) none | 26.6 | Lung fibroblast IL-9 | 20.2 |
| Ramos (B cell) | 100.0 | Lung fibroblast IL-13 | 16.3 |
| ionomycin | | | |
| B lymphocytes PWM | 35.6 | Lung fibroblast IFN | 28.1 |
| | | gamma | |
| B lymphocytes CD40L | 29.7 | Dermal fibroblast | 32.3 |
| and IL-4 | | CCD1070 rest | |
| EOL-1 dbcAMP | 10.5 | Dermal fibroblast | 57.0 |
| | | CCD1070 TNF alpha | |
| EOL-1 dbcAMP | 7.5 | Dermal fibroblast | 15.3 |
| PMA/ionomycin | | CCD1070 IL-1beta | |
| Dendritic cells none | 11.6 | Dermal fibroblast IFN | 12.1 |
| | | gamma | |
| Dendritic cells LPS | 7.7 | Dermal fibroblast IL-4 | 20.3 |
| Dendritic cells anti- | 9.6 | IBD Colitis 2 | 2.3 |
| CD40 | | | |
| Monocytes rest | 12.6 | IBD Crohn's | 2.6 |
| Monocytes LPS | 21.0 | Colon | 11.5 |
| Macrophages rest | 24.5 | Lung | 16.2 |
| Macrophages LPS | 14.8 | Thymus | 38.7 |
| HUVEC none | 25.9 | Kidney | 71.2 |
| HUVEC starved | 40.9 | | |

TABLE KH
Panel 5 Islet

| | Rel. Exp. (%) | | Rel. Exp. (%) |
|---|---|---|---|
| | Ag2248, Run | | Ag2248, Run |
| Tissue Name | 233070521 | Tissue Name | 233070521 |
| 97457_Patient- | 23.7 | 94709_Donor 2 AM - A_adipose | 10.5 |
| 02go_adipose | | | |
| 97476_Patient- | 12.3 | 94710_Donor 2 AM - B_adipose | 5.0 |
| 07sk_skeletal muscle | | | |
| 97477_Patient- | 18.9 | 94711_Donor 2 AM - C_adipose | 5.3 |
| 07ut_uterus | | | |
| 97478_Patient- | 35.6 | 94712_Donor 2 AD - A_adipose | 14.5 |
| 07pl_placenta | | | |
| 99167_Bayer Patient 1 | 25.0 | 94713_Donor 2 AD - B_adipose | 25.2 |
| 97482_Patient- | 23.2 | 94714_Donor 2 AD - C_adipose | 18.7 |
| 08ut_uterus | | | |
| 97483_Patient- | 25.5 | 94742_Donor 3 U - | 7.4 |
| 08pl_placenta | | A_Mesenchymal Stem Cells | |
| 97486_Patient- | 0.7 | 94743_Donor 3 U - | 11.2 |
| 09sk_skeletal muscle | | B_Mesenchymal Stem Cells | |
| 97487_Patient- | 23.0 | 94730_Donor 3 AM - A_adipose | 14.6 |
| 09ut_uterus | | | |
| 97488_Patient- | 15.3 | 94731_Donor 3 AM - B_adipose | 4.5 |
| 09pl_placenta | | | |
| 97492_Patient- | 15.1 | 94732_Donor 3 AM - C_adipose | 10.5 |
| 10ut_uterus | | | |
| 97493_Patient- | 52.9 | 94733_Donor 3 AD - A_adipose | 40.1 |
| 10pl_placenta | | | |
| 97495_Patient- | 9.1 | 94734_Donor 3 AD - B_adipose | 16.0 |
| 11go_adipose | | | |
| 97496_Patient- | 10.2 | 94735_Donor 3 AD - C_adipose | 14.0 |
| 11sk_skeletal muscle | | | |
| 97497_Patient- | 21.8 | 77138_Liver_HepG2untreated | 100.0 |
| 11ut_uterus | | | |
| 97498_Patient- | 36.3 | 73556_Heart_Cardiac stromal cells | 9.5 |
| 11pl_placenta | | (primary) | |
| 97500_Patient- | 21.0 | 81735_Small Intestine | 8.7 |
| 12go_adipose | | | |
| 97501_Patient- | 35.4 | 72409_Kidney_Proximal | 16.7 |
| 12sk_skeletal muscle | | Convoluted Tubule | |
| 97502_Patient- | 15.5 | 82685_Small intestine_Duodenum | 5.1 |
| 12ut_uterus | | | |
| 97503_Patient- | 9.5 | 90650_Adrenal_Adrenocortical | 18.0 |
| 12pl_placenta | | adenoma | |
| 94721_Donor 2 U - | 8.3 | 72410_Kidney_HRCE | 42.0 |
| A_Mesenchymal Stem | | | |
| Cells | | | |
| 94722_Donor 2 U - | 8.3 | 72411_Kidney_HRE | 27.0 |
| B_Mesenchymal Stem | | | |
| Cells | | | |
| 94723_Donor 2 U - | 20.4 | 73139_Uterus_Uterine smooth | 41.5 |
| C_Mesenchymal Stem | | muscle cells | |
| Cells | | | |

TABLE KI
Panel 5D

| | Rel. Exp. (%) | | Rel. Exp. (%) |
|---|---|---|---|
| | Ag2248, Run | | Ag2248, Run |
| Tissue Name | 166667616 | Tissue Name | 166667616 |
| 97457_Patient- | 44.1 | 94709_Donor 2 AM - A_adipose | 24.3 |
| 02go_adipose | | | |
| 97476_Patient- | 13.6 | 94710_Donor 2 AM - B_adipose | 7.0 |
| 07sk_skeletal muscle | | | |
| 97477_Patient- | 42.0 | 94711_Donor 2 AM - C_adipose | 3.1 |
| 07ut_uterus | | | |
| 97478_Patient- | 64.6 | 94712_Donor 2 AD - A_adipose | 13.9 |
| 07pl_placenta | | | |
| 97481_Patient- | 29.7 | 94713_Donor 2 AD - B_adipose | 39.2 |
| 08sk_skeletal muscle | | | |
| 97482_Patient- | 19.5 | 94714_Donor 2 AD - C_adipose | 35.4 |
| 08ut_uterus | | | |
| 97483_Patient- | 42.0 | 94742_Donor 3 U - | 17.2 |
| 08pl_placenta | | A_Mesenchymal Stem Cells | |
| 97486_Patient- | 7.3 | 94743_Donor 3 U - | 6.4 |
| 09sk_skeletal muscle | | B_Mesenchymal Stem Cells | |
| 97487_Patient- | 26.2 | 94730_Donor 3 AM - A_adipose | 10.0 |
| 09ut_uterus | | | |
| 97488_Patient- | 31.0 | 94731_Donor 3 AM - B_adipose | 6.5 |
| 09pl_placenta | | | |
| 97492_Patient- | 30.4 | 94732_Donor 3 AM - C_adipose | 7.6 |
| 10ut_uterus | | | |
| 97493_Patient- | 100.0 | 94733_Donor 3 AD - A_adipose | 19.5 |
| 10pl_placenta | | | |
| 97495_Patient- | 13.1 | 94734_Donor 3 AD - B_adipose | 25.3 |
| 11go_adipose | | | |
| 97496_Patient- | 9.3 | 94735_Donor 3 AD - C_adipose | 9.1 |
| 11sk_skeletal muscle | | | |
| 97497_Patient- | 30.1 | 77138_Liver_HepG2untreated | 51.1 |
| 11ut_uterus | | | |
| 97498_Patient- | 25.0 | 73556_Heart_Cardiac stromal cells | 4.1 |
| 11pl_placenta | | (primary) | |
| 97500_Patient- | 24.0 | 81735_Small Intestine | 13.2 |
| 12go_adipose | | | |
| 97501_Patient- | 70.7 | 72409_Kidney_Proximal | 21.6 |
| 12sk_skeletal muscle | | Convoluted Tubule | |
| 97502_Patient- | 13.8 | 82685_Small intestine_Duodenum | 3.7 |
| 12ut_uterus | | | |
| 97503_Patient- | 8.5 | 90650_Adrenal_Adrenocortical | 10.9 |
| 12pl_placenta | | adenoma | |
| 94721_Donor 2 U - | 12.4 | 72410_Kidney_HRCE | 22.5 |
| A_Mesenchymal Stem | | | |
| Cells | | | |
| 94722_Donor 2 U - | 19.9 | 72411_Kidney_HRE | 25.3 |
| B_Mesenchymal Stem | | | |
| Cells | | | |
| 94723_Donor 2 U - | 23.5 | 73139_Uterus_Uterine smooth | 28.7 |
| C_Mesenchymal Stem | | muscle cells | |
| Cells | | | |

TABLE KJ
Panel CNS_1

| | | | Rel. Exp. |
|---|---|---|---|
| | Rel. Exp. (%) | | (%) |
| | Ag2248, | | Ag2248, |
| | Run | | Run |
| Tissue Name | 171649039 | Tissue Name | 171649039 |
| BA4 Control | 39.8 | BA17 PSP | 29.1 |
| BA4 Control2 | 41.8 | BA17 PSP2 | 13.1 |
| BA4 | 10.7 | Sub Nigra Control | 22.8 |
| Alzheimer's2 | | | |
| BA4 Parkinson's | 49.7 | Sub Nigra Control2 | 42.6 |
| BA4 | 100.0 | Sub Nigra | 15.1 |
| Parkinson's2 | | Alzheimer's2 | |
| BA4 | 42.9 | Sub Nigra | 54.7 |
| Huntington's | | Parkinson's2 | |
| BA4 | 14.5 | Sub Nigra | 58.6 |
| Huntington's2 | | Huntington's | |
| BA4 PSP | 4.4 | Sub Nigra | 48.6 |
| | | Huntington's2 | |
| BA4 PSP2 | 20.0 | Sub Nigra PSP2 | 5.1 |
| BA4 Depression | 11.7 | Sub Nigra | 8.7 |
| | | Depression | |
| BA4 | 7.0 | Sub Nigra | 11.4 |
| Depression2 | | Depression2 | |
| BA7 Control | 71.2 | Glob Palladus | 10.4 |
| | | Control | |
| BA7 Control2 | 30.4 | Glob Palladus | 5.9 |
| | | Control2 | |
| BA7 | 9.2 | Glob Palladus | 13.8 |
| Alzheimer's2 | | Alzheimer's | |
| BA7 Parkinson's | 17.7 | Glob Palladus | 2.7 |
| | | Alzheimer's2 | |
| BA7 | 60.3 | Glob Palladus | 50.0 |
| Parkinson's2 | | Parkinson's | |
| BA7 | 44.8 | Glob Palladus | 10.7 |
| Huntington's | | Parkinson's2 | |
| BA7 | 49.0 | Glob Palladus PSP | 6.9 |
| Huntington's2 | | | |
| BA7 PSP | 34.4 | Glob Palladus PSP2 | 7.2 |
| BA7 PSP2 | 34.2 | Glob Palladus | 4.6 |
| | | Depression | |
| BA7 Depression | 16.4 | Temp Pole Control | 14.6 |
| BA9 Control | 35.8 | Temp Pole Control2 | 51.1 |
| BA9 Control2 | 59.9 | Temp Pole | 7.7 |
| | | Alzheimer's | |
| BA9 Alzheimer's | 4.0 | Temp Pole | 9.0 |
| | | Alzheimer's2 | |
| BA9 | 20.0 | Temp Pole | 18.6 |
| Alzheimer's2 | | Parkinson's | |
| BA9 Parkinson's | 36.6 | Temp Pole | 48.6 |
| | | Parkinson's2 | |
| BA9 | 57.8 | Temp Pole | 44.4 |
| Parkinson's2 | | Huntington's | |
| BA9 | 57.8 | Temp Pole PSP | 2.6 |
| Huntington's | | | |
| BA9 | 24.1 | Temp Pole PSP2 | 5.7 |
| Huntington's2 | | | |
| BA9 PSP | 12.5 | Temp Pole | 11.0 |
| | | Depression2 | |
| BA9 PSP2 | 3.8 | Cing Gyr Control | 63.7 |
| BA9 Depression | 7.3 | Cing Gyr Control2 | 37.4 |
| BA9 | 18.6 | Cing Gyr Alzheimer's | 28.5 |
| Depression2 | | | |
| BA17 Control | 40.1 | Cing Gyr | 14.3 |
| | | Alzheimer's2 | |
| BA17 Control2 | 35.1 | Cing Gyr Parkinson's | 32.8 |
| BA17 | 6.8 | Cing Gyr | 55.1 |
| Alzheimer's2 | | Parkinson's2 | |
| BA17 | 39.0 | Cing Gyr | 79.6 |
| Parkinson's | | Huntington's | |
| BA17 | 51.1 | Cing Gyr | 19.2 |
| Parkinson's2 | | Huntington's2 | |
| BA17 | 31.6 | Cing Gyr PSP | 14.2 |
| Huntington's | | | |
| BA17 | 20.2 | Cing Gyr PSP2 | 10.8 |
| Huntington's2 | | | |
| BA17 | 11.3 | Cing Gyr Depression | 4.8 |
| Depression | | | |
| BA17 | 29.5 | Cing Gyr | 19.3 |
| Depression2 | | Depression2 | |

CNS_neurodegeneration_v1.0 Summary: Ag2248/Ag2548

Two experiments with two different probe and primer sets produce results that are in very good agreement, with highest expression of the CG50307-01 gene in the occipital and parietal cortex (CTs=27-29) of the brains of control patients. While this geen does not appear to be differentially expressed in Alzheimer's disease, these results confirm confirm the expression of this gene at moderate to high levels in the brains of an independent group of patients. Please see Panel 1.3d for discussion of utility in the central nervous system.

Panel 1.3D Summary: Ag2248/Ag2548

Two experiments with two different probe and primer sets show widespread expression of the CG50307-01 gene, with highest expression seen in regions of the brain (CTs=28-29).

This gene encodes a protein that is homologous to steroid dehydrogenase. Steroid treatment is used in a number of clinical conditions including Alzheimer's disease (estrogen), treatment of symptoms associated with menopause (estrogen), multiple sclerosis (glucocorticoids), and spinal cord injury (methylprednisolone). Treatment with an antagonst of this gene product, or reduction of the levels of this gene product could slow steroid degredation and lower the necessary amount given for therapeutic effect, thusreducing peripheral side effects.

This gene is moderately expressed in a variety of metabolic tissues including pancreas, adrenal, thyroid, pituitary, adult and fetal heart, adult and fetal skeletal muscle, fetal liver, and adipose. Thus, this gene product may be a small molecule drug target for the treatment of metabolic disease, including obesity and Types 1 and 2 diabetes.

The ubiquitous expression of this gene in this panel also suggests that the protein encoded by this gene plays a role in cell survival and proliferation for a majority of cell types. Furthermore, there are significant levels of expression in the lung cancer cell line SHP-77. Thus, expression of this gene could potentially be used as a diagnostic marker for some forms of lung cancer. Modulation of the gene product may also play role in treating lung cancer.

References:

Matsumoto T, Tamaki T, Kawakami M, Yoshida M, Ando M, Yamada H. Early complications of high-dose methylprednisolone sodium succinate treatment in the follow-up of acute cervical spinal cord injury. Spine Feb. 15, 2001;26(4):426-30

STUDY DESIGN: A prospective, randomized, and double-blind study comparing high-dose methylprednisolone sodium succinate (MPSS) with placebo, in the treatment of patients with acute cervical spinal cord injury. OBJECTIVES: To evaluate the complications of high-dose MPSS in patients with acute cervical spinal cord injury when administered within 8 hours of injury. SUMMARY OF BACKGROUND DATA: High-dose therapy with MPSS has been demonstrated to improve the recovery of motor function in patients with acute cervical spinal cord injury. However, little is known about the follow-up complications. METHODS: Forty-six patients, 42 men and 4 women (mean age, 60.6 years; range, 18-84), were included in the study: 23 in the MPSS group and 23 in the placebo group. They were treated without surgery for spinal cord injury in the cervical spine, and were enrolled in the trial if a diagnosis had been made and treatment had begun within 8 hours. Complications of high-dose therapy with MPSS were compared with placebo treatment throughout the study period and up to 2 months after injury. RESULTS: The MPSS group had 13 patients (56.5%) with complications, whereas the placebo group had 8 (34.8%). The difference between the two groups was not statistically significant (P=0.139). There were eight instances of pulmonary complication with MPSS (34.8%) and one instance (4.34%) with placebo (P=0.009). There were four instances of gastrointestinal complication (17.4%) with MPSS and none with placebo (P=0.036). Pulmonary (complications were more prevalent in patients aged more than 60 years (P=0.029). CONCLUSION: Aged patients with cervical spinal injury may be more likely to have pulmonary side effects (P=0.029) after high-dose therapy with MPSS and thus deserve special care.

Holinka C F.Design and conduct of clinical trials in hormone replacement therapy. Ann N Y Acad Sci September 2001; 943:89-108

Postmenopausal hormone replacement therapy represents an area of outstanding importance in preventive medicine that greatly affects personal well-being as well as public health. The number of women living in the United States who are 50 years or older has been estimated at nearly 50 million. Many of those women are likely to be eligible for postmenopausal hormone replacement, which may consist either of estrogen replacement therapy (ERT) in women without a uterus or, more frequently, estrogen/progestin combination therapy (HRT) in women with a uterus. This chapter first presents an overview of general regulatory requirements pertaining to the design and conduct of clinical studies in support of marketing approval for a drug product. These requirements include, but are not restricted to, studies in HRT. The chapter next discusses the design and conduct of clinical trials in support of marketing approval for the indications: treatment of moderate to severe vasomotor symptoms and vulvovaginal atrophy; prevention of osteoporosis; and protection by adjunctive progestin against estrogen-induced endometrial hyperplasia/cancer in women with a uterus. Finally, data related to the potential cardioprotective action of HRT and its protection against Alzheimer's disease and colon cancer are discussed.

Burkman R T, Collins J A, Greene R A. Current perspectives on benefits and risks of hormone replacement therapy. Am J Obstet Gynecol August 2001; 185(2 Suppl):S13-23.

Hormone replacement therapy with estrogen alone or with added progestin relieves menopausal symptoms and physical changes associated with depleted endogenous estrogen levels. Estrogen replacement has also demonstrated a clear benefit in the prevention of osteoporosis. Hormone replacement therapy with added progestin maintains spinal bone density, protects against postmenopausal hip fractures, and provides these benefits even when therapy is started after age 60. More recently, additional benefits have emerged. Current estrogen and hormone replacement therapy users have a 34% reduction in the risk of colorectal cancer and a 20% to 60% reduction in the risk of Alzheimer's disease. Until recently, the body of evidence indicated that hormone replacement therapy with estrogen only reduced cardiovascular disease risk by 40% to 50% in healthy patients; whether the findings of 3 ongoing trials will change this conclusion is pending availability of the final results. The many benefits of estrogen and hormone replacement therapy must be weighed against a slight increase in the risk of breast cancer diagnosis with use for 5 or more years, but which disappears following cessation of therapy. Overall, estrogen and hormone replacement therapy improves the quality of life and increases life expectancy for most menopausal women.

Gaillard P J, van Der Meide P H, de Boer A G, Breimer D D. Glucocorticoid and type 1 interferon interactions at the blood-brain barrier: relevance for drug therapies for multiple sclerosis. Neuroreport Jul. 20, 2000 ;12(10):2189-93.

The pharmacological effect of glucocorticoids and type 1 interferons (IFNs), simultaneously used as therapeuticals for multiple sclerosis (MS), on the (inflamed) blood-brain barrier (BBB) was investigated in vitro. Although both drugs additively decreased BBB permeability, they did not prevent the increase in BBB permeability induced by lipopolysaccharide (LPS), which served as a pro-inflammatory stimulus. The beneficial clinical effect of glucocorticoid and IFN therapy for MS seems there-fore not to be mediated through a direct action at the level of the BBB. Most strikingly, however, pretreatment with type 1 IFNs (alpha and beta) potentiated the effect of glucocorticoids by two orders of magnitude. This lead us to hypothesize that type 1 IFNs may restore the dysfunctional T-helper 1 (Th1)/Th2 balance associated with MS, by a mechanism that involves an increased sensitivity for glucocorticoids.

Panel 2D Summary: Ag2248/Ag2548

The expression of the CG50307-01 gene shows good concordance between two independent runs. The highest level of expression was seen in a breast cancer sample (CTs=27-29). In addition, this gene appears to be overexpressed in ovarian, gastric, breast, uterine, lung and colon cancers relative to the normal adjacent tissues from these patients. Therefore, the expression of this gene could be of use as a diagnostic marker for the presence of these cancers. Furthermore, therapeutic inhibition of the activity of this gene product may be effective in the treatment of these cancers.

Panel 3D Summary: Ag2548

The CG50307-01 gene is expressed at a low to moderate level in most of the cells and tissues used in this panel, with highest expression in the small cell lung cancer cell line DMS-79 (CT=27.79). This ubiquitous expression suggests that the gene product plays a role in cell survival and proliferation for a majority of cell types except cell lines derived from tongue squamous cell carcinoma.

Panel 4D Summary: Ag2248

The CG50307-01 gene encodes a steroid dehydrogenase-like protein and is expressed at moderate levels (CT=28-32) in numerous immune cell types and tissues. Small molecule antagonists that block the function of the steroid dehydrogenase-like protein encoded by this gene may be useful as therapeutics that reduce or eliminate the symptoms of patients suffering from autoimmune and inflammatory diseases such as asthma, allergies, inflammatory bowel disease, lupus erythematosus, or rheumatoid arthritis. Please note that data from a second run using the probe and primer set Ag2548 is not included. The amp plot suggests that there were experimental difficulties with this run.

Panel 5 Islet Summary: Ag2248

The expression of this novel steroid dehydrogenase-like gene, CG50307-01, is highest in the liver HepG2 cell line, (CT=32.1). Lower but still significant levels of expression are seen in several placenta samples, uterine smooth muscle, adipose samples, differentiated mesenchymal stem cells, kidney and skeletal muscle from a diabetic patient. Expression in liver cells and placenta suggests that the role of this novel steroid dehydrogenase may be similar to the role of other steroid dehydrogenases which are involved in steroid and bile acid metabolism. Very low expression of this gene is also seen in a human pancreatic islet sample. Therefore, small molecule therapeutics against this gene product may be effective in disorders in which expression of this gene is dysregulated.

Panel 5D Summary: Ag2248

The expression of the CG50307-01 gene is generally similar to that in panel 5I, although the relative abundances in each of the tissues are different. This panel shows highest expression of this steroid dehydrogenase-like gene in placenta from a diabetic patient (CT=32.2), with lower expression in other placenta samples. Relative expression of this gene is also high in the skeletal muscle of a diabetic patient and in liver HepG2 cells. Low but significant levels of expression are also seen in some adipose samples and in differentiated mesenchymal stem cells, in kidney and in uterus. Expression in liver cells and placenta suggests that the role of this novel steroid dehydrogenase may be similar to the role of other steroid dehydrogenases which are involved in steroid and bile acid metabolism. Small molecule therapeutics against this gene product may be effective in disorders in which expression of this gene is dysregulated.

Panel CNS_{—}1 Summary: Ag2248

This panel confirms expression of the CG50307-01 gene in the brain. Please see Panel 1.3D for discussion of potential utility in the central nervous system.

NOV11 (CG50311-01: Novel Nonmuscle Myosin)

Expression of gene CG50311-01 was assessed using the primer-probe set Ag2546, described in Table LA. Results of the RTQ-PCR runs are shown in Tables LB, LC and LD.

TABLE LA

Probe Name Ag2546

| | | | |
|---|---|---|---|
| | | | Start |
| Primers | Sequences | Length | Position |

| | | | | |
|---|---|---|---|---|
| Forward | 5′-gttctgtgtggtcatcaatcct-3′ | (SEQ ID NO:181) | 22 | 487 |
| Probe | TET-5′-caagaacctgcccatctactctgaaga-3′-TAMRA | (SEQ ID NO:182) | 27 | 511 |
| Reverse | 5′-cttgcccttgtacatttcca-3′ | (SEQ ID NO:183) | 20 | 543 |

TABLE LB
Panel 1.3D

| | Rel. Exp. (%) | | Rel. Exp. (%) |
|---|---|---|---|
| | Ag2546, | | Ag2546, |
| Tissue Name | Run 165532775 | Tissue Name | Run 165532775 |
| Liver adenocarcinoma | 15.4 | Kidney (fetal) | 9.0 |
| Pancreas | 4.0 | Renal ca. 786-0 | 44.1 |
| Pancreatic ca. CAPAN 2 | 9.3 | Renal ca. A498 | 38.2 |
| Adrenal gland | 3.8 | Renal ca. RXF 393 | 41.5 |
| Thyroid | 5.2 | Renal ca. ACHN | 20.9 |
| Salivary gland | 6.7 | Renal ca. UO-31 | 63.7 |
| Pituitary gland | 1.4 | Renal ca. TK-10 | 8.7 |
| Brain (fetal) | 2.6 | Liver | 1.5 |
| Brain (whole) | 6.8 | Liver (fetal) | 6.1 |
| Brain (amygdala) | 6.4 | Liver ca. | 15.4 |
| | | (hepatoblast) HepG2 | |
| Brain (cerebellum) | 4.5 | Lung | 19.8 |
| Brain (hippocampus) | 4.6 | Lung (fetal) | 9.5 |
| Brain (substantia nigra) | 3.2 | Lung ca. (small cell) | 10.7 |
| | | LX-1 | |
| Brain (thalamus) | 3.7 | Lung ca. (small cell) | 14.6 |
| | | NCI-H69 | |
| Cerebral Cortex | 5.1 | Lung ca. (s. cell var.) | 19.8 |
| | | SHP-77 | |
| Spinal cord | 5.8 | Lung ca. (large | 11.1 |
| | | cell)NCI-H460 | |
| glio/astro U87-MG | 15.9 | Lung ca. (non-sm. | 3.8 |
| | | cell) A549 | |
| glio/astro U-118-MG | 100.0 | Lung ca. (non-s. cell) | 2.4 |
| | | NCI-H23 | |
| astrocytoma SW1783 | 54.7 | Lung ca. (non-s. cell) | 29.7 |
| | | HOP-62 | |
| neuro*; met SK-N-AS | 2.7 | Lung ca. (non-s. cl) | 1.6 |
| | | NCI-H522 | |
| astrocytoma SF-539 | 27.9 | Lung ca. (squam.) | 17.8 |
| | | SW 900 | |
| astrocytoma SNB-75 | 60.3 | Lung ca. (squam.) | 10.7 |
| | | NCI-H596 | |
| glioma SNB-19 | 16.3 | Mammary gland | 12.2 |
| glioma U251 | 54.0 | Breast ca.* (pl. ef) | 7.1 |
| | | MCF-7 | |
| glioma SF-295 | 28.5 | Breast ca.* (pl. ef) | 64.2 |
| | | MDA-MB-231 | |
| Heart (Fetal) | 4.0 | Breast ca.* (pl. ef) | 4.2 |
| | | T47D | |
| Heart | 9.3 | Breast ca. BT-549 | 52.5 |
| Skeletal muscle (Fetal) | 5.3 | Breast ca. MDA-N | 0.8 |
| Skeletal muscle | 6.8 | Ovary | 14.4 |
| Bone marrow | 8.2 | Ovarian ca. OVCAR-3 | 11.1 |
| Thymus | 7.7 | Ovarian ca. OVCAR-4 | 19.9 |
| Spleen | 12.2 | Ovarian ca. OVCAR-5 | 17.0 |
| Lymph node | 28.3 | Ovarian ca. OVCAR-8 | 4.9 |
| Colorectal | 13.1 | Ovarian ca. IGROV-1 | 2.8 |
| Stomach | 9.3 | Ovarian ca. (ascites) | 31.2 |
| | | SK-OV-3 | |
| Small intestine | 11.5 | Uterus | 40.6 |
| Colon ca. SW480 | 8.1 | Placenta | 9.5 |
| Colon ca.* SW620 | 7.4 | Prostate | 3.2 |
| (SW480 met) | | | |
| Colon ca. HT29 | 3.6 | Prostate ca.* (bone | 7.0 |
| | | met) PC-3 | |
| Colon ca. HCT-116 | 6.9 | Testis | 2.8 |
| Colon ca. CaCo-2 | 9.0 | Melanoma | 22.4 |
| | | Hs688(A).T | |
| CC Well to Mod Diff | 29.3 | Melanoma* (met) | 27.2 |
| (ODO3866) | | Hs688(B).T | |
| Colon ca. HCC-2998 | 6.8 | Melanoma UACC-62 | 7.1 |
| Gastric ca. (liver met) | 15.1 | Melanoma M14 | 51.4 |
| NCI-N87 | | | |
| Bladder | 21.9 | Melanoma LOX | 4.5 |
| | | IMVI | |
| Trachea | 8.5 | Melanoma* (met) | 6.6 |
| | | SK-MEL-5 | |
| Kidney | 5.8 | Adipose | 14.3 |

TABLE LC
Panel 2.2

| | Rel. Exp. (%) | | Rel. Exp. (%) |
|---|---|---|---|
| | Ag2546, Run | | Ag2546, |
| Tissue Name | 174575196 | Tissue Name | Run 174575196 |
| Normal Colon | 33.4 | Kidney Margin | 100.0 |
| | | (OD04348) | |
| Colon cancer (OD06064) | 60.7 | Kidney malignant | 7.9 |
| | | cancer (OD06204B) | |
| Colon Margin | 29.1 | Kidney normal adjacent | 10.4 |
| (OD06064) | | tissue (OD06204E) | |
| Colon cancer (OD06159) | 5.7 | Kidney Cancer | 34.4 |
| | | (OD04450-01) | |
| Colon Margin | 34.6 | Kidney Margin | 24.8 |
| (OD06159) | | (OD04450-03) | |
| Colon cancer (OD06297- | 11.7 | Kidney Cancer 8120613 | 1.0 |
| 04) | | | |
| Colon Margin | 39.5 | Kidney Margin 8120614 | 19.9 |
| (OD06297-015) | | | |
| CC Gr.2 ascend colon | 6.3 | Kidney Cancer 9010320 | 6.4 |
| (ODO3921) | | | |
| CC Margin (ODO3921) | 7.2 | Kidney Margin 9010321 | 11.1 |
| Colon cancer metastasis | 3.0 | Kidney Cancer 8120607 | 35.6 |
| (OD06104) | | | |
| Lung Margin (OD06104) | 16.0 | Kidney Margin 8120608 | 10.9 |
| Colon mets to lung | 35.6 | Normal Uterus | 90.8 |
| (OD04451-01) | | | |
| Lung Margin (OD04451- | 53.6 | Uterine Cancer 064011 | 7.6 |
| 02) | | | |
| Normal Prostate | 6.4 | Normal Thyroid | 1.1 |
| Prostate Cancer | 1.8 | Thyroid Cancer | 6.7 |
| OD04410) | | | |
| Prostate Margin | 4.0 | Thyroid Cancer | 11.5 |
| (OD04410) | | A302152 | |
| Normal Ovary | 35.8 | Thyroid Margin | 3.8 |
| | | A302153 | |
| Ovarian cancer | 18.6 | Normal Breast | 61.6 |
| (OD06283-03) | | | |
| Ovarian Margin | 30.4 | Breast Cancer | 7.6 |
| (OD06283-07) | | | |
| Ovarian Cancer | 11.9 | Breast Cancer | 40.3 |
| Ovarian cancer | 4.4 | Breast Cancer | 27.9 |
| (OD06145) | | (OD04590-01) | |
| Ovarian Margin | 22.1 | Breast Cancer Mets | 31.4 |
| (OD06145) | | (OD04590-03) | |
| Ovarian cancer | 14.0 | Breast Cancer | 26.4 |
| (OD06455-03) | | Metastasis | |
| Ovarian Margin | 15.3 | Breast Cancer | 33.4 |
| (OD06455-07) | | | |
| Normal Lung | 19.3 | Breast Cancer 9100266 | 15.9 |
| Invasive poor diff. lung | 14.1 | Breast Margin 9100265 | 30.4 |
| adeno (ODO4945-01 | | | |
| Lung Margin | 33.9 | Breast Cancer A209073 | 9.2 |
| (ODO4945-03) | | | |
| Lung Malignant Cancer | 18.6 | Breast Margin | 28.5 |
| (OD03126) | | A2090734 | |
| Lung Margin (OD03126) | 6.5 | Breast cancer | 48.0 |
| | | (OD06083) | |
| Lung Cancer | 21.5 | Breast cancer node | 35.6 |
| (OD05014A) | | metastasis (OD06083) | |
| Lung Margin | 44.8 | Normal Liver | 15.8 |
| (OD05014B) | | | |
| Lung cancer (OD06081) | 8.4 | Liver Cancer 1026 | 16.2 |
| Lung Margin (OD06081) | 19.8 | Liver Cancer 1025 | 33.2 |
| Lung Cancer (OD04237- | 4.3 | Liver Cancer 6004-T | 19.6 |
| 01) | | | |
| Lung Margin (OD04237- | 58.6 | Liver Tissue 6004-N | 4.9 |
| 02) | | | |
| Ocular Mel Met to Liver | 9.7 | Liver Cancer 6005-T | 44.8 |
| (OD04310) | | | |
| Liver Margin | 9.0 | Liver Tissue 6005-N | 64.6 |
| (ODO4310) | | | |
| Melanoma Metastasis | 3.4 | Liver Cancer | 29.5 |
| Lung Margin (OD04321) | 36.6 | Normal Bladder | 15.1 |
| Normal Kidney | 5.3 | Bladder Cancer | 15.7 |
| Kidney Ca, Nuclear | 46.7 | Bladder Cancer | 21.2 |
| grade 2 (OD04338) | | | |
| Kidney Margin | 4.6 | Normal Stomach | 54.3 |
| (OD04338) | | | |
| Kidney Ca Nuclear grade | 26.6 | Gastric Cancer 9060397 | 6.4 |
| 1/2 (OD04339) | | | |
| Kidney Margin | 15.5 | Stomach Margin | 22.4 |
| (OD04339) | | 9060396 | |
| Kidney Ca, Clear cell | 17.0 | Gastric Cancer 9060395 | 19.3 |
| type (OD04340) | | | |
| Kidney Margin | 20.3 | Stomach Margin | 35.1 |
| (OD04340) | | 9060394 | |
| Kidney Ca, Nuclear | 15.3 | Gastric Cancer 064005 | 11.4 |
| grade 3 (OD04348) | | | |

TABLE LD
Panel 4D

| | Rel. Exp. (%) | | Rel. Exp. (%) |
|---|---|---|---|
| | Ag2546, Run | | Ag2546, Run |
| Tissue Name | 164321138 | Tissue Name | 164321138 |
| Secondary Th1 act | 34.2 | HUVEC IL-1beta | 26.6 |
| Secondary Th2 act | 33.7 | HUVEC IFN gamma | 45.7 |
| Secondary Tr1 act | 34.4 | HUVEC TNF alpha + IFN | 58.6 |
| | | gamma | |
| Secondary Th1 rest | 15.7 | HUVEC TNF alpha + IL4 | 58.6 |
| Secondary Th2 rest | 28.5 | HUVEC IL-11 | 28.5 |
| Secondary Tr1 rest | 26.8 | Lung Microvascular EC | 60.7 |
| | | none | |
| Primary Th1 act | 30.8 | Lung Microvascular EC | 64.6 |
| | | TNF alpha + IL-1beta | |
| Primary Th2 act | 36.9 | Microvascular Dermal EC | 66.9 |
| | | none | |
| Primary Tr1 act | 40.9 | Microsvasular Dermal EC | 61.1 |
| | | TNF alpha + IL-1beta | |
| Primary Th1 rest | 75.8 | Bronchial epithelium | 66.9 |
| | | TNF alpha + IL-1beta | |
| Primary Th2 rest | 60.3 | Small airway epithelium | 32.8 |
| | | none | |
| Primary Tr1 rest | 49.7 | Small airway epithelium | 95.3 |
| | | TNF alpha + IL-1beta | |
| CD45RA CD4 | 42.0 | Coronery artery SMC rest | 61.1 |
| lymphocyte act | | | |
| CD45RO CD4 | 39.0 | Coronery artery SMC | 36.6 |
| lymphocyte act | | TNF alpha + IL-1beta | |
| CD8 lymphocyte act | 35.6 | Astrocytes rest | 66.0 |
| Secondary CD8 | 41.2 | Astrocytes TNF alpha + IL- | 46.0 |
| lymphocyte rest | | 1beta | |
| Secondary CD8 | 26.1 | KU-812 (Basophil) rest | 7.6 |
| lymphocyte act | | | |
| CD4 lymphocyte none | 22.2 | KU-812 (Basophil) | 29.5 |
| | | PMA/ionomycin | |
| 2ry Th1/Th2/Tr1_anti- | 38.4 | CCD1106 (Keratinocytes) | 56.6 |
| CD95 CH11 | | none | |
| LAK cells rest | 30.8 | CCD1106 (Keratinocytes) | 57.0 |
| | | TNF alpha + IL-1beta | |
| LAK cells IL-2 | 43.8 | Liver cirrhosis | 9.8 |
| LAK cells IL-2 + IL-12 | 37.6 | Lupus kidney | 6.5 |
| LAK cells IL-2 + IFN | 41.2 | NCI-H292 none | 17.7 |
| gamma | | | |
| LAK cells IL-2 + IL-18 | 38.2 | NCI-H292 IL-4 | 26.2 |
| LAK cells | 38.4 | NCI-H292 IL-9 | 26.6 |
| PMA/ionomycin | | | |
| NK Cells IL-2 rest | 30.4 | NCI-H292 IL-13 | 19.6 |
| Two Way MLR 3 day | 27.2 | NCI-H292 IFN gamma | 19.2 |
| Two Way MLR 5 day | 21.9 | HPAEC none | 46.0 |
| Two Way MLR 7 day | 26.2 | HPAEC TNF alpha + | 54.0 |
| | | IL-1beta | |
| PBMC rest | 27.5 | Lung fibroblast none | 46.7 |
| PBMC PWM | 71.2 | Lung fibroblast TNF alpha + | 28.7 |
| | | IL-1beta | |
| PBMC PHA-L | 44.8 | Lung fibroblast IL-4 | 77.4 |
| Ramos (B cell) none | 23.7 | Lung fibroblast IL-9 | 68.3 |
| Ramos (B cell) | 59.9 | Lung fibroblast IL-13 | 62.0 |
| ionomycin | | | |
| B lymphocytes PWM | 66.9 | Lung fibroblast IFN | 81.2 |
| | | gamma | |
| B lymphocytes CD40L | 47.6 | Dermal fibroblast | 77.4 |
| and IL-4 | | CCD1070 rest | |
| EOL-1 dbcAMP | 20.3 | Dermal fibroblast | 100.0 |
| | | CCD1070 TNF alpha | |
| EOL-1 dbcAMP | 25.9 | Dermal fibroblast | 54.0 |
| PMA/ionomycin | | CCD1070 IL-1beta | |
| Dendritic cells none | 23.0 | Dermal fibroblast IFN | 18.2 |
| | | gamma | |
| Dendritic cells LPS | 25.5 | Dermal fibroblast IL-4 | 27.2 |
| Dendritic cells anti- | 26.2 | IBD Colitis 2 | 2.4 |
| CD40 | | | |
| Monocytes rest | 31.6 | IBD Crohn's | 2.4 |
| Monocytes LPS | 17.7 | Colon | 22.4 |
| Macrophages rest | 33.0 | Lung | 45.4 |
| Macrophages LPS | 20.9 | Thymus | 28.5 |
| HUVEC none | 49.0 | Kidney | 39.5 |
| HUVEC starved | 85.9 | | |

Panel 1.3D Summary: Ag2546

The CG50311-01 gene is expressed at moderate levels in all cell lines and tissues in this panel, with highest expression in a glioblastoma/astrocytoma cell line (CT=25.3). There is slightly increased expression in renal and brain cancer cell lines compared to normal tissues suggesting a possible role in these cancers.

This gene is also expressed at moderate levels in all endocrine (metabolic)-related regions examined. Therefore, therapeutic modulation of this gene or its protein product may be of use in the treatment of any endocrine (metabolic)-related disease where neuronal feedback is critical.

This gene encodes a myosin homolog that is expressed at moderate levels in all brain regions examined. Nonmuscle myosin is believed to be involved in the migration of neural growth cones. Therefore, therapeutic modulation of this gene or its protein product may be of use in the treatment of any CNS disease that involves neuronal death/neurodegeneration (Alzheimer's, Parkinson's, Huntington's diseases, stroke, brain or spinal cord trauma) and may also aid in compensatory synaptogenesis.

References:

Kira M, Tanaka J, Sobue K. Caldesmon and low Mr isoform of tropomyosin are localized in neuronal growth cones. J Neurosci Res Feb. 15, 1995;40(3):294-305.

Neuronal growth cones move actively, accompanying changes in intracellular Ca2+ concentration. The movement of growth cones may partly depend on the actomyosin system, considering the presence of actin and myosin II. Yet, Ca(2+)-sensitive regulatory proteins for the actomyosin system have not been identified in growth cones. In the present study, caldesmon, an inhibitory protein on actin-myosin interaction, was detected in the growth cone fraction isolated from embryonic rat brain, using immunoblotting with the antibody to chicken gizzard caldesmon. Morphological evidence of caldesmon in growth cones of cultured rat neurons was obtained using the indirect immunofluorescence method. Since inhibition of caldesmon on actin-myosin interaction can be overcome by calmodulin and Ca2+, caldesmon may be involved in the Ca(2+)-dependent regulation in growth cone motility. Tropomyosin is another member of the actomyosin system whose function may be regulated by caldesmon in smooth and nonmuscle cells. A low Mr isoform of tropomyosin was distributed in the growth cone fraction. Using specific antibodies against tropomyosin isoforms, we further clarified morphologically that the low Mr isoform was localized in growth cones, but not the high Mr isoform. High Mr isoforms of tropomyosin were present in nonneuronal cells. Actin filaments in growth cones may be unstable, since low Mr tropomyosin binds to actin filaments with a lower affinity than high Mr isoforms. The instability of actin filaments may be suitable for the rapid movement and shape changes of growth cones.

Panel 2.2 Summary: Ag2546

The CG50311-01 gene gene is expressed at moderate levels in all the samples on this panel with slightly higher expression in normal lung, breast and stomach tissue compared to the adjacent tumor tissue. Hence, expression of this gene might be used as a marker to identify normal tissue from cancerous tissue in these organs.

Panel 4D Summary: Ag2546

The CG50311-01 gene is expressed at high levels (CTs=24.9-27.4) in a wide range of cell types with significant importance in innate and specific immunity and also other cell types associated with inflammatory diseases. The highest expression of this transcript is found in dermal and lung fibroblasts treated with cytokines, and in small airway epithelium and HUVEC. Therefore, inhibition of the function of the protein encoded by this gene throught the application of a small molecule drug may reduce or eliminate the symptoms associated with T cell, B cell, endothelial and fibroblast activity such as those found in chronic obstructive pulmonary disease, asthma, emphysema, psoriasis, inflammatory bowel disease, rheumatoid arthritis, osteoarthritis and lupus erythematosus.

NOV12a (CG50323-01: Pancreatitis-Associated Protein)

Expression of gene CG50323-01 was assessed using the primer-probe set Ag3760, described in Table IA.

TABLE IA

Probe Name Ag3760

| | | | |
|---|---|---|---|
| | | | Start |
| Primers | Sequences | Length | Position |

| | | | | |
|---|---|---|---|---|
| Forward | 5′-caattgcctccagtatttgaac-3′ | (SEQ ID NO:184) | 22 | 506 |
| Probe | TET-5′-ttgcagacatagggtaacctcacatt-3′-TAMRA | (SEQ ID NO:185) | 26 | 480 |
| Reverse | 5′-agcatttctgaggtggaaaga-3′ | (SEQ ID NO:186) | 21 | 449 |

CNS_Neurodegeneration_v1.0 Summary: Ag3760

Expression of the CG50323-01 gene is low/undetectable in all samples on this panel (CT>35).

General_screening_panel_v1.4 Summary: Ag3760

Expression of the CG50323-01 gene is low/undetectable in all samples on this panel (CT>35).

Panel 4.1D Summary: Ag3760

Expression of the CG50323-01 gene is low/undetectable in all samples on this panel (CT>35).

### Example 2

Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table M1 shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus.

TABLE M1

| NOVX | | |
|---|---|---|
| Clone | Forward Primer | Reverse Primer |
| NOV1c- | GGATCCAGAATTCTGCAAAATCTTACGACTTTGG | CGGCCGATAGCAGAAGACATCCCACATTTCACTCTTG |
| NOV1j | (SEQ ID NO:187) | (SEQ ID NO:188) |
| NOV3b | TGCGCGCTCGTCGTCCTC | GGAGGCCACAGGAGCAGGATCA |
| | (SEQ ID NO:189) | (SEQ ID NO:190) |
| NOV5b | AGATCTCTGGGCGCAACGGTCATCTGTAACAAGATCC | CTCGAGCTTGCACGTGTACATCTCCGTGCGCTCG |
| | (SEQ ID NO:191) | (SEQ ID NO:192) |
| NOV6b | GGATCCAGCCCTGGCCAGGCCGTGTGCAACTTCG | CTCGAGTGTGTTCCCCGGGCTGGGGGCAGGCTGC |
| | (SEQ ID NO:193) | (SEQ ID NO:194) |
| NOV7b | ATGTCTGTGGCCATGGTAGAGTCAGG | ATCATGAACCTCAACTCCTCAGGAACC |
| and | (SEQ ID NO:195) | (SEQ ID NO:196) |
| NOV7c | | |
| NOV8 | CAAGAGCAGGTTTGAGATGTTCTC | CCAAGGTTGACCACCTCCAT |
| | (SEQ ID NO:197) | (SEQ ID NO:198) |
| NOV10b | ATCTACGGAGTCCCTTTGGCCACATAA | TCCAAATGTCAGAATATCGAGGTTCCC |
| | (SEQ ID NO:199) | (SEQ ID NO:200) |
| NOV11 | CCGCCTGTGTTCCATGGCTT | GTCATTCTGCTGCCGGTTGGTAG |
| | (SEQ ID NO:201) | (SEQ ID NO:202) |
| NOV12a | CCATGGCCCTGCCAAGTGTATCTT | TTACAATTGCCTCCAGTATTTGAACTTGCA |
| | (SEQ ID NO:203) | (SEQ ID NO:204) |
| NOV12b | AGCTTGAAGAACCCCAGAGGGAACTGCCCTCTGC | CTCGAGCAATTGCCTCCAGTATTTGAACTTGC |
| and | (SEQ ID NO:205) | (SEQ ID NO:206) |
| NOV12c | | |

Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table M2 shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE M2

| NOVX Clone | Bacterial Clone (Physical clone) |
|---|---|
| NOV3a | 124906::133267070.698458.L7 |
| NOV8 | SC87760822_A.698299.L11 |
| NOV10b | 124893::CG50307-01.698453.H17 |

Real Time Quantitative PCR

Relative expression levels of the mRNA of the invention across a panel of 92 human samples was determined by real-time quantitative PCR analysis. These samples represent multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. Table M3 shows the primers/probe used for this reaction. The primers and probe were designed to specifically identify the gene of the invention irresepective of the presence of related human genes like splice forms, homologs and paralogs.

TABLE M3

| NOVX | | | |
|---|---|---|---|
| Clone | Forward Primer | Reverse Primer | Probe |
| NOV8 | GCACTTGAAGAGCTGTCATAGC | TACCCTGAGTCTCTTGATTCCA | TET-5′-CTCTATGACTGCCAGCAAATCACACG-3′-TAMRA |
| | (SEQ ID NO:207) | (SEQ ID NO:208) | (SEQ ID NO:209) |

### Example 3

SNP Analysis of NOVX Clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations. Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of Novel SNP Confirmation:

SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxiderivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

Results

Variants are reported individually but any combination of all or a select subset of variants are also included as contemplated NOVX embodiments of the invention.

NOV1a SNP Data

The DNA and protein sequences for the novel single nucleotide polymorphic variants of the Stablin-like gene of NOV1a are reported in Table N1. Variants are reported individually but any combination of all or a select subset of variants are also included. In summary, there are 5 variants reported.

TABLE N1
cSNP and Coding Variants for NOV1a

| | Base | | | |
|---|---|---|---|---|
| | Position of | | | |
| Variant | cSNP | Wild Type | Variant | Amino Acid Change |
| 13376228 | 4185 | T | C | silent (no change) |
| 13376229 | 4524 | T | C | silent |
| 13376230 | 4654 | G | A | Gly → Ser at aa 1552 |
| 13376231 | 4671 | A | G | silent |
| 13376232 | 4820 | T | C | Leu → Pro at aa 1607 |

NOV2a SNP Data

The DNA and protein sequences for the novel single nucleotide polymorphic variants of the Polydom-like gene of NOV2a are reported in Table N2. Variants are reported individually but any combination of all or a select subset of variants are also included. In summary, there are 10 variants reported.

TABLE N2
cSNP and Coding Variants for NOV2a

| | Base | | | |
|---|---|---|---|---|
| | Position of | | | |
| Variant | cSNP | Wild Type | Variant | Amino Acid Change |
| 13374700 | 717 | A | G | Glu → Gly at aa 214 |
| 13374701 | 2303 | A | G | Asn → Asp at aa 743 |
| 13374256 | 7348 | T | C | silent |
| 13376233 | 7370 | C | T | Pro → ser at aa 2432 |
| 13376234 | 8665 | G | A | silent |
| 13376235 | 8827 | C | T | silent |
| 13376236 | 9018 | A | G | His → Arg at aa 2981 |
| 13376237 | 9551 | A | G | Thr → Ala at aa 3159 |
| 13376238 | 9790 | T | G | silent |
| 13376239 | 10025 | G | T | Gly → End at aa |
| | | | | 3317 |

NOV3a SNP Data

The DNA and protein sequences for the novel single nucleotide polymorphic variants of the Transmembrane-like gene of NOV3a are reported in Table N3. Variants are reported individually but any combination of all or a select subset of variants are also included. In summary, there are 4 variants reported.

TABLE N3
cSNP and Coding Variants for NOV3a

| | Base | | | |
|---|---|---|---|---|
| | Position of | | | |
| Variant | cSNP | Wild Type | Variant | Amino Acid Change |
| 13376243 | 145 | A | G | Ile → Val at aa 49 |
| 13376242 | 336 | G | A | Trp → End at aa 112 |
| 13376241 | 494 | G | A | Gly → Asp at aa 165 |
| 13376240 | 495 | C | T | silent |

NOV4 SNP Data

The DNA and protein sequences for the novel single nucleotide polymorphic variants of the Serine Protease-like gene of NOV4 are reported in Table N4. Variants are reported individually but any combination of all or a select subset of variants are also included. In summary, there are 3 variants reported.

TABLE N4
cSNP and Coding Variants for NOV4

| | Base Position of | | | |
|---|---|---|---|---|
| Variant | cSNP | Wild Type | Variant | Amino Acid Change |
| 13376246 | 122 | G | A | Val → Ile at aa 37 |
| 13376245 | 258 | A | G | His → Arg at aa 82 |
| 13376244 | 296 | C | T | Arg → Cys at aa 95 |

NOV5a SNP Data

The DNA and protein sequences for the novel single nucleotide polymorphic variants of the Wnt7a-like gene of NOV5a are reported in Table N5. Variants are reported individually but any combination of all or a select subset of variants are also included. In summary, there are 2 variants reported.

TABLE N5
cSNP and Coding Variants for NOV5a

| | Base Position | Wild | | Amino Acid |
|---|---|---|---|---|
| Variant | of cSNP | Type | Variant | Change |
| 13376247 | 315 | G | A | silent |
| 13376248 | 459 | T | C | silent |

NOV6a SNP Data

The DNA and protein sequences for the novel single nucleotide polymorphic variants of the Apical Endosomal Glycoprotein-like gene of NOV6a are reported in Table N6. Variants are reported individually but any combination of all or a select subset of variants are also included. In summary, there is 1 variant reported.

TABLE N6
cSNP and Coding Variants for NOV6a

| | Base Position | Wild | | Amino Acid |
|---|---|---|---|---|
| Variant | of cSNP | Type | Variant | Change |
| 13376249 | 3477 | C | T | Pro → Ser at aa 1147 |

NOV7a SNP Data

The DNA and protein sequences for the novel single nucleotide polymorphic variants of the ADAM13-like gene of NOV7a are reported in Table N7. Variants are reported individually but any combination of all or a select subset of variants are also included. In summary, there are 2 variants reported.

TABLE N7
cSNP and Coding Variants for NOV7a

| | Base Position | Wild | | Amino Acid |
|---|---|---|---|---|
| Variant | of cSNP | Type | Variant | Change |
| 13374267 | 2130 | G | A | Val → Ile at aa 710 |
| 13374266 | 2153 | G | C | silent |

NOV8 SNP Data

The DNA and protein sequences for the novel single nucleotide polymorphic variants of the Leucine Rich Containing F-Box Protein-like gene of NOV8 are reported in Table N8. Variants are reported individually but any combination of all or a select subset of variants are also included. In summary, there are 2 variants reported.

TABLE N8
cSNP and Coding Variants for NOV8a

| | Base Position | Wild | | Amino Acid |
|---|---|---|---|---|
| Variant | of cSNP | Type | Variant | Change |
| 13373958 | 366 | T | C | Ile → Thr at aa 117 |
| 13373959 | 452 | C | T | Pro → Ser at aa 146 |

NOV10a SNP Data

The DNA and protein sequences for the novel single nucleotide polymorphic variants of the Steroid dehydrogenase-like gene of NOV10a are reported in Table N9. Variants are reported individually but any combination of all or a select subset of variants are also included. In summary, there are 2 variants reported.

TABLE N9
cSNP and Coding Variants for NOV10a

| | Base Position | Wild | | Amino Acid |
|---|---|---|---|---|
| Variant | of cSNP | Type | Variant | Change |
| 13375812 | 465 | A | G | Ile → Val at aa95 |
| 13375811 | 1162 | C | G | Ser → Cys at aa327 |

NOV11 SNP Data

The DNA and protein sequences for the novel single nucleotide polymorphic variants of the Myosin Heavy Chain-like gene of NOV11 are reported in Table N10. Variants are reported individually but any combination of all or a select subset of variants are also included. In summary, there are 4 variants reported.

TABLE N10
cSNP and Coding Variants for NOV11

| | Base Position | Wild | | Amino Acid |
|---|---|---|---|---|
| Variant | of cSNP | Type | Variant | Change |
| 13374341 | 5008 | G | T | silent |
| 13374342 | 5012 | A | G | Ile → Val at aa1625 |
| 13376300 | 6808 | C | T | silent |
| 13376299 | 7323 | C | T | silent |

NOV12a SNP Data

The DNA and protein sequences for the novel single nucleotide polymorphic variants of the Pacreatitis Associated Protein-like gene of NOV12a are reported in Table N11. Variants are reported individually but any combination of all or a select subset of variants are also included. In summary, there are 8 variants

TABLE N11
cSNP and Coding Variants for NOV12a

| | Base Position | Wild | | Amino Acid |
|---|---|---|---|---|
| Variant | of cSNP | Type | Variant | Change |
| 13373957 | 68 | T | C | silent |
| 13373956 | 127 | C | T | Ala → Val at aa 42 |
| 13373955 | 178 | A | G | Asp → Gly at aa 59 |
| 13373954 | 182 | A | G | silent |
| 13373953 | 227 | G | A | silent |
| 13373952 | 314 | C | T | silent |
| 13373951 | 341 | A | G | silent |
| 13373950 | 441 | A | G | Arg → Gly at aa 147 |

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 36, and/or 38;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 36, and/or 38, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, 36, and/or 38; and
(d) a variant of an amino acid sequence selected from the group consisting of SEQ ID NOS: 2, 4, 6, 8, 10, 12, 14, 16, 18, 20, 22, 24, 26, 28, 30, 32, 34, 36, and/or 38 wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS: 2, 4, 6, 8, 10, 12, 14, 16, 18, 20, 22, 24, 26, 28, 30, 32, 34, 36, and/or 38.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, and/or 37.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 36, and/or 38;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 36, and/or 38, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, 36, and/or 38,
(d) a variant of an amino acid sequence selected from the group consisting of SEQ ID NOS: 2, 4, 6, 8, 10, 12, 14, 16, 18, 20, 22, 24, 26, 28, 30, 32, 34, 36, and/or 38, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28, 30, 32, 34, 36, and/or 38, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, and/or 37.

9. The nucleic acid molecule of claim 5, wherein said nucleic acid molecule comprises a nucleotide sequence selected from the group consisting of
(a) a nucleotide sequence selected from the group consisting of SEQ ID NOS: 1, 3, 5, 7, 9, 11, 13, 15, 17, 19, 21, 23, 25, 27, 29, 31, 33, 35, and/or 37;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, 31, 33, 35, and/or 37, provided that no more than 20% of the nucleotides differ from said nucleotide sequence;
(c) a nucleic acid fragment of (a); and
(d) a nucleic acid fragment of (a).

10. The nucleic acid molecule of claim 5, wherein said nucleic acid molecule hybridizes under stringent conditions to a nucleotide sequence chosen from the group consisting of SEQ ID NOS: 1, 3, 5, 7, 9, 11, 13, 15, 17, 19, 21, 23, 25, 27, 29, 31, 33, 35, and/or 37, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28, 30, 32, 34, 36, and/or 38, or a biologically active fragment thereof.

41. A method of treating a pathological state in a mammal, the method comprising administering to the mammal the antibody of claim 15 in an amount sufficient to alleviate the pathological state.

42. An isolated nucleic acid molecule comprising SEQ ID NO: 210, except that the nucleotide at position 887 is a nucleotide other than thymine, or the nucleotide at position 1144 is a nucleotide other than adenosine.

43. The nucleic acid molecule of claim 42, wherein the nucleotide at position 887 is other than thymine.

44. The nucleic acid molecule of claim 43, wherein the nucleotide at position 887 is cytosine.

45. An isolated nucleic acid molecule comprising SEQ ID NO: 210, except that the nucleotide at position 1034 is a nucleotide other than cytosine, or the nucleotide at position 1244 is a nucleotide other than thymine.

46. The nucleic acid molecule of claim 45, wherein the nucleotide at position 1034 is other than cytosine.

47. The nucleic acid molecule of claim 46, wherein the nucleotide at position 1034 is thymine.

48. An isolated nucleic acid molecule comprising SEQ ID NO: 210, except that the nucleotide at position 1223 is a nucleotide other than cytosine, or the nucleotide at position 1416 is a nucleotide other than adenine, or the nucleotide at position 1629 is other than thymine.

49. The nucleic acid molecule of claim 48, wherein the nucleotide at position 1223 is other than cytosine.

50. The nucleic acid molecule of claim 49, wherein the nucleotide at position 1223 is thymine.

51. An isolated nucleic acid molecule comprising SEQ ID NO: 210, except that the nucleotide at position 832 is a nucleotide other than adenine, or the nucleotide at position 2003 is a nucleotide other than thymine.

52. The nucleic acid molecule of claim 51, wherein the nucleotide at position 832 is other than adenine.

53. The nucleic acid molecule of claim 52, wherein the nucleotide at position 832 is guanine.

54. A polypeptide comprising SEQ ID NO: 211, except that the amino acid at position 325 is other than a glutamine.

55. The polypeptide of claim 54, wherein the amino acid at position 325 is leucine.

56. A polypeptide comprising SEQ ID NO: 211, except that the amino acid at position 416 is other than asparagine, or the amino acid at position 487 is other than cysteine.

57. The polypeptide of claim 56, wherein the amino acid at position 416 is tyrosine.

58. A polypeptide comprising SEQ ID NO: 211, except that the amino acid at position 221 is other than tyrosine.

59. The polypeptide of claim 58 wherein the amino acid at position 221 is cysteine.
